(19) United States
(12) Patent Application Publication
(10) Pub. No.: US 2024/0228996 A1
(43) Pub. Date: Jul. 11, 2024

(54) POLYPEPTIDES HAVING ALPHA-AMYLASE ACTIVITY AND POLYNUCLEOTIDES ENCODING SAME

(71) Applicant: Novozymes A/S, Bagsvaerd (DK)

(72) Inventors: Sara Maria Landvik, Vedbaek (DK); Sarah Schultheis Elliott, Raleigh, NC (US); Marie Thryøse Kruse, Vaerloese (DK); Tine Hoff, Vaerloese (DK); Ming Li, Holte (DK); Tianqi Sun, Beijing (CN)

(73) Assignee: Novozymes A/S, Bagsvaerd (DK)

(21) Appl. No.: 17/797,381

(22) PCT Filed: Feb. 9, 2021
(86) PCT No.: PCT/US2021/017210

§ 371 Date: Aug. 3, 2022

(30) Foreign Application Priority Data
Feb. 10, 2020 (WO) PCT/CN2020/074600
Sep. 4, 2020 (WO) PCT/CN2020/113453

(51) Int. Cl. C12N 9/30; C12N 1/16; C12P 7/06

(52) U.S. Cl. CPC C12N 9/242; C12N 1/165; C12P 7/06; C12Y 302/01001

(57) ABSTRACT

The present invention relates to polypeptides having alpha-amylase activity, alpha-amylase catalytic domains, and starch binding modules, and polynucleotides encoding the polypeptides, alpha-amylase catalytic domains, and starch binding modules, and to nucleic acid constructs, vectors, and host cells comprising the polynucleotides as well as methods of producing and using the polypeptides, alpha-amylase catalytic domains, and starch binding modules. The present invention relates to processes for producing fermentation products from starch-containing material. The invention also relates to an enzyme blend or composition, or a recombinant host cell or fermenting organism suitable for use in a process of the invention.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to polypeptides having alpha-amylase activity, alpha-amylase catalytic domains, and starch binding modules, and polynucleotides encoding the polypeptides, alpha-amylase catalytic domains, and starch binding modules, and to nucleic acid constructs, vectors, and host cells comprising the polynucleotides as well as methods of producing and using the polypeptides, alpha-amylase catalytic domains, and starch binding modules. The present invention relates to processes for producing fermentation products from starch-containing material. The invention also relates to an enzyme blend or composition, or a recombinant host cell or fermenting organism suitable for use in a process of the invention.

### Description of the Related Art

Production of fermentation products, such as ethanol, from starch-containing material is well-known in the art. The most commonly used process, often referred to as a “conventional process”, and includes liquefying gelatinized starch at high temperature using typically a bacterial alpha-amylase, followed by simultaneous saccharification and fermentation carried out in the presence of a glucoamylase and a fermentation organism. Alpha-amylases are used commercially for a variety of purposes such as in the initial stages of starch processing (e.g., liquefaction); in wet milling processes; and in alcohol production from carbohydrate sources. They are also used as cleaning agents or adjuncts in detergent matrices; in the textile industry for starch desizing; in baking applications; in the beverage industry; in oil fields in drilling processes; in recycling processes, e.g., for de-inking paper; and in animal feed.

Alpha-amylases (alpha-1,4-glucan-4-glucanohydrolases, E.C. 3.2.1.1) constitute a group of enzymes, which catalyze hydrolysis of starch and other linear and branched 1,4-glucosidic oligo- and polysaccharides.

SEQ ID NO: 10 of WO2017/029238 is 91% identical to the alpha-amylase shown in SEQ ID NO: 2.

SEQ ID NO: 9 of WO2017/029238 and SEQ ID NO: 165 of WO2006069290 and US2014/127753 are each 84.9% identical to the alpha-amylase shown in SEQ ID NO: 5.

SEQ ID NO: 165 of WO2006069290 and US2014/127753 are 92.9% identical to the alpha-amylase shown in SEQ ID NO: 8.

SEQ ID NO: 27 of WO2017112631, WO2017112635, and WO2017112643 is 81.6% identical to the alpha-amylase shown in SEQ ID NO: 11

SEQ ID NO: 10 of WO2011049945, SEQ ID NO: 1 of CN109182304, SEQ ID NO: 28 and SEQ ID NO: 29 of WO2009149283 are each 67.4% identical to the alpha-amylase shown in SEQ ID NO: 14 (P84UXD).

SEQ ID NO: 1 of CN109182304 is 67.6% identical to the alpha-amylase shown in SEQ ID NO: 44.

SEQ ID NO: 6 of WO2017173190 is 66.6% identical to the alpha-amylase shown in SEQ ID NO: 47.

SEQ ID NO: 1 of CN109182304 is 67% identical to the alpha-amylase shown in SEQ ID NO: 50.

SEQ ID NO: 8 of WO2018002360 is 65.8% identical to the alpha-amylase shown in SEQ ID NO: 53.

SEQ ID NO: 32 of WO2009149283 is 69.1% identical to the alpha-amylase shown in SEQ ID NO: 56.

SEQ ID NO: 32 of WO2009149283 is 69.1% identical to the alpha-amylase shown in SEQ ID NO: 56.

SEQ ID NO: 32 of WO2009149283 is 69.1% identical to the alpha-amylase shown in SEQ ID NO: 59.

Despite significant improvement of fermentation product production processes over the past decade a significant amount of residual starch material is not converted into the desired fermentation product, such as ethanol. At least some of the unconverted residual starch material, e.g., sugars and dextrins, is in the form of non-fermentable Maillard products.

Therefore, there is still a desire and need for providing processes for producing fermentation products, such as ethanol, from starch-containing material that can provide a higher fermentation product yield, or other advantages, compared to a conventional process.

### SUMMARY OF THE INVENTION

The present invention provides isolated or purified polypeptides having alpha-amylase activity and polynucleotides encoding the polypeptides.

Accordingly, the present invention relates to isolated or purified polypeptides having alpha-amylase activity selected from the group consisting of:

(i)
(a) a polypeptide having at least 92%, at least 93%, at least 94%, at least 95%, at least 96%, at least 97%, at least 98%, at least 99%, or 100% sequence identity to SEQ ID NO: 2;
(b) a polypeptide having at least 92%, at least 93%, at least 94%, at least 95%, at least 96%, at least 97%, at least 98%, at least 99%, or 100% sequence identity to the sequence of SEQ ID NO: 3;
(c) a polypeptide having at least 92%, at least 93%, at least 94%, at least 95%, at least 96%, at least 97%, at least 98%, at least 99%, or 100% sequence identity to a mature polypeptide of SEQ ID NO: 2;
(d) a polypeptide encoded by a polynucleotide that hybridizes under medium, medium-high, or high stringency conditions with the full-length complement of the mature polypeptide coding sequence of SEQ ID NO: 1;
(e) a polypeptide encoded by a polynucleotide having at least 92%, at least 93%, at least 94%, at least 95%, at least 96%, at least 97%, at least 98%, at least 99%, or 100% sequence identity to the mature polypeptide coding sequence of SEQ ID NO: 1;
(f) a fragment of the polypeptide of (a), (b), (c), (d), or (e) that has alpha-amylase activity;
   (ii)
(a) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 5;
(b) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 6;
(c) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 5;
(d) a polypeptide encoded by a polynucleotide that hybridizes under medium, medium-high, or high stringency conditions with the full-length complement of the mature polypeptide coding sequence of SEQ ID NO: 4, or the cDNA sequence thereof;
(e) a polypeptide encoded by a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 4, or the cDNA sequence thereof;
(f) a fragment of the polypeptide of (a), (b), (c), (d), or (e) that has alpha-amylase activity;
   (iii)
(a) a polypeptide having at least 93%, at least 94%, at least 95%, at least 96%, at least 97%, at least 98%, at least 99%, or 100% sequence identity to SEQ ID NO: 8;
(b) a polypeptide having at least 93%, at least 94%, at least 95%, at least 96%, at least 97%, at least 98%, at least 99%, or 100% sequence identity to the sequence of SEQ ID NO: 9;
(c) a polypeptide having at least 93%, at least 94%, at least 95%, at least 96%, at least 97%, at least 98%, at least 99%, or 100% sequence identity to a mature polypeptide of SEQ ID NO: 8;
(d) a polypeptide encoded by a polynucleotide that hybridizes under medium, medium-high, or high stringency conditions with the full-length complement of the mature polypeptide coding sequence of SEQ ID NO: 7, or the cDNA sequence thereof;
(e) a polypeptide encoded by a polynucleotide having at least 93%, at least 94%, at least 95%, at least 96%, at least 97%, at least 98%, at least 99%, or 100% sequence identity to the mature polypeptide coding sequence of SEQ ID NO: 7, or the cDNA sequence thereof;
(f) a fragment of the polypeptide of (a), (b), (c), (d), or (e) that has alpha-amylase activity;
   (iv)
(a) a polypeptide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
(b) a polypeptide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 12;
(c) a polypeptide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 11;
(d) a polypeptide encoded by a polynucleotide that hybridizes under medium, medium-high, or high stringency conditions with the full-length complement of the mature polypeptide coding sequence of SEQ ID NO: 10;
(e) a polypeptide encoded by a polynucleotide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 10;
(f) a fragment of the polypeptide of (a), (b), (c), (d), or (e) that has alpha-amylase activity;
   (v)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 15;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 14;
(d) a polypeptide encoded by a polynucleotide that hybridizes under medium, medium-high, or high stringency conditions with the full-length complement of the mature polypeptide coding sequence of SEQ ID NO: 13;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 13;
(f) a fragment of the polypeptide of (a), (b), (c), (d), or (e) that has alpha-amylase activity;
   (vi)
(a) a polypeptide comprising, consisting essentially of, or consisting of SEQ ID NO: 41, a mature polypeptide of SEQ ID NO: 41, or SEQ ID NO: 42;
(b) a polypeptide encoded by a polynucleotide that hybridizes under medium, medium-high, or high stringency conditions with the full-length complement of the mature polypeptide coding sequence of SEQ ID NO: 40;
(c) a polypeptide encoded by a polynucleotide having the mature polypeptide coding sequence of SEQ ID NO: 40;
(d) a fragment of the polypeptide of (a), (b), (c) or (d) that has alpha-amylase activity;
   (vii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 45;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 44;
(d) a polypeptide encoded by a polynucleotide that hybridizes under medium, medium-high, or high stringency conditions with the full-length complement of the mature polypeptide coding sequence of SEQ ID NO: 43;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 43; or
(f) a fragment of the polypeptide of (a), (b), (c), (d), or (e) that has alpha-amylase activity;
   (viii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 48;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 47;
(d) a polypeptide encoded by a polynucleotide that hybridizes under medium, medium-high, or high stringency conditions with the full-length complement of the mature polypeptide coding sequence of SEQ ID NO: 46;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 46; or
(f) a fragment of the polypeptide of (a), (b), (c), (d), or (e) that has alpha-amylase activity;
   (ix)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to SEQ ID NO: 50;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 51;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 50;
(d) a polypeptide encoded by a polynucleotide that hybridizes under medium, medium-high, or high stringency conditions with the full-length complement of the mature polypeptide coding sequence of SEQ ID NO: 49;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 49; or
(f) a fragment of the polypeptide of (a), (b), (c), (d), or (e) that has alpha-amylase activity; and
   (x)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 53;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to the sequence of SEQ ID NO: 54;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 53;
(d) a polypeptide encoded by a polynucleotide that hybridizes under medium, medium-high, or high stringency conditions with the full-length complement of the mature polypeptide coding sequence of SEQ ID NO: 52;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 52; or
(f) a fragment of the polypeptide of (a), (b), (c), (d), or (e) that has alpha-amylase activity.
   (xi)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 56;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 57;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to a mature polypeptide of SEQ ID NO: 56;
(d) a polypeptide encoded by a polynucleotide that hybridizes under medium, medium-high, or high stringency conditions with the full-length complement of the mature polypeptide coding sequence of SEQ ID NO: 55;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 55; or
(f) a fragment of the polypeptide of (a), (b), (c), (d), or (e) that has alpha-amylase activity; and
   (xii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 59;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 60;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 59;
(d) a polypeptide encoded by a polynucleotide that hybridizes under medium, medium-high, or high stringency conditions with the full-length complement of the mature polypeptide coding sequence of SEQ ID NO: 58;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 58; or
(f) a fragment of the polypeptide of (a), (b), (c), (d), or (e) that has alpha-amylase activity.

The present invention also relates to isolated or purified polypeptides comprising a catalytic domain selected from the group consisting of:

(i)
(a) a catalytic domain having at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 18 to 497 of SEQ ID NO: 5;
(b) a catalytic domain encoded by a polynucleotide that hybridizes under medium, medium-high, high, or very high stringency conditions with the full-length complement of nucleotides 52 to 1943 of SEQ ID NO: 4, or the cDNA sequence thereof;
(c) a catalytic domain encoded by a polynucleotide having at least at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 52 to 1943 of SEQ ID NO: 4, or the cDNA sequence thereof; and
(d) a fragment of the catalytic domain of (a), (b), or (c) that has alpha-amylase activity;
   (ii)
(a) a catalytic domain having at least 94%, at least 95%, at least 96%, at least 97%, at least 98%, at least 99%, or 100% sequence identity to amino acids 22 to 495 of SEQ ID NO: 8;
(b) a catalytic domain encoded by a polynucleotide that hybridizes under medium, medium-high, high, or very high stringency conditions with the full-length complement of nucleotides 64 to 1925 of SEQ ID NO: 7, or the cDNA sequence thereof;
(c) a catalytic domain encoded by a polynucleotide having at least at least 94%, at least 95%, at least 96%, at least 97%, at least 98%, at least 99%, or 100% sequence identity to nucleotides 64 to 1925 of SEQ ID NO: 7, or the cDNA sequence thereof; and
(d) a fragment of the catalytic domain of (a), (b), or (c) that has alpha-amylase activity;
   (iii)
(a) a catalytic domain having at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 20 to 496 of SEQ ID NO: 11;
(b) a catalytic domain encoded by a polynucleotide that hybridizes under medium, medium-high, high, or very high stringency conditions with the full-length complement of nucleotides 58 to 1488 of SEQ ID NO: 10;
(c) a catalytic domain encoded by a polynucleotide having at least at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 58 to 1488 of SEQ ID NO: 10; and
(d) a fragment of the catalytic domain of (a), (b), or (c) that has alpha-amylase activity; and
   (iv)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 30 to 469 of SEQ ID NO: 14;
(b) a catalytic domain encoded by a polynucleotide that hybridizes under medium, medium-high, high, or very high stringency conditions with the full-length complement of nucleotides 88 to 1407 of SEQ ID NO: 13;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 88 to 1407 of SEQ ID NO: 13;
(d) a fragment of the catalytic domain of (a), (b), or (c) that has alpha-amylase activity;
   (v)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity sequence identity to amino acids 30 to 469 of SEQ ID NO: 41;
(b) a catalytic domain encoded by a polynucleotide that hybridizes under medium, medium-high, high, or very high stringency conditions with the full-length complement of nucleotides 82 to 1395 of SEQ ID NO: 40;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 82 to 1395 of SEQ ID NO: 40; and
(d) a fragment of the catalytic domain of (a), (b), or (c) that has alpha-amylase activity;
   (vi)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 44;
(b) a catalytic domain encoded by a polynucleotide that hybridizes under medium, medium-high, high, or very high stringency conditions with the full-length complement of nucleotides 133 to 1401 of SEQ ID NO: 43;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 133 to 1401 of SEQ ID NO: 43; and
(d) a fragment of the catalytic domain of (a), (b), or (c) that has alpha-amylase activity;
   (vii)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 47;
(b) a catalytic domain encoded by a polynucleotide that hybridizes under medium, medium-high, high, or very high stringency conditions with the full-length complement of nucleotides 133 to 1401 of SEQ ID NO: 46;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 133 to 1401 of SEQ ID NO: 46; and
(d) a fragment of the catalytic domain of (a), (b), or (c) that has alpha-amylase activity;
   (viii)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 50;
(b) a catalytic domain encoded by a polynucleotide that hybridizes under medium, medium-high, high, or very high stringency conditions with the full-length complement of nucleotides 133 to 1401 of SEQ ID NO: 49;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 133 to 1401 of SEQ ID NO: 49; and
(d) a fragment of the catalytic domain of (a), (b), or (c) that has alpha-amylase activity;
   (ix)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 53;
(b) a catalytic domain encoded by a polynucleotide that hybridizes under medium, medium-high, high, or very high stringency conditions with the full-length complement of nucleotides 133 to 1401 of SEQ ID NO: 52;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 133 to 1401 of SEQ ID NO: 52; and
(d) a fragment of the catalytic domain of (a), (b), or (c) that has alpha-amylase activity;
   (x)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 56;
(b) a catalytic domain encoded by a polynucleotide that hybridizes under medium, medium-high, high, or very high stringency conditions with the full-length complement of nucleotides 133 to 1401 of SEQ ID NO: 55;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 133 to 1401 of SEQ ID NO: 55; and
(d) a fragment of the catalytic domain of (a), (b), or (c) that has alpha-amylase activity; and
   (xi)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 59;
(b) a catalytic domain encoded by a polynucleotide that hybridizes under medium, medium-high, high, or very high stringency conditions with the full-length complement of nucleotides 133 to 1401 of SEQ ID NO: 58;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 133 to 1401 of SEQ ID NO: 58; and
(d) a fragment of the catalytic domain of (a), (b), or (c) that has alpha-amylase activity.

The present invention also relates to isolated or purified polypeptides comprising a starch binding module selected from the group consisting of:

(i)
(a) a starch binding module having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 532 to 632 of SEQ ID NO: 5;
(b) a starch binding module encoded by a polynucleotide that hybridizes under medium, medium-high, high, or very high stringency conditions with the full-length complement of nucleotides 2046 to 2348 of SEQ ID NO: 4, or the cDNA sequence thereof;
(c) a starch binding module encoded by a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 2046 to 2348 of SEQ ID NO: 4, or the cDNA sequence thereof; and
(d) a fragment of the starch binding module of (a), (b), or (c) that has binding activity;
   (ii)
(a) a starch binding module having at least 91%, at least 92%, at least 93%, at least 94%, at least 95%, at least 96%, at least 97%, at least 98%, at least 99%, or 100% sequence identity to amino acids 532 to 632 of SEQ ID NO: 8;
(b) a starch binding module encoded by a polynucleotide that hybridizes under medium, medium-high, high, or very high stringency conditions with the full-length complement of nucleotides 2034 to 2336 of SEQ ID NO: 7, or the cDNA sequence thereof;
(c) a starch binding module encoded by a polynucleotide having sat least 91%, at least 92%, at least 93%, at least 94%, at least 95%, at least 96%, at least 97%, at least 98%, at least 99%, or 100% sequence identity to nucleotides 2034 to 2336 of SEQ ID NO: 7, or the cDNA sequence thereof; and
(d) a fragment of the starch binding module of (a), (b), or (c) that has binding activity; and
   (iii)
(a) a starch binding module having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 508 to 601 of SEQ ID NO: 11;
(b) a starch binding module encoded by a polynucleotide that hybridizes under medium, medium-high, high, or very high stringency conditions with the full-length complement of nucleotides 1504 to 1827 of SEQ ID NO: 10;
(c) a starch binding module encoded by a polynucleotide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1504 to 1827 of SEQ ID NO: 10;
(d) a fragment of the starch binding module of (a), (b), or (c) that has binding activity;
   (iv)
(a) a starch binding module having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity sequence identity to amino acids 554 to 653 of SEQ ID NO: 41;
(b) a starch binding module encoded by a polynucleotide that hybridizes under medium, medium-high, high, or very high stringency conditions with the full-length complement of nucleotides 1660 to 1959 of SEQ ID NO: 40;
(c) a starch binding module encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1660 to 1959 of SEQ ID NO: 40; and
(d) a fragment of the starch binding module of (a), (b), or (c) that has binding activity;
   (v)
(a) a starch binding module having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 478 to 569 of SEQ ID NO: 44;
(b) a starch binding module encoded by a polynucleotide that hybridizes under medium, medium-high, high, or very high stringency conditions with the full-length complement of nucleotides 1432 to 1707 of SEQ ID NO: 43;
(c) a starch binding module encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1432 to 1707 of SEQ ID NO: 43; and
(d) a fragment of the starch binding module of (a), (b), or (c) that has binding activity;
   (vi)
(a) a starch binding module having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 470 to 565 of SEQ ID NO: 47, amino acids 475 to 571 of SEQ ID NO: 50, or amino acids 655 to 750 of SEQ ID NO: 53;
(b) a starch binding module encoded by a polynucleotide that hybridizes under medium, medium-high, high, or very high stringency conditions with the full-length complement of nucleotides 1408 to 1695 of SEQ ID NO: 46, nucleotides 1423 to 1713 of SEQ ID NO: 49, or nucleotides 1963 to 2250 of SEQ ID NO: 52;
(c) a starch binding module encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1408 to 1695 of SEQ ID NO: 46, nucleotides 1423 to 1713 of SEQ ID NO: 49, or nucleotides 1963 to 2250 of SEQ ID NO: 52; and
(d) a fragment of the starch binding module of (a), (b), or (c) that has binding activity;
   (vi)
(a) a starch binding module having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 563 to 654 of SEQ ID NO: 53;
(b) a starch binding module encoded by a polynucleotide that hybridizes under medium, medium-high, high, or very high stringency conditions with the full-length complement of nucleotides 1687 to 1962 of SEQ ID NO: 52;
(c) a starch binding module encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1687 to 1962 of SEQ ID NO: 52; and
(d) a fragment of the starch binding module of (a), (b), or (c) that has binding activity
   (vii)
(a) a starch binding module having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 476 to 569 of SEQ ID NO: 56;
(b) a starch binding module encoded by a polynucleotide that hybridizes under medium, medium-high, high, or very high stringency conditions with the full-length complement of nucleotides 1426 to 1707 of SEQ ID NO: 55;
(c) a starch binding module encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1426 to 1707 of SEQ ID NO: 55; and
(d) a fragment of the starch binding module of (a), (b), or (c) that has binding activity; and
   (viii)
(a) a starch binding module having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 479 to 572 of SEQ ID NO: 59;
(b) a starch binding module encoded by a polynucleotide that hybridizes under medium, medium-high, high, or very high stringency conditions with the full-length complement of nucleotides 1435 to 1716 of SEQ ID NO: 58;
(c) a starch binding module encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1435 to 1716 of SEQ ID NO: 58; and
(d) a fragment of the starch binding module of (a), (b), or (c) that has binding activity.

The present invention also relates to isolated or purified polynucleotides encoding the polypeptides of the present invention; nucleic acid constructs; recombinant expression vectors; recombinant host cells comprising the polynucleotides; and methods of producing the polypeptides.

The present invention relates to processes of producing fermentation products, such as ethanol, from starch-containing material using a fermenting organism.

In an aspect the invention relates to a process for producing fermentation products from starch-containing material comprising the steps of:
i) liquefying the starch-containing material at a temperature above the initial gelatinization temperature using an alpha-amylase;
ii) saccharifying using a carbohydrate-source generating enzyme;
iii) fermenting using a fermenting organism;
wherein at least one polypeptide having alpha-amylase activity of the present invention is present or added during fermentation or simultaneous saccharification and fermentation.

In another aspect the present invention relates to an enzyme composition comprising at least one polypeptide having alpha-amylase activity of the present invention.

In another aspect the invention relates to a recombinant host cell comprising at least one polypeptide having alpha-amylase activity of the present invention.

### BRIEF DESCRIPTION OF THE FIGURES

FIG. 1 shows amylase activity measured for Seq. ID 2, 5, 8, 11, 14, 16 and 41 as well as a blank. Error bars were constructed using 1 standard deviation from the mean.

FIG. 2 shows % residual activity after incubation at pH 4 or 5 for 1 day (18-24H), 32° C. (Stability method 1). Error bars were constructed using 1 standard deviation from the mean.

FIG. 3 shows % residual activity after incubation at pH 4 with or without 100 mg/mL corn starch for 1 day (18-24H), 32° C.(stability method 2). Error bars were constructed using 1 standard deviation from the mean. If no error bars occur, there is only one data point.

FIG. 4 shows alpha-amylase activity at pH 5 for the alpha-amylases shown in SEQ ID NO: 45, SEQ ID NO: 48, SEQ ID NO: 51, SEQ ID NO: 54, SEQ ID NO: 57 and SEQ ID NO: 60.

FIG. 5 shows alpha-amylase activity at pH 4 for the alpha-amylases shown in SEQ ID NO: 45, SEQ ID NO: 48, SEQ ID NO: 51, SEQ ID NO: 54, SEQ ID NO: 57 and SEQ ID NO: 60.

FIG. 6 shows ethanol stability (15% (v/v) EtOH, pH 5, 24H, 32° C.) for the alpha-amylases shown in SEQ ID NO: 6, SEQ ID NO: 9, SEQ ID NO: 12, SEQ ID NO: 15, SEQ ID NO: 18, SEQ ID NO: 42, SEQ ID NO: 48, SEQ ID NO: 51, SEQ ID NO: 54, SEQ ID NO: 57 and SEQ ID NO: 60.

FIG. 7 shows residual starch level following 54 h of simultaneous saccharification and fermentation that was treated with either 5 or 20 ug (per gram of dry solid) of alpha-amylases of the invention. The control treatment, no alpha-amylase addition, is shown as 0 ug dose. Error bars represent the standard error of three replicates.

### DEFINITIONS

In accordance with this detailed description, the following definitions apply. Note that the singular forms “a,” “an,” and “the” include plural references unless the context clearly dictates otherwise.

Reference to “about” a value or parameter herein includes aspects that are directed to that value or parameter per se. For example, description referring to “about X” includes the aspect

Unless defined otherwise or clearly indicated by context, all technical and scientific terms used herein have the same meaning as commonly understood by one of ordinary skill in the art to which this invention belongs.

Alpha-amylase: The term “alpha-amylase” means an 1,4-alpha-D-glucan glucanohydrolase, EC. 3.2.1.1, which catalyze hydrolysis of starch and other linear and branched 1,4-glucosidic oligo- and polysaccharides. For purposes of the present invention, alpha-amylase activity is determined according to the procedure described in the Examples. In one aspect, the polypeptides of the present invention have at least 20%, e.g., at least 40%, at least 50%, at least 60%, at least 70%, at least 80%, at least 90%, at least 95%, or at least 100% of the alpha-amylase activity of the mature polypeptide of SEQ ID NO: 2, SEQ ID NO: 5, SEQ ID NO: 8, SEQ ID NO: 11, SEQ ID NO: 14, SEQ ID NO: 41, SEQ ID NO: 44, SEQ ID NO: 47, SEQ ID NO: 50 or SEQ ID NO: 53.

Alpha-amylases of the present invention used in the processes and compositions of the present invention are preferably the mature form, e.g., in one aspect, the mature polypeptide is amino acids 21 to 603 of SEQ ID NO: 2. Amino acids 1 to 20 of SEQ ID NO: 2 are a signal peptide. In another aspect, the mature polypeptide is SEQ ID NO: 3. In another aspect, the mature polypeptide is amino acids 18 to 632 of SEQ ID NO: 5. Amino acids 1 to 17 of SEQ ID NO: 5 are a signal peptide. In another aspect, the mature polypeptide is SEQ ID NO: 6. In another aspect, the mature polypeptide is amino acids 22 to 632 of SEQ ID NO: 8. Amino acids 1 to 21 of SEQ ID NO: 8 are a signal peptide. In another aspect, the mature polypeptide is SEQ ID NO: 9. In another aspect, the mature polypeptide is amino acids 20 to 609 of SEQ ID NO: 11. Amino acids 1 to 19 of SEQ ID NO: 11 are a signal peptide. In another aspect, the mature polypeptide is SEQ ID NO: 12. In another aspect, the mature polypeptide is amino acids 30 to 469 of SEQ ID NO: 14. In another aspect, the mature polypeptide is amino acids 30 to 470 of SEQ ID NO: 14. In another aspect, the mature polypeptide is amino acids 30-475 of SEQ ID NO: 14. Amino acids 1 to 29 of SEQ ID NO: 14 are a signal peptide. In another aspect, the mature polypeptide is SEQ ID NO: 15. In another aspect, the mature polypeptide is amino acids 28 to 659 of SEQ ID NO: 41. Amino acids 1 to 27 of SEQ ID NO: 41 are a signal peptide. In another aspect, the mature polypeptide is SEQ ID NO: 42. In another aspect, the mature polypeptide is amino acids 28 to 575 of SEQ ID NO: 44. In another aspect, the mature polypeptide is SEQ ID NO: 45. In another aspect, the mature polypeptide is amino acids 28 to 572 of SEQ ID NO: 47. In another aspect, the mature polypeptide is SEQ ID NO: 48. In another aspect, the mature polypeptide is amino acids 28 to 577 of SEQ ID NO: 50. In another aspect, the mature polypeptide is SEQ ID NO: 51. In another aspect, the mature polypeptide is amino acids 28 to 757 of SEQ ID NO: 53. In another aspect, the mature polypeptide is SEQ ID NO: 54. In another aspect, the mature polypeptide is amino acids 28 to 575 of SEQ ID NO: 56. In another aspect, the mature polypeptide is SEQ ID NO: 57. In another aspect, the mature polypeptide is amino acids 28 to 578 of SEQ ID NO: 59. In another aspect, the mature polypeptide is SEQ ID NO: 60.

Auxiliary Activity 9 polypeptide (previously named GH61): The term “Auxiliary Activity 9 polypeptide” or “AA9 polypeptide” means a polypeptide classified as a lytic polysaccharide monooxygenase (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). AA9 polypeptides were formerly classified into the glycoside hydrolase Family 61 (GH61) according to Henrissat, 1991, Biochem. J. 280: 309-316, and Henrissat and Bairoch, 1996, Biochem. J. 316: 695-696.

AA9 polypeptides enhance the hydrolysis of a cellulosic material by an enzyme having cellulolytic activity. Cellulolytic enhancing activity can be determined by measuring the increase in reducing sugars or the increase of the total of cellobiose and glucose from the hydrolysis of a cellulosic material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of an AA9 polypeptide for 1-7 days at a suitable temperature, such as 40° C.-80° C., e.g., 40° C., 45° C., 50° C., 55° C., 60° C., 65° C., 70° C., 75° C., or 80° C. and a suitable pH, such as 4-9, e.g., 4.5, 5.0, 5.5, 6.0, 6.5, 7.0, 7.5, 8.0, 8.5, or 9.0, compared to a control hydrolysis with equal total protein loading without cellulolytic enhancing activity (1-50 mg of cellulolytic protein/g of cellulose in PCS).

AA9 polypeptide enhancing activity can be determined using a mixture of CELLUCLAST™ 1.5L (Novozymes A/S, Bagsværd, Denmark) and beta-glucosidase as the source of the cellulolytic activity, wherein the beta-glucosidase is present at a weight of at least 2-5% protein of the cellulase protein loading. In one aspect, the beta-glucosidase is an Aspergillus oryzae beta-glucosidase (e.g., recombinantly produced in Aspergillus oryzae according to WO 02/095014). In another aspect, the beta-glucosidase is an Aspergillus fumigatus beta-glucosidase (e.g., recombinantly produced in Aspergillus oryzae as described in WO 02/095014).

AA9 polypeptide enhancing activity can also be determined by incubating an AA9 polypeptide with 0.5% phosphoric acid swollen cellulose (PASC), 100 mM sodium acetate pH 5, 1 mM MnSO₄, 0.1% gallic acid, 0.025 mg/ml of Aspergillus fumigatus beta-glucosidase, and 0.01% TRITON® X-100 (4-(1,1,3,3-tetramethylbutyl)phenyl-polyethylene glycol) for 24-96 hours at 40° C. followed by determination of the glucose released from the PASC.

AA9 polypeptide enhancing activity can also be determined according to WO 2013/028928 for high temperature compositions.

AA9 polypeptides enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

The AA9 polypeptide can also be used in the presence of a soluble activating divalent metal cation according to WO 2008/151043 or WO 2012/122518, e.g., manganese or copper.

The AA9 polypeptide can be used in the presence of a dioxy compound, a bicyclic compound, a heterocyclic compound, a nitrogen-containing compound, a quinone compound, a sulfur-containing compound, or a liquor obtained from a pretreated cellulosic or hemicellulosic material such as pretreated corn stover (WO 2012/021394, WO 2012/021395, WO 2012/021396, WO 2012/021399, WO 2012/021400, WO 2012/021401, WO 2012/021408, and WO 2012/021410).

Beta-glucanase: The term “beta-glucanase” means a (1,3)-(1,4)-β-D-glucan 4-glucanohydrolase (E.C. 3.2.1.73) that catalyzes the hydrolysis of (1,4)-β-D-glucosidic linkages in β-D-glucans containing (1,3)- and (1,4)-bonds. Thus, the beta-glucanase acts on lichenin and cereal β-D-glucans, but not on β-D-glucans containing only 1,3- or 1,4-bonds. Beta-glucanase activity can be determined using the beta-glucanase activity (AZCL-beta-glucan assay) as defined in the Enzyme Assay section.

Beta-glucosidase: The term “beta-glucosidase” means beta-D-glucoside glucohydrolase (E.C. 3.2.1.21) that catalyzes the hydrolysis of terminal non-reducing beta-D-glucose residues with the release of beta-D-glucose.

For purposes of the present invention, beta-glucosidase activity is determined using p-nitrophenyl-beta-D-glucopyranoside as substrate according to the procedure of Venturi et al., 2002, Extracellular beta-D-glucosidase from Chaetomium thermophilum var. coprophilum: production, purification and some biochemical properties, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20 (polyoxyethylene sorbitan monolaurate).

Binding module: The term “starch binding module” means the region of an enzyme that mediates binding of the enzyme to amorphous regions of a substrate selected from the group consisting of starch granules, its soluble components amylose and amylopectin, as well as derived maltooligosaccharides, such as maltose, maltoheptose, and maltodecose. The starch binding module (CBD) is typically found either at the N-terminal or at the C-terminal extremity of an alpha-amylase.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cellobiohydrolase: The term “cellobiohydrolase” means a 1,4-beta-D-glucan cellobiohydrolase (E.C. 3.2.1.91) that catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain (Teeri, 1997, Crystalline cellulose degradation: New insight into the function of cellobiohydrolases, Trends in Biotechnology 15: 160-167; Teeri et al., 1998, Trichoderma reesei cellobiohydrolases: why so efficient on crystalline cellulose?, Biochem. Soc. Trans. 26: 173-178).

Cellobiohydrolase activity is determined according to the procedures described by Lever et al., 1972, Anal. Biochem. 47: 273-279; van Tilbeurgh et al., 1982, FEBS Letters, 149: 152-156; van Tilbeurgh and Claeyssens, 1985, FEBS Letters, 187: 283-288; and Tomme et al., 1988, Eur. J. Biochem. 170: 575-581. In the present invention, the Tomme et al. method can be used to determine cellobiohydrolase activity.

Cellulolytic composition, cellulolytic enzymes or cellulase means a preparation comprising one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24: 452-481. Total cellulolytic activity is usually measured using insoluble substrates, including Whatman No1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Measurement of cellulase activities, Pure Appl. Chem. 59: 257-68).

Cellulolytic enzyme activity is determined by measuring the increase in hydrolysis of a cellulosic material by cellulolytic enzyme(s) under the following conditions: 1-50 mg of cellulolytic enzyme protein/g of cellulose in Pretreated Corn Stover (“PCS”) (or other pretreated cellulosic material) for 3-7 days at a suitable temperature, e.g., 50° C., 55° C., or 60° C., compared to a control hydrolysis without addition of cellulolytic enzyme protein. Typical conditions are 1 ml reactions, washed or unwashed PCS, 5% insoluble solids, 50 mM sodium acetate pH 5, 1 mM MnSO₄, 50° C., 55° C., or 60° C., 72 hours, sugar analysis by AMINEX® HPX-87H column (Bio-Rad Laboratories, Inc., Hercules, CA, USA).

Coding sequence: The term “coding sequence” or “coding region” means a polynucleotide sequence, which specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a sequence of genomic DNA, cDNA, a synthetic polynucleotide, and/or a recombinant polynucleotide.

Control sequences: The term “control sequences” means nucleic acid sequences necessary for expression of a polynucleotide encoding a polypeptide of the present invention. Each control sequence may be native (i.e., from the same gene) or heterologous (i.e., from a different gene) to the polynucleotide encoding the polypeptide or native or heterologous to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Disruption: The term “disruption” means that a coding region and/or control sequence of a referenced gene is partially or entirely modified (such as by deletion, insertion, and/or substitution of one or more nucleotides) resulting in the absence (inactivation) or decrease in expression, and/or the absence or decrease of enzyme activity of the encoded polypeptide. The effects of disruption can be measured using techniques known in the art such as detecting the absence or decrease of enzyme activity using from cell-free extract measurements referenced herein; or by the absence or decrease of corresponding mRNA (e.g., at least 25% decrease, at least 50% decrease, at least 60% decrease, at least 70% decrease, at least 80% decrease, or at least 90% decrease); the absence or decrease in the amount of corresponding polypeptide having enzyme activity (e.g., at least 25% decrease, at least 50% decrease, at least 60% decrease, at least 70% decrease, at least 80% decrease, or at least 90% decrease); or the absence or decrease of the specific activity of the corresponding polypeptide having enzyme activity (e.g., at least 25% decrease, at least 50% decrease, at least 60% decrease, at least 70% decrease, at least 80% decrease, or at least 90% decrease). Disruptions of a particular gene of interest can be generated by methods known in the art, e.g., by directed homologous recombination (see Methods in Yeast Genetics (1997 edition), Adams, Gottschling, Kaiser, and Stems, Cold Spring Harbor Press (1998)).

Endogenous gene: The term “endogenous gene” means a gene that is native to the referenced host cell. “Endogenous gene expression” means expression of an endogenous gene.

Endoglucanase: The term “endoglucanase” means an endo-1,4-(1,3; 1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components.

Endoglucanase activity can be determined by measuring reduction in substrate viscosity or increase in reducing ends determined by a reducing sugar assay (Zhang et al., 2006, Biotechnology Advances 24: 452-481). For purposes of the present invention, endoglucanase activity is determined using carboxymethyl cellulose (CMC) as substrate according to the procedure of Ghose, 1987, Pure and Appl. Chem. 59: 257-268, at pH 5, 40° C.

Expression: The term “expression” includes any step involved in the production of the polypeptide including, but not limited to, transcription, post-transcriptional modification, translation, post-translational modification, and secretion. Expression can be measured—for example, to detect increased expression—by techniques known in the art, such as measuring levels of mRNA and/or translated polypeptide.

Expression vector: The term “expression vector” means a linear or circular DNA molecule that comprises a polynucleotide encoding a polypeptide and is operably linked to control sequences that provide for its expression.

Family 61 glycoside hydrolase (now known as AA9): The term “Family 61 glycoside hydrolase” or “Family GH61” or “GH61” means a polypeptide falling into the glycoside hydrolase Family 61 according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696. The enzymes in this family were originally classified as a glycoside hydrolase family based on measurement of very weak endo-1,4-beta-D-glucanase activity in one family member. The structure and mode of action of these enzymes are non-canonical and they cannot be considered as bona fide glycosidases. However, they are kept in the CAZy classification on the basis of their capacity to enhance the breakdown of lignocellulose when used in conjunction with a cellulase or a mixture of cellulases.

Fermentable medium: The term “fermentable medium” or “fermentation medium” refers to a medium comprising one or more (e.g., two, several) sugars, such as glucose, fructose, sucrose, cellobiose, xylose, xylulose, arabinose, mannose, galactose, and/or soluble oligosaccharides, wherein the medium is capable, in part, of being converted (fermented) by a host cell into a desired product, such as ethanol. In some instances, the fermentation medium is derived from a natural source, such as sugar cane, starch, or cellulose, and may be the result of pretreating the source by enzymatic hydrolysis (saccharification). The term fermentation medium is understood herein to refer to a medium before the fermenting organism is added, such as, a medium resulting from a saccharification process, as well as a medium used in a simultaneous saccharification and fermentation process (SSF).

Fragment: The term “fragment” means a polypeptide, a catalytic domain, or a starch binding module having one or more (e.g., several) amino acids absent from the amino and/or carboxyl terminus of a mature polypeptide or domain; wherein the fragment has alpha-amylase or starch binding activity. In some embodiments, a fragment contains at least 512 amino acid residues (e.g., amino acids 1 to 512 of SEQ ID NO: 2 or SEQ ID NO: 3), at least 542 amino acid residues (e.g., amino acids 1 to 542 of SEQ ID NO: 2 or SEQ ID NO: 3), or at least 572 amino acid residues (e.g., amino acids 1 to 572 of SEQ ID NO: 2 or SEQ ID NO: 3). In some embodiments, a fragment contains at least 401 amino acid residues (e.g., amino acids 21 to 422 of SEQ ID NO: 2), at least 424 amino acid residues (e.g., amino acids 21 to 445 of SEQ ID NO: 2), or at least 448 amino acid residues (e.g., amino acids 21 to 469 of SEQ ID NO: 2). In some embodiments, a fragment contains at least 81 amino acid residues (e.g., amino acids 507 to 588 of SEQ ID NO: 2), at least 86 amino acid residues (e.g., amino acids 507 to 593 of SEQ ID NO: 2), or at least 91 amino acid residues (e.g., amino acids 507 to 598 of SEQ ID NO: 2). In some embodiments, a fragment contains at least 537 amino acid residues (e.g., amino acids 1 to 537 of SEQ ID NO: 5 or SEQ ID NO: 6), at least 568 amino acid residues (e.g., amino acids 1 to 568 of SEQ ID NO: 5 or SEQ ID NO: 6), or at least 600 amino acid residues (e.g., amino acids 1 to 600 of SEQ ID NO: 5 or SEQ ID NO: 6). In some embodiments, a fragment contains at least 407 amino acid residues (e.g., amino acids 18 to 425 of SEQ ID NO: 5), at least 431 amino acid residues (e.g., amino acids 18 to 449 of SEQ ID NO: 5), or at least 455 amino acid residues (e.g., amino acids 18 to 473 of SEQ ID NO: 5). In some embodiments, a fragment contains at least 85 amino acid residues (e.g., amino acids 532 to 617 of SEQ ID NO: 5), at least 90 amino acid residues (e.g., amino acids 532 to 622 of SEQ ID NO: 5), or at least 95 amino acid residues (e.g., amino acids 532 to 627 of SEQ ID NO: 5). In some embodiments, a fragment contains at least 537 amino acid residues (e.g., amino acids 1 to 537 of SEQ ID NO: 8 or SEQ ID NO: 9), at least 568 amino acid residues (e.g., amino acids 1 to 568 of SEQ ID NO: 8 or SEQ ID NO: 9), or at least 600 amino acid residues (e.g., amino acids 1 to 600 of SEQ ID NO: 8 or SEQ ID NO: 9). In some embodiments, a fragment contains at least 402 amino acid residues (e.g., amino acids 22 to 424 of SEQ ID NO: 8), at least 425 amino acid residues (e.g., amino acids 22 to 447 of SEQ ID NO: 8), or at least 449 amino acid residues (e.g., amino acids 22 to 471 of SEQ ID NO: 8). In some embodiments, a fragment contains at least 85 amino acid residues (e.g., amino acids 532 to 617 of SEQ ID NO: 8), at least 90 amino acid residues (e.g., amino acids 532 to 622 of SEQ ID NO: 8), or at least 95 amino acid residues (e.g., amino acids 532 to 627 of SEQ ID NO: 8). In some embodiments, a fragment contains at least 517 amino acid residues (e.g., amino acids 1 to 517 of SEQ ID NO: 11 or SEQ ID NO: 12), at least 548 amino acid residues (e.g., amino acids 1 to 548 of SEQ ID NO: 11 or SEQ ID NO: 12), or at least 578 amino acid residues (e.g., amino acids 1 to 578 of SEQ ID NO: 11 or SEQ ID NO: 12). In some embodiments, a fragment contains at least 404 amino acid residues (e.g., amino acids 20 to 424 of SEQ ID NO: 11), at least 428 amino acid residues (e.g., amino acids 20 to 448 of SEQ ID NO: 11), or at least 452 amino acid residues (e.g., amino acids 20 to 472 of SEQ ID NO: 11). In some embodiments, a fragment contains at least 79 amino acid residues (e.g., amino acids 508 to 587 of SEQ ID NO: 11), at least 83 amino acid residues (e.g., amino acids 508 to 591 of SEQ ID NO: 11), or at least 88 amino acid residues (e.g., amino acids 508 to 596 of SEQ ID NO: 11). In some embodiments, a fragment contains at least 403 amino acid residues (e.g., amino acids 1 to 403 of SEQ ID NO: 14 or SEQ ID NO: 15), at least 427 amino acid residues (e.g., amino acids 1 to 427 of SEQ ID NO: 14 or SEQ ID NO: 15), or at least 451 amino acid residues (e.g., amino acids 1 to 451 of SEQ ID NO: 14 or SEQ ID NO: 15). In some embodiments, a fragment contains at least 373 amino acid residues (e.g., amino acids 30 to 403 of SEQ ID NO: 14), at least 395 amino acid residues (e.g., amino acids 30 to 425 of SEQ ID NO: 14), or at least 417 amino acid residues (e.g., amino acids 30 to 447 of SEQ ID NO: 14). In some embodiments, a fragment contains at least 560 amino acid residues (e.g., amino acids 1 to 560 of SEQ ID NO: 41 or SEQ ID NO: 42), at least 593 amino acid residues (e.g., amino acids 1 to 593 of SEQ ID NO: 41 or SEQ ID NO: 42, or at least 626 residues (e.g., amino acids 1 to 626 of SEQ ID NO: 41 or SEQ ID NO: 42).

In some embodiments, a fragment contains at least 371 amino acid residues (e.g., amino acids 28 to 399 of SEQ ID NO: 41), at least 393 amino acid residues (e.g., amino acids 28 to 421 of SEQ ID NO: 41), or at least 415 amino acid residues (e.g., amino acids 28 to 443 of SEQ ID NO: 41). In some embodiments, a fragment contains at least 84 amino acid residues (e.g., amino acids 554 to 638 of SEQ ID NO: 41), at least 89 amino acid residues (e.g., amino acids 554 to 643 of SEQ ID NO: 41), or at least 94 amino acid residues (e.g., amino acids 554 to 649 of SEQ ID NO: 41). In some embodiments, a fragment contains at least 488 amino acids (e.g., amino acids 87 to 575 of SEQ ID NO: 44 or amino acids 1 to 488 of SEQ ID NO: 45), at least 517 amino acids (e.g., amino acids 58 to 575 of SEQ ID NO: 44 or amino acids 1 to 517 of SEQ ID NO: 45), or at least 546 amino acids (e.g., 29 to 575 of SEQ ID NO: 44 or amino acids 1 to 546 of SEQ ID NO: 45). In some embodiments, a fragment contains at least 359 amino acids (e.g., amino acids 108 to 467 of SEQ ID NO: 44 or amino acids 81 to 440 of SEQ ID NO: 45), at least 380 amino acids (e.g., amino acids 87 to 467 of SEQ ID NO: 44 or amino acids 60 to 440 of SEQ ID NO: 45), or at least 401 amino acids (e.g., amino acids 66 to 467 of SEQ ID NO: 44 or amino acids 39 to 440 of SEQ ID NO: 45). In some embodiments, a fragment contains at least 79 amino acids (e.g., amino acids 490 to 569 of SEQ ID NO: 44 or amino acids 463 to 542 of SEQ ID NO: 45), at least 82 amino acids (e.g., amino acids 487 to 569 of SEQ ID NO: 44 or amino acids 460 to 542 of SEQ ID NO: 45), or at least 87 amino acids (e.g., amino acids 482 to 569 of SEQ ID NO: 44 or amino acids 455 to 542 of SEQ ID NO: 45). In some embodiments, a fragment contains at least 486 amino acids (e.g., amino acids 86 to 572 of SEQ ID NO: 47 or amino acids 1 to 486 of SEQ ID NO: 48), at least 514 amino acids (e.g., amino acids 58 to 572 of SEQ ID NO: 47 or amino acids 1 to 514 of SEQ ID NO: 48), or at least 543 amino acids (e.g., amino acids 29 to 572 of SEQ ID NO: 47 or amino acids 1 to 572 of SEQ ID NO: 48). In some embodiments, a fragment contains at least 359 amino acids (e.g., amino acids 108 to 467 of SEQ ID NO: 47 or amino acids 81 to 440 of SEQ ID NO: 48), at least 380 amino acids (e.g., amino acids 87 to 467 of SEQ ID NO: 47 or amino acids 60 to 440 of SEQ ID NO: 48), or at least 401 amino acids (e.g., amino acids 66 to 467 of SEQ ID NO: 47 or amino acids 39 to 440 of SEQ ID NO: 48). In some embodiments, a fragment contains at least 81 amino acids (e.g., amino acids 484 to 565 of SEQ ID NO: 47 or amino acids 457 to 538 of SEQ ID NO: 48), at least 86 amino acids (e.g., amino acids 479 to 565 of SEQ ID NO: 47 or amino acids 460 to 538 of SEQ ID NO: 48), or at least 91 amino acids (e.g., amino acids 474 to 565 of SEQ ID NO: 47 or amino acids 447 to 538 of SEQ ID NO: 48).

In some embodiments, a fragment contains at least 490 amino acids (e.g., amino acids 87 to 577 of SEQ ID NO: 50 or amino acids 1 to 490 of SEQ ID NO: 51), at least 519 amino acids (e.g., amino acids 58 to 577 of SEQ ID NO: 50 or amino acids 1 to 519 of SEQ ID NO: 51), or at least 548 amino acids (e.g., amino acids 29 to 577 of SEQ ID NO: 50 or amino acids 1 to 548 of SEQ ID NO: 51). In some embodiments, a fragment contains at least 359 amino acids (e.g., amino acids 108 to 467 of SEQ ID NO: 50 or amino acids 81 to 440 of SEQ ID NO: 51), at least 380 amino acids (e.g., amino acids 87 to 467 of SEQ ID NO: 50 or amino acids 60 to 440 of SEQ ID NO: 51), or at least 401 amino acids (e.g., amino acids 66 to 467 of SEQ ID NO: 50 or amino acids 39 to 440 of SEQ ID NO: 51). In some embodiments, a fragment contains at least 81 amino acids (e.g., amino acids 490 to 571 of SEQ ID NO: 50 or amino acids 463 to 544 of SEQ ID NO: 51), at least 86 amino acids (e.g., amino acids 485 to 571 of SEQ ID NO: 50 or amino acids 460 to 544 of SEQ ID NO: 51), or at least 91 amino acids (e.g., amino acids 480 to 571 of SEQ ID NO: 50 or amino acids 447 to 544 of SEQ ID NO: 51).

In some embodiments, a fragment contains at least 643 amino acids (e.g., amino acids 114 to 757 of SEQ ID NO: 53 or amino acids 1 to 643 of SEQ ID NO: 54), at least 681 amino acids (e.g., amino acids 76 to 757 of SEQ ID NO: 53 or amino acids 1 to 681 of SEQ ID NO: 54), or at least 719 amino acids (e.g., amino acids 38 to 757 of SEQ ID NO: 53 or amino acids 1 to 719 of SEQ ID NO: 54). In some embodiments, a fragment contains at least 359 amino acids (e.g., amino acids 108 to 467 of SEQ ID NO: 53 or amino acids 81 to 440 of SEQ ID NO: 54), at least 380 amino acids (e.g., amino acids 87 to 467 of SEQ ID NO: 53 or amino acids 60 to 440 of SEQ ID NO: 54), or at least 401 amino acids (e.g., amino acids 66 to 467 of SEQ ID NO: 53 or amino acids 39 to 440 of SEQ ID NO: 54). In some embodiments, a fragment contains at least 74 amino acids (e.g., amino acids 488 to 562 of SEQ ID NO: 53 or amino acids 461 to 535 of SEQ ID NO: 54), at least 79 amino acids (e.g., amino acids 483 to 562 of SEQ ID NO: 53 or amino acids 456 to 535 of SEQ ID NO: 54), or at least 83 amino acids (e.g., amino acids 479 to 562 of SEQ ID NO: 53 or amino acids 452 to 535 of SEQ ID NO: 54). In some embodiments, a fragment contains at least 78 amino acids (e.g., amino acids 576 to 654 of SEQ ID NO: 53 or amino acids 549 to 627 of SEQ ID NO: 54), at least 82 amino acids (e.g., amino acids 572 to 654 of SEQ ID NO: 53 or amino acids 545 to 627 of SEQ ID NO: 54), or at least 87 amino acids (e.g., amino acids 567 to 654 of SEQ ID NO: 53 or amino acids 540 to 627 of SEQ ID NO: 54). In some embodiments, a fragment contains at least 81 amino acids (e.g., amino acids 669 to 750 of SEQ ID NO: 53 or amino acids 642 to 723 of SEQ ID NO: 54), at least 86 amino acids (e.g., amino acids 664 to 750 of SEQ ID NO: 53 or amino acids 637 to 723 of SEQ ID NO: 54), or at least 91 amino acids (e.g., amino acids 659 to 750 of SEQ ID NO: 53 or amino acids 632 to 723 of SEQ ID NO: 54).

In some embodiments, a fragment contains at least 488 amino acids (e.g., amino acids 87 to 575 of SEQ ID NO: 56 or amino acids 1 to 488 of SEQ ID NO: 57), at least 517 amino acids (e.g., amino acids 58 to 575 of SEQ ID NO: 56 or amino acids 1 to 517 of SEQ ID NO: 57), or at least 546 amino acids (e.g., amino acids 29 to 575 of SEQ ID NO: 56 or amino acids 1 to 546 of SEQ ID NO: 57). In some embodiments, a fragment contains at least 359 amino acids (e.g., amino acids 108 to 467 of SEQ ID NO: 56 or amino acids 81 to 440 of SEQ ID NO: 57), at least 380 amino acids (e.g., amino acids 87 to 467 of SEQ ID NO: 56 or amino acids 60 to 440 of SEQ ID NO: 57), or at least 401 amino acids (e.g., amino acids 66 to 467 of SEQ ID NO: 56 or amino acids 39 to 440 of SEQ ID NO: 57). In some embodiments, a fragment contains at least 79 amino acids (e.g., amino acids 496 to 569 of SEQ ID NO: 56 or amino acids 463 to 542 of SEQ ID NO: 57), at least 84 amino acids (e.g., amino acids 481 to 569 of SEQ ID NO: 56 or amino acids 458 to 542 of SEQ ID NO: 57), or at least 88 amino acids (e.g., amino acids 481 to 569 of SEQ ID NO: 56 or amino acids 454 to 542 of SEQ ID NO: 57).

In some embodiments, a fragment contains at least 491 amino acids (e.g., amino acids 87 to 578 of SEQ ID NO: 59 or amino acids 1 to 491 of SEQ ID NO: 60), at least 520 amino acids (e.g., amino acids 58 to 578 of SEQ ID NO: 59 or amino acids 1 to 520 of SEQ ID NO: 60), or at least 549 amino acids (e.g., amino acids 29 to 578 of SEQ ID NO: 59 or amino acids 1 to 549 of SEQ ID NO: 60). In some embodiments, a fragment contains at least 359 amino acids (e.g., amino acids 108 to 467 of SEQ ID NO: 59 or amino acids 81 to 440 of SEQ ID NO: 60), at least 380 amino acids (e.g., amino acids 87 to 467 of SEQ ID NO: 59 or amino acids 60 to 440 of SEQ ID NO: 60), or at least 401 amino acids (e.g., amino acids 66 to 467 of SEQ ID NO: 59 or amino acids 39 to 440 of SEQ ID NO: 60). In some embodiments, a fragment contains at least 79 amino acids (e.g., amino acids 499 to 572 of SEQ ID NO: 59 or amino acids 466 to 545 of SEQ ID NO: 60), at least 84 amino acids (e.g., amino acids 484 to 572 of SEQ ID NO: 59 or amino acids 461 to 545 of SEQ ID NO: 60), or at least 88 amino acids (e.g., amino acids 484 to 572 of SEQ ID NO: 59 or amino acids 457 to 545 of SEQ ID NO: 60).

Fusion polypeptide: The term “fusion polypeptide” is a polypeptide in which one polypeptide is fused at the N-terminus or the C-terminus of a polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Glucoamylase: The term “glucoamylase” (1,4-alpha-D-glucan glucohydrolase, EC 3.2.1.3) is defined as an enzyme that catalyzes the release of D-glucose from the non-reducing ends of starch or related oligo- and polysaccharide molecules. For purposes of the present invention, glucoamylase activity may be determined according to the procedures known in the art, such as those described in the Examples of US Provisional Patent Application No. 62/703,103, filed Jul. 25, 2018.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, or domain of a polypeptide or nucleic acid is not naturally associated with the polypeptide or nucleic acid, i.e., the control sequence is from a gene other than the gene encoding the mature polypeptide of SEQ ID NO: 2.

Heterologous polynucleotide: The term “heterologous polynucleotide” is defined herein as a polynucleotide that is not native to the host cell; a native polynucleotide in which structural modifications have been made to the coding region; a native polynucleotide whose expression is quantitatively altered as a result of a manipulation of the DNA by recombinant DNA techniques, e.g., a different (foreign) promoter; or a native polynucleotide in a host cell having one or more extra copies of the polynucleotide to quantitatively alter expression. A “heterologous gene” is a gene comprising a heterologous polynucleotide.

Host cell: The term “host cell” means any cell type that is susceptible to transformation, transfection, transduction, and the like with a nucleic acid construct or expression vector comprising a polynucleotide described herein. In some embodiments, the host cell is an isolated recombinant host cell that is partially or completely separated from at least one other component with, including but not limited to, proteins, nucleic acids, cells, etc. The term “host cell” encompasses any progeny of a parent cell that is not identical to the parent cell due to mutations that occur during replication. The term “recombinant cell” is defined herein as a non-naturally occurring host cell comprising one or more (e.g., two, several) heterologous polynucleotides.

Hybrid polypeptide: The term “hybrid polypeptide” means a polypeptide comprising domains from two or more polypeptides, e.g., a starch binding module or domain from one polypeptide and a catalytic domain from another polypeptide. The domains may be fused at the N-terminus or the C-terminus.

Hybridization: The term “hybridization” means the pairing of substantially complementary strands of nucleic acids, using standard Southern blotting procedures. Hybridization may be performed under medium, medium-high, high or very high stringency conditions. Medium stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 55° C. Medium-high stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 60° C. High stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 65° C. Very high stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 70° C.

Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture broth containing the secreted polypeptide.

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide. In one aspect, the mature polypeptide is amino acids 21 to 603 of SEQ ID NO:

2. In one aspect, the mature polypeptide is SEQ ID NO: 3. In one aspect, the mature polypeptide is amino acids 18 to 632 of SEQ ID NO: 5. In one aspect, the mature polypeptide is SEQ ID NO: 6. In one aspect, the mature polypeptide is amino acids 22 to 632 of SEQ ID NO: 8. In one aspect, the mature polypeptide is SEQ ID NO: 9. In one aspect, the mature polypeptide is amino acids 20 to 609 of SEQ ID NO: 11. In one aspect, the mature polypeptide is amino acids 20 to 601 of SEQ ID NO: 11. In one aspect, the mature polypeptide is SEQ ID NO: 12. In one aspect, the mature polypeptide is amino acids 30 to 469 of SEQ ID NO: 14. In one aspect, the mature polypeptide is amino acids 30 to 475 of SEQ ID NO: 14. In one aspect, the mature polypeptide is SEQ ID NO: 15. In one aspect, the mature polypeptide is amino acids 21 to 586 of SEQ ID NO: 16. In one aspect, the mature polypeptide is SEQ ID NO: 17. In one aspect, the mature polypeptide is amino acids 28 to 659 of SEQ ID NO: 41. In one aspect, the mature polypeptide is SEQ ID NO: 42. In another aspect, the mature polypeptide is amino acids 28 to 575 of SEQ ID NO: 44. In another aspect, the mature polypeptide is SEQ ID NO: 45. In another aspect, the mature polypeptide is amino acids 28 to 572 of SEQ ID NO: 47. In another aspect, the mature polypeptide is SEQ ID NO: 48. In another aspect, the mature polypeptide is amino acids 28 to 577 of SEQ ID NO: 50. In another aspect, the mature polypeptide is SEQ ID NO: 51. In another aspect, the mature polypeptide is amino acids 28 to 757 of SEQ ID NO: 53. In another aspect, the mature polypeptide is SEQ ID NO: 54. In another aspect, the mature polypeptide is amino acids 28 to 575 of SEQ ID NO: 56. In another aspect, the mature polypeptide is SEQ ID NO: 57. In another aspect, the mature polypeptide is amino acids 28 to 578 of SEQ ID NO: 59. In another aspect, the mature polypeptide is SEQ ID NO: 60.

Mature polypeptide coding sequence: The term “mature polypeptide coding sequence” means a polynucleotide that encodes a mature polypeptide having alpha-amylase activity. In one aspect, the mature polypeptide coding sequence is nucleotides 61 to 1812 of SEQ ID NO: 1. In one aspect, the mature polypeptide coding sequence is nucleotides 52 to 2351 of SEQ ID NO: 4, or the cDNA sequence thereof. In one aspect, the mature polypeptide coding sequence is nucleotides 64 to 2339 of SEQ ID NO: 7, or the cDNA sequence thereof. In one aspect, the mature polypeptide coding sequence is nucleotides 58 to 1830 of SEQ ID NO: 10. In one aspect, the mature polypeptide coding sequence is nucleotides 88 to 1428 of SEQ ID NO: 13. In one aspect, the mature polypeptide coding sequence is nucleotides 82 to 1977 of SEQ ID NO: 40. In one aspect, the mature polypeptide coding sequence is nucleotides 82 to 1725 of SEQ ID NO: 43. In one aspect, the mature polypeptide coding sequence is nucleotides 82 to 1716 of SEQ ID NO: 46. In one aspect, the mature polypeptide coding sequence is nucleotides 82 to 1731 of SEQ ID NO: 49. In one aspect, the mature polypeptide coding sequence is nucleotides 82 to 2271 of SEQ ID NO: 52. In one aspect, the mature polypeptide coding sequence is nucleotides 82 to 1728 of SEQ ID NO: 55. In one aspect, the mature polypeptide coding sequence is nucleotides 82 to 1737 of SEQ ID NO: 58.

Native: The term “native” means a nucleic acid or polypeptide naturally occurring in a host cell.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Polypeptide having cellulolytic enhancing activity: The term “polypeptide having cellulolytic enhancing activity” means a GH61 polypeptide that catalyzes the enhancement of the hydrolysis of a cellulosic material by enzyme having cellulolytic activity. For purposes of the present invention, cellulolytic enhancing activity is determined by measuring the increase in reducing sugars or the increase of the total of cellobiose and glucose from the hydrolysis of a cellulosic material by cellulolytic enzyme under the following conditions: 1-50 mg of total protein/g of cellulose in PCS, wherein total protein is comprised of 50-99.5% w/w cellulolytic enzyme protein and 0.5-50% w/w protein of a GH61 polypeptide having cellulolytic enhancing activity for 1-7 days at a suitable temperature, e.g., 50° C., 55° C., or 60° C., and pH, e.g., 5.0 or 5.5, compared to a control hydrolysis with equal total protein loading without cellulolytic enhancing activity (1-50 mg of cellulolytic protein/g of cellulose in PCS). In an aspect, a mixture of CELLUCLAST® 1.5L (Novozymes A/S, Bagsværd, Denmark) in the presence of 2-3% of total protein weight Aspergillus oryzae beta-glucosidase (recombinantly produced in Aspergillus oryzae according to WO 02/095014) or 2-3% of total protein weight Aspergillus fumigatus beta-glucosidase (recombinantly produced in Aspergillus oryzae as described in WO 2002/095014) of cellulase protein loading is used as the source of the cellulolytic activity.

The GH61 polypeptide having cellulolytic enhancing activity enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

Protease: The term “protease” is defined herein as an enzyme that hydrolyses peptide bonds. It includes any enzyme belonging to the EC 3.4 enzyme group (including each of the thirteen subclasses thereof). The EC number refers to Enzyme Nomenclature 1992 from NC-IUBMB, Academic Press, San Diego, California, including supplements 1-5 published in Eur. J. Biochem. 223: 1-5 (1994); Eur. J. Biochem. 232: 1-6 (1995); Eur. J. Biochem. 237: 1-5 (1996); Eur. J. Biochem. 250: 1-6 (1997); and Eur. J. Biochem. 264: 610-650 (1999); respectively. The term “subtilases” refer to a sub-group of serine protease according to Siezen et al., 1991, Protein Engng. 4: 719-737 and Siezen et al., 1997, Protein Science 6: 501-523. Serine proteases or serine peptidases is a subgroup of proteases characterised by having a serine in the active site, which forms a covalent adduct with the substrate. Further the subtilases (and the serine proteases) are characterised by having two active site amino acid residues apart from the serine, namely a histidine and an aspartic acid residue. The subtilases may be divided into 6 sub-divisions, i.e. the Subtilisin family, the Thermitase family, the Proteinase K family, the Lantibiotic peptidase family, the Kexin family and the Pyrolysin family. The term “protease activity” means a proteolytic activity (EC 3.4). Protease activity may be determined using methods described in the art (e.g., US 2015/0125925) or using commercially available assay kits (e.g., Sigma-Aldrich).

Pullulanase: The term “pullulanase” means a starch debranching enzyme having pullulan 6-glucano-hydrolase activity (EC 3.2.1.41) that catalyzes the hydrolysis the a-1,6-glycosidic bonds in pullulan, releasing maltotriose with reducing carbohydrate ends. For purposes of the present invention, pullulanase activity can be determined according to a PHADEBAS assay or the sweet potato starch assay described in WO2016/087237.

Purified: The term “purified” means a nucleic acid or polypeptide that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment).

Signal peptide: The term “signal peptide” is defined herein as a peptide linked (fused) in frame to the amino terminus of a polypeptide having biological activity and directs the polypeptide into the cell's secretory pathway. Signal sequences may be determined using techniques known in the art (See, e.g., Zhang and Henzel, 2004, Protein Science 13: 2819-2824). The polypeptides described herein may comprise any suitable signal peptide known in the art, or any signal peptide described in U.S. Provisional application No. 62/883,519, filed Aug. 6, 2019 (incorporated herein by reference).

Subsequence: The term “subsequence” means a polynucleotide having one or more (e.g., several) nucleotides absent from the 5′ and/or 3′ end of a mature polypeptide coding sequence; wherein the subsequence encodes a fragment having alpha-amylase activity. In one aspect, a subsequence contains at least 1536 nucleotides (e.g., nucleotides 1 to 1536 of SEQ ID NO: 1), at least 1626 nucleotides (e.g., nucleotides 1 to 1626 of SEQ ID NO: 1), or at least 1716 nucleotides (e.g., nucleotides 1 to 1716 of SEQ ID NO: 1). In another aspect, a subsequence contains at least 1203 nucleotides (e.g., nucleotides 63 to 1266 of SEQ ID NO: 1), at least 1272 nucleotides (e.g., nucleotides 63 to 1335 of SEQ ID NO: 1), or at least 1344 nucleotides (e.g., nucleotides 63 to 1407 of SEQ ID NO: 1). In another aspect, a subsequence contains at least 243 nucleotides (e.g., nucleotides 1521 to 1764 of SEQ ID NO: 1), at least 258 nucleotides (e.g., nucleotides 1521 to 1779 of SEQ ID NO: 1), or at least 273 nucleotides (e.g., nucleotides 1521 to 1794 of SEQ ID NO: 1). In another aspect, a subsequence contains at least 1611 nucleotides (e.g., nucleotides 1 to 1611 of SEQ ID NO: 4), at least 1704 nucleotides (e.g., nucleotides 1 to 1704 of SEQ ID NO: 4), or at least 1800 nucleotides (e.g., nucleotides 1 to 1800 of SEQ ID NO: 4). In another aspect, a subsequence contains at least 1221 nucleotides (e.g., nucleotides 54 to 1275 of SEQ ID NO: 4), at least 1293 nucleotides (e.g., nucleotides 54 to 1347 of SEQ ID NO: 4), or at least 1365 nucleotides (e.g., nucleotides 54 to 1419 of SEQ ID NO: 4). In another aspect, a subsequence contains at least 255 nucleotides (e.g., nucleotides 1596 to 1851 of SEQ ID NO: 4), at least 270 nucleotides (e.g., nucleotides 1596 to 1866 of SEQ ID NO: 4), or at least 285 nucleotides (e.g., nucleotides 1596 to 1881 of SEQ ID NO: 4). In another aspect, a subsequence contains at least 1611 nucleotides (e.g., nucleotides 1 to 1611 of SEQ ID NO: 7), at least 1704 nucleotides (e.g., nucleotides 1 to 1704 of SEQ ID NO: 7), or at least 1800 nucleotides (e.g., nucleotides 1 to 1800 of SEQ ID NO: 7). In another aspect, a subsequence contains at least 1206 nucleotides (e.g., nucleotides 66 to 1272 of SEQ ID NO: 7), at least 1275 nucleotides (e.g., nucleotides 66 to 1341 of SEQ ID NO: 7), or at least 1347 nucleotides (e.g., nucleotides 66 to 1413 of SEQ ID NO: 7). In another aspect, a subsequence contains at least 255 nucleotides (e.g., nucleotides 1596 to 1851 of SEQ ID NO: 7), at least 270 nucleotides (e.g., nucleotides 1596 to 1866 of SEQ ID NO: 7), or at least 285 nucleotides (e.g., nucleotides 1596 to 1881 of SEQ ID NO: 7). In another aspect, a subsequence contains at least 1551 nucleotides (e.g., nucleotides 1 to 1551 of SEQ ID NO: 10), at least 1644 nucleotides (e.g., nucleotides 1 to 1644 of SEQ ID NO: 10), or at least 1734 nucleotides (e.g., nucleotides 1 to 173 of SEQ ID NO: 10). In another aspect, a subsequence contains at least 1212 nucleotides (e.g., nucleotides 60 to 1272 of SEQ ID NO: 10), at least 1284 nucleotides (e.g., nucleotides 60 to 1344 of SEQ ID NO: 10), or at least 1356 nucleotides (e.g., nucleotides 60 to 1416 of SEQ ID NO: 10). In another aspect, a subsequence contains at least 237 nucleotides (e.g., nucleotides 1524 to 1761 of SEQ ID NO: 10), at least 249 nucleotides (e.g., nucleotides 1524 to 1773 of SEQ ID NO: 10), or at least 264 nucleotides (e.g., nucleotides 1524 to 1788 of SEQ ID NO: 10). In another aspect, a subsequence contains at least 1209 nucleotides (e.g., nucleotides 1 to 1209 of SEQ ID NO: 13), at least 1281 nucleotides (e.g., nucleotides 1 to 1281 of SEQ ID NO: 13), or at least 1353 nucleotides (e.g., nucleotides 1 to 1353 of SEQ ID NO: 13). In another aspect, a subsequence contains at least 1119 nucleotides (e.g., nucleotides 90 to 1209 of SEQ ID NO: 13), at least 1185 nucleotides (e.g., nucleotides 90 to 1275 of SEQ ID NO: 13), or at least 1251 nucleotides (e.g., nucleotides 90 to 1341 of SEQ ID NO: 13).

In another aspect, a subsequence contains at least 1680 nucleotides (e.g., nucleotides 1 to 1680 of SEQ ID NO: 40), at least 1779 nucleotides (e.g., nucleotides 1 to 1779 of SEQ ID NO: 40), or at least 1878 nucleotides (e.g., nucleotides 1 to 1878 of SEQ ID NO: 40). In some embodiments, a subsequence contains at least 1113 nucleotides (e.g., nucleotides 84 to 1197 of SEQ ID NO: 40), at least 1179 nucleotides (e.g., nucleotides 84 to 1263 of SEQ ID NO: 40), or at least 1245 nucleotides (e.g., nucleotides 84 to 1329 of SEQ ID NO: 40). In some embodiments, a subsequence contains at least 252 nucleotides (e.g., nucleotides 1162 to 1914 of SEQ ID NO: 40), at least 267 nucleotides (e.g., nucleotides 1662 to 1929 of SEQ ID NO: 40), or at least nucleotides (e.g., nucleotides 1662 to 1947 of SEQ ID NO: 40). In some embodiments, a subsequence contains at least 1464 nucleotides (e.g., nucleotides 261 to 1725 of SEQ ID NO: 44 or nucleotides 1 to 1464 of SEQ ID NO: 45), at least 1551 nucleotides (e.g., nucleotides 174 to 1725 of SEQ ID NO: 44 or nucleotides 1 to 1551 of SEQ ID NO: 45), or at least 1638 nucleotides (e.g., 97 to 1725 of SEQ ID NO: 44 or nucleotides 1 to 1638 of SEQ ID NO: 45). In some embodiments, a subsequence contains at least 1077 nucleotides (e.g., nucleotides 324 to 1401 of SEQ ID NO: 44 or nucleotides 243 to 1320 of SEQ ID NO: 45), at least 1140 nucleotides (e.g., nucleotides 261 to 1401 of SEQ ID NO: 44 or nucleotides 180 to 1320 of SEQ ID NO: 45), or at least 1203 nucleotides (e.g., nucleotides 198 to 1401 of SEQ ID NO: 44 or nucleotides 117 to 1320 of SEQ ID NO: 45). In some embodiments, a subsequence contains at least 237 nucleotides (e.g., nucleotides 1470 to 1707 of SEQ ID NO: 44 or nucleotides 1389 to 1626 of SEQ ID NO: 45), at least 246 nucleotides (e.g., nucleotides 1461 to 1707 of SEQ ID NO: 44 or nucleotides 1380 to 1626 of SEQ ID NO: 45), or at least 261 nucleotides (e.g., nucleotides 1446 to 1707 of SEQ ID NO: 44 or nucleotides 1365 to 1626 of SEQ ID NO: 45). In some embodiments, a subsequence contains at least 1458 nucleotides (e.g., nucleotides 258 to 1716 of SEQ ID NO: 47 or nucleotides 1 to 1458 of SEQ ID NO: 48), at least 1542 nucleotides (e.g., nucleotides 174 to 1716 of SEQ ID NO: 47 or nucleotides 1 to 1542 of SEQ ID NO: 48), or at least 1629 nucleotides (e.g., nucleotides 261 to 1716 of SEQ ID NO: 47 or nucleotides 1 to 1716 of SEQ ID NO: 48). In some embodiments, a subsequence contains at least 1077 nucleotides (e.g., nucleotides 324 to 1401 of SEQ ID NO: 47 or nucleotides 243 to 1320 of SEQ ID NO: 48), at least 1140 nucleotides (e.g., nucleotides 261 to 1401 of SEQ ID NO: 47 or nucleotides 180 to 1320 of SEQ ID NO: 48), or at least 1203 nucleotides (e.g., nucleotides 198 to 1401 of SEQ ID NO: 47 or nucleotides 117 to 1320 of SEQ ID NO: 48). In some embodiments, a subsequence contains at least 243 nucleotides (e.g., nucleotides 1452 to 1695 of SEQ ID NO: 47 or nucleotides 1371 to 1614 of SEQ ID NO: 48), at least 258 nucleotides (e.g., nucleotides 1437 to 1695 of SEQ ID NO: 47 or nucleotides 1380 to 1614 of SEQ ID NO: 48), or at least 273 nucleotides (e.g., nucleotides 1422 to 1695 of SEQ ID NO: 47 or nucleotides 1341 to 1614 of SEQ ID NO: 48). In some embodiments, a subsequence contains at least 1470 nucleotides (e.g., nucleotides 261 to 1731 of SEQ ID NO: 50 or nucleotides 1 to 1470 of SEQ ID NO: 51), at least 1557 nucleotides (e.g., nucleotides 174 to 1731 of SEQ ID NO: 50 or nucleotides 1 to 1557 of SEQ ID NO: 51), or at least 1644 nucleotides (e.g., nucleotides 97 to 1731 of SEQ ID NO: 50 or nucleotides 1 to 1644 of SEQ ID NO: 51). In some embodiments, a subsequence contains at least 1077 nucleotides (e.g., nucleotides 324 to 1401 of SEQ ID NO: 50 or nucleotides 243 to 1320 of SEQ ID NO: 51), at least 1140 nucleotides (e.g., nucleotides 261 to 1401 of SEQ ID NO: 50 or nucleotides 180 to 1320 of SEQ ID NO: 51), or at least 1203 nucleotides (e.g., nucleotides 198 to 1401 of SEQ ID NO: 50 or nucleotides 117 to 1320 of SEQ ID NO: 51). In some embodiments, a subsequence contains at least 243 nucleotides (e.g., nucleotides 1470 to 1713 of SEQ ID NO: 50 or nucleotides 1389 to 1632 of SEQ ID NO: 51), at least 258 nucleotides (e.g., nucleotides 1455 to 1713 of SEQ ID NO: 50 or nucleotides 1380 to 1638 of SEQ ID NO: 51), or at least 273 nucleotides (e.g., nucleotides 1440 to 1713 of SEQ ID NO: 50 or nucleotides 1341 to 1614 of SEQ ID NO: 51). In some embodiments, a subsequence contains at least 1929 nucleotides (e.g., nucleotides 342 to 2271 of SEQ ID NO: 53 or nucleotides 1 to 1929 of SEQ ID NO: 54), at least 2043 nucleotides (e.g., nucleotides 228 to 2271 of SEQ ID NO: 53 or nucleotides 1 to 2043 of SEQ ID NO: 54), or at least 2157 nucleotides (e.g., nucleotides 114 to 2271 of SEQ ID NO: 53 or nucleotides 1 to 2157 of SEQ ID NO: 54). In some embodiments, a subsequence contains at least 1077 nucleotides (e.g., nucleotides 324 to 1401 of SEQ ID NO: 53 or nucleotides 243 to 1320 of SEQ ID NO: 54), at least 1140 nucleotides (e.g., nucleotides 261 to 1401 of SEQ ID NO: 53 or nucleotides 180 to 1320 of SEQ ID NO: 54), or at least 1203 nucleotides (e.g., nucleotides 198 to 1401 of SEQ ID NO: 53 or nucleotides 117 to 1320 of SEQ ID NO: 54). In some embodiments, a subsequence contains at least 222 nucleotides (e.g., nucleotides 1464 to 1686 of SEQ ID NO: 53 or nucleotides 1383 to 1605 of SEQ ID NO: 54), at least 237 nucleotides (e.g., nucleotides 1449 to 1686 of SEQ ID NO: 53 or nucleotides 1368 to 1605 of SEQ ID NO: 54), or at least 249 nucleotides (e.g., nucleotides 1437 to 1686 of SEQ ID NO: 53 or nucleotides 1356 to 1605 of SEQ ID NO: 54). In some embodiments, a subsequence contains at least 234 nucleotides (e.g., nucleotides 1728 to 1962 of SEQ ID NO: 53 or nucleotides 1647 to 1881 of SEQ ID NO: 54), at least 246 nucleotides (e.g., nucleotides 1716 to 1962 of SEQ ID NO: 53 or nucleotides 1635 to 1881 of SEQ ID NO: 54), or at least 261 nucleotides (e.g., nucleotides 1701 to 1962 of SEQ ID NO: 53 or nucleotides 1620 to 1881 of SEQ ID NO: 54). In some embodiments, a subsequence contains at least 243 nucleotides (e.g., nucleotides 2007 to 2250 of SEQ ID NO: 53 or nucleotides 1926 to 2169 of SEQ ID NO: 54), at least 258 nucleotides (e.g., nucleotides 1992 to 2250 of SEQ ID NO: 53 or nucleotides 1911 to 2169 of SEQ ID NO: 54), or at least 273 nucleotides (e.g., nucleotides 1977 to 2250 of SEQ ID NO: 53 or nucleotides 1896 to 2169 of SEQ ID NO: 54).

In some embodiments, a subsequence contains at least 1464 nucleotides (e.g., nucleotides 261 to 1725 of SEQ ID NO: 55), at least 1551 nucleotides (e.g., nucleotides 174 to 1725 of SEQ ID NO: 55), or at least 1638 nucleotides (e.g., nucleotides 87 to 1725 of SEQ ID NO: 55). In some embodiments, a subsequence contains at least 1077 nucleotides (e.g., nucleotides 324 to 1401 of SEQ ID NO: 55), at least 1140 nucleotides (e.g., nucleotides 261 to 1401 of SEQ ID NO: 55), or at least 1203 nucleotides (e.g., nucleotides 198 to 1401 of SEQ ID NO: 55). In some embodiments, a subsequence contains at least 237 nucleotides (e.g., nucleotides 1488 to 1707 of SEQ ID NO: 55), at least 252 nucleotides (e.g., nucleotides 1443 to 1707 of SEQ ID NO: 55), or at least 264 nucleotides (e.g., nucleotides 1443 to 1707 of SEQ ID NO: 55).

In some embodiments, a subsequence contains at least 1473 nucleotides (e.g., nucleotides 261 to 1734 of SEQ ID NO: 58), at least 1560 nucleotides (e.g., nucleotides 174 to 1725 of SEQ ID NO: 58), or at least 1647 nucleotides (e.g., nucleotides 87 to 1734 of SEQ ID NO: 58). In some embodiments, a subsequence contains at least 1077 nucleotides (e.g., nucleotides 324 to 1401 of SEQ ID NO: 58), at least 1140 nucleotides (e.g., nucleotides 261 to 1401 of SEQ ID NO: 58), or at 1203 nucleotides (e.g., nucleotides 198 to 1401 of SEQ ID NO: 58). In some embodiments, a subsequence contains at least 237 nucleotides (e.g., nucleotides 1497 to 1716 of SEQ ID NO: 58), at least 252 nucleotides (e.g., nucleotides 1452 to 1716 of SEQ ID NO: 58), or at least 264 nucleotides (e.g., nucleotides 1452 to 1716 of SEQ ID NO: 58).

Trehalase: The term “trehalase” means an enzyme which degrades trehalose into its unit monosaccharides (i.e., glucose). Trehalases are classified in EC 3.2.1.28 (alpha, alpha-trehalase) and EC. 3.2.1.93 (alpha, alpha-phosphotrehalase). The EC classes are based on recommendations of the Nomenclature Committee of the International Union of Biochemistry and Molecular Biology (IUBMB). Description of EC classes can be found on the internet, e.g., on “http://www.expasy.org/enzyme/”. Trehalases are enzymes that catalyze the following reactions:
EC 3.2.1.28: Alpha, alpha-trehalose+H₂O⇔2 D-glucose;
EC 3.2.1. 93: Alpha, alpha-trehalose 6-phosphate+H₂O⇔D-glucose+D-glucose 6-phosphate.

Trehalase activity may be determined according to procedures known in the art.

Variant: The term “variant” means a polypeptide having alpha-amylase activity comprising a man-made mutation, i.e., a substitution, insertion, and/or deletion (e.g., truncation), at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. In some embodiments, the variant includes an insertion of one or more (e.g., several) amino acids, e.g., 1-5 amino acids, adjacent to the amino acid occupying the position.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### DETAILED DESCRIPTION OF THE INVENTION

The present invention relates to polypeptides having alpha-amylase activity, alpha-amylase catalytic domains, and starch binding modules, and polynucleotides encoding the polypeptides, alpha-amylase catalytic domains, and starch binding modules, and to nucleic acid constructs, vectors, and host cells comprising the polynucleotides as well as methods of producing and using the polypeptides, alpha-amylase catalytic domains, and starch binding modules. The present invention relates to processes of producing fermentation products, such as ethanol from starch-containing material using a fermenting organism, wherein a polypeptide having alpha-amylase activity of the present invention is present or added during saccharification, fermentation, or simultaneous saccharification and fermentation.

### Polypeptides Having Alpha-Amylase Activity

In some embodiments, the present invention relates to isolated or purified polypeptides having a sequence identity of at least 92%, at least 93%, at least 94%, at least 95%, at least 96%, at least 97%, at least 98%, at least 99%, or 100% to the mature polypeptide of SEQ ID NO: 2, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 2. In some embodiments, the present invention provides alpha-amylase variants of SEQ ID NO: 2 having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 2, wherein the variant has at least 1, 2, 3, 4, 5, 6, 7, 8, 9, or 10 substitutions, deletions, or insertions at positions corresponding to positions other than positions D223, E247, and D314 of SEQ ID NO: 2, and wherein the variant has alpha-amylase activity.

The polypeptide preferably comprises, consists essentially of, or consists of the amino acid sequence of SEQ ID NO: 2 or the mature polypeptide thereof; or is a fragment thereof having alpha-amylase activity. In one aspect, the mature polypeptide is SEQ ID NO: 3. In one aspect, the mature polypeptide is amino acids 21 to 493 of SEQ ID NO: 2. In one aspect, the mature polypeptide is amino acids 21 to 603 of SEQ ID NO: 2. In one aspect, the mature polypeptide is amino acids 18 to 603 of SEQ ID NO: 2.

In some embodiments, the present invention relates to isolated or purified polypeptides having a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 5, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 5. In some embodiments, the present invention provides alpha-amylase variants of SEQ ID NO: 5 having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 5, wherein the variant has at least 1, 2, 3, 4, 5, 6, 7, 8, 9, or 10 substitutions, deletions, or insertions at positions corresponding to positions other than positions D223, E247, and D314 of SEQ ID NO: 5, and wherein the variant has alpha-amylase activity.

The polypeptide preferably comprises, consists essentially of, or consists of the amino acid sequence of SEQ ID NO: 5 or the mature polypeptide thereof; or is a fragment thereof having alpha-amylase activity. In one aspect, the mature polypeptide is SEQ ID NO: 6. In one aspect, the mature polypeptide is amino acids 18 to 497 of SEQ ID NO: 5. In one aspect, the mature polypeptide is amino acids 18 to 497 of SEQ ID NO: 5., the mature polypeptide is amino acids 18 to 632 of SEQ ID NO: 5.

In some embodiments, the present invention relates to isolated or purified polypeptides having a sequence identity of at least 93%, at least 94%, at least 95%, at least 96%, at least 97%, at least 98%, at least 99%, or 100% to the mature polypeptide of SEQ ID NO: 8, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 8. In some embodiments, the present invention provides alpha-amylase variants of SEQ ID NO: 8 having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 8, wherein the variant has at least 1, 2, 3, 4, 5, 6, 7, 8, 9, or 10 substitutions, deletions, or insertions at positions corresponding to positions other than positions D223, E247, and D316 of SEQ ID NO: 8, and wherein the variant has alpha-amylase activity.

The polypeptide preferably comprises, consists essentially of, or consists of the amino acid sequence of SEQ ID NO: 8 or the mature polypeptide thereof; or is a fragment thereof having alpha-amylase activity. In one aspect, the mature polypeptide is SEQ ID NO: 9. In one aspect, the mature polypeptide is amino acids 22 to 495 of SEQ ID NO: 8. In one aspect, the mature polypeptide is amino acids 22 to 632 of SEQ ID NO: 8.

In some embodiments, the present invention relates to isolated or purified polypeptides having a sequence identity of at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 11, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 11. In some embodiments, the present invention provides alpha-amylase variants of SEQ ID NO: 11 having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 11, wherein the variant has at least 1, 2, 3, 4, 5, 6, 7, 8, 9, or 10 substitutions, deletions, or insertions at positions corresponding to positions other than positions D225, E249, and D316 of SEQ ID NO: 11, and wherein the variant has alpha-amylase activity.

The polypeptide preferably comprises, consists essentially of, or consists of the amino acid sequence of SEQ ID NO: 11 or the mature polypeptide thereof; or is a fragment thereof having alpha-amylase activity. In one aspect, the mature polypeptide is SEQ ID NO: 12. In one aspect, the mature polypeptide is amino acids 20 to 496 of SEQ ID NO: 11. In one aspect, the mature polypeptide is amino acids 22 to 609 of SEQ ID NO: 11.

In some embodiments, the present invention relates to isolated or purified polypeptides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 14, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 14. In some embodiments, the present invention provides alpha-amylase variants of SEQ ID NO: 14 having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 14, wherein the variant has at least 1, 2, 3, 4, 5, 6, 7, 8, 9, or 10 substitutions, deletions, or insertions at positions corresponding to positions other than positions D217, E251, and D312 of SEQ ID NO: 14, and wherein the variant has alpha-amylase activity.

The polypeptide preferably comprises, consists essentially of, or consists of the amino acid sequence of SEQ ID NO: 14 or the mature polypeptide thereof; or is a fragment thereof having alpha-amylase activity. In one aspect, the mature polypeptide is SEQ ID NO: 15. In one aspect, the mature polypeptide is amino acids 30 to 475 of SEQ ID NO: 14.

In some embodiments, the present invention relates to isolated or purified polypeptides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 16, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 16.

The polypeptide preferably comprises, consists essentially of, or consists of the amino acid sequence of SEQ ID NO: 16 or the mature polypeptide thereof; or is a fragment thereof having alpha-amylase activity. In one aspect, the mature polypeptide is SEQ ID NO: 17. In one aspect, the mature polypeptide is amino acids 21-586 of SEQ ID NO: 16.

In some embodiments, the present invention relates to isolated or purified polypeptides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 41, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 41. In some embodiments, the present invention provides alpha-amylase variants of SEQ ID NO: 41 having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 41, wherein the variant has at least 1, 2, 3, 4, 5, 6, 7, 8, 9, or 10 substitutions, deletions, or insertions at positions corresponding to positions other than positions D184, E216, and D277 of SEQ ID NO: 41, and wherein the variant has alpha-amylase activity.

The polypeptide preferably comprises, consists essentially of, or consists of the amino acid sequence of SEQ ID NO: 41 or the mature polypeptide thereof; or is a fragment thereof having alpha-amylase activity. In one aspect, the mature polypeptide is SEQ ID NO: 42. In one aspect, the mature polypeptide is amino acids 28 to 659 of SEQ ID NO: 41.

In some embodiments, the present invention relates to isolated or purified polypeptides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 44, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 44. In some embodiments, the present invention provides alpha-amylase variants of SEQ ID NO: 44 having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 44, wherein the variant has at least 1, 2, 3, 4, 5, 6, 7, 8, 9, or 10 substitutions, deletions, or insertions at positions corresponding to positions other than positions D215, E249, and D310 of SEQ ID NO: 44, and wherein the variant has alpha-amylase activity.

The polypeptide preferably comprises, consists essentially of, or consists of the amino acid sequence of SEQ ID NO: 44 or the mature polypeptide thereof; or is a fragment thereof having alpha-amylase activity. In one aspect, the mature polypeptide is SEQ ID NO: 45. In one aspect, the mature polypeptide is amino acids 28 to 575 of SEQ ID NO: 44.

In some embodiments, the present invention relates to isolated or purified polypeptides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 47, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 47. In some embodiments, the present invention provides alpha-amylase variants of SEQ ID NO: 47 having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 47, wherein the variant has at least 1, 2, 3, 4, 5, 6, 7, 8, 9, or 10 substitutions, deletions, or insertions at positions corresponding to positions other than positions D215, E249, and D310 of SEQ ID NO: 47, and wherein the variant has alpha-amylase activity.

The polypeptide preferably comprises, consists essentially of, or consists of the amino acid sequence of SEQ ID NO: 47 or the mature polypeptide thereof; or is a fragment thereof having alpha-amylase activity. In one aspect, the mature polypeptide is SEQ ID NO: 48. In one aspect, the mature polypeptide is amino acids 28 to 572 of SEQ ID NO: 47.

In some embodiments, the present invention relates to isolated or purified polypeptides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 50, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 50. In some embodiments, the present invention provides alpha-amylase variants of SEQ ID NO: 50 having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 50, wherein the variant has at least 1, 2, 3, 4, 5, 6, 7, 8, 9, or 10 substitutions, deletions, or insertions at positions corresponding to positions other than positions D215, E249, and D310 of SEQ ID NO: 50, and wherein the variant has alpha-amylase activity.

The polypeptide preferably comprises, consists essentially of, or consists of the amino acid sequence of SEQ ID NO: 50 or the mature polypeptide thereof; or is a fragment thereof having alpha-amylase activity. In one aspect, the mature polypeptide is SEQ ID NO: 51. In one aspect, the mature polypeptide is amino acids 28 to 577 of SEQ ID NO: 50.

In some embodiments, the present invention relates to isolated or purified polypeptides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 53, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 53. In some embodiments, the present invention provides alpha-amylase variants of SEQ ID NO: 53 having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 53, wherein the variant has at least 1, 2, 3, 4, 5, 6, 7, 8, 9, or 10 substitutions, deletions, or insertions at positions corresponding to positions other than positions D215, E249, and D310 of SEQ ID NO: 53, and wherein the variant has alpha-amylase activity.

The polypeptide preferably comprises, consists essentially of, or consists of the amino acid sequence of SEQ ID NO: 53 or the mature polypeptide thereof; or is a fragment thereof having alpha-amylase activity. In one aspect, the mature polypeptide is SEQ ID NO: 54. In one aspect, the mature polypeptide is amino acids 28 to 757 of SEQ ID NO: 53.

In some embodiments, the present invention relates to isolated or purified polypeptides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 56, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 56. In some embodiments, the present invention provides alpha-amylase variants of SEQ ID NO: 56 having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 56, wherein the variant has at least 1, 2, 3, 4, 5, 6, 7, 8, 9, or 10 substitutions, deletions, or insertions at positions corresponding to positions other than positions D215, E249, and D310 of SEQ ID NO: 56, and wherein the variant has alpha-amylase activity.

The polypeptide preferably comprises, consists essentially of, or consists of the amino acid sequence of SEQ ID NO: 56 or the mature polypeptide thereof; or is a fragment thereof having alpha-amylase activity. In one aspect, the mature polypeptide is SEQ ID NO: 57. In one aspect, the mature polypeptide is amino acids 28 to 575 of SEQ ID NO: 56.

In some embodiments, the present invention relates to isolated or purified polypeptides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 59, which have alpha-amylase activity. In one aspect, the polypeptides differ by up to 10 amino acids, e.g., 1, 2, 3, 4, 5, 6, 7, 8, 9, or 10, from the mature polypeptide of SEQ ID NO: 59. In some embodiments, the present invention provides alpha-amylase variants of SEQ ID NO: 59 having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 59, wherein the variant has at least 1, 2, 3, 4, 5, 6, 7, 8, 9, or 10 substitutions, deletions, or insertions at positions corresponding to positions other than positions D215, E249, and D310 of SEQ ID NO: 59, and wherein the variant has alpha-amylase activity.

The polypeptide preferably comprises, consists essentially of, or consists of the amino acid sequence of SEQ ID NO: 59 or the mature polypeptide thereof; or is a fragment thereof having alpha-amylase activity. In one aspect, the mature polypeptide is SEQ ID NO: 60. In one aspect, the mature polypeptide is amino acids 28 to 578 of SEQ ID NO: 59.

In some embodiments, the present invention relates to isolated or purified polypeptides having alpha-amylase activity encoded by polynucleotides that hybridize under medium stringency conditions, medium-high stringency conditions, high stringency conditions, or very high stringency conditions with the full-length complement of the mature polypeptide coding sequence of SEQ ID NO: 1, SEQ ID NO: 4, SEQ ID NO: 7, SEQ ID NO: 10, SEQ ID NO: 13, SEQ ID NO: 40, SEQ ID NO: 43, SEQ ID NO: 46, SEQ ID NO: 49, SEQ ID NO: 52, SEQ ID NO: 55, SEQ ID NO: 58, or a subsequence of any thereof, or the cDNA thereof (Sambrook et al., 1989, Molecular Cloning, A Laboratory Manual, 2d edition, Cold Spring Harbor, New York).

The polynucleotide of SEQ ID NO: 1, SEQ ID NO: 4, SEQ ID NO: 7, SEQ ID NO: 10, SEQ ID NO: 13, SEQ ID NO: 40, SEQ ID NO: 43, SEQ ID NO: 46, SEQ ID NO: 49, SEQ ID NO: 52, SEQ ID NO: 55, SEQ ID NO: 58, or a subsequence of any thereof, as well as the mature polypeptide of SEQ ID NO: 2, SEQ ID NO: 5, SEQ ID NO: 8, SEQ ID NO: 11, SEQ ID NO: 14, SEQ ID NO: 41, SEQ ID NO: 44, SEQ ID NO: 47, SEQ ID NO: 50, SEQ ID NO: 53, SEQ ID NO: 56, SEQ ID NO: 59, or a fragment of any thereof, may be used to design nucleic acid probes to identify and clone DNA encoding polypeptides having alpha-amylase activity from strains of different genera or species according to methods well known in the art. Such probes can be used for hybridization with the genomic DNA or cDNA of a cell of interest, following standard Southern blotting procedures, to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having alpha-amylase activity. Genomic or other DNA from such other strains may be separated by agarose or polyacrylamide gel electrophoresis, or other separation techniques. DNA from the libraries or the separated DNA may be transferred to and immobilized on nitrocellulose or another suitable carrier material. To identify a clone or DNA that hybridizes with SEQ ID NO: 1, SEQ ID NO: 4, SEQ ID NO: 7, SEQ ID NO: 10, SEQ ID NO: 13, SEQ ID NO: 40, SEQ ID NO: 43, SEQ ID NO: 46, SEQ ID NO: 49, SEQ ID NO: 52, SEQ ID NO: 55, SEQ ID NO: 58 or a subsequence thereof, the carrier material is used in a Southern blot.

For purposes of the present invention, hybridization indicates that the polynucleotides hybridize to a labeled nucleic acid probe corresponding to (i) SEQ ID NO: 1, SEQ ID NO: 4, SEQ ID NO: 7, SEQ ID NO: 10, SEQ ID NO: 13, SEQ ID NO: 40, SEQ ID NO: 43, SEQ ID NO: 46, SEQ ID NO: 49, SEQ ID NO: 52, SEQ ID NO: 55, or SEQ ID NO: 58; (ii) the mature polypeptide coding sequence of SEQ ID NO: 1, SEQ ID NO: 4, SEQ ID NO: 7, SEQ ID NO: 10, SEQ ID NO: 13, SEQ ID NO: 40, SEQ ID NO: 43, SEQ ID NO: 46, SEQ ID NO: 49, SEQ ID NO: 52, SEQ ID NO: 55, or SEQ ID NO: 58; (iii) the cDNA sequence of SEQ ID NO: 4 or SEQ ID NO: 7; (iv) the full-length complement of any thereof; or (v) a subsequence of any thereof; under medium to very high stringency conditions. Molecules to which the nucleic acid probe hybridizes under these conditions can be detected using, for example, X-ray film or any other detection means known in the art.

In some embodiments, the nucleic acid probe is nucleotides 61 to 1479, nucleotides 1519 to 1809, nucleotides 61 to 1809, or nucleotides 1 to 1809 of SEQ ID NO: 1. In another aspect, the nucleic acid probe is a polynucleotide that encodes the mature polypeptide of SEQ ID NO: 2; or a fragment thereof. In another aspect, the nucleic acid probe is SEQ ID NO: 1. In some embodiments, the nucleic acid probe is nucleotides 52 to 1943, nucleotides 2046 to 2348, nucleotides 52 to 2348, or nucleotides 1 to 2348 of SEQ ID NO: 4. In another aspect, the nucleic acid probe is a polynucleotide that encodes the mature polypeptide of SEQ ID NO: 5; or a fragment thereof. In another aspect, the nucleic acid probe is SEQ ID NO: 4, or the cDNA sequence thereof. In some embodiments, the nucleic acid probe is nucleotides 64 to 1925, nucleotides 2034 to 2336, nucleotides 64 to 2336, or nucleotides 1 to 2336 of SEQ ID NO: 7. In another aspect, the nucleic acid probe is a polynucleotide that encodes the mature polypeptide of SEQ ID NO: 8; or a fragment thereof. In another aspect, the nucleic acid probe is SEQ ID NO: 7, or the cDNA sequence thereof. In some embodiments, the nucleic acid probe is nucleotides 58 to 1488, nucleotides 1504 to 1827, nucleotides 58 to 1827, or nucleotides 1 to 1827 of SEQ ID NO: 10. In another aspect, the nucleic acid probe is a polynucleotide that encodes the mature polypeptide of SEQ ID NO: 11; or a fragment thereof. In another aspect, the nucleic acid probe is SEQ ID NO: 10. In some embodiments, the nucleic acid probe is nucleotides 88 to 1407, nucleotides 1408 to 1428, nucleotides 88 to 1428, or nucleotides 1 to 1428 of SEQ ID NO: 13. In another aspect, the nucleic acid probe is a polynucleotide that encodes the mature polypeptide of SEQ ID NO: 14; or a fragment thereof. In another aspect, the nucleic acid probe is SEQ ID NO: 13. In some embodiments, the nucleic acid probe is nucleotides 1 to 1977, nucleotides 1 to 81, nucleotides 82 to 1977, or nucleotides 82 to 1395 of SEQ ID NO: 40. In another aspect, the nucleic acid probe is a polynucleotide that encodes the mature polypeptide of SEQ ID NO: 41; or a fragment thereof. In another aspect, the nucleic acid probe is SEQ ID NO: 40. In some embodiments, the nucleic acid probe is nucleotides 1 to 1725, nucleotides 1 to 81, nucleotides 82 to 1725, nucleotides 133 to 1401, nucleotides 1402 to 1431, or nucleotides 1432 to 1707 of SEQ ID NO: 43. In another aspect, the nucleic acid probe is a polynucleotide that encodes the mature polypeptide of SEQ ID NO: 44; or a fragment thereof. In another aspect, the nucleic acid probe is SEQ ID NO: 43. In some embodiments, the nucleic acid probe is nucleotides 1 to 1716, nucleotides 1 to 18, nucleotides 82 to 1716, nucleotides 133 to 1401, or nucleotides 1408 to 1695 of SEQ ID NO: 46. In another aspect, the nucleic acid probe is a polynucleotide that encodes the mature polypeptide of SEQ ID NO: 47; or a fragment thereof. In another aspect, the nucleic acid probe is SEQ ID NO: 46. In some embodiments, the nucleic acid probe is nucleotides 1 to 1731, nucleotides 1 to 81, nucleotides 82 to 1731, nucleotides 133 to 1401, nucleotides 1402 to 1422, or nucleotides 1423 to 1713 of SEQ ID NO: 49. In another aspect, the nucleic acid probe is a polynucleotide that encodes the mature polypeptide of SEQ ID NO: 50; or a fragment thereof. In another aspect, the nucleic acid probe is SEQ ID NO: 49. In some embodiments, the nucleic acid probe is nucleotides 1 to 2271, nucleotides 1 to 81, nucleotides 82 to 2271, nucleotides 133 to 1401, nucleotides 1402 to 1422, nucleotides 1423 to 1686, nucleotides 1687 to 1962, or nucleotides 1963 to 2250 of SEQ ID NO: 52. In another aspect, the nucleic acid probe is a polynucleotide that encodes the mature polypeptide of SEQ ID NO: 53; or a fragment thereof. In another aspect, the nucleic acid probe is SEQ ID NO: 52. In another aspect, the nucleic acid probe is a polynucleotide that encodes the mature polypeptide of SEQ ID NO: 56; or a fragment thereof. In another aspect, the nucleic acid probe is SEQ ID NO: 55. In another aspect, the nucleic acid probe is a polynucleotide that encodes the mature polypeptide of SEQ ID NO: 59; or a fragment thereof. In another aspect, the nucleic acid probe is SEQ ID NO: 58.

In some embodiments, the present invention relates to isolated polypeptides having alpha-amylase activity encoded by polynucleotides having a sequence identity of at least 92%, at least 93%, at least 94%, at least 95%, at least 96%, at least 97%, at least 98%, at least 99%, or 100% to the mature polypeptide coding sequence of SEQ ID NO: 1. In some embodiments, the present invention relates to isolated polypeptides having alpha-amylase activity encoded by polynucleotides having a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 4, or the cDNA sequence thereof. In some embodiments, the present invention relates to isolated polypeptides having alpha-amylase activity encoded by polynucleotides having a sequence identity of at least 93%, at least 94%, at least 95%, at least 96%, at least 97%, at least 98%, at least 99%, or 100% to the mature polypeptide coding sequence of SEQ ID NO: 7, or the cDNA sequence thereof. In some embodiments, the present invention relates to isolated polypeptides having alpha-amylase activity encoded by polynucleotides having a sequence identity of at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 10. In some embodiments, the present invention relates to isolated polypeptides having alpha-amylase activity encoded by polynucleotides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 13. In some embodiments, the present invention relates to isolated polypeptides having alpha-amylase activity encoded by polynucleotides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 40. In some embodiments, the present invention relates to isolated polypeptides having alpha-amylase activity encoded by polynucleotides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 43. In some embodiments, the present invention relates to isolated polypeptides having alpha-amylase activity encoded by polynucleotides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 46. In some embodiments, the present invention relates to isolated polypeptides having alpha-amylase activity encoded by polynucleotides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 49. In some embodiments, the present invention relates to isolated polypeptides having alpha-amylase activity encoded by polynucleotides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 52. In some embodiments, the present invention relates to isolated polypeptides having alpha-amylase activity encoded by polynucleotides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 55. In some embodiments, the present invention relates to isolated polypeptides having alpha-amylase activity encoded by polynucleotides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 58.

The polynucleotide encoding the polypeptide preferably comprises, consists essentially of, or consists of nucleotides 61 to 1809 of SEQ ID NO: 1. The polynucleotide encoding the polypeptide preferably comprises, consists essentially of, or consists of nucleotides 53 to 2348 of SEQ ID NO: 4, or the cDNA sequence thereof. The polynucleotide encoding the polypeptide preferably comprises, consists essentially of, or consists of nucleotides 64 to 2336 of SEQ ID NO: 7, or the cDNA sequence thereof. The polynucleotide encoding the polypeptide preferably comprises, consists essentially of, or consists of nucleotides 58 to 1827 of SEQ ID NO: 10. The polynucleotide encoding the polypeptide preferably comprises, consists essentially of, or consists of nucleotides 88 to 1407 of SEQ ID NO: 13. The polynucleotide encoding the polypeptide preferably comprises, consists essentially of, or consists of nucleotides 82 to 1977 of SEQ ID NO: 40. The polynucleotide encoding the polypeptide preferably comprises, consists essentially of, or consists of nucleotides 82 to 1725 of SEQ ID NO: 43. The polynucleotide encoding the polypeptide preferably comprises, consists essentially of, or consists of nucleotides 82 to 1716 of SEQ ID NO: 46. The polynucleotide encoding the polypeptide preferably comprises, consists essentially of, or consists of nucleotides 82 to 1731 of SEQ ID NO: 49. The polynucleotide encoding the polypeptide preferably comprises, consists essentially of, or consists of nucleotides 82 to 2271 of SEQ ID NO: 52. The polynucleotide encoding the polypeptide preferably comprises, consists essentially of, or consists of nucleotides 82 to 1728 of SEQ ID NO: 55. The polynucleotide encoding the polypeptide preferably comprises, consists essentially of, or consists of nucleotides 82 to 1737 of SEQ ID NO: 58.

In some embodiments, the present invention relates to a polypeptide derived from a mature polypeptide of SEQ ID NO: 2, SEQ ID NO: 5, SEQ ID NO: 8, SEQ ID NO: 11, SEQ ID NO: 14, SEQ ID NO: 16, SEQ ID NO: 41, SEQ ID NO: 44, SEQ ID NO: 47, SEQ ID NO: 50, SEQ ID NO: 53, SEQ ID NO: 56, or SEQ ID NO: 59 by substitution, deletion or addition of one or several amino acids in the mature polypeptide of SEQ ID NO: 2, SEQ ID NO: 5, SEQ ID NO: 8, SEQ ID NO: 11, SEQ ID NO: 14, SEQ ID NO: 16, SEQ ID NO: 41, SEQ ID NO: 44, SEQ ID NO: 47, SEQ ID NO: 50, SEQ ID NO: 53, SEQ ID NO: 56, or SEQ ID NO: 59. In some embodiments, the present invention relates to variants of the mature polypeptide of SEQ ID NO: 2, SEQ ID NO: 5, SEQ ID NO: 8, SEQ ID NO: 11, SEQ ID NO: 14, SEQ ID NO: 16, SEQ ID NO: 41, SEQ ID NO: 44, SEQ ID NO: 47, SEQ ID NO: 50, SEQ ID NO: 53, SEQ ID NO: 56, or SEQ ID NO: 59 comprising a substitution, deletion, and/or insertion at one or more (e.g., several) positions. In one aspect, the number of amino acid substitutions, deletions and/or insertions introduced into the mature polypeptide of SEQ ID NO: 2, SEQ ID NO: 5, SEQ ID NO: 8, SEQ ID NO: 11, SEQ ID NO: 14, SEQ ID NO: 16, SEQ ID NO: 41, SEQ ID NO: 44, SEQ ID NO: 47, SEQ ID NO: 50, SEQ ID NO: 53, SEQ ID NO: 56, or SEQ ID NO: 59 is up to 10, e.g., 1, 2, 3, 4, 5, 6, 7, 8, 9, or 10.

In one aspect, a variant of the mature polypeptide of SEQ ID NO: 2 comprises:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 2; and
(b) at least 1, 2, 3, 4, 5, 6, 7, 8, 9, or 10 substitutions, deletions, or insertions at positions corresponding to positions other than positions D223, E247, and D314 of SEQ ID NO: 2; and wherein the variant has alpha-amylase activity. In an embodiment, the variant of the mature polypeptide of SEQ ID NO: 2 comprises at least 1, 2, 3, 4, 5, 6, 7, 8, 9, or 10 conservative substitutions at positions corresponding to positions other than D223, E247, and D314 of SEQ ID NO: 2, wherein the variant has alpha-amylase activity.

In one aspect, a variant of the mature polypeptide of SEQ ID NO: 5 comprises:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 5; and
(b) at least 1, 2, 3, 4, 5, 6, 7, 8, 9, or 10 substitutions, deletions, or insertions at positions corresponding to positions other than positions D223, E247, and D314 of SEQ ID NO: 5; and wherein the variant has alpha-amylase activity. In an embodiment, the variant of the mature polypeptide of SEQ ID NO: 5 comprises at least 1, 2, 3, 4, 5, 6, 7, 8, 9, or 10 conservative substitutions at positions corresponding to positions other than D223, E247, and D314 of SEQ ID NO: 5, wherein the variant has alpha-amylase activity.

In one aspect, a variant of the mature polypeptide of SEQ ID NO: 8 comprises:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 8; and
(b) at least 1, 2, 3, 4, 5, 6, 7, 8, 9, or 10 substitutions, deletions, or insertions at positions corresponding to positions other than positions D223, E247, and D314 of SEQ ID NO: 8; and wherein the variant has alpha-amylase activity. In an embodiment, the variant of the mature polypeptide of SEQ ID NO: 8 comprises at least 1, 2, 3, 4, 5, 6, 7, 8, 9, or 10 conservative substitutions at positions corresponding to positions other than D223, E247, and D314 of SEQ ID NO: 8, wherein the variant has alpha-amylase activity.

In one aspect, a variant of the mature polypeptide of SEQ ID NO: 11 comprises:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 11; and
(b) at least 1, 2, 3, 4, 5, 6, 7, 8, 9, or 10 substitutions, deletions, or insertions at positions corresponding to positions other than positions D225, E249, and D316 of SEQ ID NO: 11; and wherein the variant has alpha-amylase activity. In an embodiment, the variant of the mature polypeptide of SEQ ID NO: 11 comprises at least 1, 2, 3, 4, 5, 6, 7, 8, 9, or 10 conservative substitutions at positions corresponding to positions other than D225, E249, and D316 of SEQ ID NO: 11, wherein the variant has alpha-amylase activity.

In one aspect, a variant of the mature polypeptide of SEQ ID NO: 14 comprises:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 14; and
(b) at least 1, 2, 3, 4, 5, 6, 7, 8, 9, or 10 substitutions, deletions, or insertions at positions corresponding to positions other than positions D217, E251, and D312 of SEQ ID NO: 14; and wherein the variant has alpha-amylase activity. In an embodiment, the variant of the mature polypeptide of SEQ ID NO: 14 comprises at least 1, 2, 3, 4, 5, 6, 7, 8, 9, or 10 conservative substitutions at positions corresponding to positions other than D217, E251, and D312 of SEQ ID NO: 14, wherein the variant has alpha-amylase activity.

In one aspect, a variant of the mature polypeptide of SEQ ID NO: 41 comprises:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 41; and
(b) at least 1, 2, 3, 4, 5, 6, 7, 8, 9, or 10 substitutions, deletions, or insertions at positions corresponding to positions other than positions D184, E216, and D277 of SEQ ID NO: 41; and wherein the variant has alpha-amylase activity. In an embodiment, the variant of the mature polypeptide of SEQ ID NO: 41 comprises at least 1, 2, 3, 4, 5, 6, 7, 8, 9, or 10 conservative substitutions at positions corresponding to positions other than D184, E216, and D277 of SEQ ID NO: 41, wherein the variant has alpha-amylase activity.

In one aspect, a variant of the mature polypeptide of SEQ ID NO: 44 comprises:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 44; and
(b) at least 1, 2, 3, 4, 5, 6, 7, 8, 9, or 10 substitutions, deletions, or insertions at positions corresponding to positions other than positions D215, E249, and D310 of SEQ ID NO: 44; and wherein the variant has alpha-amylase activity. In an embodiment, the variant of the mature polypeptide of SEQ ID NO: 44 comprises at least 1, 2, 3, 4, 5, 6, 7, 8, 9, or 10 conservative substitutions at positions corresponding to positions other than D215, E249, and D310 of SEQ ID NO: 44, wherein the variant has alpha-amylase activity.

In one aspect, a variant of the mature polypeptide of SEQ ID NO: 47 comprises:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 47; and
(b) at least 1, 2, 3, 4, 5, 6, 7, 8, 9, or 10 substitutions, deletions, or insertions at positions corresponding to positions other than positions D215, E249, and D310 of SEQ ID NO: 47; and wherein the variant has alpha-amylase activity. In an embodiment, the variant of the mature polypeptide of SEQ ID NO: 47 comprises at least 1, 2, 3, 4, 5, 6, 7, 8, 9, or 10 conservative substitutions at positions corresponding to positions other than D215, E249, and D310 of SEQ ID NO: 47, wherein the variant has alpha-amylase activity.

In one aspect, a variant of the mature polypeptide of SEQ ID NO: 50 comprises:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 50; and
(b) at least 1, 2, 3, 4, 5, 6, 7, 8, 9, or 10 substitutions, deletions, or insertions at positions corresponding to positions other than positions D215, E249, and D310 of SEQ ID NO: 50; and wherein the variant has alpha-amylase activity. In an embodiment, the variant of the mature polypeptide of SEQ ID NO: 50 comprises at least 1, 2, 3, 4, 5, 6, 7, 8, 9, or 10 conservative substitutions at positions corresponding to positions other than D215, E249, and D310 of SEQ ID NO: 50, wherein the variant has alpha-amylase activity.

In one aspect, a variant of the mature polypeptide of SEQ ID NO: 53 comprises:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 53; and
(b) at least 1, 2, 3, 4, 5, 6, 7, 8, 9, or 10 substitutions, deletions, or insertions at positions corresponding to positions other than positions D215, E249, and D310 of SEQ ID NO: 53; and wherein the variant has alpha-amylase activity. In an embodiment, the variant of the mature polypeptide of SEQ ID NO: 53 comprises at least 1, 2, 3, 4, 5, 6, 7, 8, 9, or 10 conservative substitutions at positions corresponding to positions other than D215, E249, and D310 of SEQ ID NO: 53, wherein the variant has alpha-amylase activity.

In one aspect, a variant of the mature polypeptide of SEQ ID NO: 56 comprises:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 56; and
(b) at least 1, 2, 3, 4, 5, 6, 7, 8, 9, or 10 substitutions, deletions, or insertions at positions corresponding to positions other than positions D215, E249, and D310 of SEQ ID NO: 56; and wherein the variant has alpha-amylase activity. In an embodiment, the variant of the mature polypeptide of SEQ ID NO: 56 comprises at least 1, 2, 3, 4, 5, 6, 7, 8, 9, or 10 conservative substitutions at positions corresponding to positions other than D215, E249, and D310 of SEQ ID NO: 56, wherein the variant has alpha-amylase activity.

In one aspect, a variant of the mature polypeptide of SEQ ID NO: 59 comprises:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 59; and
(b) at least 1, 2, 3, 4, 5, 6, 7, 8, 9, or 10 substitutions, deletions, or insertions at positions corresponding to positions other than positions D215, E249, and D310 of SEQ ID NO: 59; and wherein the variant has alpha-amylase activity. In an embodiment, the variant of the mature polypeptide of SEQ ID NO: 59 comprises at least 1, 2, 3, 4, 5, 6, 7, 8, 9, or 10 conservative substitutions at positions corresponding to positions other than D215, E249, and D310 of SEQ ID NO: 59, wherein the variant has alpha-amylase activity.

In an embodiment, the polypeptide has an N-terminal extension and/or C-terminal extension of 1-10 amino acids, e.g., 1, 2, 3, 4, 5, 6, 7, 8, 9, or 10 amino acids. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for alpha-amylase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide. Essential amino acids in the polypeptides having alpha-amylase activity of the present invention are shown in Table 1 below:

TABLE 1
Active Site and Catalytic Residues of Polypeptides
Having Alpha-Amylase Activity

| | Position of | Position of | Position of Stabilizer |
|---|---|---|---|
| Alpha-amylase | Nucleophile | Acid-base | of Transition State |
| SEQ ID NO: 2 | D223 | E247 | D314 |
| SEQ ID NO: 5 | D223 | E247 | D314 |
| SEQ ID NO: 8 | D223 | E247 | D314 |
| SEQ ID NO: 11 | D225 | E249 | D316 |
| SEQ ID NO: 14 | D217 | E251 | D312 |
| SEQ ID NO: 41 | D184 | E216 | D277 |
| SEQ ID NO: 44 | D215 | E249 | D310 |
| SEQ ID NO: 47 | D215 | E249 | D310 |
| SEQ ID NO: 50 | D215 | E249 | D310 |
| SEQ ID NO: 53 | D215 | E249 | D310 |
| SEQ ID NO: 56 | D215 | E249 | D310 |
| SEQ ID NO: 59 | D215 | E249 | D310 |

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

In some embodiments, the polypeptide is a fragment containing at least 512 amino acid residues (e.g., amino acids 1 to 512 of the mature polypeptide of SEQ ID NO: 2), at least 542 amino acid residues (e.g., amino acids 1 to 542 of the mature polypeptide of SEQ ID NO: 2), or at least 572 amino acid residues (e.g., amino acids 1 to 572 of the mature polypeptide of SEQ ID NO: 2). In some embodiments, the polypeptide is a fragment containing at least 401 amino acid residues (e.g., amino acids 21 to 402 of the mature polypeptide of SEQ ID NO: 2), at least 424 amino acid residues (e.g., amino acids 21 to 445 of the mature polypeptide of SEQ ID NO: 2), or at least 448 amino acid residues (e.g., amino acids 21 to 469 of the mature polypeptide of SEQ ID NO: 2). In some embodiments, the polypeptide is a fragment containing at least 81 amino acid residues (e.g., amino acids 507 to 588 of the mature polypeptide of SEQ ID NO: 2), at least 86 amino acid residues (e.g., amino acids 507 to 593 of the mature polypeptide of SEQ ID NO: 2), or at least 91 amino acid residues (e.g., amino acids 507 to 598 of the mature polypeptide of SEQ ID NO: 2).

In some embodiments, the polypeptide is a fragment contains at least 537 amino acid residues (e.g., amino acids 1 to 537 of the mature polypeptide of SEQ ID NO: 5), at least 568 amino acid residues (e.g., amino acids 1 to 568 of the mature polypeptide of SEQ ID NO: 5), or at least 600 amino acid residues (e.g., amino acids 1 to 600 of the mature polypeptide of SEQ ID NO: 5). In some embodiments, the polypeptide is a fragment containing at least 407 amino acid residues (e.g., amino acids 18 to 425 of the mature polypeptide of SEQ ID NO: 5), at least 431 amino acid residues (e.g., amino acids 18 to 449 of the mature polypeptide of SEQ ID NO: 5), or at least 455 amino acid residues (e.g., amino acids 18 to 473 of the mature polypeptide of SEQ ID NO: 5). In some embodiments, the polypeptide is a fragment containing at least 85 amino acid residues (e.g., amino acids 532 to 617 of the mature polypeptide of SEQ ID NO: 5), at least 90 amino acid residues (e.g., amino acids 532 to 622 of the mature polypeptide of SEQ ID NO: 5), or at least 95 amino acid residues (e.g., amino acids 532 to 627 of the mature polypeptide of SEQ ID NO: 5).

In some embodiments, the polypeptide is a fragment containing at least 537 amino acid residues (e.g., amino acids 1 to 537 of the mature polypeptide of SEQ ID NO: 8), at least 568 amino acid residues (e.g., amino acids 1 to 568 of the mature polypeptide of SEQ ID NO: 8), or at least 600 amino acid residues (e.g., amino acids 1 to 600 of the mature polypeptide of SEQ ID NO: 8). In some embodiments, the polypeptide is a fragment containing at least 402 amino acid residues (e.g., amino acids 22 to 424 of the mature polypeptide of SEQ ID NO: 8), at least 425 amino acid residues (e.g., amino acids 22 to 447 of the mature polypeptide of SEQ ID NO: 8), or at least 449 amino acid residues (e.g., amino acids 22 to 471 of the mature polypeptide of SEQ ID NO: 8). In some embodiments, the polypeptide is a fragment containing at least 85 amino acid residues (e.g., amino acids 532 to 617 of the mature polypeptide of SEQ ID NO: 8), at least 90 amino acid residues (e.g., amino acids 532 to 622 of the mature polypeptide of SEQ ID NO: 8), or at least 95 amino acid residues (e.g., amino acids 532 to 627 of the mature polypeptide of SEQ ID NO: 8).

In some embodiments, the polypeptide is a fragment containing at least 517 amino acid residues (e.g., amino acids 1 to 517 of the mature polypeptide of SEQ ID NO: 11), at least 548 amino acid residues (e.g., amino acids 1 to 548 of SEQ ID NO: 11), or at least 578 amino acid residues (e.g., amino acids 1 to 578 of the mature polypeptide of SEQ ID NO: 11). In some embodiments, the polypeptide is a fragment containing at least 404 amino acid residues (e.g., amino acids 20 to 424 of the mature polypeptide of SEQ ID NO: 11), at least 428 amino acid residues (e.g., amino acids 20 to 448 of SEQ ID NO: 11), or at least 452 amino acid residues (e.g., amino acids 20 to 472 of the mature polypeptide of SEQ ID NO: 11). In some embodiments, the polypeptide is a fragment containing at least 79 amino acid residues (e.g., amino acids 508 to 587 of the mature polypeptide of SEQ ID NO: 11), at least 83 amino acid residues (e.g., amino acids 508 to 591 of the mature polypeptide of SEQ ID NO: 11), or at least 88 amino acid residues (e.g., amino acids 508 to 596 of the mature polypeptide of SEQ ID NO: 11).

In some embodiments, the polypeptide is a fragment containing at least 403 amino acid residues (e.g., amino acids 1 to 403 of the mature polypeptide of SEQ ID NO: 14), at least 427 amino acid residues (e.g., amino acids 1 to 427 of the mature polypeptide of SEQ ID NO: 14), or at least 451 amino acid residues (e.g., amino acids 1 to 451 of the mature polypeptide of SEQ ID NO: 14). In some embodiments, the polypeptide is a fragment containing at least 373 amino acid residues (e.g., amino acids 30 to 403 of the mature polypeptide of SEQ ID NO: 14), at least 395 amino acid residues (e.g., amino acids 30 to 425 of the mature polypeptide of SEQ ID NO: 14), or at least 417 amino acid residues (e.g., amino acids 30 to 447 of the mature polypeptide of SEQ ID NO: 14).

In some embodiment, the polypeptide is fragment a containing at least 560 amino acid residues (e.g., amino acids 1 to 560 of the mature polypeptide of SEQ ID NO: 41), at least 593 amino acid residues (e.g., amino acids 1 to 593 of the mature polypeptide of SEQ ID NO: 41), or at least 626 residues (e.g., amino acids 1 to 626 of the mature polypeptide of SEQ ID NO: 41). In some embodiments, the polypeptide is a fragment containing at least 371 amino acid residues (e.g., amino acids 28 to 399 of the mature polypeptide of SEQ ID NO: 41), at least 393 amino acid residues (e.g., amino acids 28 to 421 of the mature polypeptide of SEQ ID NO: 41), or at least 415 amino acid residues (e.g., amino acids 28 to 443 of the mature polypeptide of SEQ ID NO: 41). In some embodiments, the polypeptide is a fragment containing at least 84 amino acid residues (e.g., amino acids 554 to 638 of the mature polypeptide of SEQ ID NO: 41), at least 89 amino acid residues (e.g., amino acids 554 to 643 of the mature polypeptide of SEQ ID NO: 41), or at least 94 amino acid residues (e.g., amino acids 554 to 649 of the mature polypeptide of SEQ ID NO: 41). In some embodiments, the polypeptide is a fragment containing at least 488 amino acids (e.g., amino acids 87 to 575 of SEQ ID NO: 44 or amino acids 1 to 488 of SEQ ID NO: 45), at least 517 amino acids (e.g., amino acids 58 to 575 of SEQ ID NO: 44 or amino acids 1 to 517 of SEQ ID NO: 45), or at least 546 amino acids (e.g., 29 to 575 of SEQ ID NO: 44 or amino acids 1 to 546 of SEQ ID NO: 45). In some embodiments, the polypeptide is a fragment containing at least 359 amino acids (e.g., amino acids 108 to 467 of SEQ ID NO: 44 or amino acids 81 to 440 of SEQ ID NO: 45), at least 380 amino acids (e.g., amino acids 87 to 467 of SEQ ID NO: 44 or amino acids 60 to 440 of SEQ ID NO: 45), or at least 401 amino acids (e.g., amino acids 66 to 467 of SEQ ID NO: 44 or amino acids 39 to 440 of SEQ ID NO: 45). In some embodiments, the polypeptide is a fragment containing at least 79 amino acids (e.g., amino acids 490 to 569 of SEQ ID NO: 44 or amino acids 463 to 542 of SEQ ID NO: 45), at least 82 amino acids (e.g., amino acids 487 to 569 of SEQ ID NO: 44 or amino acids 460 to 542 of SEQ ID NO: 45), or at least 87 amino acids (e.g., amino acids 482 to 569 of SEQ ID NO: 44 or amino acids 455 to 542 of SEQ ID NO: 45). In some embodiments, the polypeptide is a fragment containing at least 486 amino acids (e.g., amino acids 86 to 572 of SEQ ID NO: 47 or amino acids 1 to 486 of SEQ ID NO: 48), at least 514 amino acids (e.g., amino acids 58 to 572 of SEQ ID NO: 47 or amino acids 1 to 514 of SEQ ID NO: 48), or at least 543 amino acids (e.g., amino acids 29 to 572 of SEQ ID NO: 47 or amino acids 1 to 572 of SEQ ID NO: 48). In some embodiments, the polypeptide is a fragment containing at least 359 amino acids (e.g., amino acids 108 to 467 of SEQ ID NO: 47 or amino acids 81 to 440 of SEQ ID NO: 48), at least 380 amino acids (e.g., amino acids 87 to 467 of SEQ ID NO: 47 or amino acids 60 to 440 of SEQ ID NO: 48), or at least 401 amino acids (e.g., amino acids 66 to 467 of SEQ ID NO: 47 or amino acids 39 to 440 of SEQ ID NO: 48). In some embodiments, the polypeptide is a fragment containing at least 81 amino acids (e.g., amino acids 484 to 565 of SEQ ID NO: 47 or amino acids 457 to 538 of SEQ ID NO: 48), at least 86 amino acids (e.g., amino acids 479 to 565 of SEQ ID NO: 47 or amino acids 460 to 538 of SEQ ID NO: 48), or at least 91 amino acids (e.g., amino acids 474 to 565 of SEQ ID NO: 47 or amino acids 447 to 538 of SEQ ID NO: 48). In some embodiments, the polypeptide is a fragment containing at least 490 amino acids (e.g., amino acids 87 to 577 of SEQ ID NO: 50 or amino acids 1 to 490 of SEQ ID NO: 51), at least 519 amino acids (e.g., amino acids 58 to 577 of SEQ ID NO: 50 or amino acids 1 to 519 of SEQ ID NO: 51), or at least 548 amino acids (e.g., amino acids 29 to 577 of SEQ ID NO: 50 or amino acids 1 to 548 of SEQ ID NO: 51). In some embodiments, the polypeptide is a fragment containing at least 359 amino acids (e.g., amino acids 108 to 467 of SEQ ID NO: 50 or amino acids 81 to 440 of SEQ ID NO: 51), at least 380 amino acids (e.g., amino acids 87 to 467 of SEQ ID NO: 50 or amino acids 60 to 440 of SEQ ID NO: 51), or at least 401 amino acids (e.g., amino acids 66 to 467 of SEQ ID NO: 50 or amino acids 39 to 440 of SEQ ID NO: 51). In some embodiments, the polypeptide is a fragment containing at least 81 amino acids (e.g., amino acids 490 to 571 of SEQ ID NO: 50 or amino acids 463 to 544 of SEQ ID NO: 51), at least 86 amino acids (e.g., amino acids 485 to 571 of SEQ ID NO: 50 or amino acids 460 to 544 of SEQ ID NO: 51), or at least 91 amino acids (e.g., amino acids 480 to 571 of SEQ ID NO: 50 or amino acids 447 to 544 of SEQ ID NO: 51). In some embodiments, the polypeptide is a fragment containing at least 643 amino acids (e.g., amino acids 114 to 757 of SEQ ID NO: 53 or amino acids 1 to 643 of SEQ ID NO: 54), at least 681 amino acids (e.g., amino acids 76 to 757 of SEQ ID NO: 53 or amino acids 1 to 681 of SEQ ID NO: 54), or at least 719 amino acids (e.g., amino acids 38 to 757 of SEQ ID NO: 53 or amino acids 1 to 719 of SEQ ID NO: 54). In some embodiments, the polypeptide is a fragment containing at least 359 amino acids (e.g., amino acids 108 to 467 of SEQ ID NO: 53 or amino acids 81 to 440 of SEQ ID NO: 54), at least 380 amino acids (e.g., amino acids 87 to 467 of SEQ ID NO: 53 or amino acids 60 to 440 of SEQ ID NO: 54), or at least 401 amino acids (e.g., amino acids 66 to 467 of SEQ ID NO: 53 or amino acids 39 to 440 of SEQ ID NO: 54). In some embodiments, the polypeptide is a fragment containing at least 74 amino acids (e.g., amino acids 488 to 562 of SEQ ID NO: 53 or amino acids 461 to 535 of SEQ ID NO: 54), at least 79 amino acids (e.g., amino acids 483 to 562 of SEQ ID NO: 53 or amino acids 456 to 535 of SEQ ID NO: 54), or at least 83 amino acids (e.g., amino acids 479 to 562 of SEQ ID NO: 53 or amino acids 452 to 535 of SEQ ID NO: 54). In some embodiments, the polypeptide is a fragment containing at least 78 amino acids (e.g., amino acids 576 to 654 of SEQ ID NO: 53 or amino acids 549 to 627 of SEQ ID NO: 54), at least 82 amino acids (e.g., amino acids 572 to 654 of SEQ ID NO: 53 or amino acids 545 to 627 of SEQ ID NO: 54), or at least 87 amino acids (e.g., amino acids 567 to 654 of SEQ ID NO: 53 or amino acids 540 to 627 of SEQ ID NO: 54). In some embodiments, the polypeptide is a fragment containing at least 81 amino acids (e.g., amino acids 669 to 750 of SEQ ID NO: 53 or amino acids 642 to 723 of SEQ ID NO: 54), at least 86 amino acids (e.g., amino acids 664 to 750 of SEQ ID NO: 53 or amino acids 637 to 723 of SEQ ID NO: 54), or at least 91 amino acids (e.g., amino acids 659 to 750 of SEQ ID NO: 53 or amino acids 632 to 723 of SEQ ID NO: 54).

In some embodiments, the polypeptide is a fragment containing at least 488 amino acids (e.g., amino acids 87 to 575 of SEQ ID NO: 56 or amino acids 1 to 488 of SEQ ID NO: 57), at least 517 amino acids (e.g., amino acids 58 to 575 of SEQ ID NO: 56 or amino acids 1 to 517 of SEQ ID NO: 57), or at least 546 amino acids (e.g., amino acids 29 to 575 of SEQ ID NO: 56 or amino acids 1 to 546 of SEQ ID NO: 57). In some embodiments, the polypeptide is a fragment containing at least 359 amino acids (e.g., amino acids 108 to 467 of SEQ ID NO: 56 or amino acids 81 to 440 of SEQ ID NO: 57), at least 380 amino acids (e.g., amino acids 87 to 467 of SEQ ID NO: 56 or amino acids 60 to 440 of SEQ ID NO: 57), or at least 401 amino acids (e.g., amino acids 66 to 467 of SEQ ID NO: 56 or amino acids 39 to 440 of SEQ ID NO: 57). In some embodiments, the polypeptide is a fragment containing at least 79 amino acids (e.g., amino acids 496 to 569 of SEQ ID NO: 56 or amino acids 463 to 542 of SEQ ID NO: 57), at least 84 amino acids (e.g., amino acids 481 to 569 of SEQ ID NO: 56 or amino acids 458 to 542 of SEQ ID NO: 57), or at least 88 amino acids (e.g., amino acids 481 to 569 of SEQ ID NO: 56 or amino acids 454 to 542 of SEQ ID NO: 57).

In some embodiments, the polypeptide is a fragment containing at least 491 amino acids (e.g., amino acids 87 to 578 of SEQ ID NO: 59 or amino acids 1 to 491 of SEQ ID NO: 60), at least 520 amino acids (e.g., amino acids 58 to 578 of SEQ ID NO: 59 or amino acids 1 to 520 of SEQ ID NO: 60), or at least 549 amino acids (e.g., amino acids 29 to 578 of SEQ ID NO: 59 or amino acids 1 to 549 of SEQ ID NO: 60). In some embodiments, the polypeptide is a fragment containing at least 359 amino acids (e.g., amino acids 108 to 467 of SEQ ID NO: 59 or amino acids 81 to 440 of SEQ ID NO: 60), at least 380 amino acids (e.g., amino acids 87 to 467 of SEQ ID NO: 59 or amino acids 60 to 440 of SEQ ID NO: 60), or at least 401 amino acids (e.g., amino acids 66 to 467 of SEQ ID NO: 59 or amino acids 39 to 440 of SEQ ID NO: 60). In some embodiments, the polypeptide is a fragment containing at least 79 amino acids (e.g., amino acids 499 to 572 of SEQ ID NO: 59 or amino acids 466 to 545 of SEQ ID NO: 60), at least 84 amino acids (e.g., amino acids 484 to 572 of SEQ ID NO: 59 or amino acids 461 to 545 of SEQ ID NO: 60), or at least 88 amino acids (e.g., amino acids 484 to 572 of SEQ ID NO: 59 or amino acids 457 to 545 of SEQ ID NO: 60).

The polypeptide may be a hybrid polypeptide or a fusion polypeptide. The hybrid polypeptide or fusion polypeptide comprises, consists essentially of, or consists of a catalytic domain of the present invention, or polypeptide of the present invention and a starch binding module of the present invention, optionally joined by a linker.

The polypeptides of the present invention have improved activity on starch, for instance corn starch.

The polypeptides of the present invention have improved stability at low pH (e.g., acidic, e.g., less than 5.0), in particular the polypeptides of the present invention have improved stability at about pH 4.0 compared to SEQ ID NO: 41.

The polypeptides of the present invention retain greater than about 75% of their activity on starch (e.g., corn starch) at low pH (e.g., less than about 5.0, preferably less than about 4.0). In some embodiments, the polypeptides of the present invention retain about at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of their residual activity at pH of less than or equal to 5.0, less than or equal to 4.9, less than or equal to 4.8, less than or equal to 4.7, less than or equal to 4.6, less than or equal to 4.5, less than or equal to 4.4, less than or equal to 4.3, less than or equal to 4.2, less than or equal to 4.1, or less than or equal to 4.0.

### Sources of Polypeptides Having Alpha-Amylase Activity

A polypeptide having alpha-amylase activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly. In an embodiment, the polypeptide having alpha-amylase activity is of fungal origin. In an embodiment, the polypeptide having alpha-amylase activity is of bacterial origin.

In another aspect, the polypeptide having alpha-amylase activity of the present invention may be obtained from microorganisms of the genus Penicillium, e.g., a polypeptide obtained from Penicillium oxalicum, Penicillum sclerotiorum, or Penicillium wotroi, or of the genus Talaromyces, e.g., a polypeptide obtained from Talaromyces helicus, of the genus Lactobacillus, e.g., a polypeptide obtained from Lactobacillus amylovorous, of the genus Valsaria, e.g., a polypeptide obtained from Valsaria rubricosa, or of the genus Bacillus, e.g., a polypeptide obtained from Bacillus amyloliquefaciens.

In an aspect, the polypeptide having alpha-amylase activity is a Penicillium oxalicum polypeptide, for instance, the Penicillium oxalicum polypeptide having alpha-amylase activity of SEQ ID NO: 2 or SEQ ID NO: 3, or a polypeptide having at least 92%, at least 93%, at least 94%, at least 95%, at least 96%, at least 97%, at least 98%, at least 99% amino acid sequence identity to SEQ ID NO: 2 or SEQ ID NO: 3.

In an aspect, the polypeptide having alpha-amylase activity is a Penicillium sclerotiorum polypeptide, for instance, the Penicillium sclerotiorum polypeptide having alpha-amylase activity of SEQ ID NO: 5 or SEQ ID NO: 6, or a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 5 or SEQ ID NO: 6.

In an aspect, the polypeptide having alpha-amylase activity is a Penicillium wotroi polypeptide, for instance, the Penicillium wotroi polypeptide having alpha-amylase activity of SEQ ID NO: 8 or SEQ ID NO: 9, or a polypeptide having at least 93%, at least 94%, at least 95%, at least 96%, at least 97%, at least 98%, at least 99% amino acid sequence identity to SEQ ID NO: 8 or SEQ ID NO: 9.

In an aspect, the polypeptide having alpha-amylase activity is a Talaromyces helicus polypeptide, for instance, the Talaromyces helicus polypeptide having alpha-amylase activity of SEQ ID NO: 11 or SEQ ID NO: 12, or a polypeptide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 11 or SEQ ID NO: 12.

In an aspect, the polypeptide having alpha-amylase activity is a Lactobacillus amylovorous polypeptide, for instance, the Lactobacillus amylovorous polypeptide having alpha-amylase activity of SEQ ID NO: 14, SEQ ID NO: 15, SEQ ID NO: 44, SEQ ID NO: 45, SEQ ID NO: 47, SEQ ID NO: 48, SEQ ID NO: 49, SEQ ID NO: 50, SEQ ID NO: 53, SEQ ID NO: 54, SEQ ID NO: 56, SEQ ID NO: 57, SEQ ID NO: 59, SEQ ID NO: 60 or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 14 or SEQ ID NO: 15, SEQ ID NO: 44, SEQ ID NO: 45, SEQ ID NO: 47, SEQ ID NO: 48, SEQ ID NO: 49, SEQ ID NO: 50, SEQ ID NO: 53, SEQ ID NO: 54, SEQ ID NO: 56, SEQ ID NO: 57, SEQ ID NO: 59, or SEQ ID NO: 60. In an aspect, the polypeptide having alpha-amylase activity is a recombinant polypeptide comprising a Lactobacillus amylovorus catalytic domain having alpha-amylase activity of amino acids 30 to 469 of SEQ ID NO: 14 or of amino acids 1 to 440 of SEQ ID NO: 15, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 30 to 469 of SEQ ID NO: 14 or of amino acids 1 to 440 of SEQ ID NO: 15, and a His-tag (e.g., C-terminal) comprising at least one, at least two, at least three, at least four, at least five, or at least six C-terminal histidine residues. In one embodiment, the recombinant polypeptide comprises a C-terminal His-tag consisting of amino acids 470-475 of SEQ ID NO: SEQ ID NO: 14 or amino acids 441 to 446 of SEQ ID NO: 15. In an embodiment, the recombinant polypeptide has a heterologous secretion signal, such as the Bacillus licheniformis secretion signal consisting of an amino acid sequence of amino acids 1-29 of SEQ ID NO: 14 or an amino acid sequence having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 1-29 of SEQ ID NO: 14.

In an aspect, the polypeptide having alpha-amylase activity is a recombinant polypeptide comprising:
(i) a Lactobacillus amylovorus catalytic domain having alpha-amylase activity of amino acids 45 to 467 of SEQ ID NO: 44, SEQ ID NO: 47, SEQ ID NO: 50, SEQ ID NO: 53, SEQ ID NO: 56, or SEQ ID NO: 59, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 45 to 467 of SEQ ID NO: 44, SEQ ID NO: 47, SEQ ID NO: 50, SEQ ID NO: 53, SEQ ID NO: 56 or SEQ ID NO: 59;
(ii) at least one starch binding module selected from the group consisting of amino acids 478 to 569 of SEQ ID NO: 44, amino acids 470 to 565 of SEQ ID NO: 47, amino acids 475 to 571 of SEQ ID NO: 50, amino acids 563 to 654 of SEQ ID NO: 53, amino acids 655 to 750 of SEQ ID NO: 53, amino acids 476 to 569 of SEQ ID NO: 56, amino acids 479 to 572, and combinations thereof;
(iii) an optional linker connecting the C-terminus of the catalytic domain of (i) to the N-terminus of the at least one starch binding module of (ii), wherein the optional linker is selected from the group consisting of amino acids 468 to 477 of SEQ ID NO: 44, amino acids 468 to 474 of SEQ ID NO: 53, amino acids 468 to 475 of SEQ ID NO: 56, and amino acids 468 to 478 of SEQ ID NO: 59; and;
(iv) an optional His-tag (e.g., C-terminal) comprising at least one, at least two, at least three, at least four, at least five, or at least six C-terminal histidine residues.

In one embodiment, the recombinant polypeptide comprises a C-terminal His-tag consisting of amino acids 570-575 of SEQ ID NO: 44. In one embodiment, the recombinant polypeptide comprises a linker consisting of amino acids 468 to 477 of SEQ ID NO: 44. In one embodiment, the recombinant polypeptide comprises a linker consisting of amino acids 468 to 474 of SEQ ID NO: 53. In one embodiment, the recombinant polypeptide comprises a linker consisting of amino acids 468 to 475 of SEQ ID NO: 56. In one embodiment, the recombinant polypeptide comprises a linker consisting of amino acids 468 to 478 of SEQ ID NO: 59. In one embodiment, the recombinant polypeptide comprises a C-terminal His-tag consisting of amino acids 567 to 572 of SEQ ID NO: 47. In one embodiment, the recombinant polypeptide comprises a C-terminal His-tag consisting of amino acids 572 to 577 of SEQ ID NO: 50. In one embodiment, the recombinant polypeptide comprises a C-terminal His-tag consisting of amino acids 752 to 757 of SEQ ID NO: 53. In one embodiment, the recombinant polypeptide comprises a C-terminal His-tag consisting of amino acids 570 to 575 of SEQ ID NO: 56. In one embodiment, the recombinant polypeptide comprises a C-terminal His-tag consisting of amino acids 537 to 578 of SEQ ID NO: 59.

In aspect, the polypeptide having alpha-amylase activity is a Valsaria rubricosa polypeptide, for instance, the Valsaria rubricosa polypeptide having alpha-amylase activity of SEQ ID NO: 16 or SEQ ID NO: 17, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 16 or SEQ ID NO: 17.

In an aspect, the polypeptide having alpha-amylase activity is a recombinant polypeptide comprising:
(a) a Bacillus amyloliquefaciens catalytic domain having alpha-amylase activity of amino acids 28 to 465 of SEQ ID NO: 41, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 28 to 465 of SEQ ID NO: 41, wherein the catalytic domain has at least one, at least two, at least three, at least four, at least five, at least six, at least seven, at least eight, at least nine, or at least 10 substitutions compared to amino acids 28 to 465 of SEQ ID NO: 41, wherein the substitutions (e.g., optionally conservative substitutions) are at positions corresponding to positions other than D184, E216, and D277 of SEQ ID NO: 41;
(b) an optional linker comprising amino acids 466 to 553 of SEQ ID NO: 41, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 466 to 553 of SEQ ID NO: 41;
(c) a starch binding module comprising amino acids 554 to 653 of SEQ ID NO: 41, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 554 to 653 of SEQ ID NO: 41; and
(d) an optional His-tag (e.g., C-terminal) comprising at least one, at least two, at least three, at least four, at least five, or at least six C-terminal histidine residues. In one embodiment, the recombinant polypeptide comprises a C-terminal His-tag consisting of amino acids 470-475 of SEQ ID NO: SEQ ID NO: 14 or amino acids 654 to 659 of SEQ ID NO: 41. In an embodiment, the recombinant polypeptide has a heterologous secretion signal, such as the Bacillus clausii secretion signal consisting of an amino acid sequence of amino acids 1-27 of SEQ ID NO: 41 or an amino acid sequence having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 1-27 of SEQ ID NO: 41.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptides may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Catalytic Domains

In some embodiments, the present invention also relates to catalytic domains having a sequence identity of at least 86%, at least 87%, at least 88%, at least 89%, at least 90%, at least 91%, at least 92%, at least 93%, at least 94%, at least 95%, at least 96%, at least 97%, at least 98%, at least 99%, or 100% to amino acids 18 to 497 of SEQ ID NO: 5. In one aspect, the catalytic domains comprise amino acid sequences that differ by up to 10 amino acids, e.g., 1, 2, 3, 4, 5, 6, 7, 8, 9, or 10, from amino acids 18 to 497 of SEQ ID NO: 5. In another aspect, the catalytic domain comprises a variant of amino acids 18 to 497 of SEQ ID NO: 5 having a sequence identity of at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity, to amino acids 18 to 497 of SEQ ID NO: 5, and at least 1, 2, 3, 4, 6, 7, 8, 9, or 10 substitutions at positions corresponding to positions other than D223, E247 and D314 of SEQ ID NO: 5.

The catalytic domain preferably comprises, consists essentially of, or consists of amino acids 18 to 497 of SEQ ID NO: 5; or is a fragment thereof having alpha-amylase activity.

In some embodiments, the present invention also relates to catalytic domains encoded by polynucleotides that hybridize under medium stringency conditions, medium-high stringency conditions, high stringency conditions, or very high stringency conditions with the full-length complement of nucleotides 52 to 1943 of SEQ ID NO: 4 or the cDNA thereof (Sambrook et al., 1989, supra).

In some embodiments, the present invention also relates to catalytic domains encoded by polynucleotides having a sequence identity of at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 52 to 1943 of SEQ ID NO: 4, or the cDNA sequence thereof.

The polynucleotide encoding the catalytic domain preferably comprises, consists essentially of, or consists of nucleotides 52 to 1943 of SEQ ID NO: 4.

In some embodiments, the present invention relates to a catalytic domain derived from amino acids 18 to 497 of SEQ ID NO: 5 or amino acids 1 to 480 of SEQ ID NO: 6 by substitution, deletion or addition of one or several amino acids in the amino acids 18 to 497 of SEQ ID NO: 5 or amino acids 1 to 480 of SEQ ID NO: 6. In some embodiments, the present invention also relates to catalytic domain variants of amino acids 18 to 497 of SEQ ID NO: 5 comprising a substitution, deletion, and/or insertion at one or more (e.g., several) positions. In some embodiments, the catalytic domain is a variant of amino acids 18 to 497 of SEQ ID NO: 5 comprising a substitution, deletion, and/or insertion at one or more (e.g., several) positions other than at positions corresponding to D223, E247 and D314 in SEQ ID NO: 5. In one aspect, the number of amino acid substitutions, deletions and/or insertions introduced into the sequence of amino acids 18 to 497 of SEQ ID NO: 5 is up to 10, e.g., 1, 2, 3, 4, 5, 6, 8, 9, or 10. In an aspect, the catalytic domain is a variant of amino acids 18 to 497 of SEQ ID NO: 5 comprising up to 10 substitutions, e.g., 1, 2, 3, 4, 5, 6, 7, 8, 9, or 10 in amino acids 18 to 497 of SEQ ID NO: 5 at positions other than positions corresponding to D223, E247 and D314 in SEQ ID NO: 5.

In some embodiments, the present invention also relates to catalytic domains having a sequence identity of at least 94%, at least 95%, at least 96%, at least 97%, at least 98%, at least 99%, or 100% to amino acids 22 to 495 of SEQ ID NO: 8. In one aspect, the catalytic domains comprise amino acid sequences that differ by up to 10 amino acids, e.g., 1, 2, 3, 4, 5, 6, 7, 8, 9, or 10, from amino acids 22 to 495 of SEQ ID NO: 8. In another aspect, the catalytic domain comprises a variant of amino acids 22 to 495 of SEQ ID NO: 5 having a sequence identity of at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity, to amino acids 22 to 495 of SEQ ID NO: 8, and at least 1, 2, 3, 4, 6, 7, 8, 9, or 10 substitutions at positions corresponding to positions other than D223, E247 and D314 of SEQ ID NO: 8.

The catalytic domain preferably comprises, consists essentially of, or consists of amino acids 18 to 497 of SEQ ID NO: 8; or is a fragment thereof having alpha-amylase activity.

In some embodiments, the present invention also relates to catalytic domains encoded by polynucleotides that hybridize under medium stringency conditions, medium-high stringency conditions, high stringency conditions, or very high stringency conditions with the full-length complement of nucleotides 64 to 1925 of SEQ ID NO: 7 or the cDNA thereof (Sambrook et al., 1989, supra).

In some embodiments, the present invention also relates to catalytic domains encoded by polynucleotides having a sequence identity at least 94%, at least 95%, at least 96%, at least 97%, at least 98%, at least 99%, or 100% to nucleotides 64 to 1925 of SEQ ID NO: 7, or the cDNA sequence thereof.

The polynucleotide encoding the catalytic domain preferably comprises, consists essentially of, or consists of nucleotides 64 to 1925 of SEQ ID NO: 7.

In some embodiments, the present invention relates to a catalytic domain derived from amino acids 22 to 495 of SEQ ID NO: 8 or amino acids 1 to 474 of SEQ ID NO: 9 by substitution, deletion or addition of one or several amino acids in the amino acids 22 to 495 of SEQ ID NO: 8 or amino acids 1 to 474 of SEQ ID NO: 9. In some embodiments, the present invention also relates to catalytic domain variants of amino acids 22 to 495 of SEQ ID NO: 8 comprising a substitution, deletion, and/or insertion at one or more (e.g., several) positions. In some embodiments, the catalytic domain is a variant of amino acids 22 to 495 of SEQ ID NO: 8 comprising a substitution, deletion, and/or insertion at one or more (e.g., several) positions other than at positions corresponding to positions D223, E247 and D314 in SEQ ID NO: 8. In one aspect, the number of amino acid substitutions, deletions and/or insertions introduced into the sequence of amino acids 22 to 495 of SEQ ID NO: 8 is up to 10, e.g., 1, 2, 3, 4, 5, 6, 8, 9, or 10. In an aspect, the catalytic domain is a variant of amino acids 22 to 495 of SEQ ID NO: 8 comprising up to 10 substitutions, e.g., 1, 2, 3, 4, 5, 6, 7, 8, 9, or 10 in amino acids 22 to 495 of SEQ ID NO: 8 at positions other than positions corresponding to D223, E247 and D314 in SEQ ID NO: 8.

In some embodiments, the present invention also relates to catalytic domains having a sequence identity of at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to amino acids 20 to 496 of SEQ ID NO: 11. In one aspect, the catalytic domains comprise amino acid sequences that differ by up to 10 amino acids, e.g., 1, 2, 3, 4, 5, 6, 7, 8, 9, or 10, from amino acids 20 to 496 of SEQ ID NO: 11. In another aspect, the catalytic domain comprises a variant of amino acids 20 to 496 of SEQ ID NO: 11 having a sequence identity of at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity, to amino acids 20 to 496 of SEQ ID NO: 11, and at least 1, 2, 3, 4, 6, 7, 8, 9, or 10 substitutions at positions corresponding to positions other than D225, E249 and D316 of SEQ ID NO: 11.

The catalytic domain preferably comprises, consists essentially of, or consists of amino acids 20 to 496 of SEQ ID NO: 11; or is a fragment thereof having alpha-amylase activity.

In some embodiments, the present invention also relates to catalytic domains encoded by polynucleotides that hybridize under medium stringency conditions, medium-high stringency conditions, high stringency conditions, or very high stringency conditions with the full-length complement of nucleotides 58 to 1488 of SEQ ID NO: 10, or the cDNA thereof (Sambrook et al., 1989, supra).

In some embodiments, the present invention also relates to catalytic domains encoded by polynucleotides having a sequence identity at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 58 to 1488 of SEQ ID NO: 10.

The polynucleotide encoding the catalytic domain preferably comprises, consists essentially of, or consists of nucleotides 58 to 1488 of SEQ ID NO: 10.

In some embodiments, the present invention relates to a catalytic domain derived from amino acids 20 to 496 of SEQ ID NO: 11 or amino acids 1 to 477 of SEQ ID NO: 12 by substitution, deletion or addition of one or several amino acids in the amino acids 20 to 496 of SEQ ID NO: 11 or amino acids 1 to 477 of SEQ ID NO: 12. In some embodiments, the present invention also relates to catalytic domain variants of amino acids 20 to 496 of SEQ ID NO: 11 comprising a substitution, deletion, and/or insertion at one or more (e.g., several) positions. In some embodiments, the catalytic domain is a variant of amino acids 20 to 496 of SEQ ID NO: 11 comprising a substitution, deletion, and/or insertion at one or more (e.g., several) positions other than at positions corresponding to positions D223, E247 and D314 in SEQ ID NO: 11. In one aspect, the number of amino acid substitutions, deletions and/or insertions introduced into the sequence of amino acids 20 to 496 of SEQ ID NO: 11 is up to 10, e.g., 1, 2, 3, 4, 5, 6, 8, 9, or 10. In an aspect, the catalytic domain is a variant of amino acids 20 to 496 of SEQ ID NO: 11 comprising up to 10 substitutions, e.g., 1, 2, 3, 4, 5, 6, 7, 8, 9, or 10 in amino acids 20 to 496 of SEQ ID NO: 11 at positions other than positions corresponding to D223, E247 and D314 in SEQ ID NO: 11.

In some embodiments, the present invention also relates to catalytic domains having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 30 to 469 of SEQ ID NO: 14. In one aspect, the catalytic domains comprise amino acid sequences that differ by up to 10 amino acids, e.g., 1, 2, 3, 4, 5, 6, 7, 8, 9, or 10, from amino acids 30 to 469 of SEQ ID NO: 14. In an aspect, the catalytic domain is a variant of amino acids 30 to 469 of SEQ ID NO: 14 comprising up to 10 substitutions, e.g., 1, 2, 3, 4, 5, 6, 7, 8, 9, or 10 in amino acids 30 to 469 of SEQ ID NO: 14 at positions other than positions corresponding to D217, E251 and D312 in SEQ ID NO: 14.

The catalytic domain preferably comprises, consists essentially of, or consists of amino acids 30 to 469 of SEQ ID NO: 14; or is a fragment thereof having alpha-amylase activity.

In some embodiments, the present invention also relates to catalytic domains encoded by polynucleotides that hybridize under medium stringency conditions, medium-high stringency conditions, high stringency conditions, or very high stringency conditions with the full-length complement of nucleotides 88 to 1407 of SEQ ID NO: 13, or the cDNA thereof (Sambrook et al., 1989, supra).

In some embodiments, the present invention also relates to catalytic domains encoded by polynucleotides having a sequence identity at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 88 to 1407 of SEQ ID NO: 13.

The polynucleotide encoding the catalytic domain preferably comprises, consists essentially of, or consists of nucleotides 88 to 1407 of SEQ ID NO: 13.

In some embodiments, the present invention relates to a catalytic domain derived from amino acids 30 to 469 of SEQ ID NO: 14 or amino acids 1 to 440 of SEQ ID NO: 15 by substitution, deletion or addition of one or several amino acids in the amino acids 30 to 469 of SEQ ID NO: 14 or amino acids 1 to 440 of SEQ ID NO: 15. In some embodiments, the present invention also relates to catalytic domain variants of amino acids 30 to 469 of SEQ ID NO: 14 comprising a substitution, deletion, and/or insertion at one or more (e.g., several) positions. In some embodiments, the catalytic domain is a variant of amino acids 30 to 469 of SEQ ID NO: 14 comprising a substitution, deletion, and/or insertion at one or more (e.g., several) positions other than at positions corresponding to positions D217, E251 and D312 in SEQ ID NO: 14. In one aspect, the number of amino acid substitutions, deletions and/or insertions introduced into the sequence of amino acids 30 to 469 of SEQ ID NO: 14 is up to 10, e.g., 1, 2, 3, 4, 5, 6, 8, 9, or 10.

In some embodiments, the present invention also relates to catalytic domains having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 28 to 465 of SEQ ID NO: 41. In one aspect, the catalytic domains comprise amino acid sequences that differ by up to 10 amino acids, e.g., 1, 2, 3, 4, 5, 6, 7, 8, 9, or 10, from amino acids 28 to 465 of SEQ ID NO: 41. In an aspect, the catalytic domain is a variant of amino acids 28 to 465 of SEQ ID NO: 41 comprising up to 10 substitutions, e.g., 1, 2, 3, 4, 5, 6, 7, 8, 9, or 10 in amino acids 28 to 465 of SEQ ID NO: 41 at positions other than positions corresponding to D184, E216 and D277 in SEQ ID NO: 41.

The catalytic domain preferably comprises, consists essentially of, or consists of amino acids 28 to 465 of SEQ ID NO: 41; or is a fragment thereof having alpha-amylase activity.

In some embodiments, the present invention also relates to catalytic domains encoded by polynucleotides that hybridize under medium stringency conditions, medium-high stringency conditions, high stringency conditions, or very high stringency conditions with the full-length complement of nucleotides 82 to 1395 of SEQ ID NO: 40, or the cDNA thereof (Sambrook et al., 1989, supra).

In some embodiments, the present invention also relates to catalytic domains encoded by polynucleotides having a sequence identity at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 82 to 1395 of SEQ ID NO: 40.

The polynucleotide encoding the catalytic domain preferably comprises, consists essentially of, or consists of nucleotides 82 to 1395 of SEQ ID NO: 40.

In some embodiments, the present invention relates to a catalytic domain derived from amino acids 28 to 465 of SEQ ID NO: 41 by substitution, deletion or addition of one or several amino acids in the amino acids 28 to 465 of SEQ ID NO: 14. In some embodiments, the present invention also relates to catalytic domain variants of amino acids 28 to 465 of SEQ ID NO: 41 comprising a substitution, deletion, and/or insertion at one or more (e.g., several) positions. In some embodiments, the catalytic domain is a variant of amino acids 28 to 465 of SEQ ID NO: 41 comprising a substitution, deletion, and/or insertion at one or more (e.g., several) positions other than at positions corresponding to positions D184, E216 and D277 in SEQ ID NO: 41. In one aspect, the number of amino acid substitutions, deletions and/or insertions introduced into the sequence of amino acids 28 to 465 of SEQ ID NO: 14 is up to 10, e.g., 1, 2, 3, 4, 5, 6, 8, 9, or 10.

In some embodiments, the present invention also relates to catalytic domains having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 44. In one aspect, the catalytic domains comprise amino acid sequences that differ by up to 10 amino acids, e.g., 1, 2, 3, 4, 5, 6, 7, 8, 9, or 10, from amino acids 45 to 467 of SEQ ID NO: 44. In an aspect, the catalytic domain is a variant of amino acids 45 to 467 of SEQ ID NO: 44 comprising up to 10 substitutions, e.g., 1, 2, 3, 4, 5, 6, 7, 8, 9, or 10 in amino acids 45 to 467 of SEQ ID NO: 44 at positions other than positions corresponding to D215, E249 and D310 in SEQ ID NO: 44.

The catalytic domain preferably comprises, consists essentially of, or consists of amino acids 45 to 467 of SEQ ID NO: 44; or is a fragment thereof having alpha-amylase activity.

In some embodiments, the present invention also relates to catalytic domains encoded by polynucleotides that hybridize under medium stringency conditions, medium-high stringency conditions, high stringency conditions, or very high stringency conditions with the full-length complement of nucleotides 82 to 1725 of SEQ ID NO: 43, or the cDNA thereof (Sambrook et al., 1989, supra).

In some embodiments, the present invention also relates to catalytic domains encoded by polynucleotides having a sequence identity at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 82 to 1725 of SEQ ID NO: 43.

The polynucleotide encoding the catalytic domain preferably comprises, consists essentially of, or consists of nucleotides 82 to 1725 of SEQ ID NO: 43.

In some embodiments, the present invention relates to a catalytic domain derived from amino acids 45 to 467 of SEQ ID NO: 44 by substitution, deletion or addition of one or several amino acids in the amino acids 45 to 467 of SEQ ID NO: 44. In some embodiments, the present invention also relates to catalytic domain variants of amino acids 45 to 467 of SEQ ID NO: 44 comprising a substitution, deletion, and/or insertion at one or more (e.g., several) positions. In some embodiments, the catalytic domain is a variant of amino acids 45 to 467 of SEQ ID NO: 44 comprising a substitution, deletion, and/or insertion at one or more (e.g., several) positions other than at positions corresponding to positions D215, E249 and D310 in SEQ ID NO: 44. In one aspect, the number of amino acid substitutions, deletions and/or insertions introduced into the sequence of amino acids 45 to 467 of SEQ ID NO: 44 is up to 10, e.g., 1, 2, 3, 4, 5, 6, 8, 9, or 10.

In some embodiments, the present invention also relates to catalytic domains having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 47. In one aspect, the catalytic domains comprise amino acid sequences that differ by up to 10 amino acids, e.g., 1, 2, 3, 4, 5, 6, 7, 8, 9, or 10, from amino acids 45 to 467 of SEQ ID NO: 47. In an aspect, the catalytic domain is a variant of amino acids 45 to 467 of SEQ ID NO: 47 comprising up to 10 substitutions, e.g., 1, 2, 3, 4, 5, 6, 7, 8, 9, or 10 in amino acids 45 to 467 of SEQ ID NO: 47 at positions other than positions corresponding to D215, E249 and D310 in SEQ ID NO: 47.

The catalytic domain preferably comprises, consists essentially of, or consists of amino acids 45 to 467 of SEQ ID NO: 47; or is a fragment thereof having alpha-amylase activity.

In some embodiments, the present invention also relates to catalytic domains encoded by polynucleotides that hybridize under medium stringency conditions, medium-high stringency conditions, high stringency conditions, or very high stringency conditions with the full-length complement of nucleotides 82 to 1716 of SEQ ID NO: 46, or the cDNA thereof (Sambrook et al., 1989, supra).

In some embodiments, the present invention also relates to catalytic domains encoded by polynucleotides having a sequence identity at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 82 to 1716 of SEQ ID NO: 46.

The polynucleotide encoding the catalytic domain preferably comprises, consists essentially of, or consists of nucleotides 82 to 1716 of SEQ ID NO: 46.

In some embodiments, the present invention relates to a catalytic domain derived from amino acids 45 to 467 of SEQ ID NO: 47 by substitution, deletion or addition of one or several amino acids in the amino acids 45 to 467 of SEQ ID NO: 47. In some embodiments, the present invention also relates to catalytic domain variants of amino acids 45 to 467 of SEQ ID NO: 47 comprising a substitution, deletion, and/or insertion at one or more (e.g., several) positions. In some embodiments, the catalytic domain is a variant of amino acids 45 to 467 of SEQ ID NO: 47 comprising a substitution, deletion, and/or insertion at one or more (e.g., several) positions other than at positions corresponding to positions D215, E249 and D310 in SEQ ID NO: 47. In one aspect, the number of amino acid substitutions, deletions and/or insertions introduced into the sequence of amino acids 45 to 467 of SEQ ID NO: 47 is up to 10, e.g., 1, 2, 3, 4, 5, 6, 8, 9, or 10.

In some embodiments, the present invention also relates to catalytic domains having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 50. In one aspect, the catalytic domains comprise amino acid sequences that differ by up to 10 amino acids, e.g., 1, 2, 3, 4, 5, 6, 7, 8, 9, or 10, from amino acids 45 to 467 of SEQ ID NO: 50. In an aspect, the catalytic domain is a variant of amino acids 45 to 467 of SEQ ID NO: 50 comprising up to 10 substitutions, e.g., 1, 2, 3, 4, 5, 6, 7, 8, 9, or 10 in amino acids 45 to 467 of SEQ ID NO: 50 at positions other than positions corresponding to D215, E249 and D310 in SEQ ID NO: 50.

The catalytic domain preferably comprises, consists essentially of, or consists of amino acids 45 to 467 of SEQ ID NO: 50; or is a fragment thereof having alpha-amylase activity.

In some embodiments, the present invention also relates to catalytic domains encoded by polynucleotides that hybridize under medium stringency conditions, medium-high stringency conditions, high stringency conditions, or very high stringency conditions with the full-length complement of nucleotides 82 to 1731 of SEQ ID NO: 49, or the cDNA thereof (Sambrook et al., 1989, supra).

In some embodiments, the present invention also relates to catalytic domains encoded by polynucleotides having a sequence identity at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 82 to 1731 of SEQ ID NO: 49.

The polynucleotide encoding the catalytic domain preferably comprises, consists essentially of, or consists of nucleotides 82 to 1731 of SEQ ID NO: 49.

In some embodiments, the present invention relates to a catalytic domain derived from amino acids 45 to 467 of SEQ ID NO: 50 by substitution, deletion or addition of one or several amino acids in the amino acids 45 to 467 of SEQ ID NO: 50. In some embodiments, the present invention also relates to catalytic domain variants of amino acids 45 to 467 of SEQ ID NO: 50 comprising a substitution, deletion, and/or insertion at one or more (e.g., several) positions. In some embodiments, the catalytic domain is a variant of amino acids 45 to 467 of SEQ ID NO: 50 comprising a substitution, deletion, and/or insertion at one or more (e.g., several) positions other than at positions corresponding to positions D215, E249 and D310 in SEQ ID NO: 50. In one aspect, the number of amino acid substitutions, deletions and/or insertions introduced into the sequence of amino acids 45 to 467 of SEQ ID NO: 50 is up to 10, e.g., 1, 2, 3, 4, 5, 6, 8, 9, or 10.

In some embodiments, the present invention also relates to catalytic domains having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 53. In one aspect, the catalytic domains comprise amino acid sequences that differ by up to 10 amino acids, e.g., 1, 2, 3, 4, 5, 6, 7, 8, 9, or 10, from amino acids 45 to 467 of SEQ ID NO: 53. In an aspect, the catalytic domain is a variant of amino acids 45 to 467 of SEQ ID NO: 53 comprising up to 10 substitutions, e.g., 1, 2, 3, 4, 5, 6, 7, 8, 9, or 10 in amino acids 45 to 467 of SEQ ID NO: 53 at positions other than positions corresponding to D215, E249 and D310 in SEQ ID NO: 53.

The catalytic domain preferably comprises, consists essentially of, or consists of amino acids 45 to 467 of SEQ ID NO: 53; or is a fragment thereof having alpha-amylase activity.

In some embodiments, the present invention also relates to catalytic domains encoded by polynucleotides that hybridize under medium stringency conditions, medium-high stringency conditions, high stringency conditions, or very high stringency conditions with the full-length complement of nucleotides 82 to 2271 of SEQ ID NO: 52, or the cDNA thereof (Sambrook et al., 1989, supra).

In some embodiments, the present invention also relates to catalytic domains encoded by polynucleotides having a sequence identity at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 82 to 2271 of SEQ ID NO: 52.

The polynucleotide encoding the catalytic domain preferably comprises, consists essentially of, or consists of nucleotides 82 to 2271 of SEQ ID NO: 52.

In some embodiments, the present invention relates to a catalytic domain derived from amino acids 45 to 467 of SEQ ID NO: 53 by substitution, deletion or addition of one or several amino acids in the amino acids 45 to 467 of SEQ ID NO: 53. In some embodiments, the present invention also relates to catalytic domain variants of amino acids 45 to 467 of SEQ ID NO: 53 comprising a substitution, deletion, and/or insertion at one or more (e.g., several) positions. In some embodiments, the catalytic domain is a variant of amino acids 45 to 467 of SEQ ID NO: 53 comprising a substitution, deletion, and/or insertion at one or more (e.g., several) positions other than at positions corresponding to positions D215, E249 and D310 in SEQ ID NO: 53. In one aspect, the number of amino acid substitutions, deletions and/or insertions introduced into the sequence of amino acids 45 to 467 of SEQ ID NO: 53 is up to 10, e.g., 1, 2, 3, 4, 5, 6, 8, 9, or 10.

In some embodiments, the present invention also relates to catalytic domains having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 56. In one aspect, the catalytic domains comprise amino acid sequences that differ by up to 10 amino acids, e.g., 1, 2, 3, 4, 5, 6, 7, 8, 9, or 10, from amino acids 45 to 467 of SEQ ID NO: 56. In an aspect, the catalytic domain is a variant of amino acids 45 to 467 of SEQ ID NO: 56 comprising up to 10 substitutions, e.g., 1, 2, 3, 4, 5, 6, 7, 8, 9, or 10 in amino acids 45 to 467 of SEQ ID NO: 56 at positions other than positions corresponding to D215, E249 and D310 in SEQ ID NO: 56.

The catalytic domain preferably comprises, consists essentially of, or consists of amino acids 45 to 467 of SEQ ID NO: 56; or is a fragment thereof having alpha-amylase activity.

In some embodiments, the present invention also relates to catalytic domains encoded by polynucleotides that hybridize under medium stringency conditions, medium-high stringency conditions, high stringency conditions, or very high stringency conditions with the full-length complement of nucleotides 133 to 1401 of SEQ ID NO: 55, or the cDNA thereof (Sambrook et al., 1989, supra).

In some embodiments, the present invention also relates to catalytic domains encoded by polynucleotides having a sequence identity at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 133 to 1401 of SEQ ID NO: 55.

The polynucleotide encoding the catalytic domain preferably comprises, consists essentially of, or consists of nucleotides 133 to 1401 of SEQ ID NO: 55.

In some embodiments, the present invention relates to a catalytic domain derived from amino acids 45 to 467 of SEQ ID NO: 56 by substitution, deletion or addition of one or several amino acids in the amino acids 45 to 467 of SEQ ID NO: 56. In some embodiments, the present invention also relates to catalytic domain variants of amino acids 45 to 467 of SEQ ID NO: 56 comprising a substitution, deletion, and/or insertion at one or more (e.g., several) positions. In some embodiments, the catalytic domain is a variant of amino acids 45 to 467 of SEQ ID NO: 56 comprising a substitution, deletion, and/or insertion at one or more (e.g., several) positions other than at positions corresponding to positions D215, E249 and D310 in SEQ ID NO: 56. In one aspect, the number of amino acid substitutions, deletions and/or insertions introduced into the sequence of amino acids 45 to 467 of SEQ ID NO: 56 is up to 10, e.g., 1, 2, 3, 4, 5, 6, 8, 9, or 10.

In some embodiments, the present invention also relates to catalytic domains having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 59. In one aspect, the catalytic domains comprise amino acid sequences that differ by up to 10 amino acids, e.g., 1, 2, 3, 4, 5, 6, 7, 8, 9, or 10, from amino acids 45 to 467 of SEQ ID NO: 59. In an aspect, the catalytic domain is a variant of amino acids 45 to 467 of SEQ ID NO: 59 comprising up to 10 substitutions, e.g., 1, 2, 3, 4, 5, 6, 7, 8, 9, or 10 in amino acids 45 to 467 of SEQ ID NO: 59 at positions other than positions corresponding to D215, E249 and D310 in SEQ ID NO: 59.

The catalytic domain preferably comprises, consists essentially of, or consists of amino acids 45 to 467 of SEQ ID NO: 59; or is a fragment thereof having alpha-amylase activity.

In some embodiments, the present invention also relates to catalytic domains encoded by polynucleotides that hybridize under medium stringency conditions, medium-high stringency conditions, high stringency conditions, or very high stringency conditions with the full-length complement of nucleotides 133 to 1401 of SEQ ID NO: 58, or the cDNA thereof (Sambrook et al., 1989, supra).

In some embodiments, the present invention also relates to catalytic domains encoded by polynucleotides having a sequence identity at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 133 to 1401 of SEQ ID NO: 58.

The polynucleotide encoding the catalytic domain preferably comprises, consists essentially of, or consists of nucleotides 133 to 1401 of SEQ ID NO: 58.

In some embodiments, the present invention relates to a catalytic domain derived from amino acids 45 to 467 of SEQ ID NO: 59 by substitution, deletion or addition of one or several amino acids in the amino acids 45 to 467 of SEQ ID NO: 59. In some embodiments, the present invention also relates to catalytic domain variants of amino acids 45 to 467 of SEQ ID NO: 59 comprising a substitution, deletion, and/or insertion at one or more (e.g., several) positions. In some embodiments, the catalytic domain is a variant of amino acids 45 to 467 of SEQ ID NO: 59 comprising a substitution, deletion, and/or insertion at one or more (e.g., several) positions other than at positions corresponding to positions D215, E249 and D310 in SEQ ID NO: 59. In one aspect, the number of amino acid substitutions, deletions and/or insertions introduced into the sequence of amino acids 45 to 467 of SEQ ID NO: 59 is up to 10, e.g., 1, 2, 3, 4, 5, 6, 8, 9, or 10.

In another aspect, a polypeptide comprising a catalytic domain of the present invention may further comprise a starch binding module.

### Binding Modules

In some embodiments, the present invention also relates to polypeptides comprising a catalytic domain and a starch binding module, wherein the starch binding module has a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 532 to 632 of SEQ ID NO: 5. In one aspect, the starch binding modules comprise amino acid sequences that differ by up to 10 amino acids, e.g., 1, 2, 3, 4, 5, 6, 7, 8, 9, or 10, from amino acids 532 to 632 of SEQ ID NO: 5.

The starch binding module preferably comprises, consists essentially of, or consists of amino acids 532 to 632 of SEQ ID NO: 5; or is a fragment thereof having starch binding activity.

In some embodiments, the present invention also relates to starch binding modules encoded by polynucleotides that hybridize under medium stringency conditions, medium-high stringency conditions, high stringency conditions, or very high stringency conditions with the full-length complement of nucleotides 2046 to 2348 of SEQ ID NO: 4 or the cDNA thereof (Sambrook et al., 1989, supra).

In some embodiments, the present invention also relates to starch binding modules encoded by polynucleotides having a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 2046 to 2348 of SEQ ID NO: 4, or the cDNA thereof.

The polynucleotide encoding the starch binding module preferably comprises, consists essentially of, or consists of nucleotides 2046 to 2348 of SEQ ID NO: 4, or the cDNA thereof.

In some embodiments, the present invention relates to a starch binding module derived from amino acids 532 to 632 of SEQ ID NO: 5 or amino acids 515 to 615 of SEQ ID NO: 6 by substitution, deletion or addition of one or several amino acids in the amino acids 532 to 632 of SEQ ID NO: 5 or amino acids 515 to 615 of SEQ ID NO: 6. In some embodiments, the present invention also relates to starch binding module variants of amino acids 532 to 632 of SEQ ID NO: 5 comprising a substitution, deletion, and/or insertion at one or more (e.g., several) positions. In one aspect, the number of amino acid substitutions, deletions and/or insertions introduced into the sequence of amino acids 532 to 632 of SEQ ID NO: 5 is up to 10, e.g., 1, 2, 3, 4, 5, 6, 8, 9, or 10.

In some embodiments, the present invention also relates to polypeptides comprising a catalytic domain and a starch binding module, wherein the starch binding module has a sequence identity of at least 91%, at least 92%, at least 93%, at least 94%, at least 95%, at least 96%, at least 97%, at least 98%, at least 99%, or 100% sequence identity to amino acids 532 to 632 of SEQ ID NO: 8. In one aspect, the starch binding modules comprise amino acid sequences that differ by up to 10 amino acids, e.g., 1, 2, 3, 4, 5, 6, 7, 8, 9, or 10, from amino acids 532 to 632 of SEQ ID NO: 8.

The starch binding module preferably comprises, consists essentially of, or consists of amino acids 532 to 632 of SEQ ID NO: 8; or is a fragment thereof having starch binding activity.

In some embodiments, the present invention also relates to starch binding modules encoded by polynucleotides that hybridize under medium stringency conditions, medium-high stringency conditions, high stringency conditions, or very high stringency conditions with the full-length complement of nucleotides 2034 to 2336 of SEQ ID NO: 7 or the cDNA thereof (Sambrook et al., 1989, supra).

In some embodiments, the present invention also relates to starch binding modules encoded by polynucleotides having a sequence identity of at least 91%, at least 92%, at least 93%, at least 94%, at least 95%, at least 96%, at least 97%, at least 98%, at least 99%, or 100% to nucleotides 2034 to 2336 of SEQ ID NO: 7, or the cDNA thereof.

The polynucleotide encoding the starch binding module preferably comprises, consists essentially of, or consists of nucleotides 2034 to 2336 of SEQ ID NO: 7, or the cDNA thereof.

In some embodiments, the present invention relates to a starch binding module derived from amino acids 532 to 632 of SEQ ID NO: 8 or amino acids 511 to 611 of SEQ ID NO: 9 by substitution, deletion or addition of one or several amino acids in the amino acids 532 to 632 of SEQ ID NO: 8 or amino acids 511 to 611 of SEQ ID NO: 9. In some embodiments, the present invention also relates to starch binding module variants of amino acids 532 to 632 of SEQ ID NO: 8 comprising a substitution, deletion, and/or insertion at one or more (e.g., several) positions. In one aspect, the number of amino acid substitutions, deletions and/or insertions introduced into the sequence of amino acids 532 to 632 of SEQ ID NO: 8 is up to 10, e.g., 1, 2, 3, 4, 5, 6, 8, 9, or 10.

In some embodiments, the present invention also relates to polypeptides comprising a catalytic domain and a starch binding module, wherein the starch binding module has a sequence identity of at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 508 to 601 of SEQ ID NO: 11. In one aspect, the starch binding modules comprise amino acid sequences that differ by up to 10 amino acids, e.g., 1, 2, 3, 4, 5, 6, 7, 8, 9, or 10, from amino acids 508 to 601 of SEQ ID NO: 11.

The starch binding module preferably comprises, consists essentially of, or consists of amino acids 508 to 601 of SEQ ID NO: 11; or is a fragment thereof having starch binding activity.

In some embodiments, the present invention also relates to starch binding modules encoded by polynucleotides that hybridize under medium stringency conditions, medium-high stringency conditions, high stringency conditions, or very high stringency conditions with the full-length complement of nucleotides 1504 to 1827 of SEQ ID NO: 10 or the cDNA thereof (Sambrook et al., 1989, supra).

In some embodiments, the present invention also relates to starch binding modules encoded by polynucleotides having a sequence identity of at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 1504 to 1827 of SEQ ID NO: 10, or the cDNA thereof.

The polynucleotide encoding the starch binding module preferably comprises, consists essentially of, or consists of nucleotides 1504 to 1827 of SEQ ID NO: 10, or the cDNA thereof.

In some embodiments, the present invention relates to a starch binding module derived from amino acids 508 to 601 of SEQ ID NO: 11 or amino acids 489 to 582 of SEQ ID NO: 12 by substitution, deletion or addition of one or several amino acids in the amino acids 508 to 601 of SEQ ID NO: 11 or amino acids 489 to 582 of SEQ ID NO: 12. In some embodiments, the present invention also relates to starch binding module variants of amino acids 508 to 601 of SEQ ID NO: 11 comprising a substitution, deletion, and/or insertion at one or more (e.g., several) positions. In one aspect, the number of amino acid substitutions, deletions and/or insertions introduced into the sequence of amino acids 508 to 601 of SEQ ID NO: 11 is up to 10, e.g., 1, 2, 3, 4, 5, 6, 8, 9, or 10.

In some embodiments, the present invention relates to a starch binding module derived from amino acids 554 to 653 of SEQ ID NO: 41 by substitution, deletion or addition of one or several amino acids in the amino acids 554 to 653 of SEQ ID NO: 41. In some embodiments, the present invention also relates to starch binding module variants of amino acids 554 to 653 of SEQ ID NO: 41 comprising a substitution, deletion, and/or insertion at one or more (e.g., several) positions. In one aspect, the number of amino acid substitutions, deletions and/or insertions introduced into the sequence of amino acids 554 to 653 of SEQ ID NO: 41 is up to 10, e.g., 1, 2, 3, 4, 5, 6, 8, 9, or 10.

In some embodiments, the present invention also relates to polypeptides comprising a catalytic domain and a starch binding module, wherein the starch binding module has a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 478 to 569 of SEQ ID NO: 44 or amino acids 563 to 654 of SEQ ID NO: 53. In one aspect, the starch binding modules comprise amino acid sequences that differ by up to 10 amino acids, e.g., 1, 2, 3, 4, 5, 6, 7, 8, 9, or 10, from amino acids 478 to 569 of SEQ ID NO: 44 or amino acids 563 to 654 of SEQ ID NO: 53.

The starch binding module preferably comprises, consists essentially of, or consists of amino acids 478 to 569 of SEQ ID NO: 44 or amino acids 563 to 654 of SEQ ID NO: 53; or is a fragment thereof having starch binding activity.

In some embodiments, the present invention also relates to starch binding modules encoded by polynucleotides that hybridize under medium stringency conditions, medium-high stringency conditions, high stringency conditions, or very high stringency conditions with the full-length complement of nucleotides 1432 to 1707 of SEQ ID NO: 43, nucleotides 1687 to 1962 of SEQ ID NO: 52, or the cDNA thereof (Sambrook et al., 1989, supra).

In some embodiments, the present invention also relates to starch binding modules encoded by polynucleotides having a sequence identity of at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 1432 to 1707 of SEQ ID NO: 43, nucleotides 1687 to 1962 of SEQ ID NO: 52, or the cDNA thereof.

The polynucleotide encoding the starch binding module preferably comprises, consists essentially of, or consists of nucleotides 1432 to 1707 of SEQ ID NO: 43, nucleotides 1687 to 1962 of SEQ ID NO: 52, or the cDNA thereof.

In some embodiments, the present invention relates to a starch binding module derived from amino acids 478 to 569 of SEQ ID NO: 44 or amino acids 563 to 654 of SEQ ID NO: 53 by substitution, deletion or addition of one or several amino acids in amino acids 478 to 569 of SEQ ID NO: 44 or amino acids 563 to 654 of SEQ ID NO: 53. In some embodiments, the present invention also relates to starch binding module variants of amino acids 478 to 569 of SEQ ID NO: 44 or amino acids 563 to 654 of SEQ ID NO: 53 comprising a substitution, deletion, and/or insertion at one or more (e.g., several) positions. In one aspect, the number of amino acid substitutions, deletions and/or insertions introduced into the sequence of amino acids 478 to 569 of SEQ ID NO: 44 or amino acids 563 to 654 of SEQ ID NO: 53 is up to 10, e.g., 1, 2, 3, 4, 5, 6, 8, 9, or 10.

In some embodiments, the present invention also relates to polypeptides comprising a catalytic domain and a starch binding module, wherein the starch binding module has a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 470 to 565 of SEQ ID NO: 47, amino acids 475 to 571 of SEQ ID NO: 50, or amino acids 655 to 750 of SEQ ID NO: 53. In one aspect, the starch binding modules comprise amino acid sequences that differ by up to 10 amino acids, e.g., 1, 2, 3, 4, 5, 6, 7, 8, 9, or 10, from amino acids 470 to 565 of SEQ ID NO: 47, amino acids 475 to 571 of SEQ ID NO: 50, or amino acids 655 to 750 of SEQ ID NO: 53.

The starch binding module preferably comprises, consists essentially of, or consists of amino acids 470 to 565 of SEQ ID NO: 47, amino acids 475 to 571 of SEQ ID NO: 50, or amino acids 655 to 750 of SEQ ID NO: 53; or is a fragment thereof having starch binding activity.

In some embodiments, the present invention also relates to starch binding modules encoded by polynucleotides that hybridize under medium stringency conditions, medium-high stringency conditions, high stringency conditions, or very high stringency conditions with the full-length complement of nucleotides 1408 to 1695 of SEQ ID NO: 46, nucleotides 1423 to 1713 of SEQ ID NO: 49, or nucleotides 1963 to 2250 of SEQ ID NO: 52, or the cDNA thereof (Sambrook et al., 1989, supra).

In some embodiments, the present invention also relates to starch binding modules encoded by polynucleotides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 1408 to 1695 of SEQ ID NO: 46, nucleotides 1423 to 1713 of SEQ ID NO: 49, or nucleotides 1963 to 2250 of SEQ ID NO: 52, or the cDNA thereof.

The polynucleotide encoding the starch binding module preferably comprises, consists essentially of, or consists of nucleotides 1408 to 1695 of SEQ ID NO: 46, nucleotides 1423 to 1713 of SEQ ID NO: 49, or nucleotides 1963 to 2250 of SEQ ID NO: 52, or the cDNA thereof.

In some embodiments, the present invention relates to a starch binding module derived from amino acids 470 to 565 of SEQ ID NO: 47, amino acids 475 to 571 of SEQ ID NO: 50, or amino acids 655 to 750 of SEQ ID NO: 53 by substitution, deletion or addition of one or several amino acids in amino acids 470 to 565 of SEQ ID NO: 47, amino acids 475 to 571 of SEQ ID NO: 50, or amino acids 655 to 750 of SEQ ID NO: 53. In some embodiments, the present invention also relates to starch binding module variants of amino acids 470 to 565 of SEQ ID NO: 47, amino acids 475 to 571 of SEQ ID NO: 50, or amino acids 655 to 750 of SEQ ID NO: 53 comprising a substitution, deletion, and/or insertion at one or more (e.g., several) positions. In one aspect, the number of amino acid substitutions, deletions and/or insertions introduced into the sequence of amino acids 470 to 565 of SEQ ID NO: 47, amino acids 475 to 571 of SEQ ID NO: 50, or amino acids 655 to 750 of SEQ ID NO: 53 is up to 10, e.g., 1, 2, 3, 4, 5, 6, 8, 9, or 10.

In some embodiments, the present invention also relates to polypeptides comprising a catalytic domain and a starch binding module, wherein the starch binding module has a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 476 to 569 of SEQ ID NO: 56. In one aspect, the starch binding modules comprise amino acid sequences that differ by up to 10 amino acids, e.g., 1, 2, 3, 4, 5, 6, 7, 8, 9, or 10, from amino acids 476 to 569 of SEQ ID NO: 56.

The starch binding module preferably comprises, consists essentially of, or consists of amino acids 476 to 569 of SEQ ID NO: 56; or is a fragment thereof having starch binding activity.

In some embodiments, the present invention also relates to starch binding modules encoded by polynucleotides that hybridize under medium stringency conditions, medium-high stringency conditions, high stringency conditions, or very high stringency conditions with the full-length complement of nucleotides 1426 to 1707 of SEQ ID NO: 55 or the cDNA thereof (Sambrook et al., 1989, supra).

In some embodiments, the present invention also relates to starch binding modules encoded by polynucleotides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 1426 to 1707 of SEQ ID NO: 55, or the cDNA thereof.

The polynucleotide encoding the starch binding module preferably comprises, consists essentially of, or consists of nucleotides 1426 to 1707 of SEQ ID NO: 55, or the cDNA thereof.

In some embodiments, the present invention relates to a starch binding module derived from amino acids 476 to 569 of SEQ ID NO: 56 or amino acids 449 to 542 of SEQ ID NO: 57 by substitution, deletion or addition of one or several amino acids in the amino acids 476 to 569 of SEQ ID NO: 56 or amino acids 449 to 542 of SEQ ID NO: 57. In some embodiments, the present invention also relates to starch binding module variants of amino acids 476 to 569 of SEQ ID NO: 56 comprising a substitution, deletion, and/or insertion at one or more (e.g., several) positions. In one aspect, the number of amino acid substitutions, deletions and/or insertions introduced into the sequence of amino acids 476 to 569 of SEQ ID NO: 56 is up to 10, e.g., 1, 2, 3, 4, 5, 6, 8, 9, or 10.

In some embodiments, the present invention also relates to polypeptides comprising a catalytic domain and a starch binding module, wherein the starch binding module has a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 479 to 572 of SEQ ID NO: 59. In one aspect, the starch binding modules comprise amino acid sequences that differ by up to 10 amino acids, e.g., 1, 2, 3, 4, 5, 6, 7, 8, 9, or 10, from amino acids 479 to 572 of SEQ ID NO: 59.

The starch binding module preferably comprises, consists essentially of, or consists of amino acids 479 to 572 of SEQ ID NO: 59; or is a fragment thereof having starch binding activity.

In some embodiments, the present invention also relates to starch binding modules encoded by polynucleotides that hybridize under medium stringency conditions, medium-high stringency conditions, high stringency conditions, or very high stringency conditions with the full-length complement of nucleotides 1435 to 1716 of SEQ ID NO: 58 or the cDNA thereof (Sambrook et al., 1989, supra).

In some embodiments, the present invention also relates to starch binding modules encoded by polynucleotides having a sequence identity of at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to nucleotides 1435 to 1716 of SEQ ID NO: 58, or the cDNA thereof.

The polynucleotide encoding the starch binding module preferably comprises, consists essentially of, or consists of nucleotides 1435 to 1716 of SEQ ID NO: 58, or the cDNA thereof.

In some embodiments, the present invention relates to a starch binding module derived from amino acids 479 to 572 of SEQ ID NO: 59 or amino acids 452 to 545 of SEQ ID NO: 60 by substitution, deletion or addition of one or several amino acids in the amino acids 479 to 572 of SEQ ID NO: 59 or amino acids 452 to 545 of SEQ ID NO: 60. In some embodiments, the present invention also relates to starch binding module variants of amino acids 479 to 572 of SEQ ID NO: 59 comprising a substitution, deletion, and/or insertion at one or more (e.g., several) positions. In one aspect, the number of amino acid substitutions, deletions and/or insertions introduced into the sequence of amino acids 479 to 572 of SEQ ID NO: 59 is up to 10, e.g., 1, 2, 3, 4, 5, 6, 8, 9, or 10.

The catalytic domain may be from a hydrolase, isomerase, ligase, lyase, oxidoreductase, or transferase, e.g., an aminopeptidase, amylase, carbohydrase, carboxypeptidase, catalase, cellobiohydrolase, cellulase, cutinase, chitinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase. Preferably, the catalytic domain is from a hydrolase, such as an amylase, for example alpha-amylase. The polynucleotide encoding the catalytic domain may be obtained from any prokaryotic, eukaryotic, or other source.

The polypeptides may further comprise a linker between the catalytic domain and the starch binding module.

### Polynucleotides

The present invention also relates to isolated polynucleotides encoding a polypeptide, a catalytic domain, or starch binding module of the present invention, as described herein.

The techniques used to isolate or clone a polynucleotide are known in the art and include isolation from genomic DNA or cDNA, or a combination thereof. The cloning of the polynucleotides from genomic DNA can be effected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Penicillium, Talaromyces, or Lactobacillus, or a related organism and thus, for example, may be a species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NO: 1, SEQ ID NO: 4, SEQ ID NO: 7, SEQ ID NO: 10, SEQ ID NO: 13, SEQ ID NO: 43, SEQ ID NO: 46, SEQ ID NO: 49, SEQ ID NO: 52, SEQ ID NO: 55, or SEQ ID NO: 58 or the cDNA sequence of SEQ ID NO: 4 or SEQ ID NO: 7, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis crylllA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the polynucleotide of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rmB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis crylllA gene (WO 94/25612) and a Bacillus subtilis SP82 gene (Hue et al., 1995, J. Bacteriol. 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiol. Rev. 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase | promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the polypeptide and its source.

In some embodiments, the polypeptide is heterologous to the recombinant host cell.

In some embodiments, at least one of the one or more control sequences is heterologous to the polynucleotide encoding the polypeptide.

In some embodiments, the recombinant host cell comprises at least two copies, e.g., three, four, or five, of the polynucleotide of the present invention.

The host cell may be any microbial or plant cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolismis obligately aerobic. In contrast, vegetative growth by yeasts such as Saccharomyces cerevisiae is by budding of a unicellular thallus and carbon catabolismmay be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M.I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is a Pencillium cell. In another aspect, the cell is a Pencillium oxalicum cell. In another aspect, the cell is a Pencillium sclerotiorum cell. In another aspect, the cell is a Pencillium wotroi cell. In one aspect, the cell is a Talaromyces cell. In another aspect, the cell is a Talaromyces helicus cell. In one aspect, the cell is a Lactobacillus cell. In another aspect, the cell is a Lactobacillus amylovorus cell. In one aspect, the cell is a Bacillus cell. In another aspect, the cell is a Bacillus amyloliquefaciens cell.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid-state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide

The polypeptide may be recovered using methods known in the art. For example, the polypeptide may be recovered from the fermentation medium by conventional procedures including, but not limited to, collection, centrifugation, filtration, extraction, spray-drying, evaporation, or precipitation. In one aspect, a whole fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

### Alpha-amylase Granules

The present invention also relates to enzyme granules/particles comprising the alpha-amylase of the invention. In an embodiment, the granule comprises a core, and optionally one or more coatings (outer layers) surrounding the core.

The core may have a diameter, measured as equivalent spherical diameter (volume based average particle size), of 20-2000 μm, particularly 50-1500 μm, 100-1500 μm or 250-1200 μm.

In an embodiment, the core comprises one or more polypeptides having alpha-amylase activity of the present invention.

The core may include additional materials such as fillers, fiber materials (cellulose or synthetic fibers), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

The core may include an inert particle with the enzyme absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA).

The coating may be applied in an amount of at least 0.1% by weight of the core, e.g., at least 0.5%, at least 1%, at least 5%, at least 10%, or at least 15%. The amount may be at most 100%, 70%, 50%, 40% or 30%.

The coating is preferably at least 0.1 μm thick, particularly at least 0.5 μm, at least 1 μm or at least 5 μm. In some embodiments, the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit it is encapsulating/enclosing has few or none uncoated areas. The layer or coating should, in particular, be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

To provide acceptable protection, the salt coating is preferably at least 0.1 μm thick, e.g., at least 0.5 μm, at least 1 μm, at least 2 μm, at least 4 μm, at least 5 μm, or at least 8 μm. In a particular embodiment, the thickness of the salt coating is below 100 μm, such as below 60 μm, or below 40 μm.

The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular, having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminum. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular, alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710. Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e. a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄·7H₂O), zinc sulfate heptahydrate (ZnSO₄·7H₂O), sodium phosphate dibasic heptahydrate (Na₂HPO₄·7H₂O), magnesium nitrate hexahydrate (Mg(NO₃)₂(6H₂O)), sodium citrate dihydrate and magnesium acetate tetrahydrate.

Preferably the salt is applied as a solution of the salt, e.g., using a fluid bed.

The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granule may optionally have one or more additional coatings. Examples of suitable coating materials are polyethylene glycol (PEG), methyl hydroxy-propyl cellulose (MHPC) and polyvinyl alcohol (PVA). Examples of enzyme granules with multiple coatings are described in WO 93/07263 and WO 97/23606.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in the Handbook of Powder Technology; Particle size enlargement by C. E. Capes; Volume 1; 1980; Elsevier. Preparation methods include known feed and granule formulation technologies, e.g.,
(a) Spray dried products, wherein a liquid enzyme-containing solution is atomized in a spray drying tower to form small droplets which during their way down the drying tower dry to form an enzyme-containing particulate material. Very small particles can be produced this way (Michael S. Showell (editor); Powdered detergents; Surfactant Science Series; 1998; vol. 71; page 140-142; Marcel Dekker).
(b) Layered products, wherein the enzyme is coated as a layer around a pre-formed inert core particle, wherein an enzyme-containing solution is atomized, typically in a fluid bed apparatus wherein the pre-formed core particles are fluidized, and the enzyme-containing solution adheres to the core particles and dries up to leave a layer of dry enzyme on the surface of the core particle. Particles of a desired size can be obtained this way if a useful core particle of the desired size can be found. This type of product is described in, e.g., WO 97/23606.
(c) Absorbed core particles, wherein rather than coating the enzyme as a layer around the core, the enzyme is absorbed onto and/or into the surface of the core. Such a process is described in WO 97/39116.
(d) Extrusion or pelletized products, wherein an enzyme-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme (Michael S. Showell (editor); Powdered detergents; Surfactant Science Series; 1998; vol. 71; pages 140-142; Marcel Dekker).
(e) Prilled products, wherein an enzyme-containing powder is suspended in molten wax and the suspension is sprayed, e.g., through a rotating disk atomizer, into a cooling chamber where the droplets quickly solidify (Michael S. Showell (editor); Powdered detergents; Surfactant Science Series; 1998; vol. 71; page 140-142; Marcel Dekker). The product obtained is one wherein the enzyme is uniformly distributed throughout an inert material instead of being concentrated on its surface. U.S. Pat. Nos. 4,016,040 and 4,713,245 describe this technique.
(f) Mixer granulation products, wherein an enzyme-containing liquid is added to a dry powder composition of conventional granulating components. The liquid and the powder in a suitable proportion are mixed and as the moisture of the liquid is absorbed in the dry powder, the components of the dry powder will start to adhere and agglomerate and particles will build up, forming granulates comprising the enzyme. Such a process is described in U.S. Pat. No. 4,106,991 and related documents EP 170360, EP 304332, EP 304331, WO 90/09440 and WO 90/09428. In a particular product of this process, various high-shear mixers can be used as granulators. Granulates consisting of enzyme, fillers and binders etc. are mixed with cellulose fibers to reinforce the particles to produce a so-called T-granulate. Reinforced particles, are more robust, and release less enzymatic dust.
(g) Size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in Martin Rhodes (editor); Principles of Powder Technology; 1990; Chapter 10; John Wiley & Sons.
(h) Fluid bed granulation. Fluid bed granulation involves suspending particulates in an air stream and spraying a liquid onto the fluidized particles via nozzles. Particles hit by spray droplets get wetted and become tacky. The tacky particles collide with other particles and adhere to them to form a granule.
(i) The cores may be subjected to drying, such as in a fluid bed drier. Other known methods for drying granules in the feed or enzyme industry can be used by the skilled person. The drying preferably takes place at a product temperature of from 25 to 90° C. For some enzymes, it is important the cores comprising the enzyme contain a low amount of water before coating with the salt. If water sensitive enzymes are coated with a salt before excessive water is removed, it will be trapped within the core and may affect the activity of the enzyme negatively. After drying, the cores preferably contain 0.1-10% w/w water.

Non-dusting granulates may be produced, e.g., as disclosed in U.S. Pat. Nos. 4,106,991 and 4,661,452 and may optionally be coated by methods known in the art.

The granulate may further one or more additional enzymes. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of enzymes by the use of co-granulates is disclosed in WO 2013/188331.

The present invention also relates to protected enzymes prepared according to the method disclosed in EP 238,216.

In an embodiment, the granule further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof.

### Liquid Formulations

The present invention also relates to liquid compositions comprising the alpha-amylase of the invention. The composition may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

In some embodiments, filler(s) or carrier material(s) are included to increase the volume of such compositions. Suitable filler or carrier materials include, but are not limited to, various salts of sulfate, carbonate and silicate as well as talc, clay and the like. Suitable filler or carrier materials for liquid compositions include, but are not limited to water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials.

In an aspect, the present invention relates to liquid formulations comprising:
(A) 0.001% to 25% w/w of one or more polypeptides having alpha-amylase activity of the present invention; and
(B) water.

In another embodiment, the liquid formulation comprises 20% to 80% w/w of polyol. In one embodiment, the liquid formulation comprises 0.001% to 2.0% w/w preservative.

In another embodiment, the invention relates to liquid formulations comprising:
(A) 0.001% to 25% w/w of one or more polypeptides having alpha-amylase activity of the present invention;
(B) 20% to 80% w/w of polyol;
(C) optionally 0.001% to 2.0% w/w preservative; and
(D) water.

In another embodiment, the invention relates to liquid formulations comprising:
(A) 0.001% to 25% w/w of one or more polypeptides having alpha-amylase activity of the present invention;
(B) 0.001% to 2.0% w/w preservative;
(C) optionally 20% to 80% w/w of polyol; and
(D) water.

In another embodiment, the liquid formulation comprises one or more formulating agents, such as a formulating agent selected from the group consisting of polyol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the group consisting of sodium sulfate, dextrin, cellulose, sodium thiosulfate, kaolin and calcium carbonate. In one embodiment, the polyols is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600, more preferably selected from the group consisting of glycerol, sorbitol and propylene glycol (MPG) or any combination thereof.

In another embodiment, the liquid formulation comprises 20%-80% polyol (i.e., total amount of polyol), e.g., 25%-75% polyol, 30%-70% polyol, 35%-65% polyol, or 40%-60% polyol. In one embodiment, the liquid formulation comprises 20%-80% polyol, e.g., 25%-75% polyol, 30%-70% polyol, 35%-65% polyol, or 40%-60% polyol, wherein the polyol is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20%-80% polyol (i.e., total amount of polyol), e.g., 25%-75% polyol, 30%-70% polyol, 35%-65% polyol, or 40%-60% polyol, wherein the polyol is selected from the group consisting of glycerol, sorbitol and propylene glycol (MPG).

In another embodiment, the preservative is selected from the group consisting of sodium sorbate, potassium sorbate, sodium benzoate and potassium benzoate or any combination thereof. In one embodiment, the liquid formulation comprises 0.02% to 1.5% w/w preservative, e.g., 0.05% to 1.0% w/w preservative or 0.1% to 0.5% w/w preservative. In one embodiment, the liquid formulation comprises 0.001% to 2.0% w/w preservative (i.e., total amount of preservative), e.g., 0.02% to 1.5% w/w preservative, 0.05% to 1.0% w/w preservative, or 0.1% to 0.5% w/w preservative, wherein the preservative is selected from the group consisting of sodium sorbate, potassium sorbate, sodium benzoate and potassium benzoate or any combination thereof.

In another embodiment, the liquid formulation further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof.

### Fermentation Broth Formulations or Cell Compositions

The present invention also relates to a fermentation broth formulation or a cell composition comprising a polypeptide of the present invention. The fermentation broth formulation or the cell composition further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In some embodiments, the fermentation broth formulation or the cell composition comprises a first organic acid component comprising at least one 1-5 carbon organic acid and/or a salt thereof and a second organic acid component comprising at least one 6 or more carbon organic acid and/or a salt thereof. In some embodiments, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In some embodiments, the killed cells and/or cell debris are removed from a cell-killed whole broth to provide a composition that is free of these components.

The fermentation broth formulation or cell composition may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell composition of the present invention may be produced by a method described in WO 90/15861 or WO 2010/096673.

### Uses

Processes for Producing Fermentation Products from Gelatinized Starch-Containing Material

In this aspect, the invention relates to processes for producing fermentation products, especially ethanol, from starch-containing material, which process includes a liquefaction step and sequentially or simultaneously performed saccharification and fermentation steps.

Consequently, the invention relates to processes for producing fermentation products from starch-containing material comprising the steps of:
i) liquefying the starch-containing material at a temperature above the initial gelatinization temperature using an alpha-amylase;
ii) saccharifying using a carbohydrate-source generating enzyme;
iii) fermenting using a fermenting organism;
wherein at least one polypeptide having alpha-amylase of the present invention is present or added during fermentation or simultaneous saccharification and fermentation.

Steps ii) and iii) are carried out either sequentially or simultaneously. In a preferred embodiment steps ii) and iii) are carried out simultaneously. An optional thermostable protease, may be added before and/or during liquefaction step i).

The alpha-amylase present or added during fermentation or simultaneous saccharification and fermentation may be of fungal or bacterial origin.

The alpha-amylase present or added during fermentation or simultaneous saccharification and fermentation may be obtained from microorganisms of the genus Penicillium, e.g., a polypeptide obtained from Penicillium oxalicum, Penicillum sclerotiorum, or Penicillium wotroi. In an aspect, the polypeptide having alpha-amylase activity is a Penicillium oxalicum polypeptide, for instance, the Penicillium oxalicum polypeptide having alpha-amylase activity of SEQ ID NO: 2 or SEQ ID NO: 3, or a polypeptide having at least 92%, at least 93%, at least 94%, at least 95%, at least 96%, at least 97%, at least 98%, at least 99% amino acid sequence identity to SEQ ID NO: 2 or SEQ ID NO: 3. In an aspect, the polypeptide having alpha-amylase activity is a Penicillium oxalicum polypeptide, for instance, the Penicillium oxalicum polypeptide having alpha-amylase activity of SEQ ID NO: 2 or SEQ ID NO: 3, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 2 or SEQ ID NO: 3. In an aspect, the polypeptide having alpha-amylase activity is a Penicillium sclerotiorum polypeptide, for instance, the Penicillium sclerotiorum polypeptide having alpha-amylase activity of SEQ ID NO: 5 or SEQ ID NO: 6, or a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 5 or SEQ ID NO: 6. In an aspect, the polypeptide having alpha-amylase activity is a Penicillium sclerotiorum polypeptide, for instance, the Penicillium sclerotiorum polypeptide having alpha-amylase activity of SEQ ID NO: 5 or SEQ ID NO: 6, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 5 or SEQ ID NO: 6. In an aspect, the polypeptide having alpha-amylase activity is a Penicillium wotroi polypeptide, for instance, the Penicillium wotroi polypeptide having alpha-amylase activity of SEQ ID NO: 8 or SEQ ID NO: 9, or a polypeptide having at least 93%, at least 94%, at least 95%, at least 96%, at least 97%, at least 98%, at least 99% amino acid sequence identity to SEQ ID NO: 8 or SEQ ID NO: 9. In an aspect, the polypeptide having alpha-amylase activity is a Penicillium wotroi polypeptide, for instance, the Penicillium wotroi polypeptide having alpha-amylase activity of SEQ ID NO: 8 or SEQ ID NO: 9, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 8 or SEQ ID NO: 9.

The alpha-amylase present or added during fermentation or simultaneous saccharification and fermentation may be obtained from the genus Talaromyces, e.g., a polypeptide obtained from Talaromyces helices. In an aspect, the polypeptide having alpha-amylase activity is a Talaromyces helicus polypeptide, for instance, the Talaromyces helicus polypeptide having alpha-amylase activity of SEQ ID NO: 11 or SEQ ID NO: 12, or a polypeptide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 11 or SEQ ID NO: 12. In an aspect, the polypeptide having alpha-amylase activity is a Talaromyces helicus polypeptide, for instance, the Talaromyces helicus polypeptide having alpha-amylase activity of SEQ ID NO: 11 or SEQ ID NO: 12, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 11 or SEQ ID NO: 12.

The alpha-amylase present or added during fermentation or simultaneous saccharification and fermentation may be obtained from the genus Lactobacillus, e.g., a polypeptide obtained from Lactobacillus amylovorous. In an aspect, the polypeptide having alpha-amylase activity is a Lactobacillus amylovorous polypeptide, for instance, the Lactobacillus amylovorous polypeptide having alpha-amylase activity of SEQ ID NO: 14, SEQ ID NO: 15, SEQ ID NO: 44, SEQ ID NO: 45, SEQ ID NO: 47, SEQ ID NO: 48, SEQ ID NO: 50, SEQ ID NO: 51, SEQ ID NO: 53, SEQ ID NO: 54, SEQ ID NO: 56, SEQ ID NO: 57, SEQ ID NO: 59, SEQ ID NO: 60 or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 14, SEQ ID NO: 15, SEQ ID NO: 44, SEQ ID NO: 45, SEQ ID NO: 47, SEQ ID NO: 48, SEQ ID NO: 50, SEQ ID NO: 51, SEQ ID NO: 53, SEQ ID NO: 54, SEQ ID NO: 56, SEQ ID NO: 57, SEQ ID NO: 59, SEQ ID NO: 60. In an aspect, the polypeptide having alpha-amylase activity is a recombinant polypeptide comprising a Lactobacillus amylovorus catalytic domain having alpha-amylase activity of amino acids 30 to 469 of SEQ ID NO: 14 or of amino acids 1 to 440 of SEQ ID NO: 15, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 30 to 469 of SEQ ID NO: 14 or of amino acids 1 to 440 of SEQ ID NO: 15, and a His-tag (e.g., C-terminal) comprising at least one, at least two, at least three, at least four, at least five, or at least six C-terminal histidine residues. In one embodiment, the recombinant polypeptide comprises a C-terminal His-tag consisting of amino acids 470-475 of SEQ ID NO: SEQ ID NO: 14 or amino acids 441 to 446 of SEQ ID NO: 15.

In an aspect, the polypeptide having alpha-amylase activity is a recombinant polypeptide comprising: (i) a Lactobacillus amylovorus catalytic domain having alpha-amylase activity of amino acids 45 to 467 of SEQ ID NO: 44, SEQ ID NO: 47, SEQ ID NO: 50, SEQ ID NO: 53, SEQ ID NO: 56, or SEQ ID NO: 59, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 45 to 467 of SEQ ID NO: 44, SEQ ID NO: 47, SEQ ID NO: 50, SEQ ID NO: 53, SEQ ID NO: 56, or SEQ ID NO: 59; (ii) at least one starch binding module selected from the group consisting of amino acids 478 to 569 of SEQ ID NO: 44, amino acids 470 to 565 of SEQ ID NO: 47, amino acids 475 to 571 of SEQ ID NO: 50, amino acids 563 to 654 of SEQ ID NO: 53 amino acids 655 to 750 of SEQ ID NO: 53, amino acids 476 to 569 of SEQ ID NO: 56, amino acids 479 to 572, and combinations thereof; (iii) an optional linker connecting the C-terminus of the catalytic domain of (i) to the N-terminus of the at least one starch binding module of (ii), wherein the optional linker is selected from the group consisting of amino acids 468 to 477 of SEQ ID NO: 44, amino acids 468 to 474 of SEQ ID NO: 53, amino acids 468 to 475 of SEQ ID NO: 56, and amino acids 468 to 478 of SEQ ID NO: 57; and (iv) an optional His-tag (e.g., C-terminal) comprising at least one, at least two, at least three, at least four, at least five, or at least six C-terminal histidine residues. In one embodiment, the recombinant polypeptide comprises a C-terminal His-tag consisting of amino acids 570-575 of SEQ ID NO: 44. In one embodiment, the recombinant polypeptide comprises a linker consisting of amino acids 468 to 477 of SEQ ID NO: 44. In one embodiment, the recombinant polypeptide comprises a linker consisting of amino acids 468 to 474 of SEQ ID NO: 53. In one embodiment, the recombinant polypeptide comprises a linker consisting of amino acids 468 to 475 of SEQ ID NO: 56. In one embodiment, the recombinant polypeptide comprises a linker consisting of amino acids 468 to 478 of SEQ ID NO: 59. In one embodiment, the recombinant polypeptide comprises a C-terminal His-tag consisting of amino acids 567 to 572 of SEQ ID NO: 47. In one embodiment, the recombinant polypeptide comprises a C-terminal His-tag consisting of amino acids 572 to 577 of SEQ ID NO: 50. In one embodiment, the recombinant polypeptide comprises a C-terminal His-tag consisting of amino acids 752 to 757 of SEQ ID NO: 53. In one embodiment, the recombinant polypeptide comprises a C-terminal His-tag consisting of amino acids 570 to 575 of SEQ ID NO: 56. In one embodiment, the recombinant polypeptide comprises a C-terminal His-tag consisting of amino acids 573 to 578 of SEQ ID NO: 59. In an embodiment, the recombinant polypeptide has a heterologous secretion signal, such as the Bacillus licheniformis secretion signal consisting of an amino acid sequence of amino acids 1-29 of SEQ ID NO: 14 or an amino acid sequence having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 1-29 of SEQ ID NO: 14. In an embodiment, the recombinant polypeptide has a secretion signal, such as the secretion signal consisting of amino acids 1 to 27 of SEQ ID NO: 44, SEQ ID NO: 47, SEQ ID NO: 50, SEQ ID NO: 53, SEQ ID NO: 56, or SEQ ID NO: 59.

The alpha-amylase present or added during fermentation or simultaneous saccharification and fermentation may be obtained from the genus Bacillus, e.g., a polypeptide obtained from Bacillus amyloliquefaciens. In an aspect, the polypeptide having alpha-amylase activity is a Bacillus amyloliquefaciens polypeptide, for instance, the Bacillus amyloliquefaciens polypeptide having alpha-amylase activity of SEQ ID NO: 41 or SEQ ID NO: 42, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 41 or SEQ ID NO: 42. In an aspect, the polypeptide having alpha-amylase activity is a recombinant polypeptide comprising a Bacillus amyloliquefaciens catalytic domain having alpha-amylase activity of amino acids 28 to 465 of SEQ ID NO: 41, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 28 to 465 of SEQ ID NO: 41, an optional linker having amino acids 466 to 553 of SEQ ID NO: 41, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 466 to 553 of SEQ ID NO: 41, a starch binding module of amino acids 554 to 653 of SEQ ID NO: 41, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 554 to 653 of SEQ ID NO: 41, and a His-tag (e.g., C-terminal) comprising at least one, at least two, at least three, at least four, at least five, or at least six C-terminal histidine residues. In one embodiment, the recombinant polypeptide comprises a C-terminal His-tag consisting of amino acids 654 to 659 of SEQ ID NO: SEQ ID NO: 41. In an embodiment, the recombinant polypeptide has a heterologous secretion signal, such as the Bacillus clausii secretion signal consisting of an amino acid sequence of amino acids 1 to 27 of SEQ ID NO: 41 or an amino acid sequence having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to amino acids 1 to 27 of SEQ ID NO: 41.

The alpha-amylase present or added during fermentation or simultaneous saccharification and fermentation may be obtained from the genus Valsaria, e.g., a polypeptide obtained fromm Valsaria rubricosa. In an aspect, the polypeptide having alpha-amylase activity is a Valsaria rubricosa polypeptide, for instance, the Valsaria rubricosa polypeptide having alpha-amylase activity of SEQ ID NO: 16 or SEQ ID NO: 17, or a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity to SEQ ID NO: 16 or SEQ ID NO: 17.

A composition of the invention may suitably be used in a process of the invention. A recombinant host cell or fermenting organism of the invention may suitably be used in a process of the invention. However, the enzymes may also be added separately.

According to the invention at least polypeptide having alpha-amylase activity of the present invention is present or added during fermentation or simultaneous saccharification and fermentation, however, preferred embodiments may also include the addition of other enzyme classes during fermentation/SSF. Particularly, saccharification and/or fermentation or simultaneous saccharification and fermentation, is performed in the presence of at least one cellulase/cellulolytic composition. More particularly the cellulases/cellulolytic composition are derived from a strain of Trichoderma, in particular Trichoderma reesei, or a strain of Humicola, in particular Humicola insolens, or a strain of Chrysosporium, in particular Chrysosporium lucknowense. The cellulases/cellulolytic composition should at least comprise a beta-glucosidase, a cellobiohydrolase and an endoglucanase. In one embodiment, the cellulases/cellulolytic composition comprises one or more polypeptides selected from the group consisting of:
GH61 polypeptide having cellulolytic enhancing activity,
beta-glucosidase;
Cellobiohydrolase I;
Cellobiohydrolase II;
or a mixture of two, three, or four thereof.

Cellulases are well known in the art, and many are derived from filamentous fungi. Particularly, according to the invention, the cellulases/cellulolytic composition comprises one or more of the following components:
(i) an Aspergillus fumigatus cellobiohydrolase I;
(ii) an Aspergillus fumigatus cellobiohydrolase II;
(iii) an Aspergillus fumigatus beta-glucosidase or variant thereof; and
(iv) a Penicillium sp. GH61 polypeptide having cellulolytic enhancing activity; or homologs thereof.

More specifically the cellulases/cellulolytic composition is in one embodiment a Trichoderma reesei cellulolytic enzyme composition further comprising Penicillium emersonii GH61A polypeptide having cellulolytic enhancing activity disclosed in SEQ ID NO: 18, or polypeptide having at least 90% identity, at least 95% identity, at least 96% identity, at least 97% identity, at least 99% identity to SEQ ID NO: 18 and an Aspergillus fumigatus beta-glucosidase disclosed in SEQ ID NO: 19 or a variant thereof with the following substitutions: F100D, S283G, N456E, F512Y having at least 90% identity, at least 95% identity, at least 96% identity, at least 97% identity, at least 99% identity to SEQ ID NO: 19.

In one embodiment, the cellulolytic composition comprises a cellobiohydrolase I (CBH I), such as one derived from a strain of the genus Aspergillus, such as a strain of Aspergillus fumigatus, such as the CBHI disclosed as SEQ ID NO: 20, or CBH I having at least 90% identity, at least 95% identity, at least 96% identity, at least 97% identity, at least 99% identity to SEQ ID NO: 20.

In one embodiment, the cellulolytic composition comprises a cellobiohydrolase II (CBH II), such as one derived from a strain of the genus Aspergillus, such as a strain of Aspergillus fumigatus; such as the CBH II disclosed as SEQ ID NO: 21, or a CBH II having at least 90% identity, at least 95% identity, at least 96% identity, at least 97% identity, at least 99% identity to SEQ ID NO: 21.

Examples of suitable cellulases can be found in “Cellulolytic Composition present and/or added during Saccharification and/or Fermentation”

Examples of alpha-amylases can be found in the “Alpha-Amylase Present and/or Added During Liquefaction”-section below.

Examples of thermostable proteases can be found in the “Protease Present and/or Added During Liquefaction”-section below.

Examples of suitable optional carbohydrate-source generating enzymes, preferably thermostable carbohydrate-source generating enzymes, in particular, a thermostable glucoamylase, can be found in the “Carbohydrate-Source Generating Enzymes Present and/or Added During Liquefaction”-section below.

The pH during liquefaction may be between 4-7. In an embodiment the pH during liquefaction is from 4.5-5.0, such as between 4.5-4.8. In another embodiment liquefaction is carried out at a pH above 5.0-6.5, such as above 5.0-6.0, such as above 5.0-5.5, such as between 5.2-6.2, such as around 5.2, such as around 5.4, such as around 5.6, such as around 5.8. According to the invention the temperature is above the initial gelatinization temperature. The term “initial gelatinization temperature” refers to the lowest temperature at which solubilization of starch, typically by heating, begins. The temperature can vary for different starches.

In an embodiment the temperature during liquefaction step i) is in the range from 70-100° C., such as between 75-95° C., such as between 75-90° C., preferably between 80-90° C., such as between 82-88° C., such as around 85° C.

In an embodiment, the process of the invention further comprises, prior to the step i), the steps of:
a) reducing the particle size of the starch-containing material, preferably by dry milling;
b) forming a slurry comprising the starch-containing material and water.

The starch-containing starting material, such as whole grains, may be reduced in particle size, e.g., by milling, in order to open up the structure, to increase surface area, and allowing for further processing. Generally, there are two types of processes: wet and dry milling. In dry milling whole kernels are milled and used. Wet milling gives a good separation of germ and meal (starch granules and protein). Wet milling is often applied at locations where the starch hydrolysate is used in production of, e.g., syrups. Both dry and wet milling are well known in the art of starch processing. According to the present invention dry milling is preferred. In an embodiment the particle size is reduced to between 0.05 to 3.0 mm, preferably 0.1-0.5 mm, or so that at least 30%, preferably at least 50%, more preferably at least 70%, even more preferably at least 90% of the starch-containing material fit through a sieve with a 0.05 to 3.0 mm screen, preferably 0.1-0.5 mm screen. In another embodiment at least 50%, preferably at least 70%, more preferably at least 80%, especially at least 90% of the starch-containing material fit through a sieve with #6 screen.

The aqueous slurry may contain from 10-55 w/w-% dry solids (DS), preferably 25-45 w/w-% dry solids (DS), more preferably 30-40 w/w-% dry solids (DS) of starch-containing material. The slurry may be heated to above the initial gelatinization temperature, preferably to between 80-90° C., between pH 4-7, preferably between 4.5-5.0 or 5.0 and 6.0, for 30 minutes to 5 hours, such as around 2 hours.

The alpha-amylase, optional thermostable protease, optional carbohydrate-source generating enzyme, in particular thermostable glucoamylase, may initially be added to the aqueous slurry to initiate liquefaction (thinning). In an embodiment only a portion of the enzymes is added to the aqueous slurry, while the rest of the enzymes are added during liquefaction step i).

Liquefaction step i) is according to the invention carried out for 0.5-5 hours, such as 1-3 hours, such as typically around 2 hours.

The aqueous slurry may in an embodiment be jet-cooked to further gelatinize the slurry before being subjected to liquefaction in step i). The jet-cooking may be carried out at a temperature between 110-145° C., preferably 120-140° C., such as 125-135° C., preferably around 130° C. for about 1-15 minutes, preferably for about 3-10 minutes, especially around about 5 minutes.

### Saccharification and Fermentation

One or more carbohydrate-source generating enzymes, in particular glucoamylase, may be present and/or added during saccharification step ii) and/or fermentation step iii). The carbohydrate-source generating enzyme may preferably be a glucoamylase, but may also be an enzyme selected from the group consisting of: beta-amylase, maltogenic amylase and alpha-glucosidase. The carbohydrate-source generating enzyme added during saccharification step ii) and/or fermentation step iii) is typically different from the optional carbohydrate-source generating enzyme, in particular thermostable glucoamylase, optionally added during liquefaction step i). In a preferred embodiment the carbohydrate-source generating enzymes, in particular glucoamylase, is added together with a fungal alpha-amylase.

Examples of carbohydrate-source generating enzymes, including glucoamylases, can be found in the “Carbohydrate-Source Generating Enzyme Present and/or Added During Saccharification and/or Fermentation”-section below.

When doing sequential saccharification and fermentation, saccharification step ii) may be carried out at conditions well-known in the art. For instance, the saccharification step ii) may last up to from about 24 to about 72 hours. In an embodiment pre-saccharification is done. Pre-saccharification is typically done for 40-90 minutes at a temperature between 30-65° C., typically about 60° C. Pre-saccharification is in an embodiment followed by saccharification during fermentation in simultaneous saccharification and fermentation (“SSF). Saccharification is typically carried out at temperatures from 20-75° C., preferably from 40-70° C., typically around 60° C., and at a pH between 4 and 5, normally at about pH 4.5.

Simultaneous saccharification and fermentation (“SSF”) is widely used in industrial scale fermentation product production processes, especially ethanol production processes. When doing SSF the saccharification step ii) and the fermentation step iii) are carried out simultaneously. There is no holding stage for the saccharification, meaning that a fermenting organism, such as yeast, and enzyme(s), may be added together. However, it is also contemplated to add the fermenting organism and enzyme(s) separately. SSF is according to the invention typically carried out at a temperature from 25° C. to 40° C., such as from 28° C. to 35° C., such as from 30° C. to 34° C., preferably around about 32° C. In an embodiment fermentation is ongoing for 6 to 120 hours, in particular 24 to 96 hours. In an embodiment the pH is between 3.5-5, in particular between 3.8 and 4.3.

### Fermentation Medium

“Fermentation media” or “fermentation medium” refers to the environment in which fermentation is carried out. The fermentation medium includes the fermentation substrate, that is, the carbohydrate source that is metabolized by the fermenting organism. According to the invention the fermentation medium may comprise nutrients and growth stimulator(s) for the fermenting organism(s). Nutrient and growth stimulators are widely used in the art of fermentation and include nitrogen sources, such as ammonia; urea, vitamins and minerals, or combinations thereof.

### Fermenting Organisms

The term “Fermenting organism” refers to any organism, including bacterial and fungal organisms, especially yeast, suitable for use in a fermentation process and capable of producing the desired fermentation product. Especially suitable fermenting organisms are able to ferment, i.e., convert, sugars, such as glucose or maltose, directly or indirectly into the desired fermentation product, such as ethanol. Examples of fermenting organisms include fungal organisms, such as yeast. Preferred yeast includes strains of Saccharomyces spp., in particular, Saccharomyces cerevisiae.

Suitable concentrations of the viable fermenting organism during fermentation, such as SSF, are well known in the art or can easily be determined by the skilled person in the art. In one embodiment the fermenting organism, such as ethanol fermenting yeast, (e.g., Saccharomyces cerevisiae) is added to the fermentation medium so that the viable fermenting organism, such as yeast, count per mL of fermentation medium is in the range from 10⁵ to 10¹², preferably from 10⁷ to 10¹⁰, especially about 5×10⁷.

Examples of commercially available yeast includes, e.g., RED STAR™ and ETHANOL RED™ yeast (available from Fermentis/Lesaffre, USA), FALI (available from Fleischmann's Yeast, USA), SUPERSTART and THERMOSACC™ fresh yeast (available from Ethanol Technology, WI, USA), BIOFERM AFT and XR (available from NABC—North American Bioproducts Corporation, GA, USA), GERT STRAND (available from Gert Strand AB, Sweden), and FERMIOL (available from DSM Specialties). Other useful yeast strains are available from biological depositories such as the American Type Culture Collection (ATCC) or the Deutsche Sammlung von Mikroorganismen und Zellkulturen GmbH (DSMZ), such as, e.g., BY4741 (e.g., ATCC 201388); Y108-1 (ATCC PTA. 10567) and NRRL YB-1952 (ARS Culture Collection). Still other S. cerevisiae strains suitable as host cells DBY746, [Alpha][Eta]22, S150-2B, GPY55-15Ba, CEN.PK, USM21, TMB3500, TMB3400, VTT-A-63015, VTT-A-85068, VTT-c-79093 and their derivatives as well as Saccharomyces sp. 1400, 424A (LNH-ST), 259A (LNH-ST) and derivatives thereof.

As used herein, a “derivative” of strain is derived from a referenced strain, such as through mutagenesis, recombinant DNA technology, mating, cell fusion, or cytoduction between yeast strains. Those skilled in the art will understand that the genetic alterations, including metabolic modifications exemplified herein, may be described with reference to a suitable host organism and their corresponding metabolic reactions or a suitable source organism for desired genetic material such as genes for a desired metabolic pathway. However, given the complete genome sequencing of a wide variety of organisms and the high level of skill in the area of genomics, those skilled in the art can apply the teachings and guidance provided herein to other organisms. For example, the metabolic alterations exemplified herein can readily be applied to other species by incorporating the same or analogous encoding nucleic acid from species other than the referenced species.

The host cell or fermenting organism may be Saccharomyces strain, e.g., Saccharomyces cerevisiae strain produced using the method described and concerned in U.S. Pat. No. 8,257,959-BB. In one embodiment, the recombinant cell is a derivative of a strain Saccharomyces cerevisiae CIBTS1260 (deposited under Accession No. NRRL Y-50973 at the Agricultural Research Service Culture Collection (NRRL), Illinois 61604 U.S.A.).

The strain may also be a derivative of Saccharomyces cerevisiae strain NMI V14/004037 (See, WO2015/143324 and WO2015/143317 each incorporated herein by reference), strain nos. V15/004035, V15/004036, and V15/004037 (See, WO 2016/153924 incorporated herein by reference), strain nos. V15/001459, V15/001460, V15/001461 (See, WO2016/138437 incorporated herein by reference), strain no. NRRL Y67342 (See, WO2018/098381 incorporated herein by reference), strain nos. NRRL Y67549 and NRRL Y67700 (See, PCT/US2019/018249 incorporated herein by reference), or any strain described in WO2017/087330 (incorporated herein by reference).

The fermenting organisms may be a host cell that expresses a heterologous polypeptide having alpha-amylase activity, particularly a polypeptide having alpha-amylase acitivity (e.g., any polypeptide having alpha-amylase activity described herein). Any polypeptide having alpha-amylase activity contemplated for a process, enzyme blend, or composition described herein is also contemplated for expression by a fermenting organism or host cell.

In some embodiments, the host cells and/or fermenting organisms comprise one or more heterologous polynucleotides encoding an alpha-amylase, glucoamylase, protease and/or cellulase. Examples of alpha-amylase, glucoamylase, protease and cellulases suitable for expression in the host cells and/or fermenting organisms are described in more detail herein. The host cells and fermenting organisms described herein may utilize expression vectors comprising the coding sequence of one or more (e.g., two, several) heterologous genes linked to one or more control sequences that direct expression in a suitable cell under conditions compatible with the control sequence(s). Such expression vectors may be used in any of the cells and methods described herein. The polynucleotides described herein may be manipulated in a variety of ways to provide for expression of a desired polypeptide. Manipulation of the polynucleotide prior to its insertion into a vector may be desirable or necessary depending on the expression vector. The techniques for modifying polynucleotides utilizing recombinant DNA methods are well known in the art.

A construct or vector (or multiple constructs or vectors) comprising the one or more (e.g., two, several) heterologous genes may be introduced into a cell so that the construct or vector is maintained as a chromosomal integrant or as a self-replicating extra-chromosomal vector as described earlier.

The various nucleotide and control sequences may be joined together to produce a recombinant expression vector that may include one or more (e.g., two, several) convenient restriction sites to allow for insertion or substitution of the polynucleotide at such sites. Alternatively, the polynucleotide(s) may be expressed by inserting the polynucleotide(s) or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the cell, or a transposon, may be used.

The expression vector may contain any suitable promoter sequence that is recognized by a cell for expression of a gene described herein. The promoter sequence contains transcriptional control sequences that mediate the expression of the polypeptide. The promoter may be any polynucleotide that shows transcriptional activity in the cell of choice including mutant, truncated, and hybrid promoters, and may be obtained from genes encoding extracellular or intracellular polypeptides either homologous or heterologous to the cell.

Each heterologous polynucleotide described herein may be operably linked to a promoter that is foreign to the polynucleotide. For example, in one embodiment, the nucleic acid construct encoding the fusion protein is operably linked to a promoter foreign to the polynucleotide. The promoters may be identical to or share a high degree of sequence identity (e.g., at least about 80%, at least about 85%, at least about 90%, at least about 95%, at least about 96%, at least about 97%, at least about 98%, or at least about 99%) with a selected native promoter.

Examples of suitable promoters for directing the transcription of the nucleic acid constructs in a yeast cells, include, but are not limited to, the promoters obtained from the genes for enolase, (e.g., S. cerevisiae enolase or I. orientalis enolase (ENO1)), galactokinase (e.g., S. cerevisiae galactokinase or I. orientalis galactokinase (GAL1)), alcohol dehydrogenase/glyceraldehyde-3-phosphate dehydrogenase (e.g., S. cerevisiae alcohol dehydrogenase/glyceraldehyde-3-phosphate dehydrogenase or I. orientalis alcohol dehydrogenase/glyceraldehyde-3-phosphate dehydrogenase (ADH1, ADH2/GAP)), triose phosphate isomerase (e.g., S. cerevisiae triose phosphate isomerase or I. orientalis triose phosphate isomerase (TPI)), metallothionein (e.g., S. cerevisiae metallothionein or I. orientalis metallothionein (CUP1)), 3-phosphoglycerate kinase (e.g., S. cerevisiae 3-phosphoglycerate kinase or I. orientalis 3-phosphoglycerate kinase (PGK)), PDC1, xylose reductase (XR), xylitol dehydrogenase (XDH), L-(+)-lactate-cytochrome c oxidoreductase (CYB2), translation elongation factor-1 (TEF1), translation elongation factor-2 (TEF2), glyceraldehyde-3-phosphate dehydrogenase (GAPDH), and orotidine 5′-phosphate decarboxylase (URA3) genes. Other suitable promoters may be obtained from S. cerevisiae TDH3, HXT7, PGK1, RPL18B and CCW12 genes. Additional useful promoters for yeast host cells are described by Romanos et al., 1992, Yeast 8: 423-488.

The control sequence may also be a suitable transcription terminator sequence, which is recognized by a host cell to terminate transcription. The terminator sequence is operably linked to the 3′-terminus of the polynucleotide encoding the polypeptide. Any terminator that is functional in the yeast cell of choice may be used. The terminator may be identical to or share a high degree of sequence identity (e.g., at least about 80%, at least about 85%, at least about 90%, at least about 95%, at least about 96%, at least about 97%, at least about 98%, or at least about 99%) with the selected native terminator.

Suitable terminators for yeast host cells may be obtained from the genes for enolase (e.g., S. cerevisiae or I. orientalis enolase cytochrome C (e.g., S. cerevisiae or I. orientalis cytochrome (CYC1)), glyceraldehyde-3-phosphate dehydrogenase (e.g., S. cerevisiae or I. orientalis glyceraldehyde-3-phosphate dehydrogenase (gpd)), PDC1, XR, XDH, transaldolase (TAL), transketolase (TKL), ribose 5-phosphate ketol-isomerase (RKI), CYB2, and the galactose family of genes (especially the GAL 10 terminator). Other suitable terminators may be obtained from S. cerevisiae ENO2 or TEF1 genes. Additional useful terminators for yeast host cells are described by Romanos et al., 1992, supra.

The control sequence may also be an mRNA stabilizer region downstream of a promoter and upstream of the coding sequence of a gene which increases expression of the gene. Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a suitable leader sequence, when transcribed is a non-translated region of an mRNA that is important for translation by the host cell. The leader sequence is operably linked to the 5′-terminus of the polynucleotide encoding the polypeptide. Any leader sequence that is functional in the yeast cell of choice may be used.

Suitable leaders for yeast host cells are obtained from the genes for enolase (e.g., S. cerevisiae or I. orientalis enolase (ENO-1)), 3-phosphoglycerate kinase (e.g., S. cerevisiae or I. orientalis 3-phosphoglycerate kinase), alpha-factor (e.g., S. cerevisiae or I. orientalis alpha-factor), and alcohol dehydrogenase/glyceraldehyde-3-phosphate dehydrogenase (e.g., S. cerevisiae or I. orientalis alcohol dehydrogenase/glyceraldehyde-3-phosphate dehydrogenase (ADH2/GAP)).

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell of choice may be used. Useful polyadenylation sequences for yeast cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used. Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that allow the regulation of the expression of the polypeptide relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used.

The vectors may contain one or more (e.g., two, several) selectable markers that permit easy selection of transformed, transfected, transduced, or the like cells. A selectable marker is a gene the product of which provides for biocide or viral resistance, resistance to heavy metals, prototrophy to auxotrophs, and the like. Suitable markers for yeast host cells include, but are not limited to, ADE2, HIS3, LEU2, LYS2, MET3, TRP1, and URA3.

The vectors may contain one or more (e.g., two, several) elements that permit integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination. Potential integration loci include those described in the art (e.g., See US2012/0135481).

For autonomous replication, the vector may further comprise an origin of replication enabling the vector to replicate autonomously in the yeast cell. The origin of replication may be any plasmid replicator mediating autonomous replication that functions in a cell. The term “origin of replication” or “plasmid replicator” means a polynucleotide that enables a plasmid or vector to replicate in vivo. Examples of origins of replication for use in a yeast host cell are the 2 micron origin of replication, ARS1, ARS4, the combination of ARS1 and CEN3, and the combination of ARS4 and CEN6.

More than one copy of a polynucleotide described herein may be inserted into a host cell to increase production of a polypeptide. An increase in the copy number of the polynucleotide can be obtained by integrating at least one additional copy of the sequence into the yeast cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors described herein are well known to one skilled in the art (see, e.g., Sambrook et al., 1989, Molecular Cloning, A Laboratory Manual, 2d edition, Cold Spring Harbor, New York). Additional procedures and techniques known in the art for the preparation of recombinant cells for ethanol fermentation, are described in, e.g., WO 2016/045569, the content of which is hereby incorporated by reference.

The host cell or fermenting organism may be in the form of a composition comprising a host cell or fermenting organism (e.g., a yeast strain described herein) and a naturally occurring and/or a non-naturally occurring component.

The host cell or fermenting organism described herein may be in any viable form, including crumbled, dry, including active dry and instant, compressed, cream (liquid) form etc. In one embodiment, the host cell or fermenting organism (e.g., a Saccharomyces cerevisiae yeast strain) is dry yeast, such as active dry yeast or instant yeast. In one embodiment, the host cell or fermenting organism (e.g., a Saccharomyces cerevisiae yeast strain) is crumbled yeast. In one embodiment, the host cell or fermenting organism (e.g., a Saccharomyces cerevisiae yeast strain) is compressed yeast. In one embodiment, the host cell or fermenting organism (e.g., a Saccharomyces cerevisiae yeast strain) is cream yeast.

In one embodiment is a composition comprising a host cell or fermenting organism described herein (e.g., a Saccharomyces cerevisiae yeast strain), and one or more of the component selected from the group consisting of: surfactants, emulsifiers, gums, swelling agent, and antioxidants and other processing aids.

The compositions described herein may comprise a host cell or fermenting organism described herein (e.g., a Saccharomyces cerevisiae yeast strain) and any suitable surfactants. In one embodiment, the surfactant(s) is/are an anionic surfactant, cationic surfactant, and/or nonionic surfactant.

The compositions described herein may comprise a host cell or fermenting organism described herein (e.g., a Saccharomyces cerevisiae yeast strain) and any suitable emulsifier. In one embodiment, the emulsifier is a fatty-acid ester of sorbitan. In one embodiment, the emulsifier is selected from the group of sorbitan monostearate (SMS), citric acid esters of monodiglycerides, polyglycerolester, fatty acid esters of propylene glycol.

In one embodiment, the composition comprises a host cell or fermenting organism described herein (e.g., a Saccharomyces cerevisiae yeast strain), and Olindronal SMS, Olindronal SK, or Olindronal SPL including composition concerned in European Patent No. 1,724,336 (hereby incorporated by reference). These products are commercially available from Bussetti, Austria, for active dry yeast.

The compositions described herein may comprise a host cell or fermenting organism described herein (e.g., a Saccharomyces cerevisiae yeast strain) and any suitable gum. In one embodiment, the gum is selected from the group of carob, guar, tragacanth, arabic, xanthan and acacia gum, in particular for cream, compressed and dry yeast.

The compositions described herein may comprise a host cell or fermenting organism described herein (e.g., a Saccharomyces cerevisiae yeast strain) and any suitable swelling agent. In one embodiment, the swelling agent is methyl cellulose or carboxymethyl cellulose.

The compositions described herein may comprise a host cell or fermenting organism described herein (e.g., a Saccharomyces cerevisiae yeast strain) and any suitable anti-oxidant. In one embodiment, the antioxidant is butylated hydroxyanisol (BHA) and/or butylated hydroxytoluene (BHT), or ascorbic acid (vitamin C), particular for active dry yeast.

The host cells and fermenting organisms described herein may also comprise one or more (e.g., two, several) gene disruptions, e.g., to divert sugar metabolism from undesired products to ethanol. In some embodiments, the recombinant host cells produce a greater amount of ethanol compared to the cell without the one or more disruptions when cultivated under identical conditions. In some embodiments, one or more of the disrupted endogenous genes is inactivated. In certain embodiments, the host cell or fermenting organism provided herein comprises a disruption of one or more endogenous genes encoding enzymes involved in producing alternate fermentative products such as glycerol or other byproducts such as acetate or diols. For example, the cells provided herein may comprise a disruption of one or more of glycerol 3-phosphate dehydrogenase (GPD, catalyzes reaction of dihydroxyacetone phosphate to glycerol 3-phosphate), glycerol 3-phosphatase (GPP, catalyzes conversion of glycerol-3 phosphate to glycerol), glycerol kinase (catalyzes conversion of glycerol 3-phosphate to glycerol), dihydroxyacetone kinase (catalyzes conversion of dihydroxyacetone phosphate to dihydroxyacetone), glycerol dehydrogenase (catalyzes conversion of dihydroxyacetone to glycerol), and aldehyde dehydrogenase (ALD, e.g., converts acetaldehyde to acetate).

Modeling analysis can be used to design gene disruptions that additionally optimize utilization of the pathway. One exemplary computational method for identifying and designing metabolic alterations favoring biosynthesis of a desired product is the OptKnock computational framework, Burgard et al., 2003, Biotechnol. Bioeng. 84: 647-657.

The host cells and fermenting organisms comprising a gene disruption may be constructed using methods well known in the art, including those methods described herein. A portion of the gene can be disrupted such as the coding region or a control sequence required for expression of the coding region. Such a control sequence of the gene may be a promoter sequence or a functional part thereof, i.e., a part that is sufficient for affecting expression of the gene. For example, a promoter sequence may be inactivated resulting in no expression or a weaker promoter may be substituted for the native promoter sequence to reduce expression of the coding sequence. Other control sequences for possible modification include, but are not limited to, a leader, propeptide sequence, signal sequence, transcription terminator, and transcriptional activator.

The host cells and fermenting organisms comprising a gene disruption may be constructed by gene deletion techniques to eliminate or reduce expression of the gene. Gene deletion techniques enable the partial or complete removal of the gene thereby eliminating their expression. In such methods, deletion of the gene is accomplished by homologous recombination using a plasmid that has been constructed to contiguously contain the 5′ and 3′ regions flanking the gene.

The host cells and fermenting organisms comprising a gene disruption may also be constructed by introducing, substituting, and/or removing one or more (e.g., two, several) nucleotides in the gene or a control sequence thereof required for the transcription or translation thereof. For example, nucleotides may be inserted or removed for the introduction of a stop codon, the removal of the start codon, or a frame-shift of the open reading frame. Such a modification may be accomplished by site-directed mutagenesis or PCR generated mutagenesis in accordance with methods known in the art. See, for example, Botstein and Shortle, 1985, Science 229: 4719; Lo et al., 1985, Proc. Natl. Acad. Sci. U.S.A. 81: 2285; Higuchi et al., 1988, Nucleic Acids Res 16: 7351; Shimada, 1996, Meth. Mol. Biol. 57: 157; Ho et al., 1989, Gene 77: 61; Horton et al., 1989, Gene 77: 61; and Sarkar and Sommer, 1990, BioTechniques 8: 404.

The host cells and fermenting organisms comprising a gene disruption may also be constructed by inserting into the gene a disruptive nucleic acid construct comprising a nucleic acid fragment homologous to the gene that will create a duplication of the region of homology and incorporate construct DNA between the duplicated regions. Such a gene disruption can eliminate gene expression if the inserted construct separates the promoter of the gene from the coding region or interrupts the coding sequence such that a non-functional gene product results. A disrupting construct may be simply a selectable marker gene accompanied by 5′ and 3′ regions homologous to the gene. The selectable marker enables identification of transformants containing the disrupted gene.

The host cells and fermenting organisms comprising a gene disruption may also be constructed by the process of gene conversion (see, for example, Iglesias and Trautner, 1983, Molecular General Genetics 189: 73-76). For example, in the gene conversion method, a nucleotide sequence corresponding to the gene is mutagenized in vitro to produce a defective nucleotide sequence, which is then transformed into the recombinant strain to produce a defective gene. By homologous recombination, the defective nucleotide sequence replaces the endogenous gene. It may be desirable that the defective nucleotide sequence also comprises a marker for selection of transformants containing the defective gene.

The host cells and fermenting organisms comprising a gene disruption may be further constructed by random or specific mutagenesis using methods well known in the art, including, but not limited to, chemical mutagenesis (see, for example, Hopwood, The Isolation of Mutants in Methods in Microbiology (J. R. Norris and D. W. Ribbons, eds.) pp. 363-433, Academic Press, New York, 1970). Modification of the gene may be performed by subjecting the parent strain to mutagenesis and screening for mutant strains in which expression of the gene has been reduced or inactivated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods. Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosogaunidine (NTG)O-methyl hydroxylamine, nitrous acid, ethyl methane sulphonate (EMS), sodium bisulphite, formic acid, and nucleotide analogues. When such agents are used, the mutagenesis is typically performed by incubating the parent strain to be mutagenized in the presence of the mutagenizing agent of choice under suitable conditions, and selecting for mutants exhibiting reduced or no expression of the gene.

A nucleotide sequence homologous or complementary to a gene described herein may be used from other microbial sources to disrupt the corresponding gene in a recombinant strain of choice.

In one embodiment, the modification of a gene in the recombinant cell is unmarked with a selectable marker. Removal of the selectable marker gene may be accomplished by culturing the mutants on a counter-selection medium. Where the selectable marker gene contains repeats flanking its 5′ and 3′ ends, the repeats will facilitate the looping out of the selectable marker gene by homologous recombination when the mutant strain is submitted to counter-selection. The selectable marker gene may also be removed by homologous recombination by introducing into the mutant strain a nucleic acid fragment comprising 5′ and 3′ regions of the defective gene, but lacking the selectable marker gene, followed by selecting on the counter-selection medium. By homologous recombination, the defective gene containing the selectable marker gene is replaced with the nucleic acid fragment lacking the selectable marker gene. Other methods known in the art may also be used.

### Starch-Containing Materials

Any suitable starch-containing material may be used according to the present invention. The starting material is generally selected based on the desired fermentation product. Examples of starch-containing materials, suitable for use in a process of the invention, include whole grains, corn, wheat, barley, rye, milo, sago, cassava, tapioca, sorghum, rice, peas, beans, or sweet potatoes, or mixtures thereof or starches derived there from, or cereals. Contemplated are also waxy and non-waxy types of corn and barley. In a preferred embodiment the starch-containing material, used for ethanol production according to the invention, is corn or wheat.

### Fermentation Products

The term “fermentation product” means a product produced by a process including a fermentation step using a fermenting organism. Fermentation products contemplated according to the invention include alcohols (e.g., ethanol, methanol, butanol; polyols such as glycerol, sorbitol and inositol); organic acids (e.g., citric acid, acetic acid, itaconic acid, lactic acid, succinic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. In a preferred embodiment the fermentation product is ethanol, e.g., fuel ethanol; drinking ethanol, i.e., potable neutral spirits; or industrial ethanol or products used in the consumable alcohol industry (e.g., beer and wine), dairy industry (e.g., fermented dairy products), leather industry and tobacco industry. Preferred beer types comprise ales, stouts, porters, lagers, bitters, malt liquors, happoushu, high-alcohol beer, low-alcohol beer, low-calorie beer or light beer. Preferably processes of the invention are used for producing an alcohol, such as ethanol. The fermentation product, such as ethanol, obtained according to the invention, may be used as fuel, which is typically blended with gasoline. However, in the case of ethanol it may also be used as potable ethanol.

### Recovery

Subsequent to fermentation, or SSF, the fermentation product may be separated from the fermentation medium. The slurry may be distilled to extract the desired fermentation product (e.g., ethanol). Alternatively, the desired fermentation product may be extracted from the fermentation medium by micro or membrane filtration techniques. The fermentation product may also be recovered by stripping or other method well known in the art.

Alpha-Amylase Present and/or Added During Liquefaction

According to the invention an alpha-amylase is present and/or added during liquefaction together with an optional thermostable protease, optional carbohydrate-source generating enzyme, in particular a thermostable glucoamylase, and/or optional pullulanase.

The alpha-amylase added during liquefaction step i) may be any alpha-amylase. Preferred are bacterial alpha-amylases, which typically are stable at temperature used during liquefaction.

Any alpha-amylase herein contemplated as being present and/or added during liquefaction is also contemplated for expression by a fermenting organism or host cell.

The term “bacterial alpha-amylase” means any bacterial alpha-amylase classified under EC 3.2.1.1. A bacterial alpha-amylase used according to the invention may, e.g., be derived from a strain of the genus Bacillus, which is sometimes also referred to as the genus Geobacillus. In an embodiment the Bacillus alpha-amylase is derived from a strain of Bacillus amyloliquefaciens, Bacillus licheniformis, Bacillus stearothermophilus, or Bacillus subtilis, but may also be derived from other Bacillus sp.

Specific examples of bacterial alpha-amylases include the Bacillus stearothermophilus alpha-amylase of SEQ ID NO: 3 in WO 99/19467, the Bacillus amyloliquefaciens alpha-amylase of SEQ ID NO: 5 in WO 99/19467, and the Bacillus licheniformis alpha-amylase of SEQ ID NO: 4 in WO 99/19467 (all sequences are hereby incorporated by reference). In an embodiment the alpha-amylase may be an enzyme having a degree of identity of at least 60%, e.g., at least 70%, at least 80%, at least 90%, at least 95%, at least 96%, at least 97%, at least 98% or at least 99% to any of the sequences shown in SEQ ID NOS: 3, 4 or 5, respectively, in WO 99/19467.

In an embodiment the alpha-amylase may be an enzyme having a degree of identity of at least 60%, e.g., at least 70%, at least 80%, at least 90%, at least 91%, at least 92%, at least 93%, at least 94%, at least 95%, at least 96%, at least 97%, at least 98% or at least 99% to any of the sequences shown in SEQ ID NO: 3 in WO 99/19467 or SEQ ID NO: 22 herein.

In a preferred embodiment the alpha-amylase is derived from Bacillus stearothermophilus. The Bacillus stearothermophilus alpha-amylase may be a mature wild-type or a mature variant thereof. The mature Bacillus stearothermophilus alpha-amylases may naturally be truncated during recombinant production. For instance, the Bacillus stearothermophilus alpha-amylase may be a truncated so it has around 491 amino acids compared to SEQ ID NO: 3 in WO 99/19467.

The Bacillus alpha-amylase may also be a variant and/or hybrid. Examples of such a variant can be found in any of WO 96/23873, WO 96/23874, WO 97/41213, WO 99/19467, WO 00/60059, and WO 02/10355 (all documents are hereby incorporated by reference). Specific alpha-amylase variants are disclosed in U.S. Pat. Nos. 6,093,562, 6, 187,576, 6,297,038, and 7,713,723 (hereby incorporated by reference) and include Bacillus stearothermophilus alpha-amylase (often referred to as BSG alpha-amylase) variants having a deletion of one or two amino acids at positions R179, G180, I181 and/or G182, preferably a double deletion disclosed in WO 96/23873—see, e.g., page 20, lines 1-10 (hereby incorporated by reference), preferably corresponding to deletion of positions 1181 and G182 compared to the amino acid sequence of Bacillus stearothermophilus alpha-amylase set forth in SEQ ID NO: 3 disclosed in WO 99/19467 or SEQ ID NO: 22 herein or the deletion of amino acids R179 and G180 using SEQ ID NO: 3 in WO 99/19467 or SEQ ID NO: 22 herein for numbering (which reference is hereby incorporated by reference). Even more preferred are Bacillus alpha-amylases, especially Bacillus stearothermophilus alpha-amylases, which have a double deletion corresponding to a deletion of positions 181 and 182 and further comprise a N193F substitution (also denoted I181*+G182*+N193F) compared to the wild-type BSG alpha-amylase amino acid sequence set forth in SEQ ID NO: 3 disclosed in WO 99/19467 or SEQ ID NO: 22 herein. The bacterial alpha-amylase may also have a substitution in a position corresponding to S239 in the Bacillus licheniformis alpha-amylase shown in SEQ ID NO: 4 in WO 99/19467, or a S242 variant of the Bacillus stearothermophilus alpha-amylase of SEQ ID NO: 3 in WO 99/19467 or SEQ ID NO: 22 herein.

In an embodiment the variant is a S242A, E or Q variant, preferably a S242Q variant, of the Bacillus stearothermophilus alpha-amylase (using SEQ ID NO: 22 herein for numbering).

In an embodiment the variant is a position E188 variant, preferably E188P variant of the Bacillus stearothermophilus alpha-amylase (using SEQ ID NO: 22 herein for numbering).

The bacterial alpha-amylase may in an embodiment be a truncated alpha-amylase. Especially the truncation is so that the Bacillus stearothermophilus alpha-amylase shown in SEQ ID NO: 3 in WO 99/19467 or SEQ ID NO: 22 herein, is around 491 amino acids long, such as from 480 to 495 amino acids long.

Most importantly, a suitable alpha-amylase for use in liquefaction must have sufficient therm-stability, and thus accordingly any alpha-amylase having a T½ (min) at pH 4.5, 85° C., 0.12 mM CaCl₂)) of at least 10, such as at least 15, such as at least 20, such as at least 25, such as at least 30, such as at least 40, such as at least 50, such as at least 60, such as between 10-70, such as between 15-70, such as between 20-70, such as between 25-70, such as between 30-70, such as between 40-70, such as between 50-70, such as between 60-70, may be used.

According to the invention the alpha-amylase may be a thermostable alpha-amylase, such as a thermostable bacterial alpha-amylase, preferably from Bacillus stearothermophilus. In an embodiment the alpha-amylase used according to the invention has a T½ (min) at pH 4.5, 85° C., 0.12 mM CaCl₂) of at least 10.

In an embodiment the thermostable alpha-amylase has a T½ (min) at pH 4.5, 85° C., 0.12 mM CaCl₂), of at least 15.

In an embodiment the thermostable alpha-amylase has a T½ (min) at pH 4.5, 85° C., 0.12 mM CaCl₂), of as at least 20.

In an embodiment the thermostable alpha-amylase has a T½ (min) at pH 4.5, 85° C., 0.12 mM CaCl₂), of as at least 25.

In an embodiment the thermostable alpha-amylase has a T½ (min) at pH 4.5, 85° C., 0.12 mM CaCl₂), of as at least 30.

In an embodiment the thermostable alpha-amylase has a T½ (min) at pH 4.5, 85° C., 0.12 mM CaCl₂), of as at least 40.

In an embodiment the thermostable alpha-amylase has a T½ (min) at pH 4.5, 85° C., 0.12 mM CaCl₂), of at least 50.

In an embodiment the thermostable alpha-amylase has a T½ (min) at pH 4.5, 85° C., 0.12 mM CaCl₂), of at least 60.

In an embodiment the thermostable alpha-amylase has a T½ (min) at pH 4.5, 85° C., 0.12 mM CaCl₂), between 10-70.

In an embodiment the thermostable alpha-amylase has a T½ (min) at pH 4.5, 85° C., 0.12 mM CaCl₂), between 15-70.

In an embodiment the thermostable alpha-amylase has a T½ (min) at pH 4.5, 85° C., 0.12 mM CaCl₂), between 20-70.

In an embodiment the thermostable alpha-amylase has a T½ (min) at pH 4.5, 85° C., 0.12 mM CaCl₂), between 25-70.

In an embodiment the thermostable alpha-amylase has a T½ (min) at pH 4.5, 85° C., 0.12 mM CaCl₂), between 30-70.

In an embodiment the thermostable alpha-amylase has a T½ (min) at pH 4.5, 85° C., 0.12 mM CaCl₂), between 40-70.

In an embodiment the thermostable alpha-amylase has a T½ (min) at pH 4.5, 85° C., 0.12 mM CaCl₂), between 50-70.

In an embodiment the thermostable alpha-amylase has a T½ (min) at pH 4.5, 85° C., 0.12 mM CaCl₂), between 60-70.

In an embodiment of the invention the alpha-amylase is an bacterial alpha-amylase, preferably derived from the genus Bacillus, especially a strain of Bacillus stearothermophilus, in particular the Bacillus stearothermophilus as disclosed in WO 99/019467 as SEQ ID NO: 3 (SEQ ID NO: 22 herein) with one or two amino acids deleted at positions R179, G180, I181 and/or G182, in particular with R179 and G180 deleted, or with I181 and G182 deleted, with mutations in below list of mutations.

In preferred embodiments the Bacillus stearothermophilus alpha-amylases have double deletion I181+G182, and optional substitution N193F, further comprising mutations selected from below list.

In a preferred embodiment the alpha-amylase is selected from the following group of Bacillus stearothermophilus alpha-amylase variants (using SEQ ID NO: 22 for numbering):

$$\\ I {181}^{*} + G {182}^{*} + N 193 F + E 129 V + K 177 L + R 179 E ;$$

$$\\ I {181}^{*} + G {182}^{*} + N 193 F + V 59 A + Q 89 R + E 129 V + K 177 L + R 179 E + H 208 Y + K 220 P + N 224 L + Q 254 S$$

$$\\ I {181}^{*} + G {182}^{*} + N 193 F + V 59 A + Q 89 R + E 129 V + K 177 L + R 179 E + Q 254 S + M 284 V ;$$

$$\\ I {181}^{*} + G {182}^{*} + N 193 F + V 59 A + E 129 V + K 177 L + R 179 E + Q 254 S + M 284 V ;$$

$$\\ I {181}^{*} + G {182}^{*} + N 193 F + E 129 V + K 177 L + R 179 E + K 220 P + N 224 L + S 242 Q + Q 254 S ;$$

$$\\ I {181}^{*} + G {182}^{*} + V 59 A + E 129 V + K 177 L + R 179 E + Q 254 S + M 284 V + V 212 T + Y 268 G + N 293 Y + T 297 N ;$$

$$\\ I {181}^{*} + G {182}^{*} + V 59 A + E 129 V + K 177 L + R 179 E + Q 254 S + M 284 V + V 212 T + Y 268 G + N 293 Y + T 297 N + S 173 N + E 188 P + H 208 Y + S 242 Y + K 279 I ;$$

$$\\ I {181}^{*} + G {182}^{*} + V 59 A + E 129 V + K 177 L + R 179 S + Q 254 S + M 284 V + V 212 T + Y 268 G + N 293 Y + T 297 N + A 184 Q + E188 P + T 191 N$$

$$\\ I {181}^{*} + G {182}^{*} + V 59 A + E 129 V + K 177 L + R 179 S + Q 254 S + M 284 V + V 212 T + Y 268 G + N 293 Y + T 297 N + A 184 Q + E 188 P + T 191 N + S 242 Y + K 279 I ;$$

$$\\ I {181}^{*} + G {182}^{*} + V 59 A + E 129 V + K 177 L + R 179 E + Q 254 S + M 284 V + V 212 T + Y 268 G + N 293 Y + T 297 N + E188 P + K 279 W ;$$

$$\\ I {181}^{*} + G {182}^{*} + V 59 A + E 129 V + K 177 L + R 179 E + Q 254 S + M 284 V + V 212 T + Y 268 G + N 293 Y + T 297 N + W 115 D + D 117 Q + T 133 P ;$$

and wherein the variant has at least 85%, at least 90%, at least 95%, at least 96%, at least 97%, at least 98%, or at least 99% sequence identity to SEQ ID NO: 22.

It should be understood, that when referring to Bacillus stearothermophilus alpha-amylase and variants thereof they are normally produced in truncated form. In particular, the truncation may be so that the Bacillus stearothermophilus alpha-amylase shown in SEQ ID NO: 3 in WO 99/19467 or SEQ ID NO: 22 herein, or variants thereof, are truncated in the C-terminal and are typically around 491 amino acids long, such as from 480-495 amino acids long.

In a preferred embodiment the alpha-amylase variant may be an enzyme having a degree of identity of at least 60%, e.g., at least 70%, at least 80%, at least 90%, at least 95%, at least 91%, at least 92%, at least 93%, at least 94%, at least 95%, at least 96%, at least 97%, at least 98% or at least 99%, but less than 100% to the sequence shown in SEQ ID NO: 3 in WO 99/19467 or SEQ ID NO: 22 herein.

Protease Present and/or Added During Liquefaction

According to the invention a thermostable protease is optionally present and/or added during liquefaction together with an alpha-amylase, and optionally a carbohydrate-source generating enzyme, in particular a thermostable glucoamylase, and/or optionally a pullulanase.

Any protease herein contemplated as being present and/or added during liquefaction is also contemplated for expression by a fermenting organism or host cell.

Proteases are classified on the basis of their catalytic mechanism into the following groups: Serine proteases (S), Cysteine proteases (C), Aspartic proteases (A), Metallo proteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

In a preferred embodiment the thermostable protease used according to the invention is a “metallo protease” defined as a protease belonging to EC 3.4.24 (metalloendopeptidases); preferably EC 3.4.24.39 (acid metallo proteinases).

To determine whether a given protease is a metallo protease or not, reference is made to the above “Handbook of Proteolytic Enzymes” and the principles indicated therein. Such determination can be carried out for all types of proteases, be it naturally occurring or wild-type proteases; or genetically engineered or synthetic proteases.

Protease activity can be measured using any suitable assay, in which a substrate is employed, that includes peptide bonds relevant for the specificity of the protease in question. Assay-pH and assay-temperature are likewise to be adapted to the protease in question. Examples of assay-pH-values are pH 6, 7, 8, 9, 10, or 11. Examples of assay-temperatures are 30, 35, 37, 40, 45, 50, 55, 60, 65, 70 or 80° C.

Examples of protease substrates are casein, such as Azurine-Crosslinked Casein (AZCL-casein). Two protease assays are described below in the “Materials & Methods”-section, of which the so-called “AZCL-Casein Assay” is the preferred assay.

In an embodiment the thermostable protease has at least 20%, such as at least 30%, such as at least 40%, such as at least 50%, such as at least 60%, such as at least 70%, such as at least 80%, such as at least 90%, such as at least 95%, such as at least 100% of the protease activity of the Protease 196 variant or Protease Pfu determined by the AZCL-casein assay described in the “Materials & Methods” section.

There are no limitations on the origin of the protease used in a process of the invention as long as it fulfills the thermostability properties defined below.

In one embodiment the protease is of fungal origin.

The protease may be a variant of, e.g., a wild-type protease as long as the protease has the thermostability properties defined herein.

In a particular embodiment the thermostable protease is a variant of a metallo protease as defined above. In an embodiment the thermostable protease used in a process of the invention is of fungal origin, such as a fungal metallo protease, such as a fungal metallo protease derived from a strain of the genus Thermoascus, preferably a strain of Thermoascus aurantiacus, especially Thermoascus aurantiacus CGMCC No. 0670 (classified as EC 3.4.24.39).

In an embodiment the thermostable protease is a variant of the mature part of the metallo protease shown in SEQ ID NO: 2 disclosed in WO 2003/048353 or the mature part of SEQ ID NO: 1 in WO 2010/008841 and shown as SEQ ID NO: 23 herein further with mutations selected from below list:

$$S {5}^{*} + D 79 L + S 87 P + A 112 P + D 142 L ;$$

$$D 79 L + S 87 P + A 112 P + T 124 V + D 142 L ;$$

$$S {5}^{*} + N 26 R + D 79 L + S 87 P + A 112 P + D 142 L ;$$

$$N 26 R + T 46 R + D 79 L + S 87 P + A 112 P + D 142 L ;$$

$$T 46 R + D 79 L + S 87 P + T 116 V + D 142 L ;$$

$$D 79 L + P 81 R + S 87 P + A 112 P + D 142 L ;$$

$$A 27 K + D 79 L + S 87 P + A 112 P + T 124 V + D 142 L ;$$

$$D 79 L + Y 82 F + S 87 P + A 112 P + T 124 V + D 142 L ;$$

$$D 79 L + S 87 P + A 112 P + T 124 V + A 126 V + D 142 L ;$$

$$D 79 L + S 87 P + A 112 P + D 142 L ;$$

$$D 79 L + Y 82 F + S 87 P + A 112 P + D 142 L ;$$

$$S 38 T + D 79 L + S 87 P + A 112 P + A 126 V + D 142 L ;$$

$$D 79 L + Y 82 F + S 87 P + A 112 P + A 126 V + D 142 L ;$$

$$A 27 K + D 79 L + S 87 P + A 112 P + A 126 V + D 142 L ;$$

$$D 79 L + S 87 P + N 98 C + A 112 P + G 135 C + D 142 L ;$$

$$D 79 L + S 87 P + A 112 P + D 142 L + T 141 C + M 161 C ;$$

$$S 36 P + D 79 L + S 87 P + A 112 P + D 142 L ;$$

$$A 37 P + D 79 L + S 87 P + A 112 P + D 142 L ;$$

$$S 49 P + D 79 L + S 87 P + A 112 P + D 142 L ;$$

$$S 50 P + D 79 L + S 87 P + A 112 P + D 142 L ;$$

$$D 79 L + S 87 P + D 104 P + A 112 P + D 142 L ;$$

$$D 79 L + Y 82 F + S 87 G + A 112 P + D 142 L ;$$

$$S 70 V + D 79 L + Y 82 F + S 87 G + Y 97 W + A 112 P + D 142 L ;$$

$$D 79 L + Y 82 F + S 87 G + Y 97 W + D 104 P + A 112 P + D 142 L ;$$

$$S 70 V + D 79 L + Y 82 F + S 87 G + A 112 P + D 142 L ;$$

$$D 79 L + Y 82 F + S 87 G + D 104 P + A 112 P + D 142 L ;$$

$$D 79 L + Y 82 F + S 87 G + A 112 P + A 126 V + D 142 L ;$$

$$Y 82 F + S 87 G + S70V + D 79 L + D 104 P + A 112 P + D 142 L ;$$

$$Y 82 F + S 87 G + D 79 L + D 104 P + A 112 P + A 126 V + D 142 L ;$$

$$A 27 K + D 79 L + Y 82 F + S 87 G + D 104 P + A 112 P + A 126 V + D 142 L ;$$

$$A 27 K + Y 82 F + S 87 G + D 104 P + A 112 P + A 126 V + D 142 L ;$$

$$A 27 K + D 79 L + Y 82 F + D 104 P + A 112 P + A 126 V + D 142 L ;$$

$$A 27 K + Y 82 F + D 104 P + A 112 P + A 126 V + D 142 L ;$$

$$A 27 K + D 79 L + S 87 P + A 112 P + D 142 L ;$$

$$D 79 L + S 87 P + D 142 L ;$$

In a preferred embodiment the thermostable protease is a variant of the metallo protease disclosed as the mature part of SEQ ID NO: 2 disclosed in WO 2003/048353 or the mature part of SEQ ID NO: 1 in WO 2010/008841 or SEQ ID NO: 23 herein with the following mutations:

$$D 79 L + S 87 P + A 112 P + D 1 \; 42 L ;$$

$$D 79 L + S 87 P + D 1 \; 42 L ; or$$

$$A 27 K + D 79 L + Y 82 F + S 87 G + D 104 P + A 112 P + A 126 V + D 142 L .$$

In an embodiment the protease variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 2 disclosed in WO 2003/048353 or the mature part of SEQ ID NO: 1 in WO 2010/008841 or SEQ ID NO: 23 herein.

The thermostable protease may also be derived from any bacterium as long as the protease has the thermostability properties defined according to the invention. In one embodiment the protease is a serine protease, particularly a S8 protease. Preferred proteases are, serine proteases, particularly an S8 serine protease derived from a strain of Pyrococcus, preferably a strain of Pyrococcus furiosus, or derived from a strain of Thermococcus, preferably Themococcus thioreducens, or derived from a strain of Palaeococcus, preferably Palaeococcus ferrophilus.

In an embodiment the thermostable protease is derived from a strain of the bacterium Pyrococcus, such as a strain of Pyrococcus furiosus (pfu protease).

In an embodiment the protease is one shown as SEQ ID NO: 1 in U.S. Pat. No. 6,358,726-B1 (Takara Shuzo Company), SEQ ID NO: 24 herein.

In another embodiment the thermostable protease is one disclosed in SEQ ID NO: 24 herein or a protease having at least 80% identity, such as at least 85%, such as at least 90%, such as at least 95%, such as at least 96%, such as at least 97%, such as at least 98%, such as at least 99% identity to SEQ ID NO: 1 in U.S. Pat. No. 6,358,726-B1 or SEQ ID NO: 24 herein.

The Pyrococcus furiosus protease is a thermostable protease according to the invention. The Pyrococcus furiosus protease (PfuS) was found to have a thermostability of 110% (80° C./70° C.) and 103% (90° C./70° C.) at pH 4.5 determined as described in Example 2 herein.

In an embodiment the thermostable protease is derived from a strain of the bacterium Palaeococcus, such as a strain of Palaeococcus ferrophilus. In an embodiment the protease is the one shown as SEQ ID NO: 25 herein. In another embodiment the thermostable protease is one disclosed in SEQ ID NO: 25 herein or a protease having at least 80% identity, such as at least 85%, such as at least 90%, such as at least 95%, such as at least 96%, such as at least 97%, such as at least 98%, such as at least 99% identity to SEQ ID NO: 25.

In one embodiment a thermostable protease used in a process of the invention has a thermostability value of more than 20% determined as Relative Activity at 80° C./70° C. determined as described in Example 2.

In an embodiment the protease has a thermostability of more than 30%, more than 40%, more than 50%, more than 60%, more than 70%, more than 80%, more than 90%, more than 100%, such as more than 105%, such as more than 110%, such as more than 115%, such as more than 120% determined as Relative Activity at 80° C./70° C.

In an embodiment protease has a thermostability of between 20 and 50%, such as between 20 and 40%, such as 20 and 30% determined as Relative Activity at 80° C./70° C.

In an embodiment the protease has a thermostability between 50 and 115%, such as between 50 and 70%, such as between 50 and 60%, such as between 100 and 120%, such as between 105 and 115% determined as Relative Activity at 80° C./70° C.

In an embodiment the protease has a thermostability value of more than 10% determined as Relative Activity at 85° C./70° C. determined as described in Example 2.

In an embodiment the protease has a thermostability of more than 10%, such as more than 12%, more than 14%, more than 16%, more than 18%, more than 20%, more than 30%, more than 40%, more that 50%, more than 60%, more than 70%, more than 80%, more than 90%, more than 100%, more than 110% determined as Relative Activity at 85° C./70° C.

In an embodiment the protease has a thermostability of between 10 and 50%, such as between 10 and 30%, such as between 10 and 25% determined as Relative Activity at 85° C./70° C.

In an embodiment the protease has more than 20%, more than 30%, more than 40%, more than 50%, more than 60%, more than 70%, more than 80%, more than 90% determined as Remaining Activity at 80° C.; and/or

In an embodiment the protease has more than 20%, more than 30%, more than 40%, more than 50%, more than 60%, more than 70%, more than 80%, more than 90% determined as Remaining Activity at 84° C.

Determination of “Relative Activity” and “Remaining Activity” is done as described in Example 2.

In an embodiment the protease may have a themostability for above 90, such as above 100 at 85° C. as determined using the Zein-BCA assay as disclosed in Example 3.

In an embodiment the protease has a themostability above 60%, such as above 90%, such as above 100%, such as above 110% at 85° C. as determined using the Zein-BCA assay.

In an embodiment protease has a themostability between 60-120, such as between 70-120%, such as between 80-120%, such as between 90-120%, such as between 100-120%, such as 110-120% at 85° C. as determined using the Zein-BCA assay.

In an embodiment the thermostable protease has at least 20%, such as at least 30%, such as at least 40%, such as at least 50%, such as at least 60%, such as at least 70%, such as at least 80%, such as at least 90%, such as at least 95%, such as at least 100% of the activity of the JTP196 protease variant or Protease Pfu determined by the AZCL-casein assay.

In an embodiment the protease is derived from a strain of Thermobifida, such as the Thermobifida cellulosytica protease shown in SEQ ID NO: 35 herein, or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 35.

In an embodiment the protease is derived from a strain of Thermobifida, such as the Thermobifida fusca protease shown in SEQ ID NO: 37 herein (referred to as SEQ ID NO: 8 in WO2018/118815 A1, which is incorporated herein by reference in its entirety), or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 37.

In an embodiment the protease is derived from a strain of Thermobifida, such as the Thermobifida halotolerans protease shown in SEQ ID NO: 38 herein (referred to as SEQ ID NO: 10 in WO2018/118815 A1, which is incorporated herein by reference in its entirety), or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 38.

In an embodiment the protease is derived from a strain of Thermococcus, such as the Thermococcus nautili protease shown in SEQ ID NO: 39 herein (referred to as SEQ ID NO: 3 in WO2018/169780A1, which is incorporated herein by reference in its entirety), or one having at least 60%, such as at least 70%, such as at least 75% identity, at least 76% identity, at least 77% identity, at least 78% identity, at least 79% identity, preferably at least 80%, at least 81% identity, at least 82% identity, at least 83% identity, at least 84% identity, more preferably at least 85% identity, at least 86% identity, at least 87% identity, at least 88% identity, at least 89% identity, more preferably at least 90% identity, more preferably at least 91% identity, more preferably at least 92% identity, at least 93% identity, at least 94% identity, or at least 95% identity, such as at least 96% identity, at least 97% identity, at least 98% identity, at least 99% identity to the amino acid sequence of SEQ ID NO: 39.

Carbohydrate-Source Generating Enzyme Present and/or Added During Liquefaction

According to the invention a carbohydrate-source generating enzyme, in particular a glucoamylase, preferably a thermostable glucoamylase, may optionally be present and/or added during liquefaction together with an alpha-amylase and an optional thermostable protease. As mentioned above, a pullulanase may also be optionally be present and/or added during liquefaction step i).

Any carbohydrate-source generating enzymes (e.g., glucoamylase) herein contemplated as being present and/or added during liquefaction is also contemplated for expression by a fermenting organism or host cell.

The term “carbohydrate-source generating enzyme” includes any enzymes generating fermentable sugars. A carbohydrate-source generating enzyme is capable of producing a carbohydrate that can be used as an energy-source by the fermenting organism(s) in question, for instance, when used in a process of the invention for producing a fermentation product, such as ethanol. The generated carbohydrates may be converted directly or indirectly to the desired fermentation product, preferably ethanol. According to the invention a mixture of carbohydrate-source generating enzymes may be used. Specific examples include glucoamylase (being glucose generators), beta-amylase and maltogenic amylase (being maltose generators).

In a preferred embodiment the carbohydrate-source generating enzyme is thermostable. The carbohydrate-source generating enzyme, in particular thermostable glucoamylase, may be added together with or separately from the alpha-amylase and the thermostable protease.

In a specific and preferred embodiment the carbohydrate-source generating enzyme is a thermostable glucoamylase, preferably of fungal origin, preferably a filamentous fungi, such as from a strain of the genus Penicillium, especially a strain of Penicillium oxalicum, in particular the Penicillium oxalicum glucoamylase disclosed as SEQ ID NO: 2 in PCT/CN10/071753 published as WO 2011/127802 (which is hereby incorporated by reference) and shown in SEQ ID NO: 26 herein.

In an embodiment the thermostable glucoamylase has at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99% or 100% identity to the mature polypeptide shown in SEQ ID NO: 2 in WO 2011/127802 or SEQ ID NOs: 26 herein.

In a preferred embodiment the carbohydrate-source generating enzyme is a variant of the Penicillium oxalicum glucoamylase disclosed as SEQ ID NO: 2 in WO 2011/127802 and SEQ ID NO: 26 herein, having a K79V substitution (using the mature sequence shown in SEQ ID NO: 26 for numbering). The K79V glucoamylase variant has reduced sensitivity to protease degradation relative to the parent as disclosed in co-pending U.S. application No. 61/531,189 or PCT/US12/053779 (which are hereby incorporated by reference).

In an embodiment the carbohydrate-source generating enzyme, in particular thermostable glucoamylase, is derived from Penicillium oxalicum.

In an embodiment the thermostable glucoamylase is a variant of the Penicillium oxalicum glucoamylase disclosed as SEQ ID NO: 2 in WO 2011/127802 and shown in SEQ ID NO: 22 herein. In a preferred embodiment the Penicillium oxalicum glucoamylase is the one disclosed as SEQ ID NO: 2 in WO 2011/127802 and shown in SEQ ID NO: 26 herein having Val (V) in position 79 (using SEQ ID NO: 26 for numbering).

In an embodiment these variants have reduced sensitivity to protease degradation.

In an embodiment these variants have improved thermostability compared to the parent.

More specifically, in an embodiment the glucoamylase has a K79V substitution (using SEQ ID NO: 26 for numbering), corresponding to the PE001 variant, and further comprises at least one of the following substitutions or combination of substitutions:

$$P 11 F + T 65 A + Q 327 F ; or$$

$$P 2 N + P 4 S + P 11 F + T 65 A + Q 327 F ; or$$

$$P 11 F + D 26 C + K 33 C + T 65 A + Q 327 F ; or$$

$$P 2 N + P 4 S + P 11 F + T 65 A + Q 327 W + E 501 V + Y 504 T ; or$$

$$P 2 N + P 4 S + P 11 F + T 65 A + Q 327 F + E 501 V + Y 504 T ; or$$

$$P 11 F + T 65 A + Q 327 W + E 501 V + Y 504 T .$$

The carbohydrate-source generating enzyme, in particular, may be added in amounts from 0.1-100 micrograms EP/g, such as 0.5-50 micrograms EP/g, such as 1-25 micrograms EP/g, such as 2-12 micrograms EP/g DS.

Carbohydrate-Source Generating Enzyme present and/or added during Saccharification and/or Fermentation

According to the invention a carbohydrate-source generating enzyme, preferably a glucoamylase, may be present and/or added during saccharification and/or fermentation. In a preferred embodiment the carbohydrate-source generating enzyme is a glucoamylase, of fungal origin, preferably from a stain of Aspergillus, preferably A. niger, A. awamori, or A. oryzae; or a strain of Trichoderma, preferably T. reesei; or a strain of Talaromyces, preferably T. emersonii, or or a strain of Trametes, preferably Trametes cingulata, or a strain of Pycnoporus, or a strain of Gloeophyllum, such as a strain of Gloeophyllum sepiarium or Gloeophyllum trabeum or a strain of the Nigrofomes.

Any glucoamylase contemplated as being present and/or added during saccharification and/or fermentation is also contemplated for expression by a fermenting organism or host cell.

### Glucoamylases

According to the invention the glucoamylase present and/or added during saccharification and/or fermentation may be derived from any suitable source, e.g., derived from a microorganism or a plant. Preferred glucoamylases are of fungal or bacterial origin, selected from the group consisting of Aspergillus glucoamylases, in particular Aspergillus niger G1 or G2 glucoamylase (Boel et al. (1984), EMBO J. 3 (5), p. 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka et al. (1998) “Purification and properties of the raw-starch-degrading glucoamylases from Corticium rolfsii, Appl Microbiol Biotechnol 50:323-330), Talaromyces glucoamylases, in particular derived from Talaromyces emersonii (WO 99/28448), Talaromyces leycettanus (US patent no. Re. 32,153), Talaromyces duponti, Talaromyces thermophilus (U.S. Pat. No. 4,587,215). In a preferred embodiment the glucoamylase used during saccharification and/or fermentation is the Talaromyces emersonii glucoamylase disclosed in WO 99/28448.

Contemplated fungal glucoamylases include particularly glucoamylases derived from Talaromyces, preferably T. emersonii, or or a strain of Trametes, preferably Trametes cingulata, or a strain of Pycnoporus, or a strain of Gloeophyllum, such as a strain of Gloeophyllum sepiarium or Gloeophyllum trabeum or a strain of the Nigrofomes.

In one embodiment the glucoamylase is derived from a strain of the genus Trametes, in particular a strain of Trametes cingulata, disclosed in WO 2006/069289 or in SEQ ID NO: 27 herein. In one embodiment the glucoamylase is derived from a strain of the genus Talaromyces, in particular a strain of Talaromyces emersonii disclosed in SEQ ID NO: 28 herein.

In another embodiment the glucoamylase is derived from a strain of the genus Pycnoporus, in particular a strain of Pycnoporus sanguineus as described in WO 2011/066576 (SEQ ID NOs 2, 4 or 6) or SEQ ID NO: 29 herein, or from a strain of the genus Gloeophyllum, such as a strain of Gloeophyllum sepiarium or Gloeophyllum trabeum, in particular a strain of Gloeophyllum as described in WO 2011/068803 (SEQ ID NO: 2, 4, 6, 8, 10, 12, 14 or 16). In a preferred embodiment the glucoamylase is SEQ ID NO: 31 herein. In another embodiment the glucoamylase is SEQ ID NO: 32 herein. In an embodiment the glucoamylase is derived from a strain of the genus Nigrofomes, in particular a strain of Nigrofomes sp. disclosed in WO 2012/064351 as SEQ ID NO: 2. Contemplated are also glucoamylases which exhibit a high identity to any of the above mentioned glucoamylases, i.e., at least 70%, at least 75%, at least 80%, at least 85%, at least 90%, at least 95%, at least 96%, at least 97%, at least 98%, at least 99% or even 100% identity to any one of the mature parts of the enzyme sequences mentioned above, such as any of SEQ ID NOs: 27, 28, 29, 30, or 31 herein.

Glucoamylases may in an embodiment be added to the saccharification and/or fermentation in an amount of 0.0001-20 AGU/g DS, preferably 0.001-10 AGU/g DS, especially between 0.01-5 AGU/g DS, such as 0.1-2 AGU/g DS.

In an embodiment the glucoamylase is added as a blend further comprising an alpha-amylase. In a preferred embodiment the alpha-amylase is a fungal alpha-amylase, especially an acid fungal alpha-amylase. The alpha-amylase is typically a side activity.

In an embodiment the glucoamylase is a blend comprising Talaromyces emersonii glucoamylase disclosed in WO 99/28448 as SEQ ID NO: 7 or SEQ ID NO: 28 herein and Trametes cingulata glucoamylase disclosed in WO 06/069289 and SEQ ID NO: 27 herein.

In an embodiment the glucoamylase is a blend comprising Talaromyces emersonii glucoamylase disclosed SEQ ID NO: 28, Trametes cingulata glucoamylase disclosed as SEQ ID NO: 27, and Rhizomucor pusillus alpha-amylase with Aspergillus niger glucoamylase linker and SBD disclosed as V039 in Table 5 in WO 2006/069290 and as SEQ ID NO: 27 herein, preferably with the following substitutions: G128D+D143N.

In an embodiment the glucoamylase is a blend comprising Gloeophyllum sepiarium glucoamylase shown as SEQ ID NO: 2 in WO 2011/068803 (SEQ ID NO: 30 herein) and Rhizomucor pusillus with an Aspergillus niger glucoamylase linker and starch-binding domain (SBD), disclosed SEQ ID NO: 3 in WO 2013/006756 (SEQ ID NO: 32 herein) with the following substitutions: G128D+D143N.

In an embodiment the Rhizomucor pusillus alpha-amylase or the Rhizomucor pusillus alpha-amylase with an Aspergillus niger glucoamylase linker and starch-binding domain (SBD) has at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W +D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; G128D+Y141W+D143N+K192R; or G128D+Y141W+D143N+K192R+P219C (using SEQ ID NO: 3 in WO 2013/006756 for numbering).

Commercially available compositions comprising glucoamylase include AMG 200L; AMG 300 L; SAN™ SUPER, SAN™ EXTRA L, SPIRIZYMET PLUS, SPIRIZYMET FUEL, SPIRIZYME™ B4U, SPIRIZYMET ULTRA, SPIRIZYMET EXCEL, SPIRIZYME ACHIEVE and AMG™ E (from Novozymes A/S); OPTIDEX™ 300, GC480, GC417 (from DuPont-Genencor); AMIGASE™ and AMIGASE™ PLUS (from DSM); G-ZYMET G900, G-ZYMET and G990 ZR (from DuPont-Genencor).

Cellulolytic Composition present and/or added during Saccharification and/or Fermentation

According to the invention a cellulolytic composition is present during fermentation or simultaneous saccharification and fermentation (SSF).

The cellulolytic composition may be any cellulolytic composition, comprising a beta-glucosidase, a cellobiohydrolase and an endoglucanase.

Any cellulase described herein contemplated as being present and/or added during saccharification and/or fermentation is also contemplated for expression by a fermenting organism or host cell.

Examples of suitable cellulolytic composition can be found in WO 2008/151079 and co-pending patent application PCT/US12/052163 published as WO 2013/028928 which areincorporated by reference.

In preferred embodiments the cellulolytic composition is derived from a strain of Trichoderma, Humicola, or Chrysosporium.

In an embodiment the cellulolytic composition is derived from a strain of Trichoderma reesei, Humicola insolens and/or Chrysosporium lucknowense.

In an embodiment the cellulolytic composition comprises a beta-glucosidase, preferably one derived from a strain of the genus Aspergillus, such as Aspergillus oryzae, such as the one disclosed in WO 2002/095014 or the fusion protein having beta-glucosidase activity disclosed in WO 2008/057637, or Aspergillus fumigatus, such as one disclosed in WO 2005/047499 or SEQ ID NO: 19 herein or an Aspergillus fumigatus beta-glucosidase variant disclosed in WO 2012/044915 (Novozymes), such as one with the following substitutions F100D, S283G, N456E, F512Y; or a strain of the genus a strain Penicillium, such as a strain of the Penicillium brasilianum disclosed in WO 2007/019442, or a strain of the genus Trichoderma, such as a strain of Trichoderma reesei.

In an embodiment the cellulolytic composition comprises a GH61 polypeptide having cellulolytic enhancing activity such as one derived from the genus Thermoascus, such as a strain of Thermoascus aurantiacus, such as the one described in WO 2005/074656 as SEQ ID NO: 2; or one derived from the genus Thielavia, such as a strain of Thielavia terrestris, such as the one described in WO 2005/074647 as SEQ ID NO: 7 and SEQ ID NO: 8; or one derived from a strain of Aspergillus, such as a strain of Aspergillus fumigatus, such as the one described in WO 2010/138754 as SEQ ID NO: 1 and SEQ ID NO: 2; or one derived from a strain derived from Penicillium, such as a strain of Penicillium emersonii, such as the one disclosed in WO 2011/041397 or SEQ ID NO: 18 herein.

In an embodiment the cellulolytic composition comprises a cellobiohydrolase I (CBH I), such as one derived from a strain of the genus Aspergillus, such as a strain of Aspergillus fumigatus, such as the Cel7a CBHI disclosed in SEQ ID NO: 2 in WO 2011/057140 or SEQ ID NO: 20 herein, or a strain of the genus Trichoderma, such as a strain of Trichoderma reesei.

In an embodiment the cellulolytic composition comprises a cellobiohydrolase II (CBH II, such as one derived from a strain of the genus Aspergillus, such as a strain of Aspergillus fumigatus or SEQ ID NO: 21 herein; or a strain of the genus Trichoderma, such as Trichoderma reesei, or a strain of the genus Thielavia, such as a strain of Thielavia terrestris, such as cellobiohydrolase II CEL6A from Thielavia terrestris.

In an embodiment the cellulolytic composition comprises a GH61 polypeptide having cellulolytic enhancing activity and a beta-glucosidase.

In an embodiment the cellulolytic composition comprises a GH61 polypeptide having cellulolytic enhancing activity, a beta-glucosidase, and a CBH I.

In an embodiment the cellulolytic composition comprises a GH61 polypeptide having cellulolytic enhancing activity, a beta-glucosidase, a CBH I, and a CBH II.

In an embodiment the cellulolytic composition is a Trichoderma reesei cellulolytic enzyme composition, further comprising Thermoascus aurantiacus GH61A polypeptide having cellulolytic enhancing activity (SEQ ID NO: 2 in WO 2005/074656), and Aspergillus oryzae beta-glucosidase fusion protein (WO 2008/057637).

In an embodiment the cellulolytic composition is a Trichoderma reesei cellulolytic enzyme composition, further comprising Thermoascus aurantiacus GH61A polypeptide having cellulolytic enhancing activity (SEQ ID NO: 2 in WO 2005/074656) and Aspergillus fumigatus beta-glucosidase (SEQ ID NO: 2 of WO 2005/047499) or SEQ ID NO: 19 herein.

In an embodiment the cellulolytic composition is a Trichoderma reesei cellulolytic enzyme composition further comprising Penicillium emersonii GH61A polypeptide having cellulolytic enhancing activity disclosed in WO 2011/041397 and Aspergillus fumigatus beta-glucosidase (SEQ ID NO: 2 of WO 2005/047499) or SEQ ID NO: 18 herein or a variant thereof with the following substitutions F100D, S283G, N456E, F512Y.

In an embodiment, the cellulolytic composition, for example a Trichoderma reesei cellulolytic enzyme composition, comprises one or more polypeptides selected from the group consisting of:
beta-glucosidase;
cellobiohydrolase I; and
endoglucanase I, or a mixture of two or three thereof.

In an embodiment, the cellulolytic composition, for example a Trichoderma reesei cellulolytic enzyme composition, comprises one or more of the following components:
(i) an Aspergillus fumigatus beta-glucosidase or a variant thereof;
(ii) an Aspergillus fumigatus cellobiohydrolase I; and
(iii) a Trichoderma reesei endoglucanase I.

In an embodiment, the cellulolytic composition is a Trichoderma reesei cellulolytic composition further comprising:
(i) an Aspergillus fumigatus beta-glucosidase disclosed in SEQ ID NO: 19 or a variant thereof with the following substitutions: F100D, S283G, N456E, F512Y having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 19; (ii) a cellobiohydrolase I (CBH I), such as one derived from a strain of the genus Aspergillus, such as a strain of Aspergillus fumigatus, such as the CBHI disclosed as SEQ ID NO: 20, or a CBHI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 20; and (iii) an endoglucanase | (EGI), such as one derived from a strain of the genus Trichoderma, such as a strain of Trichoderma reesei, such as the EGI disclosed as SEQ ID NO: 36, or an EGI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 36.

In a preferred embodiment the cellulolytic composition comprising one or more of the following components:
(i) an Aspergillus fumigatus cellobiohydrolase I;
(ii) an Aspergillus fumigatus cellobiohydrolase II;
(iii) an Aspergillus fumigatus beta-glucosidase or variant thereof; and
(iv) a Penicillium sp. GH61 polypeptide having cellulolytic enhancing activity; or homologs thereof.

In an preferred embodiment the cellulolytic composition is derived from Trichoderma reesei comprising GH61A polypeptide having cellulolytic enhancing activity derived from a strain of Penicillium emersonii (SEQ ID NO: 2 in WO 2011/041397 or SEQ ID NO: 18 herein), Aspergillus fumigatus beta-glucosidase (SEQ ID NO: 2 in WO 2005/047499 or SEQ ID NO: 19 herein) variant F100D, S283G, N456E, F512Y) disclosed in WO 2012/044915; Aspergillus fumigatus Cel7A CBH1 disclosed as SEQ ID NO: 6 in WO2011/057140 (SEQ ID NO: 20 herein) and Aspergillus fumigatus CBH II disclosed as SEQ ID NO: 18 in WO 2011/057140 (SEQ ID NO: 21 herein).

In an embodiment the cellulolytic composition is dosed from 0.0001-3 mg EP/g DS, preferably, 0.0005-2 mg EP/g DS, preferably 0.001-1 mg/g DS, more preferably 0.005-0.5 mg EP/g DS, and even more preferably 0.01-0.1 mg EP/g DS.

### Enzyme Blends or Compositions

An enzyme blend or composition of the invention comprises at least one polypepeptide having alpha-amylase activity. In some embodiments, the at least one polypeptide having alpha-amylase activity in the enzyme blend or composition comprises at least one, at least two, at least three, at least four, or at least five polypeptides having alpha-amylase activity. The enzyme blend or composition may further comprise one or more cellulases, e.g., a beta-glucosidase, a cellobiohydrolase and an endoglucanase. The enzyme blend or composition may further comprise a trehalase. The enzyme blend or composition may further comprise a glucoamylase.

The invention is further defined by the following numbered paragraphs:

1. An isolated or purified polypeptide having alpha-amylase activity, selected from the group consisting of:

(i)
(a) a polypeptide having at least 92%, at least 93%, at least 94%, at least 95%, at least 96%, at least 97%, at least 98%, at least 99%, or 100% sequence identity to SEQ ID NO: 2;
(b) a polypeptide having at least 92%, at least 93%, at least 94%, at least 95%, at least 96%, at least 97%, at least 98%, at least 99%, or 100% sequence identity to the sequence of SEQ ID NO: 3;
(c) a polypeptide having at least 92%, at least 93%, at least 94%, at least 95%, at least 96%, at least 97%, at least 98%, at least 99%, or 100% sequence identity to a mature polypeptide of SEQ ID NO: 2;
(d) a polypeptide encoded by a polynucleotide that hybridizes under medium, medium-high, or high stringency conditions with the full-length complement of the mature polypeptide coding sequence of SEQ ID NO: 1;
(e) a polypeptide encoded by a polynucleotide having at least 92%, at least 93%, at least 94%, at least 95%, at least 96%, at least 97%, at least 98%, at least 99%, or 100% sequence identity to the mature polypeptide coding sequence of SEQ ID NO: 1;
(f) a fragment of the polypeptide of (a), (b), (c), (d), or (e) that has alpha-amylase activity;
   (ii)
(a) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 5;
(b) a polypeptide having at least 85%, at least 86%, at least 87%, at least 88%, at least 96%, at least 97%, at least 98%, at least 99%, or 100% sequence identity to the sequence of SEQ ID NO: 6;
(c) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 5;
(d) a polypeptide encoded by a polynucleotide that hybridizes under medium, medium-high, or high stringency conditions with the full-length complement of the mature polypeptide coding sequence of SEQ ID NO: 4, or the cDNA sequence thereof; or
(e) a polypeptide encoded by a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 4, or the cDNA sequence thereof;
(f) a fragment of the polypeptide of (a), (b), (c), (d), or (e) that has alpha-amylase activity;
   (iii)
(a) a polypeptide having at least 93%, at least 94%, at least 95%, at least 96%, at least 97%, at least 98%, at least 99%, or 100% sequence identity to SEQ ID NO: 8;
(b) a polypeptide having at least 93%, at least 94%, at least 95%, at least 96%, at least 97%, at least 98%, at least 99%, or 100% sequence identity to the sequence of SEQ ID NO: 9;
(c) a polypeptide having at least 93%, at least 94%, at least 95%, at least 96%, at least 97%, at least 98%, at least 99%, or 100% sequence identity to a mature polypeptide of SEQ ID NO: 8;
(d) a polypeptide encoded by a polynucleotide that hybridizes under medium, medium-high, or high stringency conditions with the full-length complement of the mature polypeptide coding sequence of SEQ ID NO: 7, or the cDNA sequence thereof;
(e) a polypeptide encoded by a polynucleotide having at least 93%, at least 94%, at least 95%, at least 96%, at least 97%, at least 98%, at least 99%, or 100% sequence identity to the mature polypeptide coding sequence of SEQ ID NO: 7, or the cDNA sequence thereof;
(f) a fragment of the polypeptide of (a), (b), (c), (d), or (e) that has alpha-amylase activity;
   (iv)
(a) a polypeptide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
(b) a polypeptide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 12;
(c) a polypeptide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 11;
(d) a polypeptide encoded by a polynucleotide that hybridizes under medium, medium-high, or high stringency conditions with the full-length complement of the mature polypeptide coding sequence of SEQ ID NO: 10;
(e) a polypeptide encoded by a polynucleotide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 10;
(f) a fragment of the polypeptide of (a), (b), (c), (d), or (e) that has alpha-amylase activity;
   (v)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 15;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to a mature polypeptide of SEQ ID NO: 14;
(d) a polypeptide encoded by a polynucleotide that hybridizes under medium, medium-high, or high stringency conditions with the full-length complement of the mature polypeptide coding sequence of SEQ ID NO: 13;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 13;
(f) a fragment of the polypeptide of (a), (b), (c), (d), or (e) that has alpha-amylase activity;
   (vi)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 16;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 17;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 16;
(d) a fragment of the polypeptide of (a), (b), or (c), that has alpha-amylase activity;
   (vii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to SEQ ID NO: 41;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 42;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 41;
(d) a fragment of the polypeptide of (a), (b), or (c), that has alpha-amylase activity;
   (viii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 45;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 44;
(d) a polypeptide encoded by a polynucleotide that hybridizes under medium, medium-high, or high stringency conditions with the full-length complement of the mature polypeptide coding sequence of SEQ ID NO: 43;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 43; or
(f) a fragment of the polypeptide of (a), (b), (c), (d), or (e) that has alpha-amylase activity;
   (ix)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 48;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 47;
(d) a polypeptide encoded by a polynucleotide that hybridizes under medium, medium-high, or high stringency conditions with the full-length complement of the mature polypeptide coding sequence of SEQ ID NO: 46;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 46; or
(f) a fragment of the polypeptide of (a), (b), (c), (d), or (e) that has alpha-amylase activity;
   (x)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 50;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 51;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 50;
(d) a polypeptide encoded by a polynucleotide that hybridizes under medium, medium-high, or high stringency conditions with the full-length complement of the mature polypeptide coding sequence of SEQ ID NO: 49;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 49; or
(f) a fragment of the polypeptide of (a), (b), (c), (d), or (e) that has alpha-amylase activity;
   (x)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to SEQ ID NO: 53;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 54;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 53;
(d) a polypeptide encoded by a polynucleotide that hybridizes under medium, medium-high, or high stringency conditions with the full-length complement of the mature polypeptide coding sequence of SEQ ID NO: 52;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 52; or
(f) a fragment of the polypeptide of (a), (b), (c), (d), or (e) that has alpha-amylase activity;
   (xi)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 56;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to the sequence of SEQ ID NO: 57;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 56;
(d) a polypeptide encoded by a polynucleotide that hybridizes under medium, medium-high, or high stringency conditions with the full-length complement of the mature polypeptide coding sequence of SEQ ID NO: 55;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 55; or
(f) a fragment of the polypeptide of (a), (b), (c), (d), or (e) that has alpha-amylase activity; and
   (xii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 59;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 60;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to a mature polypeptide of SEQ ID NO: 59;
(d) a polypeptide encoded by a polynucleotide that hybridizes under medium, medium-high, or high stringency conditions with the full-length complement of the mature polypeptide coding sequence of SEQ ID NO: 58;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 58; or
(f) a fragment of the polypeptide of (a), (b), (c), (d), or (e) that has alpha-amylase activity.

2. An alpha-amylase variant, selected from the group consisting of:
(i) a variant of the mature polypeptide of SEQ ID NO: 2 comprising:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 2; and
(b) at least 1, 2, 3, 4, 5, 6, 7, 8, 9, or 10 substitutions, deletions, or insertions at positions corresponding to positions other than positions D223, E247, and D314 of SEQ ID NO: 2; wherein the variant has alpha-amylase activity;
(ii) a variant of the mature polypeptide of SEQ ID NO: 5 comprising:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 5; and
(b) at least 1, 2, 3, 4, 5, 6, 7, 8, 9, or 10 substitutions, deletions, or insertions at positions corresponding to positions other than positions D223, E247, and D314 of SEQ ID NO: 5; wherein the variant has alpha-amylase activity.
(iii) a variant of the mature polypeptide of SEQ ID NO: 8 comprising:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 8; and
(b) at least 1, 2, 3, 4, 5, 6, 7, 8, 9, or 10 substitutions, deletions, or insertions at positions corresponding to positions other than positions D223, E247, and D314 of SEQ ID NO: 8; wherein the variant has alpha-amylase activity.
(iv) a variant of the mature polypeptide of SEQ ID NO: 11 comprising:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 11; and
(b) at least 1, 2, 3, 4, 5, 6, 7, 8, 9, or 10 substitutions, deletions, or insertions at positions corresponding to positions other than positions D225, E249, and D316 of SEQ ID NO: 11; and wherein the variant has alpha-amylase activity.
(v) a variant of the mature polypeptide of SEQ ID NO: 14 comprising:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 14; and
(b) at least 1, 2, 3, 4, 5, 6, 7, 8, 9, or 10 substitutions, deletions, or insertions at positions corresponding to positions other than positions D217, E251, and D312 of SEQ ID NO: 14; wherein the variant has alpha-amylase activity;
(vi) a variant of the mature polypeptide of SEQ ID NO: 41 comprising:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 41; and
(b) at least 1, 2, 3, 4, 5, 6, 7, 8, 9, or 10 substitutions, deletions, or insertions at positions corresponding to positions other than positions D184, E216, and D277 of SEQ ID NO: 41; wherein the variant has alpha-amylase activity;
(vi) a variant of the mature polypeptide of SEQ ID NO: 44 comprising:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 44; and
(b) at least 1, 2, 3, 4, 5, 6, 7, 8, 9, or 10 substitutions, deletions, or insertions at positions corresponding to positions other than positions D215, E249, and D310 of SEQ ID NO: 44; wherein the variant has alpha-amylase activity;
(vii) a variant of the mature polypeptide of SEQ ID NO: 47 comprising:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 47; and
(b) at least 1, 2, 3, 4, 5, 6, 7, 8, 9, or 10 substitutions, deletions, or insertions at positions corresponding to positions other than positions D215, E249, and D310 of SEQ ID NO: 47; wherein the variant has alpha-amylase activity;
(viii) a variant of the mature polypeptide of SEQ ID NO: 50 comprising:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 50; and
(b) at least 1, 2, 3, 4, 5, 6, 7, 8, 9, or 10 substitutions, deletions, or insertions at positions corresponding to positions other than positions D215, E249, and D310 of SEQ ID NO: 50; wherein the variant has alpha-amylase activity;
(ix) a variant of the mature polypeptide of SEQ ID NO: 53 comprising:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 50; and
(b) at least 1, 2, 3, 4, 5, 6, 7, 8, 9, or 10 substitutions, deletions, or insertions at positions corresponding to positions other than positions D215, E249, and D310 of SEQ ID NO: 53; wherein the variant has alpha-amylase activity;
(x) a variant of the mature polypeptide of SEQ ID NO: 56 comprising:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 56; and
(b) at least 1, 2, 3, 4, 5, 6, 7, 8, 9, or 10 substitutions, deletions, or insertions at positions corresponding to positions other than positions D215, E249, and D310 of SEQ ID NO: 56; wherein the variant has alpha-amylase activity; and
(xi) a variant of the mature polypeptide of SEQ ID NO: 59 comprising:
(a) at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the mature polypeptide of SEQ ID NO: 56; and
(b) at least 1, 2, 3, 4, 5, 6, 7, 8, 9, or 10 substitutions, deletions, or insertions at positions corresponding to positions other than positions D215, E249, and D310 of SEQ ID NO: 59; wherein the variant has alpha-amylase activity.

3. An isolated or purified polypeptide having alpha-amylase activity, which is:

(i)
(a) a polypeptide having at least 92%, at least 93%, at least 94%, at least 95%, at least 96%, at least 97%, at least 98%, at least 99%, or 100% sequence identity to SEQ ID NO: 2; or
(b) a fragment of the polypeptide of (a), that has alpha-amylase activity;
   (ii)
(a) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 5; or
(b) a fragment of the polypeptide of (a), that has alpha-amylase activity;
   (iii)
(a) a polypeptide having at least 93%, at least 94%, at least 95%, at least 96%, at least 97%, at least 98%, at least 99%, or 100% sequence identity to SEQ ID NO: 8; or
(b) a fragment of the polypeptide of (a), that has alpha-amylase activity;
   (iv)
(a) a polypeptide having alpha-amylase at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11; or
(b) a fragment of the polypeptide of (a), that has alpha-amylase activity;
   (v)
(a) a polypeptide having alpha-amylase at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14; or
(b) a fragment of the polypeptide of (a), that has alpha-amylase activity;
   (vi)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to SEQ ID NO: 16; or
(b) a fragment of the polypeptide of (a) that has alpha-amylase activity;
   (vii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41; or
(b) a fragment of the polypeptide of (a) that has alpha-amylase activity;
   (viii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44; or
(b) a fragment of the polypeptide of (a) that has alpha-amylase activity;
   (ix)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47; or
(b) a fragment of the polypeptide of (a) that has alpha-amylase activity;
   (x)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to SEQ ID NO: 50; or
(b) a fragment of the polypeptide of (a) that has alpha-amylase activity;
   (xi)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 53; or
(b) a fragment of the polypeptide of (a) that has alpha-amylase activity;
   (xii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 56; or
(b) a fragment of the polypeptide of (a) that has alpha-amylase activity; or
   (xiii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 59; or
(b) a fragment of the polypeptide of (a) that has alpha-amylase activity.

4. An isolated or purified polypeptide having alpha-amylase activity, which is:

(i)
(a) a polypeptide having alpha-amylase at least 92%, at least 93%, at least 94%, at least 95%, at least 96%, at least 97%, at least 98%, at least 99%, or 100% sequence identity to SEQ ID NO: 3; or
(b) a fragment of the polypeptide of (a), that has alpha-amylase activity;
   (ii)
(a) a polypeptide having alpha-amylase at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 6; or
(b) a fragment of the polypeptide of (a), that has alpha-amylase activity;
   (iii)
(a) a polypeptide having at least 93%, at least 94%, at least 95%, at least 96%, at least 97%, at least 98%, at least 99%, or 100% sequence identity to SEQ ID NO: 9; or
(b) a fragment of the polypeptide of (a), that has alpha-amylase activity;
   (iv)
(a) a polypeptide having alpha-amylase at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 12; or
(b) a fragment of the polypeptide of (a), that has alpha-amylase activity;
   (v)
(a) a polypeptide having alpha-amylase at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15; or
(b) a fragment of the polypeptide of (a), that has alpha-amylase activity;
   (vi)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 17; or
(b) a fragment of the polypeptide of (a), (b), or (c), that has alpha-amylase activity; or
   (vii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 42; or
(b) a fragment of the polypeptide of (a), (b), or (c), that has alpha-amylase activity;
   (viii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to SEQ ID NO: 45; or
(b) a fragment of the polypeptide of (a) that has alpha-amylase activity;
   (ix)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 48; or
(b) a fragment of the polypeptide of (a) that has alpha-amylase activity;
   (x)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 51; or
(b) a fragment of the polypeptide of (a) that has alpha-amylase activity;
   (xi)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 54; or
(b) a fragment of the polypeptide of (a) that has alpha-amylase activity;
   (xii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to SEQ ID NO: 57; or
(b) a fragment of the polypeptide of (a) that has alpha-amylase activity; or
   (xiii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60; or
(b) a fragment of the polypeptide of (a) that has alpha-amylase activity.

5. An isolated or purified polypeptide having alpha-amylase activity, which is:
(a) a polypeptide having at least 92%, at least 93%, at least 94%, at least 95%, at least 96%, at least 97%, at least 98%, at least 99%, or 100% sequence identity to a mature polypeptide of SEQ ID NO: 2; or
(b) a fragment of the polypeptide of (a), that has alpha-amylase activity;
   (ii)
(a) a polypeptide having alpha-amylase at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 5; or
(b) a fragment of the polypeptide of (a), that has alpha-amylase activity;
   (iii)
(a) a polypeptide having alpha-amylase at least 93%, at least 94%, at least 95%, at least 96%, at least 97%, at least 98%, at least 99%, or 100% sequence identity to a mature polypeptide of SEQ ID NO: 8; or
(b) a fragment of the polypeptide of (a), that has alpha-amylase activity;
   (iv)
(a) a polypeptide having alpha-amylase at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 11; or
(b) a fragment of the polypeptide of (a), that has alpha-amylase activity; and
   (v)
(a) a polypeptide having alpha-amylase at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 14; or
(b) a fragment of the polypeptide of (a), that has alpha-amylase activity;
   (vi)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 16; or
(b) a fragment of the polypeptide of (a) that has alpha-amylase activity;
   (vii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 41; or
(b) a fragment of the polypeptide of (a) that has alpha-amylase activity;
   (viii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 44; or
(b) a fragment of the polypeptide of (a) that has alpha-amylase activity;
   (ix)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 47; or
(b) a fragment of the polypeptide of (a) that has alpha-amylase activity;
   (x)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 50; or
(b) a fragment of the polypeptide of (a) that has alpha-amylase activity; or
   (xi)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 53; or
(b) a fragment of the polypeptide of (a) that has alpha-amylase activity;
   (xii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 56; or
(b) a fragment of the polypeptide of (a) that has alpha-amylase activity;
   (xiii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 59; or
(b) a fragment of the polypeptide of (a) that has alpha-amylase activity.

6 An isolated or purified polypeptide having alpha-amylase activity, which is:
(i) a fragment of SEQ ID NO: 2 wherein the fragment preferably contains at least 512 amino acid residues (e.g., amino acids 1 to 512 of SEQ ID NO: 2 or SEQ ID NO: 3), at least 542 amino acid residues (e.g., amino acids 1 to 542 of SEQ ID NO: 2 or SEQ ID NO: 3), at least 572 amino acid residues (e.g., amino acids 1 to 572 of SEQ ID NO: 2 or SEQ ID NO: 3), at least 401 amino acid residues (e.g., amino acids 21 to 422 of SEQ ID NO: 2), at least 424 amino acid residues (e.g., amino acids 21 to 445 of SEQ ID NO: 2), at least 448 amino acid residues (e.g., amino acids 21 to 469 of SEQ ID NO: 2), at least 81 amino acid residues (e.g., amino acids 507 to 588 of SEQ ID NO: 2), at least 86 amino acid residues (e.g., amino acids 507 to 593 of SEQ ID NO: 2), or at least 91 amino acid residues (e.g., amino acids 507 to 598 of SEQ ID NO: 2), and wherein the fragment has alpha-amylase activity;
(ii) a fragment of SEQ ID NO: 5 wherein the fragment preferably contains at least 537 amino acid residues (e.g., amino acids 1 to 537 of SEQ ID NO: 5 or SEQ ID NO: 6), at least 568 amino acid residues (e.g., amino acids 1 to 568 of SEQ ID NO: 5 or SEQ ID NO: 6), at least 600 amino acid residues (e.g., amino acids 1 to 600 of SEQ ID NO: 5 or SEQ ID NO: 6), at least 407 amino acid residues (e.g., amino acids 18 to 425 of SEQ ID NO: 5), at least 431 amino acid residues (e.g., amino acids 18 to 449 of SEQ ID NO: 5), or at least 455 amino acid residues (e.g., amino acids 18 to 473 of SEQ ID NO: 5), at least 85 amino acid residues (e.g., amino acids 532 to 617 of SEQ ID NO: 5), at least 90 amino acid residues (e.g., amino acids 532 to 622 of SEQ ID NO: 5), or at least 95 amino acid residues (e.g., amino acids 532 to 627 of SEQ ID NO: 5), and wherein the fragment has alpha-amylase activity;
(iii) a fragment of SEQ ID NO: 8 wherein the fragment preferably contains at least 537 amino acid residues (e.g., amino acids 1 to 537 of SEQ ID NO: 8 or SEQ ID NO: 9), at least 568 amino acid residues (e.g., amino acids 1 to 568 of SEQ ID NO: 8 or SEQ ID NO: 9), at least 600 amino acid residues (e.g., amino acids 1 to 600 of SEQ ID NO: 8 or SEQ ID NO: 9), at least 402 amino acid residues (e.g., amino acids 22 to 424 of SEQ ID NO: 8), at least 425 amino acid residues (e.g., amino acids 22 to 447 of SEQ ID NO: 8), at least 449 amino acid residues (e.g., amino acids 22 to 471 of SEQ ID NO: 8), at least 85 amino acid residues (e.g., amino acids 532 to 617 of SEQ ID NO: 8), at least 90 amino acid residues (e.g., amino acids 532 to 622 of SEQ ID NO: 8), or at least 95 amino acid residues (e.g., amino acids 532 to 627 of SEQ ID NO: 8), and wherein the fragment has alpha-amylase activity;
(iv) a fragment of SEQ ID NO: 11 wherein the fragment preferably contains at least 517 amino acid residues (e.g., amino acids 1 to 517 of SEQ ID NO: 11 or SEQ ID NO: 12), at least 548 amino acid residues (e.g., amino acids 1 to 548 of SEQ ID NO: 11 or SEQ ID NO: 12), at least 578 amino acid residues (e.g., amino acids 1 to 578 of SEQ ID NO: 11 or SEQ ID NO: 12), at least 404 amino acid residues (e.g., amino acids 20 to 424 of SEQ ID NO: 11), at least 428 amino acid residues (e.g., amino acids 20 to 448 of SEQ ID NO: 11), at least 452 amino acid residues (e.g., amino acids 20 to 472 of SEQ ID NO: 11), at least 79 amino acid residues (e.g., amino acids 508 to 587 of SEQ ID NO: 11), at least 83 amino acid residues (e.g., amino acids 508 to 591 of SEQ ID NO: 11), or at least 88 amino acid residues (e.g., amino acids 508 to 596 of SEQ ID NO: 11), and wherein the fragment has alpha-amylase activity; or
(v) a fragment of SEQ ID NO: 14 wherein the fragment preferably contains at least 403 amino acid residues (e.g., amino acids 1 to 403 of SEQ ID NO: 14 or SEQ ID NO: 15), at least 427 amino acid residues (e.g., amino acids 1 to 427 of SEQ ID NO: 14 or SEQ ID NO: 15), at least 451 amino acid residues (e.g., amino acids 1 to 451 of SEQ ID NO: 14 or SEQ ID NO: 15), at least 373 amino acid residues (e.g., amino acids 30 to 403 of SEQ ID NO: 14), at least 395 amino acid residues (e.g., amino acids 30 to 425 of SEQ ID NO: 14), or at least 417 amino acid residues (e.g., amino acids 30 to 447 of SEQ ID NO: 14);
(vi) a fragment of SEQ ID NO: 41 wherein the fragment preferably contains at least 560 amino acid residues (e.g., amino acids 1 to 560 of SEQ ID NO: 41 or SEQ ID NO: 42), at least 593 amino acid residues (e.g., amino acids 1 to 593 of SEQ ID NO: 41 or SEQ ID NO: 42, or at least 626 residues (e.g., amino acids 1 to 626 of SEQ ID NO: 41 or SEQ ID NO: 42), at least 371 amino acid residues (e.g., amino acids 28 to 399 of SEQ ID NO: 41), at least 393 amino acid residues (e.g., amino acids 28 to 421 of SEQ ID NO: 41), at least 415 amino acid residues (e.g., amino acids 28 to 443 of SEQ ID NO: 41), at least 84 amino acid residues (e.g., amino acids 554 to 638 of SEQ ID NO: 41), at least 89 amino acid residues (e.g., amino acids 554 to 643 of SEQ ID NO: 41), or at least 94 amino acid residues (e.g., amino acids 554 to 649 of SEQ ID NO: 41);
(vii) a fragment of SEQ ID NO: 44 wherein the fragment preferably contains at least 488 amino acids (e.g., amino acids 87 to 575 of SEQ ID NO: 44 or amino acids 1 to 488 of SEQ ID NO: 45), at least 517 amino acids (e.g., amino acids 58 to 575 of SEQ ID NO: 44 or amino acids 1 to 517 of SEQ ID NO: 45), at least 546 amino acids (e.g., 29 to 575 of SEQ ID NO: 44 or amino acids 1 to 546 of SEQ ID NO: 45), at least 359 amino acids (e.g., amino acids 108 to 467 of SEQ ID NO: 44 or amino acids 81 to 440 of SEQ ID NO: 45), at least 380 amino acids (e.g., amino acids 87 to 467 of SEQ ID NO: 44 or amino acids 60 to 440 of SEQ ID NO: 45), at least 401 amino acids (e.g., amino acids 66 to 467 of SEQ ID NO: 44 or amino acids 39 to 440 of SEQ ID NO: 45), at least 79 amino acids (e.g., amino acids 490 to 569 of SEQ ID NO: 44 or amino acids 463 to 542 of SEQ ID NO: 45), at least 82 amino acids (e.g., amino acids 487 to 569 of SEQ ID NO: 44 or amino acids 460 to 542 of SEQ ID NO: 45), or at least 87 amino acids (e.g., amino acids 482 to 569 of SEQ ID NO: 44 or amino acids 455 to 542 of SEQ ID NO: 45);
(viii) a fragment of SEQ ID NO: 47 wherein the fragment preferably contains at least 486 amino acids (e.g., amino acids 86 to 572 of SEQ ID NO: 47 or amino acids 1 to 486 of SEQ ID NO: 48), at least 514 amino acids (e.g., amino acids 58 to 572 of SEQ ID NO: 47 or amino acids 1 to 514 of SEQ ID NO: 48), at least 543 amino acids (e.g., amino acids 29 to 572 of SEQ ID NO: 47 or amino acids 1 to 572 of SEQ ID NO: 48), at least 359 amino acids (e.g., amino acids 108 to 467 of SEQ ID NO: 47 or amino acids 81 to 440 of SEQ ID NO: 48), at least 380 amino acids (e.g., amino acids 87 to 467 of SEQ ID NO: 47 or amino acids 60 to 440 of SEQ ID NO: 48), at least 401 amino acids (e.g., amino acids 66 to 467 of SEQ ID NO: 47 or amino acids 39 to 440 of SEQ ID NO: 48), at least 81 amino acids (e.g., amino acids 484 to 565 of SEQ ID NO: 47 or amino acids 457 to 538 of SEQ ID NO: 48), at least 86 amino acids (e.g., amino acids 479 to 565 of SEQ ID NO: 47 or amino acids 460 to 538 of SEQ ID NO: 48), or at least 91 amino acids (e.g., amino acids 474 to 565 of SEQ ID NO: 47 or amino acids 447 to 538 of SEQ ID NO: 48);
(ix) a fragment of SEQ ID NO: 50 wherein the fragment preferably contains at least 490 amino acids (e.g., amino acids 87 to 577 of SEQ ID NO: 50 or amino acids 1 to 490 of SEQ ID NO: 51), at least 519 amino acids (e.g., amino acids 58 to 577 of SEQ ID NO: 50 or amino acids 1 to 519 of SEQ ID NO: 51), at least 548 amino acids (e.g., amino acids 29 to 577 of SEQ ID NO: 50 or amino acids 1 to 548 of SEQ ID NO: 51), at least 359 amino acids (e.g., amino acids 108 to 467 of SEQ ID NO: 50 or amino acids 81 to 440 of SEQ ID NO: 51), at least 380 amino acids (e.g., amino acids 87 to 467 of SEQ ID NO: 50 or amino acids 60 to 440 of SEQ ID NO: 51), at least 401 amino acids (e.g., amino acids 66 to 467 of SEQ ID NO: 50 or amino acids 39 to 440 of SEQ ID NO: 51), at least 81 amino acids (e.g., amino acids 490 to 571 of SEQ ID NO: 50 or amino acids 463 to 544 of SEQ ID NO: 51), at least 86 amino acids (e.g., amino acids 485 to 571 of SEQ ID NO: 50 or amino acids 460 to 544 of SEQ ID NO: 51), or at least 91 amino acids (e.g., amino acids 480 to 571 of SEQ ID NO: 50 or amino acids 447 to 544 of SEQ ID NO: 51);
(x) a fragment of SEQ ID NO: 53 wherein the fragment preferably contains at least 643 amino acids (e.g., amino acids 114 to 757 of SEQ ID NO: 53 or amino acids 1 to 643 of SEQ ID NO: 54), at least 681 amino acids (e.g., amino acids 76 to 757 of SEQ ID NO: 53 or amino acids 1 to 681 of SEQ ID NO: 54), at least 719 amino acids (e.g., amino acids 38 to 757 of SEQ ID NO: 53 or amino acids 1 to 719 of SEQ ID NO: 54), at least 359 amino acids (e.g., amino acids 108 to 467 of SEQ ID NO: 53 or amino acids 81 to 440 of SEQ ID NO: 54), at least 380 amino acids (e.g., amino acids 87 to 467 of SEQ ID NO: 53 or amino acids 60 to 440 of SEQ ID NO: 54), at least 401 amino acids (e.g., amino acids 66 to 467 of SEQ ID NO: 53 or amino acids 39 to 440 of SEQ ID NO: 54), at least 74 amino acids (e.g., amino acids 488 to 562 of SEQ ID NO: 53 or amino acids 461 to 535 of SEQ ID NO: 54), at least 79 amino acids (e.g., amino acids 483 to 562 of SEQ ID NO: 53 or amino acids 456 to 535 of SEQ ID NO: 54), at least 83 amino acids (e.g., amino acids 479 to 562 of SEQ ID NO: 53 or amino acids 452 to 535 of SEQ ID NO: 54), at least 78 amino acids (e.g., amino acids 576 to 654 of SEQ ID NO: 53 or amino acids 549 to 627 of SEQ ID NO: 54), at least 82 amino acids (e.g., amino acids 572 to 654 of SEQ ID NO: 53 or amino acids 545 to 627 of SEQ ID NO: 54), at least 87 amino acids (e.g., amino acids 567 to 654 of SEQ ID NO: 53 or amino acids 540 to 627 of SEQ ID NO: 54), at least 81 amino acids (e.g., amino acids 669 to 750 of SEQ ID NO: 53 or amino acids 642 to 723 of SEQ ID NO: 54), at least 86 amino acids (e.g., amino acids 664 to 750 of SEQ ID NO: 53 or amino acids 637 to 723 of SEQ ID NO: 54), or at least 91 amino acids (e.g., amino acids 659 to 750 of SEQ ID NO: 53 or amino acids 632 to 723 of SEQ ID NO: 54);
(xi) a fragment of SEQ ID NO: 56 wherein the fragment preferably contains at least 488 amino acids (e.g., amino acids 87 to 575 of SEQ ID NO: 56 or amino acids 1 to 488 of SEQ ID NO: 57), at least 517 amino acids (e.g., amino acids 58 to 575 of SEQ ID NO: 56 or amino acids 1 to 517 of SEQ ID NO: 57), at least 546 amino acids (e.g., amino acids 29 to 575 of SEQ ID NO: 56 or amino acids 1 to 546 of SEQ ID NO: 57), at least 359 amino acids (e.g., amino acids 108 to 467 of SEQ ID NO: 56 or amino acids 81 to 440 of SEQ ID NO: 57), at least 380 amino acids (e.g., amino acids 87 to 467 of SEQ ID NO: 56 or amino acids 60 to 440 of SEQ ID NO: 57), at least 401 amino acids (e.g., amino acids 66 to 467 of SEQ ID NO: 56 or amino acids 39 to 440 of SEQ ID NO: 57), at least 79 amino acids (e.g., amino acids 496 to 569 of SEQ ID NO: 56 or amino acids 463 to 542 of SEQ ID NO: 57), at least 84 amino acids (e.g., amino acids 481 to 569 of SEQ ID NO: 56 or amino acids 458 to 542 of SEQ ID NO: 57), or at least 88 amino acids (e.g., amino acids 481 to 569 of SEQ ID NO: 56 or amino acids 454 to 542 of SEQ ID NO: 57);
(xii) a fragment of SEQ ID NO: 59 wherein the fragment preferably contains at least 491 amino acids (e.g., amino acids 87 to 578 of SEQ ID NO: 59 or amino acids 1 to 491 of SEQ ID NO: 60), at least 520 amino acids (e.g., amino acids 58 to 578 of SEQ ID NO: 59 or amino acids 1 to 520 of SEQ ID NO: 60), at least 549 amino acids (e.g., amino acids 29 to 578 of SEQ ID NO: 59 or amino acids 1 to 549 of SEQ ID NO: 60), at least 359 amino acids (e.g., amino acids 108 to 467 of SEQ ID NO: 59 or amino acids 81 to 440 of SEQ ID NO: 60), at least 380 amino acids (e.g., amino acids 87 to 467 of SEQ ID NO: 59 or amino acids 60 to 440 of SEQ ID NO: 60), at least 401 amino acids (e.g., amino acids 66 to 467 of SEQ ID NO: 59 or amino acids 39 to 440 of SEQ ID NO: 60), at least 79 amino acids (e.g., amino acids 499 to 572 of SEQ ID NO: 59 or amino acids 466 to 545 of SEQ ID NO: 60), at least 84 amino acids (e.g., amino acids 484 to 572 of SEQ ID NO: 59 or amino acids 461 to 545 of SEQ ID NO: 60), or at least 88 amino acids (e.g., amino acids 484 to 572 of SEQ ID NO: 59 or amino acids 457 to 545 of SEQ ID NO: 60).

7. The polypeptide of any one of paragraphs 1-6, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 2, SEQ ID NO: 5, SEQ ID NO: 8, SEQ ID NO: 11, SEQ ID NO: 14, SEQ ID NO: 41, SEQ ID NO: 44, SEQ ID NO: 47, SEQ ID NO: 50, SEQ ID NO: 53, SEQ ID NO: 56, or SEQ ID NO: 59.

8. The polypeptide of any one of paragraphs 1-7, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 3, SEQ ID NO: 6, SEQ ID NO: 9, SEQ ID NO: 12, SEQ ID NO: 15, SEQ ID NO: 42, SEQ ID NO: 45, SEQ ID NO: 48, SEQ ID NO: 51, SEQ ID NO: 54, SEQ ID NO: 57, or SEQ ID NO: 60.

9. The polypeptide of any one of paragraphs 1-8, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to a mature polypeptide of SEQ ID NO: 2, SEQ ID NO: 5, SEQ ID NO: 8, SEQ ID NO: 11, SEQ ID NO: 14, SEQ ID NO: 41, SEQ ID NO: 44, SEQ ID NO: 47, SEQ ID NO: 50, SEQ ID NO: 53, SEQ ID NO: 56 or SEQ ID NO: 59.

10. The polypeptide of any one of paragraphs 1-9, which is encoded by a polynucleotide that hybridizes under medium stringency conditions, medium-high stringency conditions, high stringency conditions, or very high stringency conditions with the full-length complement of the mature polypeptide coding sequence of SEQ ID NO: 1, SEQ ID NO: 4, SEQ ID NO: 7, SEQ ID NO: 10, SEQ ID NO: 13, SEQ ID NO: 40, SEQ ID NO: 43, SEQ ID NO: 46, SEQ ID NO: 49, SEQ ID NO: 52, SEQ ID NO: 55, or SEQ ID NO: 58, or the cDNA of any thereof.

11. The polypeptide of any one of paragraphs 1-10, which is encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 1, SEQ ID NO: 4 or the cDNA sequence thereof, SEQ ID NO: 7 or the cDNA sequence thereof, SEQ ID NO: 10, SEQ ID NO: 13, SEQ ID NO: 40, SEQ ID NO: 43, SEQ ID NO: 47, SEQ ID NO: 50, SEQ ID NO: 53, SEQ ID NO: 56, or SEQ ID NO: 59.

12. The polypeptide of any one of paragraphs 1-11, which is:
(i) a fragment of SEQ ID NO: 2 or SEQ ID NO: 3, wherein the fragment preferably contains at least 512 amino acid residues (e.g., amino acids 1 to 512 of SEQ ID NO: 2 or SEQ ID NO: 3), at least 542 amino acid residues (e.g., amino acids 1 to 542 of SEQ ID NO: 2 or SEQ ID NO: 3), at least 572 amino acid residues (e.g., amino acids 1 to 572 of SEQ ID NO: 2 or SEQ ID NO: 3), at least 401 amino acid residues (e.g., amino acids 21 to 422 of SEQ ID NO: 2), at least 424 amino acid residues (e.g., amino acids 21 to 445 of SEQ ID NO: 2), at least 448 amino acid residues (e.g., amino acids 21 to 469 of SEQ ID NO: 2), at least 81 amino acid residues (e.g., amino acids 507 to 588 of SEQ ID NO: 2), at least 86 amino acid residues (e.g., amino acids 507 to 593 of SEQ ID NO: 2), or at least 91 amino acid residues (e.g., amino acids 507 to 598 of SEQ ID NO: 2), and wherein the fragment has alpha-amylase activity;
(ii) a fragment of SEQ ID NO: 5 or SEQ ID NO: 5, wherein the fragment preferably contains at least 537 amino acid residues (e.g., amino acids 1 to 537 of SEQ ID NO: 5 or SEQ ID NO: 6), at least 568 amino acid residues (e.g., amino acids 1 to 568 of SEQ ID NO: 5 or SEQ ID NO: 6), at least 600 amino acid residues (e.g., amino acids 1 to 600 of SEQ ID NO: 5 or SEQ ID NO: 6), at least 407 amino acid residues (e.g., amino acids 18 to 425 of SEQ ID NO: 5), at least 431 amino acid residues (e.g., amino acids 18 to 449 of SEQ ID NO: 5), or at least 455 amino acid residues (e.g., amino acids 18 to 473 of SEQ ID NO: 5), at least 85 amino acid residues (e.g., amino acids 532 to 617 of SEQ ID NO: 5), at least 90 amino acid residues (e.g., amino acids 532 to 622 of SEQ ID NO: 5), or at least 95 amino acid residues (e.g., amino acids 532 to 627 of SEQ ID NO: 5), and wherein the fragment has alpha-amylase activity;
(iii) a fragment of SEQ ID NO: 8 or SEQ ID NO: 9, wherein the fragment preferably contains at least 537 amino acid residues (e.g., amino acids 1 to 537 of SEQ ID NO: 8 or SEQ ID NO: 9), at least 568 amino acid residues (e.g., amino acids 1 to 568 of SEQ ID NO: 8 or SEQ ID NO: 9), at least 600 amino acid residues (e.g., amino acids 1 to 600 of SEQ ID NO: 8 or SEQ ID NO: 9), at least 402 amino acid residues (e.g., amino acids 22 to 424 of SEQ ID NO: 8), at least 425 amino acid residues (e.g., amino acids 22 to 447 of SEQ ID NO: 8), at least 449 amino acid residues (e.g., amino acids 22 to 471 of SEQ ID NO: 8), at least 85 amino acid residues (e.g., amino acids 532 to 617 of SEQ ID NO: 8), at least 90 amino acid residues (e.g., amino acids 532 to 622 of SEQ ID NO: 8), or at least 95 amino acid residues (e.g., amino acids 532 to 627 of SEQ ID NO: 8), and wherein the fragment has alpha-amylase activity;
(iv) a fragment of SEQ ID NO: 11 or SEQ ID NO: 12, wherein the fragment preferably contains at least 517 amino acid residues (e.g., amino acids 1 to 517 of SEQ ID NO: 11 or SEQ ID NO: 12), at least 548 amino acid residues (e.g., amino acids 1 to 548 of SEQ ID NO: 11 or SEQ ID NO: 12), at least 578 amino acid residues (e.g., amino acids 1 to 578 of SEQ ID NO: 11 or SEQ ID NO: 12), at least 404 amino acid residues (e.g., amino acids 20 to 424 of SEQ ID NO: 11), at least 428 amino acid residues (e.g., amino acids 20 to 448 of SEQ ID NO: 11), at least 452 amino acid residues (e.g., amino acids 20 to 472 of SEQ ID NO: 11), at least 79 amino acid residues (e.g., amino acids 508 to 587 of SEQ ID NO: 11), at least 83 amino acid residues (e.g., amino acids 508 to 591 of SEQ ID NO: 11), or at least 88 amino acid residues (e.g., amino acids 508 to 596 of SEQ ID NO: 11), and wherein the fragment has alpha-amylase activity; or
(v) a fragment of SEQ ID NO: 14 or SEQ ID NO: 15, wherein the fragment preferably contains at least 403 amino acid residues (e.g., amino acids 1 to 403 of SEQ ID NO: 14 or SEQ ID NO: 15), at least 427 amino acid residues (e.g., amino acids 1 to 427 of SEQ ID NO: 14 or SEQ ID NO: 15), at least 451 amino acid residues (e.g., amino acids 1 to 451 of SEQ ID NO: 14 or SEQ ID NO: 15), at least 373 amino acid residues (e.g., amino acids 30 to 403 of SEQ ID NO: 14), at least 395 amino acid residues (e.g., amino acids 30 to 425 of SEQ ID NO: 14), or at least 417 amino acid residues (e.g., amino acids 30 to 447 of SEQ ID NO: 14);
(vi) a fragment of SEQ ID NO: 41 or SEQ ID NO: 42, wherein the fragment preferably contains at least 517 amino acid residues (e.g., amino acids 1 to 517 of SEQ ID NO: 41 or SEQ ID NO: 42), at least 548 amino acid residues (e.g., amino acids 1 to 548 of SEQ ID NO: 41 or SEQ ID NO: 42), at least 578 amino acid residues (e.g., amino acids 1 to 578 of SEQ ID NO: 41 or SEQ ID NO: 42), at least 404 amino acid residues (e.g., amino acids 20 to 424 of SEQ ID NO: 41), at least 428 amino acid residues (e.g., amino acids 20 to 448 of SEQ ID NO: 41), at least 452 amino acid residues (e.g., amino acids 20 to 472 of SEQ ID NO: 41), at least 79 amino acid residues (e.g., amino acids 508 to 587 of SEQ ID NO: 41), at least 83 amino acid residues (e.g., amino acids 508 to 591 of SEQ ID NO: 41), or at least 88 amino acid residues (e.g., amino acids 508 to 596 of SEQ ID NO: 41), and wherein the fragment has alpha-amylase activity;
(vii) a fragment of SEQ ID NO: 44 or SEQ ID NO: 45 wherein the fragment preferably contains at least 488 amino acids (e.g., amino acids 87 to 575 of SEQ ID NO: 44 or amino acids 1 to 488 of SEQ ID NO: 45), at least 517 amino acids (e.g., amino acids 58 to 575 of SEQ ID NO: 44 or amino acids 1 to 517 of SEQ ID NO: 45), at least 546 amino acids (e.g., 29 to 575 of SEQ ID NO: 44 or amino acids 1 to 546 of SEQ ID NO: 45), at least 359 amino acids (e.g., amino acids 108 to 467 of SEQ ID NO: 44 or amino acids 81 to 440 of SEQ ID NO: 45), at least 380 amino acids (e.g., amino acids 87 to 467 of SEQ ID NO: 44 or amino acids 60 to 440 of SEQ ID NO: 45), at least 401 amino acids (e.g., amino acids 66 to 467 of SEQ ID NO: 44 or amino acids 39 to 440 of SEQ ID NO: 45), at least 79 amino acids (e.g., amino acids 490 to 569 of SEQ ID NO: 44 or amino acids 463 to 542 of SEQ ID NO: 45), at least 82 amino acids (e.g., amino acids 487 to 569 of SEQ ID NO: 44 or amino acids 460 to 542 of SEQ ID NO: 45), or at least 87 amino acids (e.g., amino acids 482 to 569 of SEQ ID NO: 44 or amino acids 455 to 542 of SEQ ID NO: 45);
(viii) a fragment of SEQ ID NO: 47 or SEQ ID NO: 48 wherein the fragment preferably contains at least 486 amino acids (e.g., amino acids 86 to 572 of SEQ ID NO: 47 or amino acids 1 to 486 of SEQ ID NO: 48), at least 514 amino acids (e.g., amino acids 58 to 572 of SEQ ID NO: 47 or amino acids 1 to 514 of SEQ ID NO: 48), at least 543 amino acids (e.g., amino acids 29 to 572 of SEQ ID NO: 47 or amino acids 1 to 572 of SEQ ID NO: 48), at least 359 amino acids (e.g., amino acids 108 to 467 of SEQ ID NO: 47 or amino acids 81 to 440 of SEQ ID NO: 48), at least 380 amino acids (e.g., amino acids 87 to 467 of SEQ ID NO: 47 or amino acids 60 to 440 of SEQ ID NO: 48), at least 401 amino acids (e.g., amino acids 66 to 467 of SEQ ID NO: 47 or amino acids 39 to 440 of SEQ ID NO: 48), at least 81 amino acids (e.g., amino acids 484 to 565 of SEQ ID NO: 47 or amino acids 457 to 538 of SEQ ID NO: 48), at least 86 amino acids (e.g., amino acids 479 to 565 of SEQ ID NO: 47 or amino acids 460 to 538 of SEQ ID NO: 48), or at least 91 amino acids (e.g., amino acids 474 to 565 of SEQ ID NO: 47 or amino acids 447 to 538 of SEQ ID NO: 48);
(ix) a fragment of SEQ ID NO: 50 or SEQ ID NO: 51 wherein the fragment preferably contains at least 490 amino acids (e.g., amino acids 87 to 577 of SEQ ID NO: 50 or amino acids 1 to 490 of SEQ ID NO: 51), at least 519 amino acids (e.g., amino acids 58 to 577 of SEQ ID NO: 50 or amino acids 1 to 519 of SEQ ID NO: 51), at least 548 amino acids (e.g., amino acids 29 to 577 of SEQ ID NO: 50 or amino acids 1 to 548 of SEQ ID NO: 51), at least 359 amino acids (e.g., amino acids 108 to 467 of SEQ ID NO: 50 or amino acids 81 to 440 of SEQ ID NO: 51), at least 380 amino acids (e.g., amino acids 87 to 467 of SEQ ID NO: 50 or amino acids 60 to 440 of SEQ ID NO: 51), at least 401 amino acids (e.g., amino acids 66 to 467 of SEQ ID NO: 50 or amino acids 39 to 440 of SEQ ID NO: 51), at least 81 amino acids (e.g., amino acids 490 to 571 of SEQ ID NO: 50 or amino acids 463 to 544 of SEQ ID NO: 51), at least 86 amino acids (e.g., amino acids 485 to 571 of SEQ ID NO: 50 or amino acids 460 to 544 of SEQ ID NO: 51), or at least 91 amino acids (e.g., amino acids 480 to 571 of SEQ ID NO: 50 or amino acids 447 to 544 of SEQ ID NO: 51);
(x) a fragment of SEQ ID NO: 53 or SEQ ID NO: 54 wherein the fragment preferably contains at least 643 amino acids (e.g., amino acids 114 to 757 of SEQ ID NO: 53 or amino acids 1 to 643 of SEQ ID NO: 54), at least 681 amino acids (e.g., amino acids 76 to 757 of SEQ ID NO: 53 or amino acids 1 to 681 of SEQ ID NO: 54), at least 719 amino acids (e.g., amino acids 38 to 757 of SEQ ID NO: 53 or amino acids 1 to 719 of SEQ ID NO: 54), at least 359 amino acids (e.g., amino acids 108 to 467 of SEQ ID NO: 53 or amino acids 81 to 440 of SEQ ID NO: 54), at least 380 amino acids (e.g., amino acids 87 to 467 of SEQ ID NO: 53 or amino acids 60 to 440 of SEQ ID NO: 54), at least 401 amino acids (e.g., amino acids 66 to 467 of SEQ ID NO: 53 or amino acids 39 to 440 of SEQ ID NO: 54), at least 74 amino acids (e.g., amino acids 488 to 562 of SEQ ID NO: 53 or amino acids 461 to 535 of SEQ ID NO: 54), at least 79 amino acids (e.g., amino acids 483 to 562 of SEQ ID NO: 53 or amino acids 456 to 535 of SEQ ID NO: 54), at least 83 amino acids (e.g., amino acids 479 to 562 of SEQ ID NO: 53 or amino acids 452 to 535 of SEQ ID NO: 54), at least 78 amino acids (e.g., amino acids 576 to 654 of SEQ ID NO: 53 or amino acids 549 to 627 of SEQ ID NO: 54), at least 82 amino acids (e.g., amino acids 572 to 654 of SEQ ID NO: 53 or amino acids 545 to 627 of SEQ ID NO: 54), at least 87 amino acids (e.g., amino acids 567 to 654 of SEQ ID NO: 53 or amino acids 540 to 627 of SEQ ID NO: 54), at least 81 amino acids (e.g., amino acids 669 to 750 of SEQ ID NO: 53 or amino acids 642 to 723 of SEQ ID NO: 54), at least 86 amino acids (e.g., amino acids 664 to 750 of SEQ ID NO: 53 or amino acids 637 to 723 of SEQ ID NO: 54), or at least 91 amino acids (e.g., amino acids 659 to 750 of SEQ ID NO: 53 or amino acids 632 to 723 of SEQ ID NO: 54);
(xi) a fragment of SEQ ID NO: 56 or SEQ ID NO: 57 wherein the fragment preferably contains at least 488 amino acids (e.g., amino acids 87 to 575 of SEQ ID NO: 56 or amino acids 1 to 488 of SEQ ID NO: 57), at least 517 amino acids (e.g., amino acids 58 to 575 of SEQ ID NO: 56 or amino acids 1 to 517 of SEQ ID NO: 57), at least 546 amino acids (e.g., amino acids 29 to 575 of SEQ ID NO: 56 or amino acids 1 to 546 of SEQ ID NO: 57), at least 359 amino acids (e.g., amino acids 108 to 467 of SEQ ID NO: 56 or amino acids 81 to 440 of SEQ ID NO: 57), at least 380 amino acids (e.g., amino acids 87 to 467 of SEQ ID NO: 56 or amino acids 60 to 440 of SEQ ID NO: 57), at least 401 amino acids (e.g., amino acids 66 to 467 of SEQ ID NO: 56 or amino acids 39 to 440 of SEQ ID NO: 57), at least 79 amino acids (e.g., amino acids 496 to 569 of SEQ ID NO: 56 or amino acids 463 to 542 of SEQ ID NO: 57), at least 84 amino acids (e.g., amino acids 481 to 569 of SEQ ID NO: 56 or amino acids 458 to 542 of SEQ ID NO: 57), or at least 88 amino acids (e.g., amino acids 481 to 569 of SEQ ID NO: 56 or amino acids 454 to 542 of SEQ ID NO: 57); or
(xii) a fragment of SEQ ID NO: 59 or SEQ ID NO: 60 wherein the fragment preferably contains at least 491 amino acids (e.g., amino acids 87 to 578 of SEQ ID NO: 59 or amino acids 1 to 491 of SEQ ID NO: 60), at least 520 amino acids (e.g., amino acids 58 to 578 of SEQ ID NO: 59 or amino acids 1 to 520 of SEQ ID NO: 60), at least 549 amino acids (e.g., amino acids 29 to 578 of SEQ ID NO: 59 or amino acids 1 to 549 of SEQ ID NO: 60), at least 359 amino acids (e.g., amino acids 108 to 467 of SEQ ID NO: 59 or amino acids 81 to 440 of SEQ ID NO: 60), at least 380 amino acids (e.g., amino acids 87 to 467 of SEQ ID NO: 59 or amino acids 60 to 440 of SEQ ID NO: 60), at least 401 amino acids (e.g., amino acids 66 to 467 of SEQ ID NO: 59 or amino acids 39 to 440 of SEQ ID NO: 60), at least 79 amino acids (e.g., amino acids 499 to 572 of SEQ ID NO: 59 or amino acids 466 to 545 of SEQ ID NO: 60), at least 84 amino acids (e.g., amino acids 484 to 572 of SEQ ID NO: 59 or amino acids 461 to 545 of SEQ ID NO: 60), or at least 88 amino acids (e.g., amino acids 484 to 572 of SEQ ID NO: 59 or amino acids 457 to 545 of SEQ ID NO: 60).

13. The polypeptide of any one of paragraphs 1-11, which is:
(i) a variant of SEQ ID NO: 2, a variant of a mature polypeptide of SEQ ID NO: 2, or a variant of SEQ ID NO: 3 comprising a substitution, deletion, and/or insertion at one or more positions, preferably at one or more positions other than positions corresponding to D223, E247, and D314 of SEQ ID NO: 2;
(ii) a variant of SEQ ID NO: 5, a variant of a mature polypeptide of SEQ ID NO: 5, or a variant of SEQ ID NO: 6 comprising a substitution, deletion, and/or insertion at one or more positions, preferably at one or more positions other than positions corresponding to D223, E247, and D314 of SEQ ID NO: 5;
(iii) a variant of SEQ ID NO: 8, a variant of a mature polypeptide of SEQ ID NO: 8, or a variant of SEQ ID NO: 9 comprising a substitution, deletion, and/or insertion at one or more positions, preferably at one or more positions other than positions corresponding to D223, E247, and D314 of SEQ ID NO: 8;
(iv) a variant of SEQ ID NO: 11, a variant of a mature polypeptide of SEQ ID NO: 11, or a variant of SEQ ID NO: 12 comprising a substitution, deletion, and/or insertion at one or more positions, preferably at one or more positions other than positions corresponding to D225, E249, and D316 of SEQ ID NO: 11;
(v) a variant of SEQ ID NO: 14, a variant of a mature polypeptide of SEQ ID NO: 14, or a variant of SEQ ID NO: 15 comprising a substitution, deletion, and/or insertion at one or more positions, preferably at one or more positions other than positions corresponding to D217, E251, and D312 of SEQ ID NO: 14;
(vi) a variant of SEQ ID NO: 41, a variant of a mature polypeptide of SEQ ID NO: 41, or a variant of SEQ ID NO: 42 comprising a substitution, deletion, and/or insertion at one or more positions, preferably at one or more positions other than positions corresponding to D184, E216, and D277 of SEQ ID NO: 41;
(vii) a variant of SEQ ID NO: 44, a variant of a mature polypeptide of SEQ ID NO: 44, or a variant of SEQ ID NO: 45 comprising a substitution, deletion, and/or insertion at one or more positions, preferably at one or more positions in SEQ ID NO: 44 or SEQ ID NO: 45 other than positions corresponding to positions D215, E249, and D310 of SEQ ID NO: 44;
(viii) a variant of SEQ ID NO: 47, a variant of a mature polypeptide of SEQ ID NO: 47, or a variant of SEQ ID NO: 48 comprising a substitution, deletion, and/or insertion at one or more positions, preferably at one or more positions in SEQ ID NO: 47 or SEQ ID NO: 48 other than positions corresponding to positions D215, E249, and D310 of SEQ ID NO: 47;
(ix) a variant of SEQ ID NO: 50, a variant of a mature polypeptide of SEQ ID NO: 50, or a variant of SEQ ID NO: 51 comprising a substitution, deletion, and/or insertion at one or more positions, preferably at one or more positions in SEQ ID NO: 50 or SEQ ID NO: 51 other than positions corresponding to positions D215, E249, and D310 of SEQ ID NO: 50; or
(x) a variant of SEQ ID NO: 53, a variant of a mature polypeptide of SEQ ID NO: 53, or a variant of SEQ ID NO: 54 comprising a substitution, deletion, and/or insertion at one or more positions, preferably at one or more positions in SEQ ID NO: 53 or SEQ ID NO: 54 other than positions corresponding to positions D215, E249, and D310 of SEQ ID NO: 53;
(xi) a variant of SEQ ID NO: 56, a variant of a mature polypeptide of SEQ ID NO: 56, or a variant of SEQ ID NO: 57 comprising a substitution, deletion, and/or insertion at one or more positions, preferably at one or more positions in SEQ ID NO: 56 or SEQ ID NO: 57 other than positions corresponding to positions D215, E249, and D310 of SEQ ID NO: 56;
(xii) a variant of SEQ ID NO: 59, a variant of a mature polypeptide of SEQ ID NO: 59, or a variant of SEQ ID NO: 60 comprising a substitution, deletion, and/or insertion at one or more positions, preferably at one or more positions in SEQ ID NO: 59 or SEQ ID NO: 60 other than positions corresponding to positions D215, E249, and D310 of SEQ ID NO: 59.

14. The polypeptide of any one of paragraphs 1-11, comprising, consisting essentially of, or consisting of SEQ ID NO: 2, SEQ ID NO: 5, SEQ ID NO: 8, SEQ ID NO: 11, SEQ ID NO: 14, SEQ ID NO: 41, SEQ ID NO: 44, SEQ ID NO: 47, SEQ ID NO: 50, SEQ ID NO: 53, SEQ ID NO: 56, or SEQ ID NO: 59.

15. The polypeptide of any one of paragraphs 1-11, comprising, consisting essentially of, or consisting of SEQ ID NO: 3, SEQ ID NO: 6, SEQ ID NO: 9, SEQ ID NO: 12, SEQ ID NO: 15, SEQ ID NO: 42, SEQ ID NO: 45, SEQ ID NO: 48, SEQ ID NO: 51, SEQ ID NO: 54, SEQ ID NO: 57, or SEQ ID NO: 60.

16. The polypeptide of any one of paragraphs 1-11, comprising SEQ ID NO: 3, SEQ ID NO: 6, SEQ ID NO: 9, SEQ ID NO: 12, SEQ ID NO: 15, SEQ ID NO: 42, SEQ ID NO: 45, SEQ ID NO: 48, SEQ ID NO: 51, SEQ ID NO: 54, SEQ ID NO: 57, or SEQ ID NO: 60, and an N-terminal extension and/or C-terminal extension of 1-10 amino acids, e.g., 1, 2, 3, 4, 5, 6, 7, 8, 9, or 10 amino acids, wherein the N-terminal extension and/or C-terminal extension optionally comprises a tag, such as a His tag.

17. The polypeptide of any one of paragraphs 1-11, which is a mature polypeptide of SEQ ID NO: 2, SEQ ID NO: 5, SEQ ID NO: 8, SEQ ID NO: 11, SEQ ID NO: 14, SEQ ID NO: 41, SEQ ID NO: 44, SEQ ID NO: 47, SEQ ID NO: 50, SEQ ID NO: 53, SEQ ID NO: 56, or SEQ ID NO: 59.

18. An isolated or purified polypeptide comprising a catalytic domain selected from the group consisting of:

(i)
(a) a catalytic domain having at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 18 to 497 of SEQ ID NO: 5;
(b) a catalytic domain encoded by a polynucleotide that hybridizes under medium, medium-high, high, or very high stringency conditions with the full-length complement of nucleotides 52 to 1943 of SEQ ID NO: 4, or the cDNA sequence thereof;
(c) a catalytic domain encoded by a polynucleotide having at least at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 52 to 1943 of SEQ ID NO: 4, or the cDNA sequence thereof; and
(d) a fragment of the catalytic domain of (a), (b), or (c) that has alpha-amylase activity;
   (ii)
(a) a catalytic domain having at least 94%, at least 95%, at least 96%, at least 97%, at least 98%, at least 99%, or 100% sequence identity to amino acids 22 to 495 of SEQ ID NO: 8;
(b) a catalytic domain encoded by a polynucleotide that hybridizes under medium, medium-high, high, or very high stringency conditions with the full-length complement of nucleotides 64 to 1925 of SEQ ID NO: 7, or the cDNA sequence thereof;
(c) a catalytic domain encoded by a polynucleotide having at least at least 94%, at least 95%, at least 96%, at least 97%, at least 98%, at least 99%, or 100% sequence identity to nucleotides 64 to 1925 of SEQ ID NO: 7, or the cDNA sequence thereof; and
(d) a fragment of the catalytic domain of (a), (b), or (c) that has alpha-amylase activity;
   (iii)
(a) a catalytic domain having at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 20 to 496 of SEQ ID NO: 11;
(b) a catalytic domain encoded by a polynucleotide that hybridizes under medium, medium-high, high, or very high stringency conditions with the full-length complement of nucleotides 58 to 1488 of SEQ ID NO: 10;
(c) a catalytic domain encoded by a polynucleotide having at least at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 58 to 1488 of SEQ ID NO: 10; and
(d) a fragment of the catalytic domain of (a), (b), or (c) that has alpha-amylase activity;
   (iv)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 30 to 469 of SEQ ID NO: 14;
(b) a catalytic domain encoded by a polynucleotide that hybridizes under medium, medium-high, high, or very high stringency conditions with the full-length complement of nucleotides 88 to 1407 of SEQ ID NO: 13;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 88 to 1407 of SEQ ID NO: 13; and
(d) a fragment of the catalytic domain of (a), (b), or (c) that has alpha-amylase activity;
   (v)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 28 to 465 of SEQ ID NO: 41;
(b) a catalytic domain encoded by a polynucleotide that hybridizes under medium, medium-high, high, or very high stringency conditions with the full-length complement of nucleotides 82 to 1395 of SEQ ID NO: 40;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 82 to 1395 of SEQ ID NO: 13; and
(d) a fragment of the catalytic domain of (a), (b), or (c) that has alpha-amylase activity;
   (vi)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 44;
(b) a catalytic domain encoded by a polynucleotide that hybridizes under medium, medium-high, high, or very high stringency conditions with the full-length complement of nucleotides 82 to 1725 of SEQ ID NO: 43;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 82 to 1725 of SEQ ID NO: 43; and
(d) a fragment of the catalytic domain of (a), (b), or (c) that has alpha-amylase activity;
   (vii)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 47;
(b) a catalytic domain encoded by a polynucleotide that hybridizes under medium, medium-high, high, or very high stringency conditions with the full-length complement of nucleotides 82 to 1716 of SEQ ID NO: 46;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 82 to 1716 of SEQ ID NO: 46; and
(d) a fragment of the catalytic domain of (a), (b), or (c) that has alpha-amylase activity;
   (viii)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 50;
(b) a catalytic domain encoded by a polynucleotide that hybridizes under medium, medium-high, high, or very high stringency conditions with the full-length complement of nucleotides 82 to 1731 of SEQ ID NO: 49;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 82 to 1731 of SEQ ID NO: 49; and
(d) a fragment of the catalytic domain of (a), (b), or (c) that has alpha-amylase activity;
   (ix)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 53;
(b) a catalytic domain encoded by a polynucleotide that hybridizes under medium, medium-high, high, or very high stringency conditions with the full-length complement of nucleotides 82 to 2271 of SEQ ID NO: 52;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 82 to 2271 of SEQ ID NO: 52; and
(d) a fragment of the catalytic domain of (a), (b), or (c) that has alpha-amylase activity;
   (x)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 56;
(b) a catalytic domain encoded by a polynucleotide that hybridizes under medium, medium-high, high, or very high stringency conditions with the full-length complement of nucleotides 133 to 1401 of SEQ ID NO: 55;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 133 to 1401 of SEQ ID NO: 55; and
(d) a fragment of the catalytic domain of (a), (b), or (c) that has alpha-amylase activity; and
   (xi)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 59;
(b) a catalytic domain encoded by a polynucleotide that hybridizes under medium, medium-high, high, or very high stringency conditions with the full-length complement of nucleotides 133 to 1401 of SEQ ID NO: 58;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 133 to 1401 of SEQ ID NO: 58; and
(d) a fragment of the catalytic domain of (a), (b), or (c) that has alpha-amylase activity.

19. The polypeptide of paragraph 18, further comprising a starch binding module, e.g., a heterologous starch binding module.

20. The polypeptide of paragraph 18 or 19, wherein the catalytic domain has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to: (i) amino acids 18 to 497 of SEQ ID NO: 5 or amino acids 1 to 480 of SEQ ID NO: 6; (ii) amino acids 22 to 495 of SEQ ID NO: 8 or amino acids 1 to 474 of SEQ ID NO: 9; (iii) amino acids 20 to 496 of SEQ ID NO: 11 or amino acids 1 to 477 of SEQ ID NO: 12; (iv) amino acids 30 to 469 of SEQ ID NO: 14 or amino acids 1 to 449 of SEQ ID NO: 15; (v) amino acids 28 to 465 of SEQ ID NO: 41 or amino acids 1 to 437 of SEQ ID NO: 42; (vi) amino acids 45 to 467 of SEQ ID NO: 44 or amino acids 1 to 440 of SEQ ID NO: 45; (vi) amino acids 45 to 467 of SEQ ID NO: 47 or amino acids 1 to 440 of SEQ ID NO: 48; (vii) amino acids 45 to 467 of SEQ ID NO: 50 or amino acids 1 to 440 of SEQ ID NO: 51; (viii) amino acids 45 to 467 of SEQ ID NO: 53 or amino acids 1 to 440 of SEQ ID NO: 54; (ix) amino acids 45 to 467 of SEQ ID NO: 56 or amino acids 1 to 440 of SEQ ID NO: 57; (x) amino acids 45 to 467 of SEQ ID NO: 59 or amino acids 1 to 440 of SEQ ID NO: 60.

21. The polypeptide of any one of paragraphs 18-20, wherein the catalytic domain is encoded by a polynucleotide that hybridizes under medium stringency conditions, medium-high stringency conditions, high stringency conditions, or very high stringency conditions with the full-length complement of: (i) nucleotides 52 to 1943 of SEQ ID NO: 4 or the cDNA thereof; (ii) nucleotides 64 to 1925 of SEQ ID NO: 7 or the cDNA thereof; (iii) nucleotides 58 to 1488 of SEQ ID NO: 10 or the cDNA thereof; (iv) nucleotides 88 to 1407 of SEQ ID NO: 13 or the cDNA thereof, or nucleotides 82 to 1395 of SEQ ID NO: 40 or the cDNA thereof; (v) nucleotides 133 to 1401 of SEQ ID NO: 43; (vi) nucleotides 133 to 1401 of SEQ ID NO: 46; (vii) nucleotides 133 to 1401 of SEQ ID NO: 49; (viii) nucleotides 133 to 1401 of SEQ ID NO: 52; (ix) nucleotides 133 to 1401 of SEQ ID NO: 55; or (x) nucleotides 133 to 1401 of SEQ ID NO: 58.

22. The polypeptide of any one of paragraphs 18-21, wherein the catalytic domain is encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to: (i) nucleotides 52 to 1943 of SEQ ID NO: 4 or the cDNA sequence thereof; (ii) nucleotides 64 to 1925 of SEQ ID NO: 7 or the cDNA thereof; (iii) nucleotides 58 to 1488 of SEQ ID NO: 10; (iv) nucleotides 88 to 1407 of SEQ ID NO: 13; (v) nucleotides 82 to 1395 of SEQ ID NO: 40; (v) nucleotides 133 to 1401 of SEQ ID NO: 43; (vi) nucleotides 133 to 1401 of SEQ ID NO: 46; (vii) nucleotides 133 to 1401 of SEQ ID NO: 49; (viii) nucleotides 133 to 1401 of SEQ ID NO: 52; (ix) nucleotides 133 to 1401 of SEQ ID NO: 55; or (x) nucleotides 133 to 1401 of SEQ ID NO: 58.

23. The polypeptide of any one of paragraphs 18-22, wherein the catalytic domain comprises, consists essentially of, or consists of: (i) amino acids 18 to 497 of SEQ ID NO: 5 or amino acids 1 to 480 of SEQ ID NO: 6; (ii) amino acids 22 to 495 of SEQ ID NO: 8 or amino acids 1 to 474 of SEQ ID NO: 9; (iii) amino acids 20 to 496 of SEQ ID NO: 11 or amino acids 1 to 477 of SEQ ID NO: 12; (iv) amino acids 30 to 469 of SEQ ID NO: 14 or amino acids 1 to 449 of SEQ ID NO: 15.; (v) amino acids 28 to 465 of SEQ ID NO: 41 or amino acids 1 to 437 of SEQ ID NO: 42; (vi) amino acids 45 to 467 of SEQ ID NO: 44 or amino acids 1 to 440 of SEQ ID NO: 45; (vi) amino acids 45 to 467 of SEQ ID NO: 47 or amino acids 1 to 440 of SEQ ID NO: 48; (vii) amino acids 45 to 467 of SEQ ID NO: 50 or amino acids 1 to 440 of SEQ ID NO: 51; (viii) amino acids 45 to 467 of SEQ ID NO: 53 or amino acids 1 to 440 of SEQ ID NO: 54; (viii) amino acids 45 to 467 of SEQ ID NO: 56 or amino acids 1 to 440 of SEQ ID NO: 57; (ix) amino acids 45 to 467 of SEQ ID NO: 59 or amino acids 1 to 440 of SEQ ID NO: 60.

24. The polypeptide of any one of paragraphs 18-23, wherein the catalytic domain is:
(i) a variant of amino acids 21 to 493 of SEQ ID NO: 2 or a variant of amino acids 1 to 473 of SEQ ID NO: 3 comprising a substitution, deletion, and/or insertion at one or more positions, preferably at one or more positions other than positions corresponding to positions D223, E247, and D314 of SEQ ID NO: 2;
(ii) a variant of amino acids 218 to 497 of SEQ ID NO: 5 or a variant of amino acids 1 to 480 of SEQ ID NO: 6 comprising a substitution, deletion, and/or insertion at one or more positions, preferably at one or more positions other than positions corresponding to positions D223, E247, and D314 of SEQ ID NO: 5;
(iii) a variant of amino acids 22 to 495 of SEQ ID NO: 8 or a variant of amino acids 1 to 474 of SEQ ID NO: 9 comprising a substitution, deletion, and/or insertion at one or more positions, preferably at one or more positions other than positions corresponding to positions D223, E247, and D314 of SEQ ID NO: 8;
(iv) a variant of amino acids 20 to 496 of SEQ ID NO: 11 or a variant of amino acids 1 to 477 of SEQ ID NO: 12 comprising a substitution, deletion, and/or insertion at one or more positions, preferably at one or more positions other than positions corresponding to positions D225, E249, and D316 of SEQ ID NO: 11;
(v) a variant of amino acids 30 to 469 of SEQ ID NO: 14 or a variant of amino acids 1 to 440 of SEQ ID NO: 15 comprising a substitution, deletion, and/or insertion at one or more positions, preferably at one or more positions other than positions corresponding to positions D217, E251, and D312 of SEQ ID NO: 14;
(vi) a variant of amino acids 28 to 465 of SEQ ID NO: 41 or a variant of amino acids 1 to 437 of SEQ ID NO: 42 comprising a substitution, deletion, and/or insertion at one or more positions, preferably at one or more positions other than positions corresponding to positions D184, E216, and D277 of SEQ ID NO: 41;
(vii) a variant of amino acids 45 to 467 of SEQ ID NO: 44 or a variant of amino acids 1 to 440 of SEQ ID NO: 45 comprising a substitution, deletion, and/or insertion at one or more positions, preferably at one or more positions other than positions corresponding to positions D215, E249, and D310 of SEQ ID NO: 44;
(viii) a variant of amino acids 45 to 467 of SEQ ID NO: 47 or a variant of amino acids 1 to 440 of SEQ ID NO: 48 comprising a substitution, deletion, and/or insertion at one or more positions, preferably at one or more positions other than positions corresponding to positions D215, E249, and D310 of SEQ ID NO: 47;
(ix) a variant of amino acids 45 to 467 of SEQ ID NO: 50 or a variant of amino acids 1 to 440 of SEQ ID NO: 51 comprising a substitution, deletion, and/or insertion at one or more positions, preferably at one or more positions other than positions corresponding to positions D215, E249, and D310 of SEQ ID NO: 50;
(x) a variant of amino acids 45 to 467 of SEQ ID NO: 53 or a variant of amino acids 1 to 440 of SEQ ID NO: 54 comprising a substitution, deletion, and/or insertion at one or more positions, preferably at one or more positions other than positions corresponding to positions D215, E249, and D310 of SEQ ID NO: 53;
(xi) a variant of amino acids 45 to 467 of SEQ ID NO: 56 or a variant of amino acids 1 to 440 of SEQ ID NO: 57 comprising a substitution, deletion, and/or insertion at one or more positions, preferably at one or more positions other than positions corresponding to positions D215, E249, and D310 of SEQ ID NO: 56; or
(xii) a variant of amino acids 45 to 467 of SEQ ID NO: 59 or a variant of amino acids 1 to 440 of SEQ ID NO: 60 comprising a substitution, deletion, and/or insertion at one or more positions, preferably at one or more positions other than positions corresponding to positions D215, E249, and D310 of SEQ ID NO: 59.

25 The polypeptide of any one of paragraphs 18-24, wherein the catalytic domain is:
(i) a fragment of amino acids 21 to 493 of SEQ ID NO: 2 or a fragment of amino acids 1 to 473 of SEQ ID NO: 3, wherein the fragment preferably at least 401 amino acid residues (e.g., amino acids 21 to 422 of SEQ ID NO: 2 or amino acids 1 to 401 of SEQ ID NO: 3), at least 424 amino acid residues (e.g., amino acids 21 to 445 of SEQ ID NO: 2 or amino acids 1 to 424 of SEQ ID NO: 3), at least 448 amino acid residues (e.g., amino acids 21 to 469 of SEQ ID NO: 2 or amino acids 1 to 448 of SEQ ID NO: 3), and wherein the fragment has alpha-amylase activity;
(ii) a fragment of amino acids 18 to 497 of SEQ ID NO: 5 or a fragment of amino acids 1 to 480 of SEQ ID NO: 6, wherein the fragment preferably contains at least 407 amino acid residues (e.g., amino acids 18 to 425 of SEQ ID NO: 5 or amino acids 1 to 408 of SEQ ID NO: 6), at least 431 amino acid residues (e.g., amino acids 18 to 449 of SEQ ID NO: 5 or amino acids 1 to 431 of SEQ ID NO: 6), or at least 455 amino acid residues (e.g., amino acids 18 to 473 of SEQ ID NO: 5 or amino acids 1 to 455 of SEQ ID NO: 6), and wherein the fragment has alpha-amylase activity;
(iii) a fragment of amino acids 22 to 495 of SEQ ID NO: 8 or amino acids 1 to 474 of SEQ ID NO: 9, wherein the fragment preferably contains at least 402 amino acid residues (e.g., amino acids 22 to 424 of SEQ ID NO: 8 or amino acids 1 to 402 of SEQ ID NO: 9), at least 425 amino acid residues (e.g., amino acids 22 to 447 of SEQ ID NO: 8 or amino acids 1 to 425 of SEQ ID NO: 9), at least 449 amino acid residues (e.g., amino acids 22 to 471 of SEQ ID NO: 8 or amino acids 1 to 449 of SEQ ID NO: 9), and wherein the fragment has alpha-amylase activity;
(iv) a fragment of amino acids 20 to 496 of SEQ ID NO: 11 or a fragment of amino acids 1 to 477 of SEQ ID NO: 12, wherein the fragment preferably contains at least 404 amino acid residues (e.g., amino acids 20 to 424 of SEQ ID NO: 11 or amino acids 1 to 404 of SEQ ID NO: 12), at least 428 amino acid residues (e.g., amino acids 20 to 448 of SEQ ID NO: 11 or amino acids 1 to 428 of SEQ ID NO: 12), at least 452 amino acid residues (e.g., amino acids 20 to 472 of SEQ ID NO: 11 or amino acids 1 to 452 of SEQ ID NO: 12), and wherein the fragment has alpha-amylase activity;
(v) a fragment of amino acids 30 to 469 of SEQ ID NO: 14 or a fragment of amino acids 1 to 440 of SEQ ID NO: 15, wherein the fragment preferably contains at least 373 amino acid residues (e.g., amino acids 30 to 403 of SEQ ID NO: 14 or amino acids 1 to 373 of SEQ ID NO: 15), at least 395 amino acid residues (e.g., amino acids 30 to 425 of SEQ ID NO: 14 or amino acids 1 to 395 of SEQ ID NO: 15), or at least 417 amino acid residues (e.g., amino acids 30 to 447 of SEQ ID NO: 14 or amino acids 1 to 417 of SEQ ID NO: 15), and wherein the fragment has alpha-amylase activity;
(vi) a fragment of amino acids 28 to 465 of SEQ ID NO: 41 or a fragment of amino acids 1 to 437 of SEQ ID NO: 42, wherein the fragment preferably contains at least 371 amino acid residues (e.g., amino acids 28 to 399 of SEQ ID NO: 41), at least 393 amino acid residues (e.g., amino acids 28 to 421 of SEQ ID NO: 41), or at least 415 amino acid residues (e.g., amino acids 28 to 443 of SEQ ID NO: 41), and wherein the fragment has alpha-amylase activity;
(vii) a fragment of amino acids 45 to 467 of SEQ ID NO: 44 or 1 to 440 of SEQ ID NO: 45, wherein the fragment preferably contains at least 359 amino acids (e.g., amino acids 108 to 467 of SEQ ID NO: 44 or amino acids 81 to 440 of SEQ ID NO: 45), at least 380 amino acids (e.g., amino acids 87 to 467 of SEQ ID NO: 44 or amino acids 60 to 440 of SEQ ID NO: 45), or at least 401 amino acids (e.g., amino acids 66 to 467 of SEQ ID NO: 44 or amino acids 39 to 440 of SEQ ID NO: 45);
(viii) a fragment of amino acids 45 to 467 of SEQ ID NO: 47 or amino acids 1 to 440 of SEQ ID NO: 48, wherein the fragment preferably contains at least 359 amino acids (e.g., amino acids 108 to 467 of SEQ ID NO: 47 or amino acids 81 to 440 of SEQ ID NO: 48), at least 380 amino acids (e.g., amino acids 87 to 467 of SEQ ID NO: 47 or amino acids 60 to 440 of SEQ ID NO: 48), or at least 401 amino acids (e.g., amino acids 66 to 467 of SEQ ID NO: 47 or amino acids 39 to 440 of SEQ ID NO: 48);
(ix) a fragment of amino acids 45 to 467 of SEQ ID NO: 50 or amino acids 1 to 440 of SEQ ID NO: 51 wherein the fragment preferably contains at least 359 amino acids (e.g., amino acids 108 to 467 of SEQ ID NO: 50 or amino acids 81 to 440 of SEQ ID NO: 51), at least 380 amino acids (e.g., amino acids 87 to 467 of SEQ ID NO: 50 or amino acids 60 to 440 of SEQ ID NO: 51), or at least 401 amino acids (e.g., amino acids 66 to 467 of SEQ ID NO: 50 or amino acids 39 to 440 of SEQ ID NO: 51);
(x) a fragment of amino acids 45 to 467 of SEQ ID NO: 53 or amino acids 1 to 440 of SEQ ID NO: 54 wherein the fragment preferably contains at least 359 amino acids (e.g., amino acids 108 to 467 of SEQ ID NO: 53 or amino acids 81 to 440 of SEQ ID NO: 54), at least 380 amino acids (e.g., amino acids 87 to 467 of SEQ ID NO: 53 or amino acids 60 to 440 of SEQ ID NO: 54), or at least 401 amino acids (e.g., amino acids 66 to 467 of SEQ ID NO: 53 or amino acids 39 to 440 of SEQ ID NO: 54);
(xi) a fragment of amino acids 45 to 467 of SEQ ID NO: 56 or amino acids 1 to 440 of SEQ ID NO: 57 wherein the fragment preferably contains at least 359 amino acids (e.g., amino acids 108 to 467 of SEQ ID NO: 56 or amino acids 81 to 440 of SEQ ID NO: 57), at least 380 amino acids (e.g., amino acids 87 to 467 of SEQ ID NO: 56 or amino acids 60 to 440 of SEQ ID NO: 57), or at least 401 amino acids (e.g., amino acids 66 to 467 of SEQ ID NO: 56 or amino acids 39 to 440 of SEQ ID NO: 57); or
(x) a fragment of amino acids 45 to 467 of SEQ ID NO: 59 or amino acids 1 to 440 of SEQ ID NO: 60 wherein the fragment preferably contains at least 359 amino acids (e.g., amino acids 108 to 467 of SEQ ID NO: 59 or amino acids 81 to 440 of SEQ ID NO: 60), at least 380 amino acids (e.g., amino acids 87 to 467 of SEQ ID NO: 59 or amino acids 60 to 440 of SEQ ID NO: 60), or at least 401 amino acids (e.g., amino acids 66 to 467 of SEQ ID NO: 59 or amino acids 39 to 440 of SEQ ID NO: 60).

26. An isolated or purified polypeptide comprising a starch binding module and a catalytic domain, wherein the binding module is selected from the group consisting of:

(i)
(a) a starch binding module having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 532 to 632 of SEQ ID NO: 5;
(b) a starch binding module encoded by a polynucleotide that hybridizes under medium, medium-high, high, or very high stringency conditions with the full-length complement of nucleotides 2046 to 2348 of SEQ ID NO: 4, or the cDNA sequence thereof;
(c) a starch binding module encoded by a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 2046 to 2348 of SEQ ID NO: 4, or the cDNA sequence thereof; and
(d) a fragment of the starch binding module of (a), (b), or (c) that has binding activity;
   (ii)
(a) a starch binding module having at least 91%, at least 92%, at least 93%, at least 94%, at least 95%, at least 96%, at least 97%, at least 98%, at least 99%, or 100% sequence identity to amino acids 532 to 632 of SEQ ID NO: 8;
(b) a starch binding module encoded by a polynucleotide that hybridizes under medium, medium-high, high, or very high stringency conditions with the full-length complement of nucleotides 2034 to 2336 of SEQ ID NO: 7, or the cDNA sequence thereof;
(c) a starch binding module encoded by a polynucleotide having sat least 91%, at least 92%, at least 93%, at least 94%, at least 95%, at least 96%, at least 97%, at least 98%, at least 99%, or 100% sequence identity to nucleotides 2034 to 2336 of SEQ ID NO: 7, or the cDNA sequence thereof; and
(d) a fragment of the starch binding module of (a), (b), or (c) that has binding activity;
   (iii)
(a) a starch binding module having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 508 to 601 of SEQ ID NO: 11;
(b) a starch binding module encoded by a polynucleotide that hybridizes under medium, medium-high, high, or very high stringency conditions with the full-length complement of nucleotides 1504 to 1827 of SEQ ID NO: 10;
(c) a starch binding module encoded by a polynucleotide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1504 to 1827 of SEQ ID NO: 10; and
(d) a fragment of the starch binding module of (a), (b), or (c) that has binding activity;
   (iv)
(a) a starch binding module having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 554 to 653 of SEQ ID NO: 41;
(b) a starch binding module encoded by a polynucleotide that hybridizes under medium, medium-high, high, or very high stringency conditions with the full-length complement of nucleotides 1660 to 1959 of SEQ ID NO: 40;
(c) a starch binding module encoded by a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1660 to 1959 of SEQ ID NO: 40; and
(d) a fragment of the starch binding module of (a), (b), or (c) that has binding activity;
   (v)
(a) a starch binding module having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 478 to 569 of SEQ ID NO: 44 or amino acids 563 to 654 of SEQ ID NO: 53;
(b) a starch binding module encoded by a polynucleotide that hybridizes under medium, medium-high, high, or very high stringency conditions with the full-length complement of nucleotides 1432 to 1707 of SEQ ID NO: 43 or nucleotides 1687 to 1962 of SEQ ID NO: 52;
(c) a starch binding module encoded by a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1432 to 1707 of SEQ ID NO: 43 or nucleotides 1687 to 1962 of SEQ ID NO: 52; and
(d) a fragment of the starch binding module of (a), (b), or (c) that has binding activity;
   (vi)
(a) a starch binding module having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 470 to 565 of SEQ ID NO: 47, amino acids 475 to 571 of SEQ ID NO: 50, amino acids 563 to 654 of SEQ ID NO: 53, amino acids 655 to 750 of SEQ ID NO: 53, amino acids 476 to 569 of SEQ ID NO: 56, or amino acids 479 to 572 of SEQ ID NO: 59;
(b) a starch binding module encoded by a polynucleotide that hybridizes under medium, medium-high, high, or very high stringency conditions with the full-length complement of nucleotides 1408 to 1695 of SEQ ID NO: 46, nucleotides 1423 to 1713 of SEQ ID NO: 49, nucleotides 1687 to 1962 of SEQ ID NO: 52, nucleotides 1963 to 2250 of SEQ ID NO: 52, nucleotides 1426 to 1707 of SEQ ID NO: 55, or nucleotides 1535 to 1716 of SEQ ID NO: 58;
(c) a starch binding module encoded by a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1408 to 1695 of SEQ ID NO: 46, nucleotides 1423 to 1713 of SEQ ID NO: 49, nucleotides 1687 to 1962 of SEQ ID NO: 52, nucleotides 1963 to 2250 of SEQ ID NO: 52, nucleotides 1426 to 1707 of SEQ ID NO: 55, or nucleotides 1535 to 1716 of SEQ ID NO: 58; and
(d) a fragment of the starch binding module of (a), (b), or (c) that has binding activity.

27. The polypeptide of paragraph 26, wherein the catalytic domain is obtained from a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase, preferably wherein the catalytic domain is obtained from a hydrolase, preferably from an alpha-amylase.

28. The polypeptide of paragraph 26 or 27, wherein the starch binding module has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to: (i) amino acids 507 to 603 of SEQ ID NO: 2 or amino acids 487 to 583 of SEQ ID NO: 3; (ii) amino acids 532 to 632 of SEQ ID NO: 5 or amino acids 515 to 615 of SEQ ID NO: 6; (iii) amino acids 532 to 632 of SEQ ID NO: 8 or amino acids 511 to 611 of SEQ ID NO: 9; (iv) amino acids 508 to 601 of SEQ ID NO: 11 or amino acids 489 to 582 of SEQ ID NO: 12; (v) amino acids 554 to 653 of SEQ ID NO: 41; (vi) amino acids 478 to 569 of SEQ ID NO: 44; (vii) amino acids 470 to 565 of SEQ ID NO: 47; (viii) amino acids 475 to 571 of SEQ ID NO: 50; (ix) amino acids 563 to 654 of SEQ ID NO: 53; (x) amino acids 655 to 750 of SEQ ID NO: 53; (xi) amino acids 476 to 569 of SEQ ID NO: 56; or (xii) amino acids 479 to 572 of SEQ ID NO: 59.

29. The polypeptide of any one of paragraphs 26-28, wherein the starch binding module is encoded by a polynucleotide that hybridizes under medium stringency conditions, medium-high stringency conditions, high stringency conditions, or very high stringency conditions with the full-length complement of: (i) nucleotides 1519 to 1809 of SEQ ID NO: 1 or the cDNA thereof; (ii) nucleotides 2046 to 2348 of SEQ ID NO: 4 or the cDNA thereof; (iii) nucleotides 2034 to 2336 of SEQ ID NO: 7 or the cDNA thereof; (iv) nucleotides 1504 to 1827 of SEQ ID NO: 10 or the cDNA thereof; (v) nucleotides 1660 to 1959 of SEQ ID NO: 40 or the cDNA thereof; (vi) nucleotides 1432 to 1707 of SEQ ID NO: 43; (vii) nucleotides 1408 to 1695 of SEQ ID NO: 46; (viii) nucleotides 1423 to 1713 of SEQ ID NO: 49; (ix) nucleotides 1687 to 1962 of SEQ ID NO: 52; (x) nucleotides 1963 to 2250 of SEQ ID NO: 52; (xi) nucleotides 1426 to 1707 of SEQ ID NO: 55; or (xii) nucleotides 1535 to 1716 of SEQ ID NO: 58.

30. The polypeptide of any one of paragraphs 26-29, wherein the starch binding module is encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to: (i) nucleotides 1519 to 1809 of SEQ ID NO: 1; (ii) nucleotides 2046 to 2348 of SEQ ID NO: 4 or the cDNA sequence thereof; (iii) nucleotides 2034 to 2336 of SEQ ID NO: 7 or the cDNA sequence thereof; (iv) nucleotides 1504 to 1827 of SEQ ID NO: 10; or (v) nucleotides 1660 to 1959 of SEQ ID NO: 40; (vi) nucleotides 1432 to 1707 of SEQ ID NO: 43; (vii) nucleotides 1408 to 1695 of SEQ ID NO: 46; (viii) nucleotides 1423 to 1713 of SEQ ID NO: 49; (ix) nucleotides 1687 to 1962 of SEQ ID NO: 52; (x) nucleotides 1963 to 2250 of SEQ ID NO: 52; (xi) nucleotides 1426 to 1707 of SEQ ID NO: 55; or (xii) nucleotides 1535 to 1716 of SEQ ID NO: 58.

31. The polypeptide of any one of paragraphs 26-30, wherein the starch binding module comprises, consists essentially of, or consists of amino acids: (i) amino acids 507 to 603 of SEQ ID NO: 2 or amino acids 487 to 583 of SEQ ID NO: 3; (ii) amino acids 532 to 632 of SEQ ID NO: 5 or amino acids 515 to 615 of SEQ ID NO: 6; (iii) amino acids 532 to 632 of SEQ ID NO: 8 or amino acids 511 to 611 of SEQ ID NO: 9; (iv) amino acids 508 to 601 of SEQ ID NO: 11 or amino acids 489 to 582 of SEQ ID NO: 12; (v) amino acids 554 to 653 of SEQ ID NO: 41; (vi) amino acids 478 to 569 of SEQ ID NO: 44; (vii) amino acids 470 to 565 of SEQ ID NO: 47; (viii) amino acids 475 to 571 of SEQ ID NO: 50; (ix) amino acids 563 to 654 of SEQ ID NO: 53; (x) amino acids 655 to 750 of SEQ ID NO: 53; (xi) amino acids 476 to 569 of SEQ ID NO: 56; or (xii) amino acids 479 to 572 of SEQ ID NO: 59.

32. The polypeptide of any one of paragraphs 26-31, wherein the starch binding module is:
(i) a variant of amino acids 507 to 603 of SEQ ID NO: 2 or a variant of amino acids 487 to 583 of SEQ ID NO: 3 comprising a substitution, deletion, and/or insertion at one or more positions;
(ii) a variant of amino acids 532 to 632 of SEQ ID NO: 5 or a variant of amino acids 515 to 615 of SEQ ID NO: 6 comprising a substitution, deletion, and/or insertion at one or more positions;
(iii) a variant of amino acids 532 to 632 of SEQ ID NO: 8 or a variant of amino acids 511 to 611 of SEQ ID NO: 9 comprising a substitution, deletion, and/or insertion at one or more positions;
(iv) a variant of amino acids 508 to 601 of SEQ ID NO: 11 or a variant of amino acids 489 to 582 of SEQ ID NO: 12 comprising a substitution, deletion, and/or insertion at one or more positions;
(v) a variant of amino acids 554 to 653 of SEQ ID NO: 41 comprising a substitution, deletion, and/or insertion at one or more positions;
(vi) a variant of amino acids 478 to 569 of SEQ ID NO: 44 comprising a substitution, deletion, and/or insertion at one or more positions;
(vii) a variant of amino acids 470 to 565 of SEQ ID NO: 47 comprising a substitution, deletion, and/or insertion at one or more positions;
(viii) a variant of amino acids 475 to 571 of SEQ ID NO: 50 comprising a substitution, deletion, and/or insertion at one or more positions;
(ix) a variant of amino acids 563 to 654 of SEQ ID NO: 53 comprising a substitution, deletion, and/or insertion at one or more positions;
(x) a variant of amino acids 655 to 750 of SEQ ID NO: 53 comprising a substitution, deletion, and/or insertion at one or more positions;
(xi) a variant of amino acids 476 to 569 of SEQ ID NO: 56 comprising a substitution, deletion, and/or insertion at one or more positions; or
(xii) a variant of amino acids 479 to 572 of SEQ ID NO: 59 comprising a substitution, deletion, and/or insertion at one or more positions.

33. The polypeptide of any one of paragraphs 26-32, wherein the starch binding module is:
(i) a fragment of amino acids 507 to 603 of SEQ ID NO: 2 or a fragment of amino acids 487 to 583 of SEQ ID NO: 3, wherein the fragment preferably contains at least 81 amino acid residues (e.g., amino acids 507 to 588 of SEQ ID NO: 2 or amino acids 487 to 568 of SEQ ID NO: 3), at least 86 amino acid residues (e.g., amino acids 507 to 593 of SEQ ID NO: 2 or amino acids 487 to 573), or at least 91 amino acid residues (e.g., amino acids 507 to 598 of SEQ ID NO: 2 or amino acids 487 to 578), and wherein the fragment has alpha-amylase activity;
(ii) a fragment of amino acids 532 to 632 of SEQ ID NO: 5 or a fragment of amino acids 515 to 615 of SEQ ID NO: 6, wherein the fragment preferably contains at least 85 amino acid residues (e.g., amino acids 532 to 617 of SEQ ID NO: 5 or amino acids 515 to 600 of SEQ ID NO: 6), at least 90 amino acid residues (e.g., amino acids 532 to 622 of SEQ ID NO: 5 or amino acids 515 to 605 of SEQ ID NO: 6), or at least 95 amino acid residues (e.g., amino acids 532 to 627 of SEQ ID NO: 5 or amino acids 515 to 610 of SEQ ID NO: 6), and wherein the fragment has alpha-amylase activity;
(iii) a fragment of amino acids 532 to 632 of SEQ ID NO: 8 or a fragment of amino acids 511 to 611 of SEQ ID NO: 9, wherein the fragment preferably contains at least 85 amino acid residues (e.g., amino acids 532 to 617 of SEQ ID NO: 8 or amino acids 511 to 596 of SEQ ID NO: 9), at least 90 amino acid residues (e.g., amino acids 532 to 622 of SEQ ID NO: 8 or amino acids 511 to 601 of SEQ ID NO: 9), or at least 95 amino acid residues (e.g., amino acids 532 to 627 of SEQ ID NO: 8 or amino acids 511 to 606 of SEQ ID NO: 9), and wherein the fragment has alpha-amylase activity;
(iv) a fragment of amino acids 508 to 601 of SEQ ID NO: 11 or a fragment of amino acids 489 to 582 of SEQ ID NO: 12, wherein the fragment preferably contains at least 79 amino acid residues (e.g., amino acids 508 to 587 of SEQ ID NO: 11 or amino acids 489 to 568 of SEQ ID NO: 12), at least 83 amino acid residues (e.g., amino acids 508 to 591 of SEQ ID NO: 11 or amino acids 489 to 572 of SEQ ID NO: 12), or at least 88 amino acid residues (e.g., amino acids 508 to 596 of SEQ ID NO: 11 or amino acids 489 to 577 of SEQ ID NO: 12), and wherein the fragment has alpha-amylase activity; or
(v) a fragment of amino acids 554 to 653 of SEQ ID NO: 41, wherein the fragment preferably contains at least at least 84 amino acid residues (e.g., amino acids 554 to 638 of SEQ ID NO: 41), at least 89 amino acid residues (e.g., amino acids 554 to 643 of SEQ ID NO: 41), or at least 94 amino acid residues (e.g., amino acids 554 to 649 of SEQ ID NO: 41).
(vii) a fragment of amino acids 478 to 569 of SEQ ID NO: 44 or amino acids 451 to 542 of SEQ ID NO: 45 wherein the fragment preferably contains at least at least 79 amino acids (e.g., amino acids 490 to 569 of SEQ ID NO: 44 or amino acids 463 to 542 of SEQ ID NO: 45), at least 82 amino acids (e.g., amino acids 487 to 569 of SEQ ID NO: 44 or amino acids 460 to 542 of SEQ ID NO: 45), or at least 87 amino acids (e.g., amino acids 482 to 569 of SEQ ID NO: 44 or amino acids 455 to 542 of SEQ ID NO: 45);
(viii) a fragment of amino acids 470 to 565 of SEQ ID NO: 47 or amino acids 443 to 538 of SEQ ID NO: 48 wherein the fragment preferably contains at least 81 amino acids (e.g., amino acids 484 to 565 of SEQ ID NO: 47 or amino acids 457 to 538 of SEQ ID NO: 48), at least 86 amino acids (e.g., amino acids 479 to 565 of SEQ ID NO: 47 or amino acids 460 to 538 of SEQ ID NO: 48), or at least 91 amino acids (e.g., amino acids 474 to 565 of SEQ ID NO: 47 or amino acids 447 to 538 of SEQ ID NO: 48);
(ix) a fragment of amino acids 475 to 571 of SEQ ID NO: 50 or amino acids 448 to 544 of SEQ ID NO: 51 wherein the fragment preferably contains at least 81 amino acids (e.g., amino acids 490 to 571 of SEQ ID NO: 50 or amino acids 463 to 544 of SEQ ID NO: 51), at least 86 amino acids (e.g., amino acids 485 to 571 of SEQ ID NO: 50 or amino acids 460 to 544 of SEQ ID NO: 51), or at least 91 amino acids (e.g., amino acids 480 to 571 of SEQ ID NO: 50 or amino acids 447 to 544 of SEQ ID NO: 51);
(x) a fragment of amino acids 563 to 654 of SEQ ID NO: 53 or amino acids 536 to 627 of SEQ ID NO: 54 wherein the fragment preferably contains at least 78 amino acids (e.g., amino acids 576 to 654 of SEQ ID NO: 53 or amino acids 549 to 627 of SEQ ID NO: 54), at least 82 amino acids (e.g., amino acids 572 to 654 of SEQ ID NO: 53 or amino acids 545 to 627 of SEQ ID NO: 54), or at least 87 amino acids (e.g., amino acids 567 to 654 of SEQ ID NO: 53 or amino acids 540 to 627 of SEQ ID NO: 54);
(xi) a fragment of amino acids 655 to 750 of SEQ ID NO: 53 or amino acids 628 to 723 of SEQ ID NO: 54 wherein the fragment preferably contains at least 81 amino acids (e.g., amino acids 669 to 750 of SEQ ID NO: 53 or amino acids 642 to 723 of SEQ ID NO: 54), at least 86 amino acids (e.g., amino acids 664 to 750 of SEQ ID NO: 53 or amino acids 637 to 723 of SEQ ID NO: 54), or at least 91 amino acids (e.g., amino acids 659 to 750 of SEQ ID NO: 53 or amino acids 632 to 723 of SEQ ID NO: 54);
(xii) a fragment of amino acids 476 to 569 of SEQ ID NO: 56 or amino acids 449 to 542 of SEQ ID NO: 57 wherein the fragment preferably contains at least 79 amino acids (e.g., amino acids 496 to 569 of SEQ ID NO: 56 or amino acids 463 to 542 of SEQ ID NO: 57), at least 84 amino acids (e.g., amino acids 481 to 569 of SEQ ID NO: 56 or amino acids 458 to 542 of SEQ ID NO: 57), or at least 88 amino acids (e.g., amino acids 481 to 569 of SEQ ID NO: 56 or amino acids 454 to 542 of SEQ ID NO: 57); or
(xii) a fragment of amino acids 479 to 572 of SEQ ID NO: 59 or amino acids 452 to 545 of SEQ ID NO: 60, wherein the fragment preferably contains at least 79 amino acids (e.g., amino acids 499 to 572 of SEQ ID NO: 59 or amino acids 466 to 545 of SEQ ID NO: 60), at least 84 amino acids (e.g., amino acids 484 to 572 of SEQ ID NO: 59 or amino acids 461 to 545 of SEQ ID NO: 60), or at least 88 amino acids (e.g., amino acids 484 to 572 of SEQ ID NO: 59 or amino acids 457 to 545 of SEQ ID NO: 60).

34. A fusion polypeptide comprising the polypeptide of any one of paragraphs 1-33 and a second polypeptide.

35. A granule, which comprises:
(a) a core comprising the polypeptide of any one of paragraphs 1-34, and optionally,
(b) a coating consisting of one or more layer(s) surrounding the core.

36. A granule, which comprises:
(a) a core, and
(b) a coating consisting of one or more layer(s) surrounding the core, wherein the coating comprises the polypeptide of any one of paragraphs 1-34.

37. A composition comprising the polypeptide of any one of paragraphs 1-33 or the granule of paragraph 34 or 35.

38. A whole broth formulation or cell culture composition comprising the polypeptide of any one of paragraphs 1-34.

39. An isolated or purified polynucleotide encoding the polypeptide of any one of paragraphs 1-34.

40. The polynucleotide of claim 39, which comprises SEQ ID NO: 1 or nucleotides 61 to 1479 of SEQ ID NO: 1, SEQ ID NO: 4 or nucleotides 52 to 1943 of SEQ ID NO: 4 or the cDNA thereof, SEQ ID NO: 7 or nucleotides 64 to 1925 of SEQ ID NO: 7, or the cDNA thereof, SEQ ID NO: 10 or nucleotides 58 to 1488 of SEQ ID NO: 10, or SEQ ID NO: 13 or nucleotides 88 to 1407 of SEQ ID NO: 13, SEQ ID NO: 40 or nucleotides 82 to 1395 of SEQ ID NO: 40, SEQ ID NO: 43 or nucleotides 82 to 1725 of SEQ ID NO: 43, SEQ ID NO: 46 or nucleotides 82 to 1716 of SEQ ID NO: 46, SEQ ID NO: 49 or nucleotides 82 to 1731 of SEQ ID NO: 49, SEQ ID NO: 52 or nucleotides 82 to 2271 of SEQ ID NO: 52, SEQ ID NO: 55 or nucleotides 133 to 1401 of SEQ ID NO: 55, or SEQ ID NO: 58 or nucleotides 133 to 1401 of SEQ ID NO: 58.

41. A nucleic acid construct or expression vector comprising the polynucleotide of paragraph 39 or 40, wherein the polynucleotide is operably linked to one or more control sequences that direct the production of the polypeptide in an expression host.

42. A recombinant host cell comprising the polynucleotide of paragraph 39 or 40 operably linked to one or more control sequences that direct the production of the polypeptide.

43. The recombinant host cell of paragraph 42, wherein the polypeptide is heterologous to the recombinant host cell.

44. The recombinant host cell of paragraph 42 or 43, wherein at least one of the one or more control sequences is heterologous to the polynucleotide encoding the polypeptide.

45. The recombinant host cell of any one of paragraphs 42-44, which comprises at least two copies, e.g., three, four, or five, of the polynucleotide of paragraph 34 or 35.

46. The recombinant host cell of any one of paragraphs 42-45, which is a yeast recombinant host cell, e.g., a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

47. The recombinant host cell of any one of paragraphs 42-45, which is a filamentous fungal recombinant host cell, e.g., an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell, in particular, an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

48. The recombinant host cell of any one of paragraphs 42-45, which is a prokaryotic recombinant host cell, e.g., a Gram-positive cell selected from the group consisting of Bacillus, Clostridium, Enterococcus, Geobacillus, Lactobacillus, Lactococcus, Oceanobacillus, Staphylococcus, Streptococcus, or Streptomyces cells, or a Gram-negative bacteria selected from the group consisting of Campylobacter, E. coli, Flavobacterium, Fusobacterium, Helicobacter, Ilyobacter, Neisseria, Pseudomonas, Salmonella, and Ureaplasma cells, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptococcus equisimilis, Streptococcus pyogenes, Streptococcus uberis, and Streptococcus equi subsp. Zooepidemicus, Streptomyces achromogenes, Streptomyces avermitilis, Streptomyces coelicolor, Streptomyces griseus, and Streptomyces lividans cells.

49. A method of producing the polypeptide of any one of paragraphs 1-34, comprising cultivating a cell, which in its wild-type form produces the polypeptide, under conditions conducive for production of the polypeptide.

50. The method of paragraph 49, further comprising recovering the polypeptide.

51. A method of producing a polypeptide having alpha-amylase activity, comprising cultivating the recombinant host cell of any one of paragraphs 42-48 under conditions conducive for production of the polypeptide.

52. The method of paragraph 51, further comprising recovering the polypeptide.

53. A process for producing fermentation products from starch-containing material comprising the steps of:
i) liquefying the starch-containing material at a temperature above the initial gelatinization temperature using an alpha-amylase;
ii) saccharifying using a carbohydrate-source generating enzyme;
iii) fermenting using a fermenting organism;
wherein the polypeptide of any one of paragraphs 1-51 and/or the composition of paragraphs 37-38, is present or added during fermentation or simultaneous saccharification and fermentation.

54. The process of paragraph 53, wherein saccharification is performed in the presence of at least one cellulase/cellulolytic composition.

55. The process of paragraph 54, wherein the cellulases/cellulolytic composition are derived from a strain of Trichoderma, in particular Trichoderma reesei, or a strain of Humicola, in particular Humicola insolens, or a strain of Chrysosporium, in particular Chrysosporium lucknowense.

56. The process of any of paragraphs 54-55, wherein the cellulases/cellulolytic composition comprises a beta-glucosidase, a cellobiohydrolase and an endoglucanase.

57. The process of any of paragraphs 54-56, wherein the cellulases/cellulolytic composition comprises one or more polypeptides selected from the group consisting of:
beta-glucosidase;
cellobiohydrolase I; and
endoglucanase I, or a mixture of two or three thereof.

58. The process of any of paragraphs 54-57, wherein the cellulases/cellulolytic composition comprises one or more of the following components:
(i) an Aspergillus fumigatus beta-glucosidase or a variant thereof;
(ii) an Aspergillus fumigatus cellobiohydrolase I; and
(iii) a Trichoderma reesei endoglucanase I.

59. The process of any of paragraphs 54-58, wherein the cellulases/cellulolytic composition is a Trichoderma reesei cellulolytic composition further comprising:
(i) an Aspergillus fumigatus beta-glucosidase disclosed in SEQ ID NO: 19 or a variant thereof with the following substitutions: F100D, S283G, N456E, F512Y having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 19; (ii) a cellobiohydrolase I (CBH I), such as one derived from a strain of the genus Aspergillus, such as a strain of Aspergillus fumigatus, such as the CBHI disclosed as SEQ ID NO: 20, or a CBHI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 20; and
(iii) an endoglucanase I (EGI), such as one derived from a strain of the genus Trichoderma, such as a strain of Trichoderma reesei, such as the EGI disclosed as SEQ ID NO: 36, or an EGI having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 36.

60. The process of any of paragraphs 54-59, wherein the cellulases/cellulolytic composition comprises one or more polypeptides selected from the group consisting of:
GH61 polypeptide having cellulolytic enhancing activity;
beta-glucosidase;
Cellobiohydrolase I;
Cellobiohydrolase II;
or a mixture of two, three, or four thereof.

61. The process of any of paragraphs 54-60, wherein the cellulases/cellulolytic composition comprises one or more of the following components:
(i) an Aspergillus fumigatus cellobiohydrolase I;
(ii) an Aspergillus fumigatus cellobiohydrolase II;
(iii) an Aspergillus fumigatus beta-glucosidase or variant thereof; and
(iv) a Penicillium sp. GH61 polypeptide having cellulolytic enhancing activity; or homologs thereof.

62. The process of any of paragraphs 54-61, wherein the cellulases/cellulolytic composition is a Trichoderma reesei cellulolytic enzyme composition further comprising Penicillium emersonii GH61A polypeptide having cellulolytic enhancing activity disclosed in SEQ ID NO: 18, or a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 18 and an Aspergillus fumigatus beta-glucosidase disclosed in SEQ ID NO: 19 or a variant thereof with the following substitutions: F100D, S283G, N456E, F512Y having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 19.

63. The process of any of paragraphs 54-62, wherein the cellulolytic composition comprises a cellobiohydrolase I (CBH I), such as one derived from a strain of the genus Aspergillus, such as a strain of Aspergillus fumigatus, such as the CBHI disclosed as SEQ ID NO: 20, or CBH I having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 20.

64. The process of any of paragraphs 54-63, wherein the cellulolytic composition comprises a cellobiohydrolase II (CBH II), such as one derived from a strain of the genus Aspergillus, such as a strain of Aspergillus fumigatus; such as the CBH II disclosed as SEQ ID NO: 21, or a CBH II having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 21.

65. The process of any of paragraphs 53-64, wherein liquefaction is performed in the presence of a protease having a thermostability value of more than 20% determined as Relative Activity at 80° C./70° C.

66. The process of any of paragraphs 53-65, wherein liquefaction is performed in the presence of a glucoamylase.

67. The process of any of paragraphs 53-66, wherein the carbohydrate-source generating enzyme(s) is at least a glucoamylase and optionally in combination with a fungal acid alpha-amylase.

68. The process of any of paragraphs 53-67, wherein the fermentation product is an alcohol, preferably ethanol, especially fuel ethanol, potable ethanol and/or industrial ethanol.

69. The process of any of paragraphs 53-68, wherein the alpha-amylase is a bacterial or fungal alpha-amylase.

70. The process of any of paragraphs 53-69, wherein the alpha-amylase is from the genus Bacillus, such as a strain of Bacillus stearothermophilus, in particular a variant of a Bacillus stearothermophilus alpha-amylase, such as the one shown in SEQ ID NO: 22, or alpha-amylase having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 22.

71. The process of any of paragraphs 53-70, wherein the Bacillus stearothermophilus alpha-amylase comprises a deletion of two amino acids in the region corresponding to positions 179-182 using SEQ ID NO: 22 for numbering.

72. The process of paragraph 71 wherein the deletion is selected from the group consisting of 179*+180*, 179*+181*, 179*+182*, 180*+181*, 180*+182*, and 181*+182*, particularly |181*+G182*.

73. The process of any of paragraphs 53-72, wherein the alpha-amylase used in liquefying step i) comprises a substitution N193F using SEQ ID NO: 22 for numbering.

74. The process of any of paragraphs 53-73, wherein the Bacillus stearothermophilus alpha-amylase has a substitution in position S242, preferably S242Q substitution using SEQ ID NO: 22 for numbering.

75. The process of any of paragraphs 53-74 wherein the Bacillus stearothermophilus alpha-amylase has a substitution in position E188, preferably E188P substitution using SEQ ID NO: 22 for numbering.

76. The process of any of paragraphs 53-75, wherein the alpha-amylase has a T½ (min) at pH 4.5, 85° C., 0.12 mM CaCl2) of at least 10, such as at least 15, such as at least 20, such as at least 25, such as at least 30, such as at least 40, such as at least 50, such as at least 60, such as between 10-70, such as between 15-70, such as between 20-70, such as between 25-70, such as between 30-70, such as between 40-70, such as between 50-70, such as between 60-70.

77. The process of any of paragraphs 53-76, wherein the alpha-amylase is selected from the following group of Bacillus stearothermophilus alpha-amylase variants (using SEQ ID NO: 22 for numbering):

$$I {181}^{⋆} + G 18 {2}^{⋆} + N 193 F + E 129 V + K 177 L + R 1 \; 79 E ;$$

$$I {181}^{⋆} + G 18 {2}^{⋆} + N 193 F + V 59 A + Q 89 R + E 129 V + K 177 L + R 179 E + H 208 Y + K 220 P + N 224 L + Q 254 S$$

$$I {181}^{⋆} + G 18 {2}^{⋆} + N 193 F + V 59 A + Q 89 R + E 129 V + K 177 L + R 179 E + Q 254 S + M 284 V ;$$

$$I {181}^{⋆} + G 18 {2}^{⋆} + N 193 F + V 59 A + E 129 V + K 177 L + R 179 E + Q 254 S + M 284 V ;$$

$$I {181}^{⋆} + G 18 {2}^{⋆} + N 193 F + E 129 V + K 177 L + R 179 E + K 220 P + N 224 L + S 24 \; 20 + Q 254 S ;$$

$$I {181}^{⋆} + G 18 {2}^{⋆} + V 59 A + E 129 V + K 177 L + R 179 E + Q 254 S + M 284 V + V 212 T + Y 268 G + N 2 \; 93 Y + T 297 N ;$$

$$I {181}^{⋆} + G 18 {2}^{⋆} + V 59 A + E 129 V + K 177 L + R 179 E + Q 254 S + M 284 V + V 212 T + Y 268 G + N 2 \; 93 Y + T 297 N + S 173 N + E 188 P + H 208 Y + S 242 Y + K 2791 ;$$

$$I {181}^{⋆} + G 18 {2}^{⋆} + V 59 A + E 129 V + K 177 L + R 179 S + Q 254 S + M 284 V + V 212 T + Y 268 G + N 293 Y + T 297 N + A 1840 + E 188 P + T 191 N ;$$

$$I {181}^{⋆} + G 18 {2}^{⋆} + V 59 A + E 129 V + K 177 L + R 179 S + Q 254 S + M 284 V + V 212 T + Y 268 G + N 2 \; 93 Y + T 297 N + A 1840 + E 188 P + T 191 N + S 242 Y + K2 \; 791 ;$$

$$I {181}^{⋆} + G 18 {2}^{⋆} + V 59 A + E 129 V + K 177 L + R 179 E + Q 254 S + M 284 V + V 212 T + Y 268 G + N 2 \; 93 Y + T 297 N + E 188 P + K 2 \; 79 W ;$$

$$I {181}^{⋆} + G 18 {2}^{⋆} + V 59 A + E 129 V + K 177 L + R 179 E + Q 254 S + M 284 V + V 212 T + Y 268 G + N 2 \; 93 Y + T 297 N + W 115 D + D 1170 + T 1 \; 33 P ;$$

and wherein the variant has at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 22.

78. The process of any of paragraphs 53-77, wherein a protease with a thermostability value of more than 25% determined as Relative Activity at 80° C./70° C. is present in liquefaction step i).

79. The process of any of paragraphs 53-78, wherein the protease has a thermostability of more than 30%, more than 40%, more than 50%, more than 60%, more than 70%, more than 80%, more than 90%, more than 100%, such as more than 105%, such as more than 110%, such as more than 115%, such as more than 120% determined as Relative Activity at 80° C./70° C.

80. The process of any of paragraphs 53-79, wherein the protease has a thermostability of between 20% and 50%, such as between 20% and 40%, such as 20% and 30% determined as Relative Activity at 80° C./70° C.

81. The process of any of paragraphs 53-80, wherein the protease has a thermostability between 50% and 115%, such as between 50% and 70%, such as between 50% and 60%, such as between 100% and 120%, such as between 105% and 115% determined as Relative Activity at 80° C./70° C.

82. The process of any of paragraphs 53-81, wherein the protease has a thermostability of more than 10%, such as more than 12%, more than 14%, more than 16%, more than 18%, more than 20%, more than 30%, more than 40%, more that 50%, more than 60%, more than 70%, more than 80%, more than 90%, more than 100%, more than 110% determined as Relative Activity at 85° C./70° C.

83. The process of any of paragraphs 53-82, wherein the protease has thermostability of between 10% and 50%, such as between 10% and 30%, such as between 10% and 25% determined as Relative Activity at 85° C./70° C.

84. The process of any of paragraphs 53-83, wherein the protease has a thermostability above 60%, such as above 90%, such as above 100%, such as above 110% at 85° C. as determined using the Zein-BCA assay.

85. The process of any of paragraphs 53-84, wherein the protease has a thermostability between 60-120, such as between 70-120%, such as between 80-120%, such as between 90-120%, such as between 100-120%, such as 110-120% at 85° C. as determined using the Zein-BCA assay.

86. The process of any of paragraphs 53-85, wherein the protease is of fungal or bacterial origin.

87. The process of any of paragraphs 53-86, wherein the protease is a metallo protease or a serine protease.

88. The process of any of paragraphs 53-87, wherein the protease is a variant of the metallo protease derived from a strain of the genus Thermoascus, preferably a strain of Thermoascus aurantiacus, especially Thermoascus aurantiacus CGMCC No. 0670.

89. The process of any of paragraphs 53-88, wherein the protease is a variant of the metallo protease disclosed as SEQ ID NO: 23 with the following mutations:

$$\\ D 79 L + S 87 P + A 112 P + D 142 L ; D 79 L + S 87 P + D 142 L ; or$$

$$\\ A 27 K + D 79 L + Y 82 F + S 87 G + D 104 P + A 112 P + A 126 V + D 142 L ;$$

and wherein the protease has at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 23.

90. The process of any of paragraphs 53-89, wherein the protease is a serine protease, particularly an S8 serine protease derived from a strain of Pyrococcus, preferably a strain of Pyrococcus furiosus, or derived from a strain of Thermococcus, preferably Themococcus thioreducens or Thermococcus nautili, or derived from a strain of Palaeococcus, preferably Palaeococcus ferrophilus 91. The process of any of paragraphs 53-90, wherein the protease is derived from a strain of Pyrococcus, preferably a strain of Pyrococcus furiosus.

92. The process of any of paragraphs 53-91, wherein the protease is the one shown in SEQ ID NO: 24, or a protease having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 24.

93. The process of any of paragraphs 53-92, wherein the protease is derived from a strain of Thermobifida, preferably a strain of Thermobifida cellulosytica.

94. The process of claim 53-93, wherein the protease is the one shown in SEQ ID NO: 35, or a protease having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 35.

95. The process of any of paragraphs 53-94, wherein a glucoamylase is present and/or added during saccharification and/or fermentation.

96. The process of any of paragraphs 53-95, wherein the glucoamylase present and/or added during saccharification and/or fermentation is of fungal origin, preferably from a stain of Aspergillus, preferably A. niger, A. awamori, or A. oryzae; or a strain of Trichoderma, preferably T. reesei; or a strain of Talaromyces, preferably Talaromyces emersonii, or a strain of Trametes, preferably Trametes cingulata, or a strain of Pycnoporus, or a strain of Gloeophyllum, such as a strain of Gloeophyllum sepiarium or Gloeophyllum trabeum or a strain of the Nigrofomes.

97. The process of any of paragraphs 53-96, wherein the glucoamylase present and/or added during saccharification and/or fermentation is a blend comprising Talaromyces emersonii glucoamylase of SEQ ID NO: 28, or glucoamylase having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 28, a Trametes cingulata glucoamylase of SEQ ID NO: 27, or glucoamylase having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 27, and a Rhizomucor pusillus alpha-amylase with an Aspergillus niger glucoamylase linker and starch-binding domain (SBD), of SEQ ID NO: 32, and comprising the following substitutions: G128D+D143N, and having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 32.

98. The process of any of paragraphs 53-97, wherein the glucoamylase present and/or added during saccharification and/or fermentation is a blend comprising Gloeophyllum sepiarium glucoamylase shown as SEQ ID NO: 30, or a glucoamylase having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 30, and an alpha-amylase from Rhizomucor pusillus with an Aspergillus niger glucoamylase linker and starch-binding domain (SBD), disclosed SEQ ID NO: 32 with the following substitutions: G128D+D143N, and having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to SEQ ID NO: 32.

99. The process of any of paragraphs 53-98, wherein a trehalase is present and/or added during saccharification and/or fermentation.

100. The process of any of paragraphs 53-99, wherein the trehalase present and/or added during saccharification and/or fermentation is a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to the mature polypeptide of SEQ ID NO: 33 and having trehalase activity.

101. The process of any of paragraphs 53-100, wherein the trehalase present and/or added during saccharification and/or fermentation is a polypeptide having at least 70% identity, at least 71% identity, at least 72% identity, at least 73% identity, at least 74% identity, at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity to the mature polypeptide of SEQ ID NO: 34 and having trehalase activity.

102. The process of any of paragraphs 53-101, wherein saccharification and fermentation are carried out sequentially or simultaneously.

103. The process of any of paragraphs 53-102, wherein fermentation or simultaneous saccharification and fermentation (SSF) are carried out at a temperature from 25° C. to 40° C., such as from 28° C. to 35° C., such as from 30° C. to 34° C., preferably around about 32° C.

104. The process of any of paragraphs 53-103, wherein the fermentation product is recovered after fermentation, such as by distillation.

105. The process of any of paragraphs 53-104, wherein the starch-containing starting material is whole grains.

106. The process of any of paragraphs 53-105, wherein the starch-containing material is derived from corn, wheat, barley, rye, milo, sago, cassava, manioc, tapioca, sorghum, rice or potatoes.

107. The process of any of paragraphs 53-106, wherein the organism applied in fermentation is a yeast, particularly a Saccharomyces spp., more particular Saccharomyces cerevisiae.

108. An enzyme blend or enzyme composition comprising at least one polypeptide having alpha-amylase activity of any of claims 1-31.

109. The blend or composition of paragraph 108, further comprising a carbohydrate-source generating enzyme, particularly a glucoamylase.

110. The blend or composition of any of paragraphs 108-109, further comprising a cellulase/cellulolytic composition comprising a beta-glucosidase, a cellobiohydrolase and an endoglucanase.

111. The blend or composition of paragraph 110, wherein the cellulases/cellulolytic composition comprises one or more polypeptides selected from the group consisting of:
GH61 polypeptide having cellulolytic enhancing activity,
beta-glucosidase;
Cellobiohydrolase I;
Cellobiohydrolase II;
or a mixture of two, three, or four thereof.

112. The blend or composition of any of paragraphs 110-111, wherein the cellulases/cellulolytic composition comprises one or more of the following components:
(i) an Aspergillus fumigatus cellobiohydrolase I;
(ii) an Aspergillus fumigatus cellobiohydrolase II;
(iii) an Aspergillus fumigatus beta-glucosidase or variant thereof; and a Penicillium sp. GH61 polypeptide having cellulolytic enhancing activity; or homologs (iv) thereof.

113. The blend or composition of any one of paragraphs 108-112, further comprising a trehalase.

114. A recombinant host cell comprising at least one heterologous polynucleotide encoding a polypeptide having alpha-amylase activity of any one of claims 1-31.

115. The recombinant host cell of paragraph 114, wherein the at least one heterologous polynucleotide encoding the polypeptide having alpha-amylase activity encodes at least one, at least two, at least three, at least four, or at least five of the polypeptides having alpha-amylase activity of any of claims 1-31.

116. The recombinant host cell of any of paragraphs 114-115, wherein the at least one heterologous polynucleotide encoding a polypeptide having alpha-amylase activity is operably linked to a promoter that is foreign to the polynucleotide.

117. The recombinant host cell of any of paragraphs 114-116, wherein the cell further comprises a heterologous polynucleotide encoding a glucoamylase.

118. The recombinant host cell of paragraph 117, wherein the heterologous polynucleotide encoding the glucoamylase is operably linked to a promoter that is foreign to the polynucleotide.

119. The recombinant host cell of any of paragraph 114-118, wherein the cell further comprises a heterologous polynucleotide encoding an alpha-amylase.

120. The recombinant host cell of paragraph 119, wherein the heterologous polynucleotide encoding the alpha-amylase is operably linked to a promoter that is foreign to the polynucleotide.

121. The recombinant host cell of any of paragraph 114-120, wherein the cell further comprises a heterologous polynucleotide encoding a protease.

122. The recombinant host cell of paragraph 121, wherein the heterologous polynucleotide encoding the protease is operably linked to a promoter that is foreign to the polynucleotide.

123. The recombinant host cell of any of paragraph 114-122, wherein the cell further comprises a disruption to an endogenous gene encoding a glycerol 3-phosphate dehydrogenase (GPD).

124. The recombinant host cell of any of paragraphs 114-123, wherein the cell further comprises a disruption to an endogenous gene encoding a glycerol 3-phosphatase (GPP).

125. The recombinant host cell of any of paragraphs 114-124, wherein the cell is a yeast cell.

126. The recombinant host cell of any of paragraphs 114-125, wherein the cell is a Saccharomyces, Rhodotorula, Schizosaccharomyces, Kluyveromyces, Pichia, Hansenula, Rhodosporidium, Candida, Yarrowia, Lipomyces, Cryptococcus, or Dekkera sp. cell.

127. The recombinant host cell of any of paragraphs 114-126, wherein the cell is a Saccharomyces cerevisiae cell.

128. A composition comprising the recombinant host cell of any of paragraphs 114-127 and one or more naturally occurring and/or non-naturally occurring components, such as components are selected from the group consisting of: surfactants, emulsifiers, gums, swelling agents, and antioxidants.

129. Use of a recombinant host cell of any of paragraphs 114-126 in the production of ethanol.

The present invention is further described by the following examples that should not be construed as limiting the scope of the invention.

### EXAMPLES

### Materials & Methods

### Enzymes Used in the Examples:

Alpha-Amylase A (AAA): Bacillus stearothermophilus alpha-amylase of SEQ ID NO: 114 with the mutations |181*+G182*+N193F and truncated to 491 amino acids.

Alpha-Amylase 2330 (AA2330): Bacillus stearothermophilus alpha-amylase with the mutations: —I181*+G182*+V59A+E129V+K177L+R179S+Q254S+M284V+V212T+Y268G+N293Y+T297N+A184Q+E188P+T191N+N193F+S242Y+K279| truncated to 491 amino acids (SEQ ID NO: 22 herein).

Protease Pfu: Protease derived from Pyrococcus furiosus shown in SEQ ID NO: 24 herein.

Alpha-amylase blend X: Blend of AA2330 and Protease Pfu.
substitutions: G128D+D143N (activity ratio AGU:AGU:FAU(F): approx. 30:7:1).

GsAMG: Gloeophyllum sepiarium glucoamylase disclosed in SEQ ID NO: 30.

Glucoamylase BL: Blend comprising Trametes cingulata glucoamylase disclosed in SEQ ID NO: 27, and Rhizomucor pusillus alpha-amylase with Aspergillus niger glucoamylase linker and SBD disclosed as SEQ ID NO: 32).

Glucoamylase BL2: Blend comprising Talaromyces emersonii glucoamylase disclosed in SEQ ID NO: 28, Trametes cingulata glucoamylase disclosed in SEQ ID NO: 27, and Rhizomucor pusillus alpha-amylase with Aspergillus niger glucoamylase linker and SBD disclosed as SEQ ID NO: 32 with the following substitutions: G128D+D143N (activity ratio AGU:AGU:FAU(F): approx. 30:7:1).

Saccharomyces cerevisiae strain MBG5012: Saccharomyces cerevisiae strain MBG5012 (deposited under Accession No. NRRL Y67549 at the Agricultural Research Service Patent Culture Collection (NRRL), Northern Regional Research Center, 1815 University Street, Peoria, IL, USA).

SEQ ID NO: 2: polypeptide having alpha-amylase activity comprising Penicillium oxalicum catalytic domain and Aspergillus rolfsii starch binding domain.

SEQ ID NO: 5: polypeptide having alpha-amylase activity from Penicillium sclerotiorum.

SEQ ID NO: 8: polypeptide having alpha-amylase activity from Penicillium wotroi.

SEQ ID NO: 11: polypeptide having alpha-amylase activity from Talaromyces helices.

SEQ ID NO: 14: polypeptide having alpha-amylase activity from Lactobacillus amylovorus.

SEQ ID NO: 16: polypeptide having alpha-amylase activity from Valsaria rubricosa.

SEQ ID NO: 41: polypeptide having alpha-amylase activity from Bacillus amyloliquefaciens.

SEQ ID NO: 44: polypeptide having alpha-amylase activity comprising catalytic domain, linker, and starch binding module from Lactobacillus amylovorus.

SEQ ID NO: 47: hybrid polypeptide having alpha-amylase activity comprising catalytic domain from Lactobacillus amylovorus, and CBM20 starch binding module from Anoxybacillus contaminans fused to the C-terminal of the catalytic domain.

SEQ ID NO: 50: hybrid polypeptide having alpha-amylase activity comprising catalytic domain from Lactobacillus amylovorus, and a CBM20 starch binding module from Anoxybacillus contaminans fused to the C-terminal of the catalytic domain via a synthetic linker (PPPGP: SEQ ID NO: 63)

SEQ ID NO: 53: hybrid polypeptide having alpha-amylase activity comprising catalytic domain from Lactobacillus amylovorus, a X23 domain of unknown function, that might only serve as a linker, from Bacillus amyloliquefaciens fused to the C-terminal of the catalytic domain via a synthetic linker (PEPTPEP: SEQ ID NO: 58), a CBM26 starch binding module from Bacillus amyloliquefaciens fused to the C-terminal of the X23 starch binding module, and a CBM20 starch binding module from Anoxybacillus contaminans fused to the C-terminal of the CBM26 starch binding module.

SEQ ID NO: 56: hybrid polypeptide having alpha-amylase activity comprising catalytic domain from Lactobacillus amylovorus, and a CBM20 starch binding module from Anoxybacillus contaminans fused to the C-terminal of the catalytic domain via a synthetic linker (SGNSGGPT: SEQ ID NO: 64)

SEQ ID NO: 59: hybrid polypeptide having alpha-amylase activity comprising catalytic domain from Lactobacillus amylovorus, and a CBM20 starch binding module from Anoxybacillus contaminans fused to the C-terminal of the catalytic domain via a synthetic linker (SSNSCGGGGST: SEQ ID NO: 65).

### Protease Assays

### AZCL-Casein Assay

A solution of 0.2% of the blue substrate AZCL-casein is suspended in Borax/NaH2PO₄ buffer pH9 while stirring. The solution is distributed while stirring to microtiter plate (100 microL to each well), 30 microL enzyme sample is added and the plates are incubated in an Eppendorf Thermomixer for 30 minutes at 45 C and 600 rpm. Denatured enzyme sample (100 C boiling for 20 min) is used as a blank. After incubation the reaction is stopped by transferring the microtiter plate onto ice and the coloured solution is separated from the solid by centrifugation at 3000 rpm for 5 minutes at 4□C. 60 microL of supernatant is transferred to a microtiter plate and the absorbance at 595 nm is measured using a BioRad Microplate Reader.

pNA-Assay

50 microL protease-containing sample is added to a microtiter plate and the assay is started by adding 100 microL 1 mM pNA substrate (5 mg dissolved in 100 microL DMSO and further diluted to 10 mL with Borax/NaH₂PO₄ buffer pH 9.0). The increase in OD₄₀₅ at room temperature is monitored as a measure of the protease activity.

### Glucoamylase Activity (AGU)

Glucoamylase activity may be measured in Glucoamylase Units (AGU).

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| | |
|---|---|
| AMG incubation: | |
| Substrate: | maltose 23.2 mM |
| Buffer: | acetate 0.1M |
| PH: | 4.30 ± 0.05 |
| Incubation temperature: | 37° C. ± 1 |
| Reaction time: | 5 minutes |
| Enzyme working range: | 0.5-4.0 AGU/mL |
| Color reaction: | |
| GlucDH: | 430 U/L |
| Mutarotase: | 9 U/L |
| NAD: | 0.21 mM |
| Buffer: | phosphate 0.12M; 0.15M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01 [symbol] describing this analytical method in more detail is available on request from Novozymes A/S, Denmark, which folder is hereby included by reference.

### Acid Alpha-Amylase Activity (AFAU)

Acid alpha-amylase activity may be measured in AFAU (Acid Fungal Alpha-amylase Units), which are determined relative to an enzyme standard. 1 AFAU is defined as the amount of enzyme which degrades 5.260 mg starch dry matter per hour under the below mentioned standard conditions.

Acid alpha-amylase, an endo-alpha-amylase (1,4-alpha-D-glucan-glucanohydrolase, E.C. 3.2.1.1) hydrolyzes alpha-1,4-glucosidic bonds in the inner regions of the starch molecule to form dextrins and oligosaccharides with different chain lengths. The intensity of color formed with iodine is directly proportional to the concentration of starch. Amylase activity is determined using reverse colorimetry as a reduction in the concentration of starch under the specified analytical conditions.

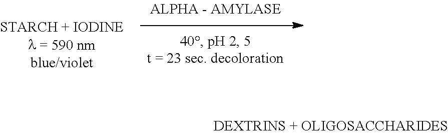

### Standard Conditions/Reaction Conditions:

Substrate: Soluble starch, approx. 0.17 g/L
Buffer: Citrate, approx. 0.03 M
Iodine (12): 0.03 g/L
CaCl2: 1.85 mM
pH 2.50+0.05
Incubation temperature: 40° C.
Reaction time: 23 seconds
Wavelength: 590 nm
Enzyme concentration: 0.025 AFAU/mL
Enzyme working range: 0.01-0.04 AFAU/mL

A folder EB-SM-0259.02/01 describing this analytical method in more detail is available upon request to Novozymes A/S, Denmark, which folder is hereby included by reference.

### Alpha-Amylase Activity (KNU)

The alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C. +/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

### Determination of FAU(F)

FAU(F) Fungal Alpha-Amylase Units (Fungamyl) is measured relative to an enzyme standard of a declared strength.

Reaction conditions

| Temperature | 37° | C. |
|---|---|---|
| pH | 7.15 | |
| Wavelength | 405 | nm |
| Reaction time | 5 | min |
| Measuring time | 2 | min |

A folder (EB-SM-0216.02) describing this standard method in more detail is available on request from Novozymes A/S, Denmark, which folder is hereby included by reference.

### Determination of Pullulanase Activity (NPUN)

Endo-pullulanase activity in NPUN is measured relative to a Novozymes pullulanase standard. One pullulanase unit (NPUN) is defined as the amount of enzyme that releases 1 micro mol glucose per minute under the standard conditions (0.7% red pullulan (Megazyme), pH 5, 40° C., 20 minutes). The activity is measured in NPUN/ml using red pullulan.

1 mL diluted sample or standard is incubated at 40° C. for 2 minutes. 0.5 mL 2% red pullulan, 0.5 M KCl, 50 mM citric acid, pH 5 are added and mixed. The tubes are incubated at 40° C. for 20 minutes and stopped by adding 2.5 ml 80% ethanol. The tubes are left standing at room temperature for 10-60 minutes followed by centrifugation 10 minutes at 4000 rpm. OD of the supernatants is then measured at 510 nm and the activity calculated using a standard curve.

The present invention is described in further detail in the following examples which are offered to illustrate the present invention, but not in any way intended to limit the scope of the invention as claimed. All references cited herein are specifically incorporated by reference for that which is described therein.

### EXAMPLES

### Example 1

### Stability of Alpha-Amylase Variants

The stability of a reference alpha-amylase (Bacillus stearothermophilus alpha-amylase with the mutations 1181*+G182*+N193F truncated to 491 amino acids (SEQ ID NO: 22 numbering)) and alpha-amylase variants thereof was determined by incubating the reference alpha-amylase and variants at pH 4.5 and 5.5 and temperatures of 75° C. and 85° C. with 0.12 mM CaCl₂) followed by residual activity determination using the EnzChek® substrate (EnzChek® Ultra Amylase assay kit, E33651, Molecular Probes).

Polypeptide enzyme samples were diluted to working concentrations of 0.5 and 1 or 5 and 10 ppm (micrograms/ml) in enzyme dilution buffer (10 mM acetate, 0.01% Triton X100, 0.12 mM CaCl₂), pH 5.0). Twenty microliters enzyme sample was transferred to 48-well PCR MTP and 180 microliters stability buffer (150 mM acetate, 150 mM MES, 0.01% Triton X100, 0.12 mM CaCl₂), pH 4.5 or 5.5) was added to each well and mixed. The assay was performed using two concentrations of enzyme in duplicates. Before incubation at 75° C. or 85° C., 20 microliters was withdrawn and stored on ice as control samples. Incubation was performed in a PCR machine at 75° C. and 85° C. After incubation samples were diluted to 15 ng/ml in residual activity buffer (100 mM Acetate, 0.01% Triton X100, 0.12 mM CaCl₂), pH 5.5) and 25 microliters diluted enzyme was transferred to black 384-MTP. Residual activity was determined using the EnzChek substrate by adding 25 microliters substrate solution (100 micrograms/ml) to each well. Fluorescence was determined every minute for 15 minutes using excitation filter at 485-P nm and emission filter at 555 nm (fluorescence reader is Polarstar, BMG). The residual activity was normalized to control samples for each setup.

Assuming logarithmic decay half life time (T½ (min)) was calculated using the equation: T½ (min)=T(min)*LN(0.5)/LN(% RA/100), where T is assay incubation time in minutes, and % RA is % residual activity determined in assay.

Using this assay setup the half life time was determined for the reference alpha-amylase and variant thereof as shown in Table 2.

TABLE 2

| | T½ (min) | T½ (min) | T½ (min) |
|---|---|---|---|
| | (pH 4.5, 75° C., | (pH 4.5, 85° C., | (pH 5.5, 85° C., |
| Mutations | 0.12 mM CaCl₂) | 0.12 mM CaCl₂) | 0.12 mM CaCl₂) |
| Reference Alpha-Amylase A | 21 | 4 | 111 |
| Reference Alpha-Amylase A with the | 32 | 6 | 301 |
| substitution V59A | | | |
| Reference Alpha-Amylase A with the | 28 | 5 | 230 |
| substitution V59E | | | |
| Reference Alpha-Amylase A with the | 28 | 5 | 210 |
| substitution V591 | | | |
| Reference Alpha-Amylase A with the | 30 | 6 | 250 |
| substitution V59Q | | | |
| Reference Alpha-Amylase A with the | 149 | 22 | ND |
| substitutions V59A + Q89R + | | | |
| G112D + E129V + K177L + R179E + | | | |
| K220P + N224L + Q254S | | | |
| Reference Alpha-Amylase A with the | >180 | 28 | ND |
| substitutions V59A + Q89R + E129V + | | | |
| K177L + R179E + H208Y + K220P + | | | |
| N224L + Q254S | | | |
| Reference Alpha-Amylase A with the | 112 | 16 | ND |
| substitutions V59A + Q89R + E129V + | | | |
| K177L + R179E + K220P + N224L + | | | |
| Q254S + D269E + D281N | | | |
| Reference Alpha-Amylase A with the | 168 | 21 | ND |
| substitutions V59A + Q89R + E129V + | | | |
| K177L + R179E + K220P + N224L + | | | |
| Q254S + 1270L | | | |
| Reference Alpha-Amylase A with the | >180 | 24 | ND |
| substitutions V59A + Q89R + E129V + | | | |
| K177L + R179E + K220P + N224L + | | | |
| Q254S + H274K | | | |
| Reference Alpha-Amylase A with the | 91 | 15 | ND |
| substitutions V59A + Q89R + E129V + | | | |
| K177L + R179E + K220P + N224L + | | | |
| Q254S + Y276F | | | |
| Reference Alpha-Amylase A with the | 141 | 41 | ND |
| substitutions V59A + E129V + | | | |
| R157Y + K177L + R179E + K220P + | | | |
| N224L + S242Q + Q254S | | | |
| Reference Alpha-Amylase A with the | >180 | 62 | ND |
| substitutions V59A + E129V + | | | |
| K177L + R179E + H208Y + K220P + | | | |
| N224L + S242Q + Q254S | | | |
| Reference Alpha-Amylase A with the | >180 | 49 | >480 |
| substitutions V59A + E129V + | | | |
| K177L + R179E + K220P + N224L + | | | |
| S242Q + Q254S | | | |
| Reference Alpha-Amylase A with the | >180 | 53 | ND |
| substitutions V59A + E129V + | | | |
| K177L + R179E + K220P + N224L + | | | |
| S242Q + Q254S + H274K | | | |
| Reference Alpha-Amylase A with the | >180 | 57 | ND |
| substitutions V59A + E129V + | | | |
| K177L + R179E + K220P + N224L + | | | |
| S242Q + Q254S + Y276F | | | |
| Reference Alpha-Amylase A with the | >180 | 37 | ND |
| substitutions V59A + E129V + | | | |
| K177L + R179E + K220P + N224L + | | | |
| S242Q + Q254S + D281N | | | |
| Reference Alpha-Amylase A with the | >180 | 51 | ND |
| substitutions V59A + E129V + | | | |
| K177L + R179E + K220P + N224L + | | | |
| S242Q + Q254S + M284T | | | |
| Reference Alpha-Amylase A with the | >180 | 45 | ND |
| substitutions V59A + E129V + | | | |
| K177L + R179E + K220P + N224L + | | | |
| S242Q + Q254S + G416V | | | |
| Reference Alpha-Amylase A with the | 143 | 21 | >480 |
| substitutions V59A + E129V + | | | |
| K177L + R179E + K220P + N224L + | | | |
| Q254S | | | |
| Reference Alpha-Amylase A with the | >180 | 22 | ND |
| substitutions V59A + E129V + | | | |
| K177L + R179E + K220P + N224L + | | | |
| Q254S + M284T | | | |
| Reference Alpha-Amylase A with the | >180 | 38 | ND |
| substitutions A91L + M961 + E129V + | | | |
| K177L + R179E + K220P + N224L + | | | |
| S242Q + Q254S | | | |
| Reference Alpha-Amylase A with the | 57 | 11 | 402 |
| substitutions E129V + K177L + | | | |
| R179E | | | |
| Reference Alpha-Amylase A with the | 174 | 44 | >480 |
| substitutions E129V + K177L + | | | |
| R179E + K220P + N224L + S242Q + | | | |
| Q254S | | | |
| Reference Alpha-Amylase A with the | >180 | 49 | >480 |
| substitutions E129V + K177L + | | | |
| R179E + K220P + N224L + S242Q + | | | |
| Q254S + Y276F + L427M | | | |
| Reference Alpha-Amylase A with the | >180 | 49 | >480 |
| substitutions E129V + K177L + | | | |
| R179E + K220P + N224L + S242Q + | | | |
| Q254S + M284T | | | |
| Reference Alpha-Amylase A with the | 177 | 36 | >480 |
| substitutions E129V + K177L + | | | |
| R179E + K220P + N224L + S242Q + | | | |
| Q254S + N376* + 1377* | | | |
| Reference Alpha-Amylase A with the | 94 | 13 | >480 |
| substitutions E129V + K177L + | | | |
| R179E + K220P + N224L + Q254S | | | |
| Reference Alpha-Amylase A with the | 129 | 24 | >480 |
| substitutions E129V + K177L + | | | |
| R179E + K220P + N224L + Q254S + | | | |
| M284T | | | |
| Reference Alpha-Amylase A with the | 148 | 30 | >480 |
| substitutions E129V + K177L + | | | |
| R179E + S242Q | | | |
| Reference Alpha-Amylase A with the | 78 | 9 | >480 |
| substitutions E129V + K177L + | | | |
| R179V | | | |
| Reference Alpha-Amylase A with the | 178 | | |
| substitutions E129V + K177L + | | | |
| R179V + K220P + N224L + S242Q + | | 31 | >480 |
| Q254S | | | |
| Reference Alpha-Amylase A with the | 66 | 17 | >480 |
| substitutions K220P + N224L + | | | |
| S242Q + Q254S | | | |
| Reference Alpha-Amylase A with the | 30 | 6 | 159 |
| substitutions K220P + N224L + | | | |
| Q254S | | | |
| Reference Alpha-Amylase A with the | 35 | 7 | 278 |
| substitution M284T | | | |
| Reference Alpha-Amylase A with the | 59 | 13 | ND |
| substitutions M284V | | | |

ND not determined

The results demonstrate that the alpha-amylase variants have a significantly greater half-life and stability than the reference alpha-amylase.

### Example 2

### Preparation of Protease Variants and Test of Thermostability

### Strains and Plasmids

E. coli DH12S (available from Gibco BRL) was used for yeast plasmid rescue. pJTP000 is a S. cerevisiae and E. coli shuttle vector under the control of TPI promoter, constructed from pJC039 described in WO 01/92502, in which the Thermoascus aurantiacus M35 protease gene (WO 03048353) has been inserted.

Saccharomyces cerevisiae YNG318 competent cells: MATa Dpep4[cir+] ura3-52, leu2-D2, his 4-539 was used for protease variants expression. It is described in J. Biol. Chem. 272 (15), pp 9720-9727, 1997.

### Media and Substrates

10X Basal solution: Yeast nitrogen base w/o amino acids (DIFCO) 66.8 g/l, succinate 100 g/l, NaOH 60 g/l.

SC-glucose: 20% glucose (i.e., a final concentration of 2%=2 g/100 ml)) 100 ml/l, 5% threonine 4 ml/l, 1% tryptophan10 ml/l, 20% casamino acids 25 ml/l, 10 X basal solution 100 ml/l. The solution is sterilized using a filter of a pore size of 0.20 micrometer. Agar (2%) and H₂O (approx. 761 ml) is autoclaved together, and the separately sterilized SC-glucose solution is added to the agar solution.

YPD: Bacto peptone 20 g/l, yeast extract 10 g/l, 20% glucose 100 ml/l.

YPD+Zn: YPD+0.25 mM ZnSO4.

PEG/LiAc solution: 40% PEG4000 50 ml, 5 M Lithium Acetate 1 ml.

96 well Zein micro titre plate:

Each well contains 200 microL of 0.05-0.1% of zein (Sigma), 0.25 mM ZnSO4 and 1% of agar in 20 mM sodium acetate buffer, pH 4.5.

### DNA Manipulations

Unless otherwise stated, DNA manipulations and transformations were performed using standard methods of molecular biology as described in Sambrook et al. (1989) Molecular cloning: A laboratory manual, Cold Spring Harbor lab. Cold Spring Harbor, NY; Ausubel, F. M. et al. (eds.) “Current protocols in Molecular Biology”, John Wiley and Sons, 1995; Harwood, C. R. and Cutting, S. M. (Eds.).

### Yeast Transformation

Yeast transformation was performed using the lithium acetate method. 0.5 microL of vector (digested by restriction endonucleases) and 1 microL of PCR fragments is mixed. The DNA mixture, 100 microL of YNG318 competent cells, and 10 microL of YEAST MAKER carrier DNA (Clontech) is added to a 12 ml polypropylene tube (Falcon 2059). Add 0.6 ml PEG/LiAc solution and mix gently. Incubate for 30 min at 30° C., and 200 rpm followed by 30 min at 42° C. (heat shock). Transfer to an eppendorf tube and centrifuge for 5 sec. Remove the supernatant and resolve in 3 ml of YPD. Incubate the cell suspension for 45 min at 200 rpm at 30° C. Pour the suspension to SC-glucose plates and incubate 30° C. for 3 days to grow colonies. Yeast total DNA are extracted by Zymoprep Yeast Plasmid Miniprep Kit (ZYMO research).

### DNA Sequencing

E. coli transformation for DNA sequencing was carried out by electroporation (BIO-RAD Gene Pulser). DNA Plasmids were prepared by alkaline method (Molecular Cloning, Cold Spring Harbor) or with the Qiagen® Plasmid Kit. DNA fragments were recovered from agarose gel by the Qiagen gel extraction Kit. PCR was performed using a PTC-200 DNA Engine. The ABI PRISM™ 310 Genetic Analyzer was used for determination of all DNA sequences.

### Construction of Protease Expression Vector

The Thermoascus M35 protease gene was amplified with the primer pair Prot F and Prot R. The resulting PCR fragments were introduced into S. cerevisiae YNG318 together with the pJC039 vector (described in WO 2001/92502) digested with restriction enzymes to remove the Humicola insolens cutinase gene.

The Plasmid in yeast clones on SC-glucose plates was recovered to confirm the internal sequence and termed as pJTP001.

### Construction of Yeast Library and Site-Directed Variants

Library in yeast and site-directed variants were constructed by SOE PCR method (Splicing by Overlap Extension, see “PCR: A practical approach”, p. 207-209, Oxford University press, eds. McPherson, Quirke, Taylor), followed by yeast in vivo recombination.

### General Primers for Amplification and Sequencing

The primers AM34 and AM35 were used to make DNA fragments containing any mutated fragments by the SOE method together with degenerated primers (AM34+Reverse primer and AM35+forward primer) or just to amplify a whole protease gene (AM34+AM35).

| PCR reaction system: | Conditions: |
|---|---|
| 48.5 microL H2O | 1 94° C. 2 min |
| 2 beads puRe Taq Ready-To-Go PCR | 2 94° C. 30 sec |
| (Amersham Biosciences) | |
| 0.5 micro L X 2 100 pmole/microL of primers | 3 55° C. 30 sec |
| 0.5 microL template DNA | 4 72° C. 90 sec |
| | 2-4 25 cycles |
| | 5 72° C. 10 min |

DNA fragments were recovered from agarose gel by the Qiagen gel extraction Kit. The resulting polypeptide fragments were mixed with the vector digest. The mixed solution was introduced into Saccharomyces cerevisiae to construct libraries or site-directed variants by in vivo recombination.

### Relative Activity Assay

Yeast clones on SC-glucose were inoculated to a well of a 96-well micro titre plate containing YPD+Zn medium and cultivated at 28° C. for 3 days. The culture supernatants were applied to a 96-well zein micro titer plate and incubated at at least 2 temperatures (ex. 60° C. and 65° C., 70° C. and 75° C., 70° C. and 80° C.) for more than 4 hours or overnight. The turbidity of zein in the plate was measured as A630 and the relative activity (higher/lower temperatures) was determined as an indicator of thermoactivity improvement. The clones with higher relative activity than the parental variant were selected and the sequence was determined.

### Remaining Activity Assay

Yeast clones on SC-glucose were inoculated to a well of a 96-well micro titre plate and cultivated at 28° C. for 3 days. Protease activity was measured at 65° C. using azo-casein (Megazyme) after incubating the culture supernatant in 20 mM sodium acetate buffer, pH 4.5, for 10 min at a certain temperature (80° C. or 84° C. with 4° C. as a reference) to determine the remaining activity. The clones with higher remaining activity than the parental variant were selected and the sequence was determined.

Azo-casein assay

20 microL of samples were mixed with 150 microL of substrate solution (4 ml of 12.5% azo-casein in ethanol in 96 ml of 20 mM sodium acetate, pH 4.5, containing 0.01% triton-100 and 0.25 mM ZnSO₄) and incubated for 4 hours or longer.

After adding 20 microL/well of 100% trichloroacetic acid (TCA) solution, the plate was centrifuge and 100 microL of supernatants were pipette out to measure A440.

Expression of Protease Variants in Aspergillus oryzae

The constructs comprising the protease variant genes were used to construct expression vectors for Aspergillus. The Aspergillus expression vectors consist of an expression cassette based on the Aspergillus niger neutral amylase II promoter fused to the Aspergillus nidulans triose phosphate isomerase non translated leader sequence (Pna2/tpi) and the Aspergillus niger amyloglucosidase terminator (Tamg). Also present on the plasmid was the Aspergillus selective marker amdS from Aspergillus nidulans enabling growth on acetamide as sole nitrogen source. The expression plasmids for protease variants were transformed into Aspergillus as described in Lassen et al. (2001), Appl. Environ. Microbiol. 67, 4701-4707. For each of the constructs 10-20 strains were isolated, polypeptide and cultivated in shake flasks.

### Purification of Expressed Variants

1. Adjust pH of the 0.22 μm filtered fermentation sample to 4.0.
2. Put the sample on an ice bath with magnetic stirring. Add (NH4)2SO4 in small aliquots (corresponding to approx. 2.0-2.2 M (NH4)2SO4 not taking the volume increase into account when adding the compound).
3. After the final addition of (NH4)2SO4, incubate the sample on the ice bath with gentle magnetic stirring for min. 45 min.
4. Centrifugation: Hitachi himac CR20G High-Speed Refrigerated Centrifuge equipped with R20A2 rotor head, 5° C., 20,000 rpm, 30 min.
5. Dissolve the formed precipitate in 200 ml 50 mM Na-acetate pH 4.0.
6. Filter the sample by vacuum suction using a 0.22 μm PES PLUS membrane (IWAKI).
7. Desalt/buffer-exchange the sample to 50 mM Na-acetate pH 4.0 using ultrafiltration (Vivacell 250 from Vivascience equipped with 5 kDa MWCO PES membrane) overnight in a cold room. Dilute the retentate sample to 200 ml using 50 mM Na-acetate pH 4.0. The conductivity of sample is preferably less than 5 mS/cm.
8. Load the sample onto a cation-exchange column equilibrated with 50 mM Na-acetate pH 4.0. Wash unbound sample out of the column using 3 column volumes of binding buffer (50 mM Na-acetate pH 4.0), and elute the sample using a linear gradient, 0-100% elution buffer (50 mM Na-acetate+1 M NaCl pH 4.0) in 10 column volumes.
9. The collected fractions are assayed by an endo-protease assay (cf. below) followed by standard SDS-PAGE (reducing conditions) on selected fractions. Fractions are pooled based on the endo-protease assay and SDS-PAGE.

### Endo-Protease Assay

1. Protazyme OL tablet/5 ml 250 mM Na-acetate pH 5.0 is dissolved by magnetic stirring (substrate: endo-protease Protazyme AK tablet from Megazyme—cat. #PRAK 11/08).
2. With stirring, 250 microL of substrate solution is transferred to a 1.5 ml Eppendorf tube.
3. 25 microL of sample is added to each tube (blank is sample buffer).
4. The tubes are incubated on a Thermomixer with shaking (1000 rpm) at 50° C. for 15 minutes.
5. 250 microL of 1 M NaOH is added to each tube, followed by vortexing.
6. Centrifugation for 3 min. at 16, 100× G and 25° C.
7. 200 microL of the supernatant is transferred to a MTP, and the absorbance at 590 nm is recorded.

### Results

TABLE 3
Relative activity of protease variants. Numbering of
substitution(s) starts from N-terminal of the mature
peptide in amino acids 1 to 177 of SEQ ID NO: 23.

| | | Relative activity |
|---|---|---|
| Variant | Substitution(s) | 65° C./60° C. |
| WT | none | 31% |
| JTP004 | S87P | 45% |
| JTP005 | A112P | 43% |
| JTP008 | R2P | 71% |
| JTP009 | D79K | 69% |
| JTP010 | D79L | 75% |
| JTP011 | D79M | 73% |
| JTP012 | D79L/S87P | 86% |
| JTP013 | D79L/S87P/A112P | 90% |
| JTP014 | D79L/S87P/A112P | 88% |
| JTP016 | A73C | 52% |
| JTP019 | A126V | 69% |
| JTP021 | M152R | 59% |

TABLE 4
Relative activity of protease variants. Numbering of substitution(s) starts from
N-terminal of the mature peptide in amino acids 1 to 177 of SEQ ID NO: 23.

| | | Relative activity | | |
|---|---|---|---|---|
| Variant | Substitution(s) and/or deletion (S) | 70° C./65° C. | 75° C./65° C. | 75° C./70° C. |
| WT | none | 59% | 17% | |
| JTP036 | D79L/S87P/D142L | 73% | 73% | |
| JTP040 | T54R/D79L/S87P | | 71% | |
| JTP042 | Q53K/D79L/S87P/1173V | | 108% | |
| JTP043 | Q53R/D79L/S87P | | 80% | |
| JTP045 | S41R/D79L/S87P | | 82% | |
| JTP046 | D79L/S87P/Q158W | | 96% | |
| JTP047 | D79L/S87P/S157K | | 85% | |
| JTP048 | D79L/S87P/D104R | | 88% | |
| JTP050 | D79L/S87P/A112P/D142L | | 88% | |
| JTP051 | S41R/D79L/S87P/A112P/D142L | | | 102% |
| JTP052 | D79L/S87P/A112P/D142L/S157K | | | 111% |
| JTP053 | S41R/D79L/S87P/A112P/D142L/S157K | | | 113% |
| JTP054 | AS5/D79L/S87P | | | 92% |
| JTP055 | AG8/D79L/S87P | | | 95% |
| JTP059 | C6R/D79L/S87P | | | 92% |
| JTP061 | T46R/D79L/S87P | | | 111% |
| JTP063 | S49R/D79L/S87P | | | 94% |
| JTP064 | D79L/S87P/N88R | | | 92% |
| JTP068 | D79L/S87P/T114P | | | 99% |
| JTP069 | D79L/S87P/S115R | | | 103% |
| JTP071 | D79L/S87P/T116V | | | 105% |
| JTP072 | N26R/D79L/S87P | | 92% | |
| JTP077 | A27K/D79L/S87P/A112P/D142L | | 106% | |
| JTP078 | A27V/D79L/S87P/A112P/D142L | | 100% | |
| JTP079 | A27G/D79L/S87P/A112P/D142L | | 104% | |

TABLE 5
Relative activity of protease variants. Numbering of substitution(s) starts from
N-terminal of the mature peptide in amino acids 1 to 177 of SEQ ID NO: 23.

| | Substitution(s) and/ | Relative activity | Remaining activity | |
|---|---|---|---|---|
| Variant | or deletion(s) | 75° C./65° C. | 80° C. | 84° C. |
| JTP082 | AS5/D79L/S87P/A112P/D142L | 129% | | 53% |
| JTP083 | T46R/D79L/S87P/A112P/D142L | 126% | | |
| JTP088 | Y43F/D79L/S87P/A112P/D142L | 119% | | |
| JTP090 | D79L/S87P/A112P/T124L/D142L | 141% | | |
| JTP091 | D79L/S87P/A112P/T124V/D142L | 154% | 43% | |
| JTP092 | AS5/N26R/D79L/S87P/A112P/D142L | | | 60% |
| JTP095 | N26R/T46R/D79L/S87P/A112P/D142L | | | 62% |
| JTP096 | T46R/D79L/S87P/T116V/D142L | | | 67% |
| JTP099 | D79L/P81R/S87P/A112P/D142L | | | 80% |
| JTP101 | A27K/D79L/S87P/A112P/T124V/D142L | | 81% | |
| JTP116 | D79L/Y82F/S87P/A112P/T124V/D142L | | 59% | |
| JTP117 | D79L/Y82F/S87P/A112P/T124V/D142L | | 94% | |
| JTP127 | D79L/S87P/A112P/T124V/A126V/D142L | | 53% | |

TABLE 6
Relative activity of protease variants. Numbering of substitution(s) starts from
N-terminal of the mature peptide in amino acids 1 to 177 of SEQ ID NO: 23.

| | | Relative activity | | |
|---|---|---|---|---|
| Variant | Substitutions | 75° C./70° C. | 80° C./70° C. | 85° C./70° C. |
| JTP050 | D79L S87P A112P D142L | 55% | 23% | 9% |
| JTP134 | D79L Y82F S87P A112P D142L | | 40% | |
| JTP135 | S38T D79L S87P A112P A126V D142L | | 62% | |
| JTP136 | D79L Y82F S87P A112P A126V D142L | | 59% | |
| JTP137 | A27K D79L S87P A112P A126V D142L | | 54% | |
| JTP140 | D79L S87P N98C A112P G135C D142L | 81% | | |
| JTP141 | D79L S87P A112P D142L T141C M161C | 68% | | |
| JTP143 | S36P D79L S87P A112P D142L | 69% | | |
| JTP144 | A37P D79L S87P A112P D142L | 57% | | |
| JTP145 | S49P D79L S87P A112P D142L | 82% | 59% | |
| JTP146 | S50P D79L S87P A112P D142L | 83% | 63% | |
| JTP148 | D79L S87P D104P A112P D142L | 76% | 64% | |
| JTP161 | D79L Y82F S87G A112P D142L | | 30% | 12% |
| JTP180 | S70V D79L Y82F S87G Y97W A112P | | 52% | |
| | D142L | | | |
| JTP181 | D79L Y82F S87G Y97W D104P A112P | | 45% | |
| | D142L | | | |
| JTP187 | S70V D79L Y82F S87G A112P D142L | | 45% | |
| JTP188 | D79L Y82F S87G D104P A112P D142L | | 43% | |
| JTP189 | D79L Y82F S87G A112P A126V D142L | | 46% | |
| JTP193 | Y82F S87G S70V D79L D104P A112P | | | 15% |
| | D142L | | | |
| JTP194 | Y82F S87G D79L D104P A112P A126V | | | 22% |
| | D142L | | | |
| JTP196 | A27K D79L Y82F S87G D104P A112P | | | 18% |
| | A126V D142L | | | |

TABLE 7
Relative activity of protease variants. Numbering of
substitution(s) starts from N-terminal of the mature
peptide in amino acids 1 to 177 of SEQ ID NO: 23.

| | | Relative activity | |
|---|---|---|---|
| Variant | Substitutions | 75° C./70° C. | 80° C./70° C. |
| JTP196 | A27K D79L Y82F | 102% | 55% |
| | S87G D104P A112P | | |
| | A126V D142L | | |
| JTP210 | A27K Y82F S87G | 107% | 36% |
| | D104P A112P A126V | | |
| | D142L | | |
| JTP211 | A27K D79L Y82F | | |
| | D104P A112P A126V | 94% | 44% |
| | D142L | | |
| JTP213 | A27K Y82F D104P | | |
| | A112P A126V D142L | 103% | 37% |

### Example 3

### Temperature Profile of Selected Variants Using Polypeptide Enzymes

Selected variants showing good thermo-stability were polypeptide and the polypeptide enzymes were used in a zein-BCA assay as described below. The remaining protease activity was determined at 60° C. after incubation of the enzyme at elevated temperatures as indicated for 60 min.

### Zein-BCA Assay:

Zein-BCA assay was performed to detect soluble protein quantification released from zein by variant proteases at various temperatures.

### Protocol:

1 Mix 10ul of 10 ug/ml enzyme solutions and 100ul of 0.025% zein solution in a micro titer plate (MTP).
2 Incubate at various temperatures for 60 min.
3 Add 10ul of 100% trichloroacetic acid (TCA) solution.
4 Centrifuge MTP at 3500 rpm for 5 min.
5 Take out 15ul to a new MTP containing 100ul of BCA assay solution (Pierce Cat #:23225, BCA Protein Assay Kit).
6 Incubate for 30 min. at 60° C.
7 Measure A562.

The results are shown in Table 8. All of the tested variants showed an improved thermo-stability as compared to the wt protease.

TABLE 8
Zein-BCA assay

| | Sample incubated 60 min at indicated temperatures (° C.) | | | | | | |
|---|---|---|---|---|---|---|---|
| | (μg/ml Bovine serum albumin equivalent peptide released) | | | | | | |
| WT/ | 60° | 70° | 75° | 80° | 85° | 90° | 95° |
| Variant | C. | C. | C. | C. | C. | C. | C. |
| WT | 94 | 103 | 107 | 93 | 58 | 38 | |
| JTP050 | 86 | 101 | 107 | 107 | 104 | 63 | 36 |
| JTP077 | 82 | 94 | 104 | 105 | 99 | 56 | 31 |
| JTP188 | 71 | 83 | 86 | 93 | 100 | 75 | 53 |
| JTP196 | 87 | 99 | 103 | 106 | 117 | 90 | 38 |

### Example 4

### Thermostability of Protease Pfu

The thermostability of the Pyrococcus furiosus protease (Pfu) purchased from Takara Bio Inc, (Japan) was tested using the same methods as in Example 2. It was found that the thermostability (Relative Activity) was 110% at (80° C./70° C.) and 103%(90°) C/70° C. at pH 4.5.

### Example 5

### Cloning and Expression of Fungal Alpha-Amylases

The alpha amylases of were derived from fungal strains obtained from public strain collections or isolated from environmental sample by standard microbiological isolation techniques. The isolated pure strains were identified, and taxonomy was assigned based on DNA sequencing of the ITS ribosomal genes (Table 9).

TABLE 9
Source of Strains

| Strain Source | Country | Mature Protein |
|---|---|---|
| Penicillium oxalicum | China, 2006 | SEQ ID NO: 3 |
| Penicillium sclerotiorum | China, 2007 | SEQ ID NO: 6 |
| Penicillium wotroi | Nicaragua, 1949 | SEQ ID NO: 9 |
| Talaromyces helicus | Italy, 2018 | SEQ ID NO: 12 |

Chromosomal DNA was isolated from pure cultures and subjected to full genome sequencing using ILLUMINA® technology. Genome sequencing, the subsequent assembly of reads and the gene discovery (i.e. annotation of gene functions) is known to the person skilled in the art and the service can be purchased commercially.

The genome sequences were analyzed for alpha-amylase from the CAZY database GH13 family (Lombard V, Golaconda Ramulu H, Drula E, Coutinho P M, Henrissat B (2014). The Carbohydrate-active enzymes database (CAZy) in 2013. Nucleic Acids Res 42:D490-D495). The genes encoding the putative GH13 family alpha-amylases were cloned by PCR amplification from genomic DNA using gene-specific primers that also append a Kozak translation initiation sequence “TCCACC” immediately 5′ of the start codon. The amplified DNA fragments were cloned into the expression vector pDAu222 as described in WO 2013024021 using BamHI and Xhol restriction sites.

The sequences of the putative GH13 alpha-amylase encoding genes cloned in the expression vector was confirmed and the expression construct was transformed into the Aspergillus oryzae strain MT3568 (WO 11/057140) to produce the secreted mature peptide with protein sequences shown in Table 9 above.

Transformants were selected on acetamide during regeneration from protoplasts and subsequently re-isolated under selection (Christensen et al., 1988, Biotechnology 6, 1419-1422 and WO 04/032648).

For production of the recombinant GH13 alpha-amylases, a single Aspergillus transformant of each construct was cultured in twenty 500 ml baffled flasks each containing 150 ml of DAP-4C-1 medium (WO 12/103350). The cultures were shaken on a rotary table at 150 RPM at 30° C. for 4 days. The culture broth was subsequently separated from cellular material by passage through a 0.22 μm filter.

### Purification of the Recombinant GH13 Alpha-Amylases

Filtrated broth was adjusted to pH7.0 and filtrated on 0.22 μm PES filter (Nalge Nunc International, Nalgene labware cat #595-4520). Following, the filtrate was added 1.8M ammonium sulphate. The filtrate was loaded onto a Phenyl Sepharose™ 6 Fast Flow column (high sub) (GE Healthcare, Piscataway, NJ, USA) equilibrated with 1.8M ammonium sulphate, 25 mM HEPES pH7.0. The bound protein was eluted with 1.0M ammonium sulphate, 25 mM HEPES PH 7.0. Fractions were collected and analyzed by SDS-PAGE. The fractions were pooled and applied to a Sephadex™ G-25 (medium) (GE Healthcare, Piscataway, NJ, USA) column equilibrated in 25 mM HEPES pH 7.0. The fractions were applied to a SOURCE™ 15Q (GE Healthcare, Piscataway, NJ, USA) column equilibrated in 25 mM HEPES pH 7.0 and bound proteins were eluted with a linear gradient from 0-1000 mM sodium chloride over 20CV. Fractions were collected and analyzed by SDS-PAGE.

### Example 6

### Expression and Purification of the Lactobacillus Amylovorus Amylase

The strain Lactobacillus amylovorus (DSM20531) was obtained from the DSMZ strain collection (Leibniz-Institut DSMZ-Deutsche Sammlung von Mikroorganismen und Zellkulturen GmbH).

A linear integration vector-system was used for the expression cloning of the amylase from Lactobacillus amylovorus (mature protein shown in SEQ ID NO: 15). The linear integration construct was a PCR fusion product made by fusion of the gene between two Bacillus subtilis homologous chromosomal regions along with a strong promoter and a chloramphenicol resistance marker. The fusion was made by SOE PCR (Horton, R. M., Hunt, H. D., Ho, S. N., Pullen, J. K. and Pease, L. R. (1989) Engineering hybrid genes without the use of restriction enzymes, gene splicing by overlap extension Gene 77: 61-68). The SOE PCR method is also described in patent application WO 2003/095658. The gene was expressed under the control of a triple promoter system (as described in WO 1999/43835), consisting of the promoters from Bacillus licheniformis alpha-amylase gene (amyL), Bacillus amyloliquefaciens alpha-amylase gene (amyQ), and the Bacillus thuringiensis crylllA promoter including stabilizing sequence.

The gene coding for chloramphenicol acetyl-transferase was used as marker (described in e.g. Diderichsen, B.; Poulsen, G. B.; Joergensen, S. T. 1993, Plasmid, “A useful cloning vector for Bacillus subtilis” 30:312. The final gene construct was integrated in the Bacillus chromosome by homologous recombination into the pectate lyase locus.

The amylase from Lactobacillus amylovorus was expressed with the Bacillus licheniformis secretion signal (shown in amino acids 1-29 of SEQ ID NO: 14) replacing the native secretion signal sequence and with His-tag (shown in amino acids 470-475 of SEQ ID NO: 14) at the C-terminal, to facilitate the purification process. The expressed DNA sequence is listed in SEQ ID NO: 13 and the encoded protein in SEQ ID NO: 14 and the theoretical expressed mature protein sequence in SEQ ID NO: 15. The amylase gene was amplified from genomic DNA of the Lactobacillus amylovorus strain with 2 specific primers based on the amylase gene sequence of the public gene: TREMBL: Q48502. The specific primers contained overhang to the 2 vector fragments. The upstream and downstream vector fragments were amplified from genomic DNA of the strain iMB1361 (described in patent application WO 2003095658). The 2 linear vector fragments and the gene fragment were assembled into one linear vector construct by SOE PCR. An aliquot of the PCR product was transformed into Bacillus subtilis. Transformants were selected on LB plates supplemented with 6 μg of chloramphenicol per ml. A recombinant Bacillus subtilis clone containing the sequence confirmed integrated expression construct was cultivated in liquid culture on a rotary shaking table in 500 mL baffled Erlenmeyer flasks each containing 100 ml yeast extract-based media. The clone was cultivated for 4 days at 30° C. The enzyme containing supernatant was harvested and the enzyme purified as described below.

Purification of the His-tagged amylase from Lactobacillus amylovorus

The pH of the supernatant was adjusted to pH 8 with 3 M Tris, left for 1 hour, and then filtered using a filtration unit equipped with a 0.2 μm filter (Nalgene). The filtered supernatant was applied to a 5 ml HisTrap™ Excel column (GE Healthcare Life Sciences) pre-equilibrated with 5 column volumes (CV) of 50 mM Tris/HCl pH 8. Unbound protein was eluted by washing the column with 8 CV of 50 mM Tris/HCl PH 8. The amylase was eluted with 50 mM HEPES pH 7-10 mM imidazole and elution was monitored by absorbance at 280 nm. The eluted amylase was desalted on a HiPrep™ 26/10 desalting column (GE Healthcare Life Sciences) pre-equilibrated with 3 CV of 50 mM HEPES PH 7-100 mM NaCl. The amylase was eluted from the column using the same buffer at a flow rate of 10 ml/minute. Relevant fractions were selected and pooled based on the chromatogram and SDS-PAGE analysis using 4-12% Bis-Tris gels (Invitrogen) and 2-(N-morpholino)ethanesulfonic acid (MES) SDS-PAGE running buffer (Invitrogen). The gel was stained with InstantBlue (Novexin) and destained using miliQ water. The concentration of the purified enzyme was determined by absorbance at 280 nm.

### Example 7: Expression and Purification of the Bacillus amyloliquefaciens Amylase

The strain Bacillus amyloliquefaciens was isolated from soil in Virginia in the USA in 2011. Chromosomal DNA from the strain was subjected to full genome sequencing using Illumina technology. The GH13 subfamily 28 amylase was identified in the genome by analysing for glycosyl hydrolase domains (according to the CAZY definition). A linear integration vector-system was used for the expression cloning of the amylase from Bacillus amyloliquefaciens. The linear integration construct was a PCR fusion product made by fusion of the gene between two Bacillus subtilis homologous chromosomal regions along with a strong promoter and a chloramphenicol resistance marker. The fusion was made by SOE PCR (Horton, R. M., Hunt, H. D., Ho, S. N., Pullen, J. K. and Pease, L. R. (1989) Engineering hybrid genes without the use of restriction enzymes, gene splicing by overlap extension Gene 77: 61-68). The SOE PCR method is also described in patent application WO 2003/095658. The gene was expressed under the control of a triple promoter system (as described in WO 1999/43835), consisting of the promoters from Bacillus licheniformis alpha-amylase gene (amyL), Bacillus amyloliquefaciens alpha-amylase gene (amyQ), and the Bacillus thuringiensis crylllA promoter including stabilizing sequence. The gene coding for chloramphenicol acetyl-transferase was used as marker (described in e.g. Diderichsen, B.; Poulsen, G. B.; Joergensen, S. T. 1993, Plasmid, “A useful cloning vector for Bacillus subtilis” 30:312. The final gene construct was integrated in the Bacillus chromosome by homologous recombination into the pectate lyase locus. The gene encoding the amylase was amplified from chromosomal DNA of the strains with gene specific primers containing overhang to the two flanking vector fragments. The amylase was expressed with a Bacillus clausii secretion signal (shown in amino acids 1 to 27 of SEQ ID NO: 41) replacing the genes native secretion signal and with a His-tag (shown in amino acids 653 to 659 of SEQ ID NO: 41 fused directly to the C-terminal of the protein. The upstream and downstream vector fragments were amplified from genomic DNA of the strain MB1361 (based on strain PL3598 described in patent application WO 2003095658). The 2 linear vector fragments and the gene fragment were assembled into one linear vector construct by SOE PCR. An aliquot of the PCR product was transformed into Bacillus subtilis. Transformants were selected on LB plates supplemented with 6 μg of chloramphenicol per ml. A recombinant Bacillus subtilis clone containing the sequence confirmed integrated expression construct was cultivated in liquid culture on a rotary shaking table in 500 mL baffled Erlenmeyer flasks each containing 100 ml yeast extract-based media. The clone was cultivated for 4 days at 30° C. The enzyme containing supernatant was harvested and the enzyme purified as described below.

Purification of the His-tagged amylase from Bacillus amyloliquefaciens

The pH of the supernatant was adjusted to pH 8 with 3 M Tris, left for 1 hour, and then filtered using a filtration unit equipped with a 0.2 μm filter (Nalgene). The filtered supernatant was applied to a 5 ml HisTrap™ Excel column (GE Healthcare Life Sciences) pre-equilibrated with 5 column volumes (CV) of 50 mM Tris/HCl pH 8. Unbound protein was eluted by washing the column with 8 CV of 50 mM Tris/HCl PH 8. The amylase was eluted with 50 mM HEPES pH 7-10 mM imidazole and elution was monitored by absorbance at 280 nm. The eluted amylase was desalted on a HiPrep™ 26/10 desalting column (GE Healthcare Life Sciences) pre-equilibrated with 3 CV of 50 mM HEPES pH 7-100 mM NaCl. The amylase was eluted from the column using the same buffer at a flow rate of 10 ml/minute. Relevant fractions were selected and pooled based on the chromatogram and SDS-PAGE analysis using 4-12% Bis-Tris gels (Invitrogen) and 2-(N-morpholino)ethanesulfonic acid (MES) SDS-PAGE running buffer (Invitrogen). The gel was stained with InstantBlue (Novexin) and destained using miliQ water. The concentration of the purified enzyme was determined by absorbance at 280 nm.

### Example 8—Evaluation of Alpha-Amylase Activity

This example describes the evaluation of amylase activity in purified enzyme samples by measuring how much starch is degraded/solubilized from corn starch within 2 hours of incubation at room temperature. Initially, enzyme samples are incubated with corn starch. If the enzyme sample contains amylase activity, small starch fragments (oligosaccharides) are released from the insoluble corn starch. The insoluble (non-degraded) corn starch is separated from the soluble degraded starch by centrifugation. The solubilized starch fragments (oligosaccharides) are then degraded to glucose using glucoamylase, and finally the amount of solubilized glucose is quantified using a standard glucose oxidase/peroxidase method (e.g. Glucose Oxidase Assay Kit K-GLOX, Megazyme). The level of free glucose is an estimate of the level of amylase activity.

The alpha-amylases used in this example are the alpha-amylases comprising an amino acid sequence shown in the mature polypeptides of SEQ ID NO: 2, SEQ ID NO: 5, SEQ ID NO: 8, SEQ ID NO: 11, SEQ ID NO: 14, SEQ ID NO: 16, and SEQ ID NO: 41 (“alpha-amylases”).

### Method

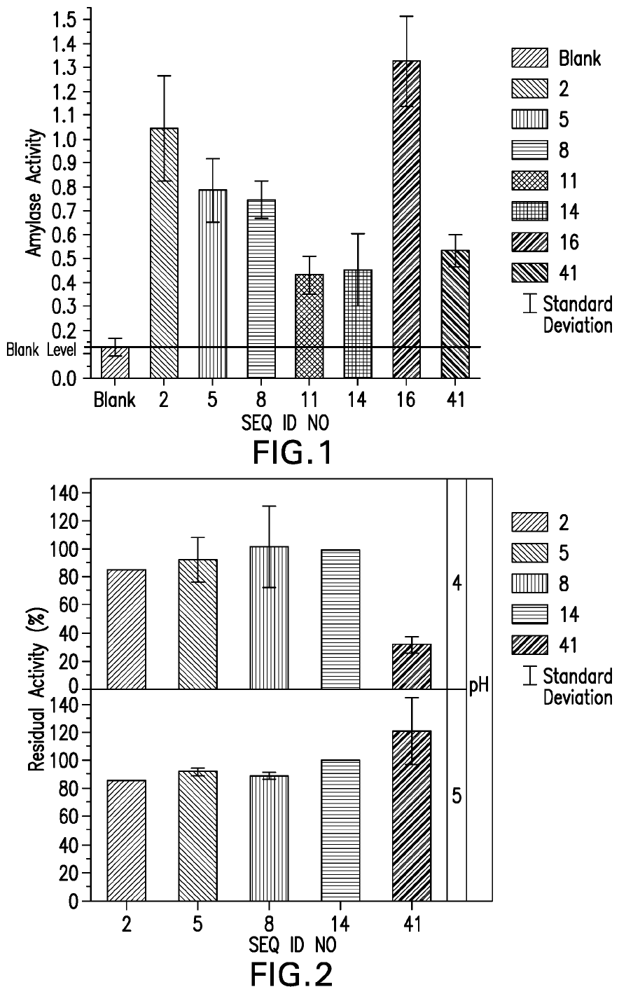

Prior to the assay, 250 nM stocks of all amylases (SEQ ID NO 2, 5, 8, 11, 14, 16 and 41) were prepared in an enzyme buffer (0.5 mM CaCl2, 0.5 mM NaCl2, 0.01% TRITON-X-100, 50% propan-1,2-diol, and 5 mM MES, pH 6). As a blank control (denoted Blank in FIG. 1) the above described enzyme buffer was applied. Amylase stock were stored cold (fridge/5° C.)prior to testing. The experiment was run in 96-well microtiter plates (e.g., NUNC® MICROWELL™ 96 well polystyrene plates). Alpha-amylases were incubated at 6.25 and 12.5 nM with 100 mg/ml corn starch (Sigma S4126) at room temperature (22-25° C.) in 0.5 mM CaCl2 and 100 mM Na-succinate (pH 5). During incubation sufficient mixing was obtained with 900 rpm (for 96-well microtiter plates). As a blank control (denoted Blank in FIG. 1), the blank control sample was added instead and incubated as described above for the amylases.

After 2 hours of incubation, residual (undegraded) corn starch was spun down (for 96-well microtiter plates with 200 μL pr. well: 2 min at 1700×g). Supernatant was withdrawn and diluted 1-343-fold (e.g., 1, 7, 49 and 343) with MILLIQ water. The diluted supernatant was subsequently mixed in a 1:1 volumetric ratio with a commercial glucoamylase from Novozymes (5 mM glucoamylase in 0.5 mM CaCl₂) and 200 mM MES, pH 6) and incubated at room temperature (22-25° C.) for 10 min with mixing (700 rpm for 96-well microtiter plate). The amount of solubilized glucose was subsequently quantified using the standard glucose oxidase/peroxidase method (e.g. Glucose Oxidase Assay Kit K-GLOX, Megazyme). This was done by running a glucose standard curve (0-0.10 mg/mL glucose dissolved in MilliQ water). The free glucose concentration was estimated for all samples by fitting to the glucose standard curve within the linear range of the standard curve. Data below the lower limit of quantification (LLOQ, see calculation below) and above the linear range of the glucose standard curve were excluded from analysis.

### Formula for Lower Limit of Quatification (LLOQ):

$$L L O Q = Mean for glucose blank + 10 \times standard deviation for glucose blank$$

$$Definition of glucose blank : 0 mg / mL glucose = MilliQ water$$

On all microtiter plates, a commercial alpha amylase is run as reference. The activity (=released glucose in mg/mL) of this reference was used to normalize the amylase activity across different microtiter plates and experiments. This normalized activity is the final calculated amylase activity shown in FIG. 1 and was calculated using Formula 1 below.

$$Amylase activity = release glucose from enzyme sample / release glucose from reference amylase \qquad Formula 1$$

### Results

FIG. 1 shows the amylase activity of all tested enzymes and a blank containing only buffer (see further explanation in method section). Results clearly shown that all tested amylases have amylase activity as the measured activity is significantly higher than the measured blank level.

### Example 9—Evaluation of pH Stability of Alpha-Amylases

This example describes the evaluation of the pH stability of the alpha-amylases comprising an amino acid sequence shown in the mature polypeptides of SEQ ID NO: 2, SEQ ID NO: 5, SEQ ID NO: 8, SEQ ID NO: 11, SEQ ID NO: 14, SEQ ID NO: 16, and SEQ ID NO: 41 (“alpha-amylases”).

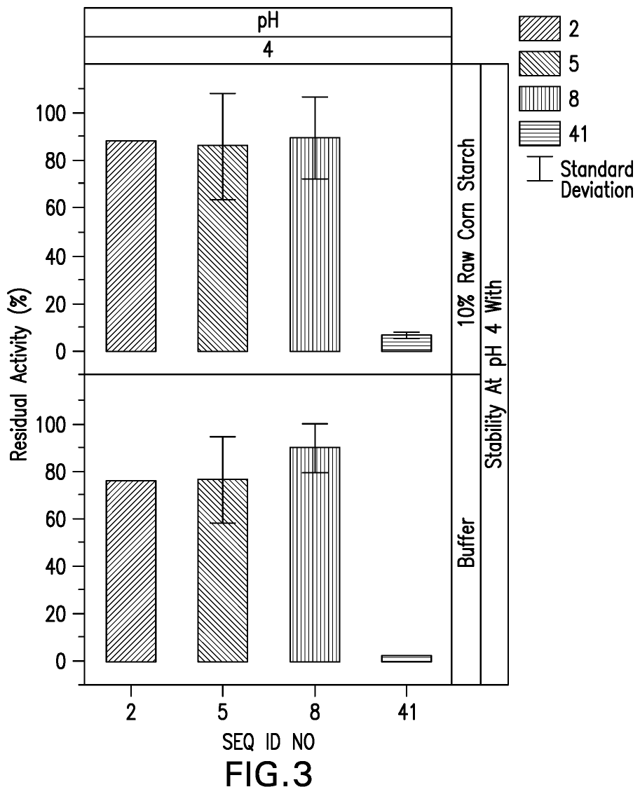

Two different tests were done (stability method 1 and stability method 2 shown in FIG. 2 and FIG. 3, respectively). In both tests, amylases are incubated at the pH values explained below (buffered solution with or without corn starch). The samples are stored in the buffered solution for a certain period of time after which the level of residual activity is estimated by comparing the amylase activity after incubation with the amylase activity a start (unstressed). The formula for residual activity (%) is shown in Formula 2 below.

$$Residual activity ( \% ) = 100 * ( Amylase activity after stress / Unstressed amylase activity ) \qquad Formula 2$$

$$Amylase activity after stress = Amylase activity after incubation at the given pH for 18 - 24 H$$

$$Unstressed amylase activity = Amylase activity before incubation ( i . e . at start , T = 0 H )$$

### Stability Method 1

Prior to the stability test, all enzyme samples are diluted to 200 μg/mL in 0.01% TRITON-X-100 (in MILLIQ water). 100 μg/mL amylase were incubated in two different stress conditions (pH 4 or pH 5) by mixing 1:1 (volumetric ratio) of enzyme stock (200 μg/mL) and the following buffers:
Stress buffer pH 4: 1 mM CaCl₂), 40 mM Na-Acetate (pH 4)
Stress buffer pH 5: 1 mM CaCl₂), 40 mM Na-Acetate (pH 5)

Both stress conditions were incubated at 32° C. over-night (18-24 H). The activity in these samples after incubation are henceforth denoted ‘Stressed activity’.

Control amylase samples were mixed to assess the activity of the unstressed enzyme. Here, 50 μg/mL amylase was incubated in 0.005% TRITON-X-100 (in MILLIQ water) over-night (18-20 H) in the fridge (5° C.). These samples are henceforth denoted ‘Unstressed activity’.

Finally, blank controls with no amylase were included. Here, samples of 0.01% TRITON-X-100 were incubated as described above for the amylase samples (both stressed and unstressed conditions).

Incubation was performed in a 96-well Nunc MicroWell polypropylene plate sealed with appropriate foil to avoid evaporation.

After incubation (18-20H), the amylase activity in the stressed conditions (‘Stressed activity’) and the unstressed condition (‘Unstressed activity’) was estimated using the standard PHADEBAS activity assay (PHADEBAS® Amylase test). Here, 4.5 mg/mL PHADEBAS® was dissolved in 1 mM CaCl₂) and 200 mM MES (pH 6) and mixed with amylase samples (stressed/unstressed) or blank controls. The volumetric ratio of Phadebas:Amylase solution: MilliQ water should be 6:2:0 or 6:1:1 depending on the level of amylase activity.

After mixing, the samples were incubated at room temperature for 15 min (no mixing) followed by intense mixing and finally settling of the insoluble Phadebas® substrate (3 min for 200 μL sample in PCR tubes, settling can also be obtained by centrifugation for e.g. larger volumes). The absorbance of the supernatant was measured at 620 nm.

The amylase activity should be in the linear activity range of the amylase standard curve and samples were diluted accordingly.

Finally, the % residual activity was calculated according to the formula given above.

### Stability Method 2

Purified enzyme samples were diluted in water with 0.01% BRIJ® 35 to a concentration of 20 μM. Blank controls contained only 0.01% BRIJ® 35 were also included. These were treated as enzyme/amylase samples below.

To assess the stability of amylases at pH 4 with and without corn starch, 10 μl of this diluted amylase was mixed with 90 μl buffer A or B:

Buffer A: 100 mM Britton-Robinson (BR) buffer, pH 4 (100 mM acetic acid, 100 mM phosphoric acid, 100 mM boric acid), 0.1 mM CaCl₂) and 0.01% BriJ®35. pH is adjusted to pH 4 using HCl.

Buffer B: 100 mg/ml corn starch, 100 mM Britton-Robinson (BR) buffer, pH 4 (100 mM acetic acid, 100 mM phosphoric acid, 100 mM boric acid), 0.1 mM CaCl₂) and 0.01% BriJ®35. pH is adjusted to pH 4 using HCl.

In total 4, plates were mixed—two with each buffer. One plate was stressed by incubation for 24 hours at 32° C., 850 rpm (‘Stressed activity’). The second plate was incubated for 2 minutes at room temperature, 850 rpm (‘Unstressed activity’). After the respective incubation, both plates were spun down for 2 min at 2000 rpm and supernatants were stored at −20° C. until analysis. Defrosted supernatants were diluted 10 times in 100 mM BR buffer (pH 7 adjusted with HCl or NaOH as needed) and enzyme activity was evaluated using a standard G7-pNP assay (Roche/Hitachi, cat. no. 11876473). G7-pNP substrate kit contains 22 mM 4,6-ethylidene-G7-pNP and 52.4 mM HEPES (2-[4-(2-hydroxyethyl)-1-piperazinyl]-ethanesulfonic acid), pH 7.0. Alpha-Glucosidase reagent contains 52.4 mM HEPES, 87 mM NaCl, 12.6 mM MgCl2, 0.075 mM CaCl2, >4 kU/L alpha-glucosidase). The substrate working solution was made by mixing 1 mL of the alpha-Glucosidase reagent with 0.2 mL of the G7-pNP substrate. This substrate working solution was made immediately before use.

Briefly, 1 volume diluted enzyme sample was mixed with 5 volumes G7-pNP solution and absorbance was measured at 405 nm for 20 min at room temperature (22-25° C.). The amylase activity was defined as the initial slopes (0-2 min) minus the initial slope for the blank control.

Finally, the % residual activity was calculated according to the formula given above.

### Results

As described above, two separate stability tests were performed (FIG. 2 and FIG. 3). Both tests focus on estimating the stability of the new alpha amylases at the lower pH range (pH 4-5) with and without corn starch. If the alpha amylases bind tightly to corn starch, corn starch is expected to increase the stability of the alpha amylase (in case it is unstable). The data are shown in FIG. 2 and FIG. 3, respectively. Data from both experiments clearly show that all the alpha-amylases tested retain greater than about 75% residual activity at low pH (either pH 4 or 5), indicating that they are stable at industrial applications at lower pH ranges, such as during the saccharification, fermentation, or simultaneous saccharification and fermentation steps of a conventional ethanol production process using starch-containing material, such as corn, as a substrate.

Furthermore, data in FIG. 3 indicate that the stability increased in the presence of corn starch indicating that these alpha amylases bind to corn starch and are well suited for applications with corn starch at the lower pH range.

### Example 10—Evaluation of Alpha-Amylases on Ethanol Yield and Kinetics During Simultaneous Saccharification and Fermentation (SSF) of Corn Mash

This example describes how to evaluate alpha-amylases for improved ethanol and kinetics during simultaneous saccharification and fermentation (SSF) using corn mash derived from conventional dry grind ethanol plant. Particularly, the ethanol and kinetics during fermentation are compared among the polypeptides having alpha-amylase activity of the mature polypeptides of SEQ ID NO: 2, SEQ ID NO: 5, SEQ ID NO: 8, SEQ ID NO: 11, SEQ ID NO: 14, SEQ ID NO: 16, SEQ ID NO: 41, SEQ ID NO: 44, SEQ ID NO: 47, SEQ ID NO: 50, SEQ ID NO: 53, SEQ ID NO: 56, or SEQ ID NO: 59 (“alpha-amylases”).

### Yeast Preparation

Cryo-preserved culture of a fermenting organism, e.g., Saccharomyces cerevisiae strain MBG5012, can be grown in liquid YPD media (Yeast extract, 10 g. Peptone, 20 g. Dextrose, 60 g. dissolve in 1 L of distilled water). Cultivation can be done aseptically in a sterile 125-ml Erlenmeyer flask filled with 50 ml YPD media and inoculated with 100 μl of cryo-preserved culture. Flask can be incubated in a shaking incubator at 32° C. for 16 h with shaking at 150 rpm. The YPD grown seed cultures (40 ml) can be centrifuged at 3,500 rpm for 10 min at 22° C., and the resulting cell pellet can be washed and resuspended in tap water. The resuspended cells can be used to inoculate the corn mash at the beginning of simultaneous saccharification and fermentation (SSF).

### Corn Mash

Liquefied corn mash obtained from a commercial ethanol plant that was treated with Alpha-Amylase Blend X at 85-91° C. to solubilize and hydrolyze starch from corn flour can be pH adjusted to 5.0 using either 0.5 M NaOH or 40% (v/v) H2SO4. Urea and Lactrol can be added to corn mash to the final concentration of 1000 ppm and 3 ppm, respectively, to achieve total dry solids of between 33-36%.

### Simultaneous Saccharification and Fermentation (SSF)

Fermentations can be carried out in 125 mL Erlenmyer flasks (or bottles) with caps having a 0.5 mm hole. Flasks can be filled with 60 g of corn mash and inoculated with seed culture at 10 million cells per gram mash. A glucoamylase (GsAMG) is then added to the flasks at 70 ug and 0.7 ug enzyme protein per g of dry corn solids, respectively. The alpha-amylases are added to the flasks at 32 μg enzyme protein per g of dry corn solids. All flasks contain the same glucoamylase and one of the alpha-amylases. Flasks can be incubated in an incubator at 32° C. while shaking at 100-120 rpm for 54 hours.

### Ethanol, Sugars and Organic Acid Analyses

At the following time points (6, 24, 48 and 54 hours) during the SSF, 4 mL samples can be taken from each flask, and 80 ul of 40% (v/v) H₂SO₄ are added to stop the reaction. These mixtures are vortexed, and centrifuged at 3,500 rpm for 10 min at 22° C. The resulting supernatant can be filtered through a 0.2 μm syringe filter. Filtered samples were stored at 4° C. prior to and during HPLC analysis. Analysis of ethanol can be conducted using an HPLC (Agilent 1100/1200 series) machine equipped with a guard column (Bio-Rad, Micro-Guard Cation H+Cartridge, 30×4.6 mm) and an analytical column (Bio-Rad, Aminex HPX-87H, 300×7.8 mm) using 5 mM Sulfuric Acid as a mobile phase with a flow rate of 0.8 mL/min. Column temperature can be maintained at 65° C., and analytes can be detected using a Refractive Index detector at 55° C.

### Results

Ethanol kinetics during 54 hour SSF that are treated with the alpha-amylases can be reported in a graph. Final ethanol tiers after 54 hours of SSF that are treated with the alpha-amylases can be reported in a graph.

### Example 11—Expression of Lactobacillus amylovorus Amylase Hybrids

Four expression constructs were made expressing the catalytic domain of the alpha-amylase from Lactobacillus amylovorus fused to different starch binding domains (SEQ ID NO: 43, SEQ ID NO: 46, SEQ ID NO: 49, SEQ ID NO: 52). The genes were ordered as synthetic cloned genes at TWIST Bioscience.

The synthetic genes were inserted into a Bacillus expression vector as described in WO 12/02557. Briefly, the DNA encoding alpha-amylases with starch binding domains were cloned in frame to a Bacillus clausii secretion signal (with the following amino acid sequence: MKKPLGKIVASTALLISVAFSSSIASA (SEQ ID NO: 60). The Bacillus clausii secretion signal replaced the native secretion signal in the genes. An affinity tag sequence was introduced in the C-terminal of each gene to ease the purification process (His-tag; with the following amino acid sequence: HHHHHH (SEQ ID NO: 61). The final expression plasmids were transformed into a Bacillus subtilis expression host. The gene constructs were integrated by homologous recombination into the Bacillus subtilis chromosome by homologous recombination into the pectate lyase locus. The genes were expressed under the control of a triple promoter system (as described in WO 99/43835). Transformants were selected on LB plates supplemented with 6 ug of chloramphenicol per ml. A recombinant Bacillus subtilis clone of each construct containing the sequence confirmed integrated expression construct was cultivated in liquid culture on a rotary shaking table in 500 mL baffled Erlenmeyer flasks each containing 100 ml yeast extract-based media. The clones were cultivated for 4 days at 30° C. The enzyme containing supernatants were harvested and the enzymes purified as described in Example 12.

### Example 12—Purification of the His-Tagged Amylases from Lactobacillus amylovorus

The pH of the supernatant was adjusted to pH 8 with 3 M Tris, left for 1 hour, and then filtered using a filtration unit equipped with a 0.2 μm filter (Nalgene). The filtered supernatant was applied to a 5 ml HISTRAP™ Excel column (GE Healthcare Life Sciences) pre-equilibrated with 5 column volumes (CV) of 50 mM Tris/HCl PH 8. Unbound protein was eluted by washing the column with 8 CV of 50 mM Tris/HCl PH 8. The amylase was eluted with 50 mM HEPES pH 7-10 mM imidazole and elution was monitored by absorbance at 280 nm. The eluted amylase was desalted on a HIPREP™ 26/10 desalting column (GE Healthcare Life Sciences) pre-equilibrated with 3 CV of 50 mM HEPES PH 7-100 mM NaCl. The amylase was eluted from the column using the same buffer at a flow rate of 10 ml/minute. Relevant fractions were selected and pooled based on the chromatogram and SDS-PAGE analysis using 4-12% Bis-Tris gels (Invitrogen) and 2-(N-morpholino)ethanesulfonic acid (MES) SDS-PAGE running buffer (Invitrogen). The gel was stained with INSTANTBLUE (Novexin) and destained using miliQ water. The concentration of the purified enzyme was determined by absorbance at 280 nm.

### Example 13—Expression of Lactobacillus amylovorus Amylase Hybrids

Two expression constructs were made expressing the catalytic domain of the alpha-amylase from Lactobacillus amylovorus fused to different starch binding domains (SEQ ID NO: 55 and SEQ ID NO: 58). The genes were ordered as synthetic cloned genes at TWIST Bioscience.

The synthetic genes were inserted into a Bacillus expression vector as described in WO 12/02557 (incorporated by reference herein in its entirety). Briefly, the DNA encoding alpha-amylases with starch binding domains were cloned in frame to a Bacillus clausii secretion signal (with the following amino acid sequence: MKKPLGKIVASTALLISVAFSSSIASA (SEQ ID NO: 60). The Bacillus clausii secretion signal replaced the native secretion signal in the genes. An affinity tag sequence was introduced in the C-terminal of each gene to ease the purification process (His-tag; with the following amino acid sequence: HHHHHH (SEQ ID NO: 61). The final expression plasmids were transformed into a Bacillus subtilis expression host. The gene constructs were integrated by homologous recombination into the Bacillus subtilis chromosome by homologous recombination into the pectate lyase locus. The genes were expressed under the control of a triple promoter system (as described in WO 99/43835; incorporated herein by reference in its entirety). Transformants were selected on LB plates supplemented with 6 ug of chloramphenicol per ml. A recombinant Bacillus subtilis clone of each construct containing the sequence confirmed integrated expression construct was cultivated in liquid culture on a rotary shaking table in 500 mL baffled Erlenmeyer flasks each containing 100 ml yeast extract-based media. The clones were cultivated for 1 day at 37° C. The enzyme containing supernatants were harvested and the enzymes purified as described in Example 14.

### Example 14—Purification of the His-Tagged Amylases from Lactobacillus amylovorus (SEQ ID NO: 57 and SEQ ID NO: 60)

The pH of the supernatant was adjusted to pH 8 with 3 M Tris, left for 1 hour, and then filtered using a filtration unit equipped with a 0.22 μm filter (Millipore). The filtered supernatant was applied to a 5 ml HISTRAP™ HP column (GE Healthcare Life Sciences) pre-equilibrated with 5 column volumes (CV) of 50 mM Tris-HCl PH 8, 100 mM NaCl. Unbound protein was eluted by washing the column with 5 CV of 50 mM Tris-HCl PH 8, 100 mM NaCl; 5CV of 50 mM HEPES-NaOH pH 7, 100 mM NaCl, 10 mM imidazole; 3CV of 50 mM HEPES-NaOH PH 7, 100 mM NaCl, 50 mM imidazole sequentially. The amylase was eluted with 50 mM HEPES PH 7, 100 mM NaCl, 500 mM imidazole and elution was monitored by absorbance at 280 nm. The eluted amylase was desalted on a HIPREP™ 26/10 desalting column (GE Healthcare Life Sciences) pre-equilibrated with 3 CV of 50 mM HEPES pH 7, 100 mM NaCl. The amylase was eluted from the column using the same buffer at a flow rate of 5 ml/minute. Relevant fractions were selected and pooled based on the chromatogram and SDS-PAGE analysis using 12% Mini-PROTEAN TGX Stain-free gels (BIO-RAD). The concentration of the purified enzyme was determined by absorbance at 280 nm.

### Example 15—Evaluation of Alpha-Amylase Activity

This example describes the evaluation of amylase activity in purified enzyme samples by measuring how much starch is degraded/solubilized from corn starch within 2 hours of incubation at room temperature. Initially, enzyme samples are incubated with corn starch. If the enzyme sample contains amylase activity, small starch fragments (oligosaccharides) are released from the insoluble corn starch. The insoluble (non-degraded) corn starch is separated from the soluble degraded starch by centrifugation. The solubilized starch fragments (oligosaccharides) are then degraded to glucose using glucoamylase, and finally the amount of solubilized glucose is quantified using a standard glucose oxidase/peroxidase method (e.g. Glucose Oxidase Assay Kit K-GLOX, MEGAZYME). The level of free glucose is an estimate of the level of amylase activity.

The alpha-amylases used in this example are the alpha-amylases comprising an amino acid sequence shown in the mature polypeptides of SEQ ID NO: 45, SEQ ID NO: 48, SEQ ID NO: 51, SEQ ID NO: 54, SEQ ID NO: 57, and SEQ ID NO: 60 (“alpha-amylases”).

### Method

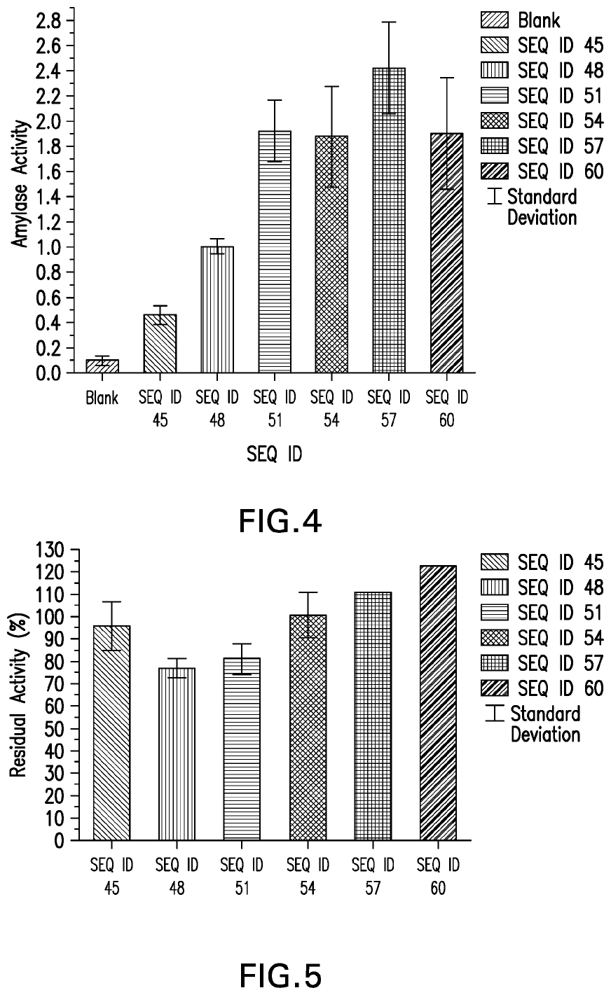

Prior to the assay, 250 nM stocks of all amylases (SEQ ID Nos: 45, 48, 51, 54, 57, and 60) were prepared in an enzyme buffer (0.5 mM CaCl2, 0.5 mM NaCl2, 0.01% TRITON-X-100, 50% propan-1,2-diol, and 5 mM MES, pH 6). As a blank control (denoted Blank in FIG. 4) the above described enzyme buffer was applied. Amylase stock were stored cold (fridge/5° C.)prior to testing. The experiment was run in 96-well microtiter plates (e.g., NUNC® MICROWELL™ 96 well polystyrene plates). Alpha-amylases were incubated at 6.25 and 12.5 nM with 100 mg/ml corn starch (Sigma S4126) at room temperature (22-25° C.) in 0.5 mM CaCl2 and 100 mM Na-succinate (pH 5). During incubation, sufficient mixing was obtained with 900 rpm (for 96-well microtiter plates). As a blank control (denoted Blank in FIG. 4), the blank control sample was added instead and incubated as described above for the amylases.

After 2 hours of incubation, residual (undegraded) corn starch was spun down (for 96-well microtiter plates with 200 μL pr. well: 2 min at 1700×g). Supernatant was withdrawn and diluted 1-343-fold (e.g., 1, 7, 49 and 343) with MILLIQ water. The diluted supernatant was subsequently mixed in a 1:1 volumetric ratio with a commercial glucoamylase from Novozymes (10 mM glucoamylase in 0.5 mM CaCl₂) and 200 mM MES, pH 6) and incubated at room temperature (22-25° C.) for 10 min with mixing (700 rpm for 96-well microtiter plate). The amount of solubilized glucose was subsequently quantified using the standard glucose oxidase/peroxidase method (e.g. Glucose Oxidase Assay Kit K-GLOX, MEGAZYME). This was done by running a glucose standard curve (0-0.10 mg/mL glucose dissolved in MILLIQ water). The free glucose concentration was estimated for all samples by fitting to the glucose standard curve within the linear range of the standard curve. Data below the lower limit of quantification (LLOQ, see calculation below) and above the linear range of the glucose standard curve were excluded from analysis.

$$Formula for lower limit of quantification ( LLOQ )$$

$$L L O Q = Mean for glucose blank + 10 \times standard deviation for glucose blank$$

$$Definition of glucose blank : 0 mg / mL glucose = MilliQ water$$

On all microtiter plates, a commercial alpha amylase is run as reference. The activity (=released glucose in mg/mL) of this reference was used to normalize the amylase activity across different microtiter plates and experiments. This normalized activity is the final calculated amylase activity shown in FIG. 4 and was calculated using Formula 2 below.

$$Am 2 lase activity = release glucose from enzyme sample / release glucose from reference amylase \qquad Formula 1$$

### Results

FIG. 4 shows the amylase activity of all tested enzymes and a blank containing only buffer (see further explanation in method section). Results clearly shown that all tested amylases have amylase activity as the measured activity is significantly higher than the measured blank level.

### Example 16—Evaluation of pH 4 Stability of Alpha-Amylases

This example describes the evaluation of the pH 4 stability of the alpha-amylases comprising an amino acid sequence shown in the mature polypeptides of SEQ ID NO: 45, SEQ ID NO: 48, SEQ ID NO: 51, SEQ ID NO: 54, SEQ ID NO: 57, and SEQ ID NO: 60 (“alpha-amylases”).

Purified amylases were diluted in water with 0.01% Trition-X-100 to a concentration of 2-5 M. For the assay, the enzyme samples (2-5 UM) were diluted 10X into either stress buffer (222 mM sodium acetate buffer pH 4, 0.56 mM CaCl2 and 0.01% Brij-35) or dilution buffer (0.01% Trition-X-100). Samples in the stress buffer were incubated at 32° C. (850 rpm) for 24H (hereafter called stressed samples). The other samples mixed with dilution buffer were stored at 5° C. until further analysis, max. 4 days (hereafter called unstressed samples).

After incubation, the stressed samples were diluted 10-50X with assay buffer (500 mM Hepes pH 7, 0.5 mM CaCl2, 0.01% Brij-35). The unstressed samples were diluted between 10-100X to get minimum four different dilutions and thus minimum 4 different concentrations. Enzyme activity was evaluated for all diluted samples (stressed and unstressed) using G7-pNP assay protocol (Roche/Hitachi, cat. no. 11876473). Briefly, 20 μl diluted enzyme sample was mixed with 100 μl G7-pNP solution and absorbance was measured at 405 nm for 20 min at room temperature. Initial slopes (lag time: 2 min, max absorbance=1.5) were calculated. For the unstressed samples, a nonlinear fit (e.g. Michaelis Menten) was made using initial slopes as Y and enzyme concentration as X. Based on this fit, the concentration of residual active enzyme was estimated in the stressed samples. The level of residual activity (RA %) was calculated by dividing the estimated concentration of residual active enzyme with the initial concentration of enzyme (at start) and multiplying with 100.

### Results

Data are shown in FIG. 5. Data clearly show that all the alpha-amylases tested retain greater than about 75% residual activity at low pH (either pH 4), indicating that they are stable at industrial applications at lower pH ranges, such as during the saccharification, fermentation, or simultaneous saccharification and fermentation steps of a conventional ethanol production process using starch-containing material, such as corn, as a substrate.

### Example 16—Evaluation of Ethanol Stability of Alpha-Amylases

This example describes the evaluation of the Ethanol stability of the alpha-amylases comprising an amino acid sequence shown in the mature polypeptides of SEQ ID NO: 6, SEQ ID NO: 9, SEQ ID NO: 12, SEQ ID NO: 15, SEQ ID NO: 18, SEQ ID NO: 42, SEQ ID NO: 45, SEQ ID NO: 48, SEQ ID NO: 51, SEQ ID NO: 54, SEQ ID NO: 57, and SEQ ID NO: 60 (“alpha-amylases”).

Purified amylases were diluted in water with 0.01% TRITION-X-100 to a concentration of 2-5 μM. For the assay, the enzyme samples (2-5 μM) were diluted 10X into either stress buffer (222 mM sodium acetate buffer pH 5, 16.7% (v/v) EtOH, 0.56 mM CaCl2 and 0.01% Brij-35) or dilution buffer (0.01% TRITION-X-100). Samples in the stress buffer were incubated at 32° C. (850 rpm) for 24H (hereafter called stressed samples). The other samples mixed with dilution buffer were stored at 5° C. until further analysis, max. 4 days (hereafter called unstressed samples).

After incubation, the stressed samples were diluted 10-50X with assay buffer (500 mM HEPES pH 7, 0.5 mM CaCl2, 0.01% Brij-35). The unstressed samples were diluted between 10-100X in the same assay buffer to get minimum four different dilutions and thus minimum 4 different concentrations. Enzyme activity was evaluated for all diluted samples (stressed and unstressed) using G7-pNP assay protocol (Roche/Hitachi, cat. no. 11876473). Briefly, 20 u| diluted enzyme sample was mixed with 100 μl G7-pNP solution and absorbance was measured at 405 nm for 20 min at room temperature. Initial slopes (lag time: 2 min, max absorbance=1.5) were calculated. For the unstressed samples, a nonlinear fit (e.g. Michaelis Menten) was made using initial slopes as Y and enzyme concentration as X. Based on this fit, the concentration of residual active enzyme was estimated in the stressed samples. The level of residual activity (RA %) was calculated by dividing the estimated concentration of residual active enzyme with the initial concentration of enzyme (at start) and multiplying with 100.

### Results

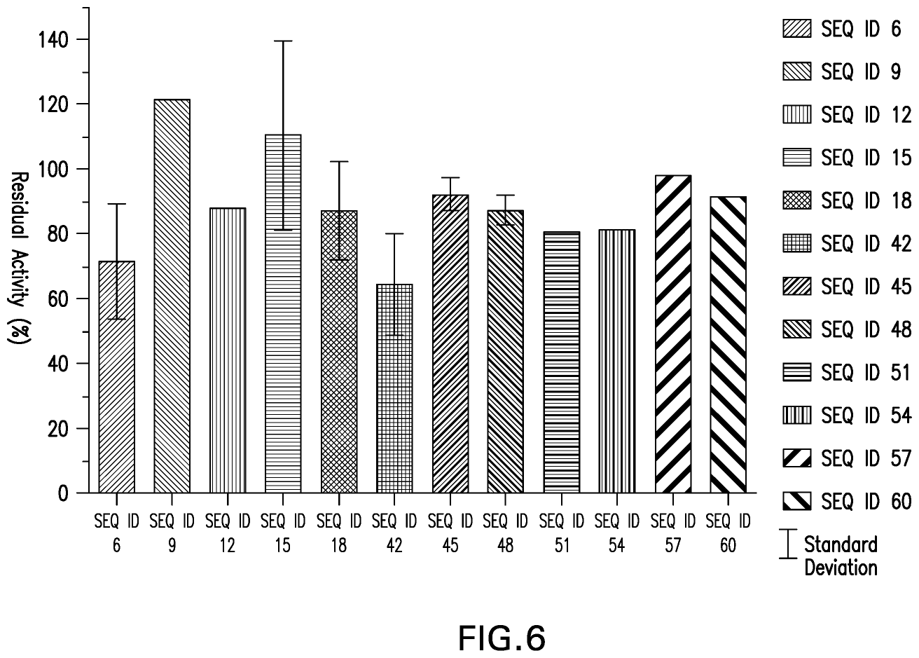

Data are shown in FIG. 6. Data clearly show that all the alpha-amylases tested retain greater than 65% residual activity after 24H with 15% EtOH (32° C., pH 5), indicating that they are stable at industrial applications including EtOH up to at least 15% (v/v) EtOH such as during fermentation or simultaneous saccharification and fermentation steps of a conventional ethanol production process using starch-containing material, such as corn, as a substrate.

### Example 17—Impact of Newly Identified Alpha-Amylases in Reducing the Residual Starch Level Following Simultaneous Saccharification and Fermentation (SSF) of Corn Mash

This example showed the amount of starch remained following 54 h SSF with various alpha-amylases of the present invention (SEQ ID NOs: 3, 6, 9, 12, 15, 18, 42, 45, 48, 51, 54, 57 and 60; collectively “alpha-amylases”). For this assay, starch is considered insoluble in aqueous solution, so any soluble glucose/maltodextrin was removed via two additional washes using deionized water. Residual starch level, when treated with various alpha-amylases, can be used to determine the overall effectiveness of alpha-amylases in accessing starch in a simultaneous saccharification and fermentation step of process for producing a fermentation product.

### Yeast Preparation:

One gram of dry ETHANOL RED™ yeast strain (available from Lesaffre, USA), was resuspended into 20 mL of tap water and placed at 32° C. for 40 minutes before adding to each tube. A total of 83 uL of this yeast suspension was used to inoculate each tube containing corn mash at the beginning of simultaneous saccharification and fermentation (SSF).

### Corn Mash:

Liquefied corn mash was obtained from a commercial ethanol plant that was treated with a typical enzyme mix containing an alpha-amylase (Alpha-Amylase A (AAA); Novozymes) at 85-91° C. to solubilize and hydrolyze starch from corn flour. The pH of corn mash was adjusted to 5.0 using 50% NaOH. Urea and Lactrol were added to corn mash to the final concentration of 1000 ppm and 3 ppm, respectively. The total final dry solids level was 32.29%.

### Simultaneous Saccharification and Fermentation (SSF)

All fermentations were carried out in 15 mL round bottom tubes with caps having a 0.5 mm drilled hole. Tubes were filled with an average of 4.12 gram of corn mash and inoculated with 83 uL of prepared yeast suspension. A glucoamylase (Glucoamylase BL2; Novozymes) were added to the tubes at 0.60 AGU per g of dry corn solids. The alpha-amylases were added to the tubes at either 5 ug or 20 μg enzyme protein per g of dry corn solids. All tubes contained the same glucoamylase and one amylase. For a control, no a-amylase was added. Three replicates (tubes) tubes were used for each treatment, and these tubes were incubated in an incubator at 32° C. for 54 hours. The content of each tube was mixed well by vortex at 7, 22, 31, 46 and 54 h.

### Sample Preparation and Residual Starch Assay

After 54 h of SSF, 50 ul of 40% sulfuric acid was added to each tube and mixed well by vortex to stop the fermentation. These tubes were centrifuged at 3,500×g for 10 min at 22° C. The supernatant was removed for other analysis and the remaining pellet was washed twice each time with 4 mL of di-ionized water. Similar centrifugation setting was used between the two water washes. Following the second water wash, the supernatant was removed and the pellet was resuspended well in 3.5 mL of 50 mM sodium acetate/2.5 mM calcium chloride (pH 5.0) and 50 uL of Glucoamylase BL (Novozymes), an enzyme cocktail containing a mixture of alpha-amylase and glucoamylase to break down the starch in the pellet sample. The mixture was incubated at 50° C. for 23 h. Following the incubation, the content in each tube was mixed well by vortex, centrifuged, and the supernatant was filtered through a 0.2 μm syringe filter. Filtered samples were stored at 4° C. prior to and during HPLC analysis. A negative control, containing only the 50 mM sodium acetate buffer and 50 uL of Glucoamylase BL, was used to determine to background glucose level in the assay. Analysis of glucose level was conducted using an HPLC (Agilent 1100/1200 series) machine equipped with a guard column (Bio-Rad, Micro-Guard Cation H+Cartridge, 30×4.6 mm) and an analytical column (Bio-Rad, Aminex HPX-87H, 300×7.8 mm) using 5 mM Sulfuric Acid as a mobile phase with a flow rate of 0.8 mL/min. Column temperature was maintained at 65° C., and ethanol was detected using a Refractive Index detector at 55° C.

The amount of starch per gram of mash (as-is) in each tube was calculated according to the following formula:

$$\% starch per gram of corn mash = ( Gs - Gn ) \times V \times 0.9 / M \times 100$$

### Where:

Gs=glucose concentration of a sample (g/L)
Gn=glucose concentration of the Negative Control (g/L)
V=0.00355 L (the volume of enzyme/buffer cocktail mix; 3.55 mL)
0.9=conversion factor between starch and glucose (0.9 gram starch=1 gram glucose)
M=the weight of wet corn mash (g) at the end of 54 h SSF
100=Percent factor

### Results

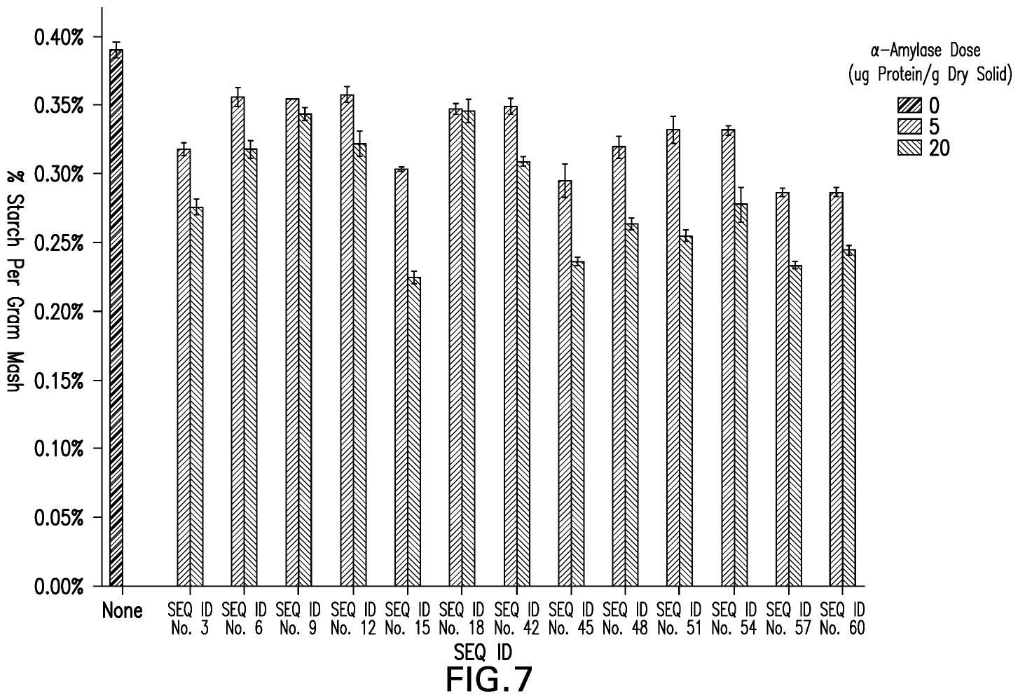

The ability of the tested alpha-amylases in breaking down additional amount of starch during the 54 h of SSF are shown in FIG. 7. Treatment with these alpha-amylases at 5 ug dose (per gram dry solid) resulted in significant reduction of starch in the remaining pellet samples when comparing to the Control (0 ug added). In addition, an additional level of starch reduction was observed in majority of alpha-amylases when used at a higher dose (20 ug). Out of thirteen alpha-amylases, only two SEQ ID NO: 9 and SEQ ID NO: 18 did not show additional starch utilization at 20 ug dose. Overall, these results strongly support that the addition of these alpha-amylases in the SSF allowed breaking down additional starch present in the insoluble fraction of corn mash.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. An isolated or purified polypeptide having alpha-amylase activity, selected from the group consisting of:
(i)
(a) a polypeptide having at least 92%, at least 93%, at least 94%, at least 95%, at least 96%, at least 97%, at least 98%, at least 99%, or 100% sequence identity to SEQ ID NO: 2;
(b) a polypeptide having at least 92%, at least 93%, at least 94%, at least 95%, at least 96%, at least 97%, at least 98%, at least 99%, or 100% sequence identity to the sequence of SEQ ID NO: 3;
(c) a polypeptide having at least 92%, at least 93%, at least 94%, at least 95%, at least 96%, at least 97%, at least 98%, at least 99%, or 100% sequence identity to a mature polypeptide of SEQ ID NO: 2;
(d) a polypeptide encoded by a polynucleotide that hybridizes under medium, medium-high, or high stringency conditions with the full-length complement of the mature polypeptide coding sequence of SEQ ID NO: 1;
(e) a polypeptide encoded by a polynucleotide having at least 92%, at least 93%, at least 94%, at least 95%, at least 96%, at least 97%, at least 98%, at least 99%, or 100% sequence identity to the mature polypeptide coding sequence of SEQ ID NO: 1;
(f) a fragment of the polypeptide of (a), (b), (c), (d), or (e) that has alpha-amylase activity;
(ii)
(a) a polypeptide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 5;
(b) a polypeptide having at least 85%, at least 86%, at least 87%, at least 88%, at least 96%, at least 97%, at least 98%, at least 99%, or 100% sequence identity to the sequence of SEQ ID NO: 6;
(c) a polypeptide having at least 85%, at least 86%, at least 87%, at least 88%, at least 96%, at least 97%, at least 98%, at least 99%, or 100% sequence identity to a mature polypeptide of SEQ ID NO: 5;
(d) a polypeptide encoded by a polynucleotide that hybridizes under medium, medium-high, or high stringency conditions with the full-length complement of the mature polypeptide coding sequence of SEQ ID NO: 4, or the cDNA sequence thereof;
(e) a polypeptide encoded by a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 4, or the cDNA sequence thereof;
(f) a fragment of the polypeptide of (a), (b), (c), (d), or (e) that has alpha-amylase activity;
(iii)
(a) a polypeptide having at least 93%, at least 94%, at least 95%, at least 96%, at least 97%, at least 98%, at least 99%, or 100% sequence identity to SEQ ID NO: 8;
(b) a polypeptide having at least 93%, at least 94%, at least 95%, at least 96%, at least 97%, at least 98%, at least 99%, or 100% sequence identity to the sequence of SEQ ID NO: 9;
(c) a polypeptide having at least 93%, at least 94%, at least 95%, at least 96%, at least 97%, at least 98%, at least 99%, or 100% sequence identity to a mature polypeptide of SEQ ID NO: 8;
(d) a polypeptide encoded by a polynucleotide that hybridizes under medium, medium-high, or high stringency conditions with the full-length complement of the mature polypeptide coding sequence of SEQ ID NO: 7, or the cDNA sequence thereof;
(e) a polypeptide encoded by a polynucleotide having at least 93%, at least 94%, at least 95%, at least 96%, at least 97%, at least 98%, at least 99%, or 100% sequence identity to the mature polypeptide coding sequence of SEQ ID NO: 7, or the cDNA sequence thereof;
(f) a fragment of the polypeptide of (a), (b), (c), (d), or (e) that has alpha-amylase activity;
(iv)
(a) a polypeptide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
(b) a polypeptide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 12;
(c) a polypeptide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 11;
(d) a polypeptide encoded by a polynucleotide that hybridizes under medium, medium-high, or high stringency conditions with the full-length complement of the mature polypeptide coding sequence of SEQ ID NO: 10;
(e) a polypeptide encoded by a polynucleotide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 10;
(f) a fragment of the polypeptide of (a), (b), (c), (d), or (e) that has alpha-amylase activity;
(v)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to the sequence of SEQ ID NO: 15;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to a mature polypeptide of SEQ ID NO: 14;
(d) a polypeptide encoded by a polynucleotide that hybridizes under medium, medium-high, or high stringency conditions with the full-length complement of the mature polypeptide coding sequence of SEQ ID NO: 13;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 13;
(f) a fragment of the polypeptide of (a), (b), (c), (d), or (e) that has alpha-amylase activity;
(vi)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 16;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 17;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to a mature polypeptide of SEQ ID NO: 16;
(d) a fragment of the polypeptide of (a), (b), or (c), that has alpha-amylase activity;
(vii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 42;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 42;
(d) a fragment of the polypeptide of (a), (b), or (c), that has alpha-amylase activity;
(viii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to the sequence of SEQ ID NO: 45;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 44;
(d) a polypeptide encoded by a polynucleotide that hybridizes under medium, medium-high, or high stringency conditions with the full-length complement of the mature polypeptide coding sequence of SEQ ID NO: 43;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 43; or
(f) a fragment of the polypeptide of (a), (b), (c), (d), or (e) that has alpha-amylase activity;
(ix)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 48;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to a mature polypeptide of SEQ ID NO: 47;
(d) a polypeptide encoded by a polynucleotide that hybridizes under medium, medium-high, or high stringency conditions with the full-length complement of the mature polypeptide coding sequence of SEQ ID NO: 46;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 46; or
(f) a fragment of the polypeptide of (a), (b), (c), (d), or (e) that has alpha-amylase activity;
(x)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 50;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 51;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to a mature polypeptide of SEQ ID NO: 50;
(d) a polypeptide encoded by a polynucleotide that hybridizes under medium, medium-high, or high stringency conditions with the full-length complement of the mature polypeptide coding sequence of SEQ ID NO: 49;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 49; or
(f) a fragment of the polypeptide of (a), (b), (c), (d), or (e) that has alpha-amylase activity;
(xi)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 53;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 54;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 53;
(d) a polypeptide encoded by a polynucleotide that hybridizes under medium, medium-high, or high stringency conditions with the full-length complement of the mature polypeptide coding sequence of SEQ ID NO: 52;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 52; or
(f) a fragment of the polypeptide of (a), (b), (c), (d), or (e) that has alpha-amylase activity
(xii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 56;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 57;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 56;
(d) a polypeptide encoded by a polynucleotide that hybridizes under medium, medium-high, or high stringency conditions with the full-length complement of the mature polypeptide coding sequence of SEQ ID NO: 55;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 55; or
(f) a fragment of the polypeptide of (a), (b), (c), (d), or (e) that has alpha-amylase activity; and
(xiii)
(a) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 59;
(b) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 60;
(c) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 59;
(d) a polypeptide encoded by a polynucleotide that hybridizes under medium, medium-high, or high stringency conditions with the full-length complement of the mature polypeptide coding sequence of SEQ ID NO: 58;
(e) a polypeptide encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 58; or
(f) a fragment of the polypeptide of (a), (b), (c), (d), or (e) that has alpha-amylase activity.

2. The polypeptide of claim 1, having:
(a) at least 92%, at least 93%, at least 94%, at least 95%, at least 96%, at least 97%, at least 98%, at least 99% or 100% sequence identity to SEQ ID NO: 2;
(b) at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 5;
(c) at least 93%, at least 94%, at least 95%, at least 96%, at least 97%, at least 98%, at least 99%, or 100% sequence identity to SEQ ID NO: 8;
(d) at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 11;
(e) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14;
(f) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 16;
(g) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 41;
(h) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 44;
(i) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 47;
(j) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 50;
(k) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 53;
(l) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 56; or
(m) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 59.

3. The polypeptide of claim 1, having:
(a) at least 92%, at least 93%, at least 94%, at least 95%, at least 96%, at least 97%, at least 98%, at least 99% or 100% sequence identity to SEQ ID NO: 3;
(b) at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 6;
(c) at least 93%, at least 94%, at least 95%, at least 96%, at least 97%, at least 98%, at least 99%, or 100% sequence identity to SEQ ID NO: 9;
(d) at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 12;
(e) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 15;
(f) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 17;
(g) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the sequence of SEQ ID NO: 42;
(h) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 45;
(i) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 48;
(j) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 51;
(k) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 54;
(l) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 57; or
(m) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 60.

4. The polypeptide of claim 1, having:
(a) at least 92%, at least 93%, at least 94%, at least 95%, at least 96%, at least 97%, at least 98%, at least 99% or 100% sequence identity to a mature polypeptide of SEQ ID NO: 2;
(b) at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 5;
(c) at least 93%, at least 94%, at least 95%, at least 96%, at least 97%, at least 98%, at least 99%, or 100% sequence identity to a mature polypeptide of SEQ ID NO: 8;
(d) at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 11;
(e) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 14;
(f) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 16;
(g) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 41;
(h) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 44;
(i) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 47;
(j) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 50;
(k) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 96%, at least 97%, at least 98%, at least 99%, or 100% sequence identity to a mature polypeptide of SEQ ID NO: 53;
(j) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 56; or
(k) a polypeptide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to a mature polypeptide of SEQ ID NO: 59.

5. The polypeptide of claim 1, comprising, consisting essentially of, or consisting of:
(a) SEQ ID NO: 2 or a mature polypeptide thereof; or SEQ ID NO: 3;
(b) SEQ ID NO: 5 or a mature polypeptide thereof; or SEQ ID NO: 6;
(c) SEQ ID NO: 8 or a mature polypeptide thereof; or SEQ ID NO: 9;
(d) SEQ ID NO: 11 or a mature polypeptide thereof; or SEQ ID NO: 12;
(e) SEQ ID NO: 14 or a mature polypeptide thereof; or SEQ ID NO: 15;
(f) SEQ ID NO: 16 or a mature polypeptide thereof; or SEQ ID NO: 17;
(g) SEQ ID NO: 41 or a mature polypeptide thereof; or SEQ ID NO: 42;
(h) SEQ ID NO: 44 or a mature polypeptide thereof; or SEQ ID NO: 45;
(i) SEQ ID NO: 47 or a mature polypeptide thereof; or SEQ ID NO: 48;
(j) SEQ ID NO: 50 or a mature polypeptide thereof; or SEQ ID NO: 51;
(k) SEQ ID NO: 53 or a mature polypeptide thereof; or SEQ ID NO: 54;
(I) SEQ ID NO: 56 or a mature polypeptide thereof; or SEQ ID NO: 57; or
(m) SEQ ID NO: 59 or a mature polypeptide thereof; or SEQ ID NO: 60.

6. An isolated or purified polypeptide comprising a catalytic domain selected from the group consisting of:
(i)
(a) a catalytic domain having at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 18 to 497 of SEQ ID NO: 5;
(b) a catalytic domain encoded by a polynucleotide that hybridizes under medium, medium-high, high, or very high stringency conditions with the full-length complement of nucleotides 52 to 1943 of SEQ ID NO: 4, or the cDNA sequence thereof;
(c) a catalytic domain encoded by a polynucleotide having at least at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 52 to 1943 of SEQ ID NO: 4, or the cDNA sequence thereof; and
(d) a fragment of the catalytic domain of (a), (b), or (c) that has alpha-amylase activity;
(ii)
(a) a catalytic domain having at least 94%, at least 95%, at least 96%, at least 97%, at least 98%, at least 99%, or 100% sequence identity to amino acids 22 to 495 of SEQ ID NO: 8;
(b) a catalytic domain encoded by a polynucleotide that hybridizes under medium, medium-high, high, or very high stringency conditions with the full-length complement of nucleotides 64 to 1925 of SEQ ID NO: 7, or the cDNA sequence thereof;
(c) a catalytic domain encoded by a polynucleotide having at least at least 94%, at least 95%, at least 96%, at least 97%, at least 98%, at least 99%, or 100% sequence identity to nucleotides 64 to 1925 of SEQ ID NO: 7, or the cDNA sequence thereof; and
(d) a fragment of the catalytic domain of (a), (b), or (c) that has alpha-amylase activity;
(iii)
(a) a catalytic domain having at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 20 to 496 of SEQ ID NO: 11;
(b) a catalytic domain encoded by a polynucleotide that hybridizes under medium, medium-high, high, or very high stringency conditions with the full-length complement of nucleotides 58 to 1488 of SEQ ID NO: 10;
(c) a catalytic domain encoded by a polynucleotide having at least at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 58 to 1488 of SEQ ID NO: 10; and
(d) a fragment of the catalytic domain of (a), (b), or (c) that has alpha-amylase activity;
(iv)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 30 to 469 of SEQ ID NO: 14;
(b) a catalytic domain encoded by a polynucleotide that hybridizes under medium, medium-high, high, or very high stringency conditions with the full-length complement of nucleotides 88 to 1407 of SEQ ID NO: 13;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 88 to 1407 of SEQ ID NO: 13; and
(d) a fragment of the catalytic domain of (a), (b), or (c) that has alpha-amylase activity;
(v)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 28 to 465 of SEQ ID NO: 41;
(b) a catalytic domain encoded by a polynucleotide that hybridizes under medium, medium-high, high, or very high stringency conditions with the full-length complement of nucleotides 82 to 1395 of SEQ ID NO: 40;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 82 to 1395 of SEQ ID NO: 40; and
(d) a fragment of the catalytic domain of (a), (b), or (c) that has alpha-amylase activity;
(vi)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 44;
(b) a catalytic domain encoded by a polynucleotide that hybridizes under medium, medium-high, high, or very high stringency conditions with the full-length complement of nucleotides 133 to 1401 of SEQ ID NO: 43;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 133 to 1401 of SEQ ID NO: 43; and
(d) a fragment of the catalytic domain of (a), (b), or (c) that has alpha-amylase activity;
(vii)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 47;
(b) a catalytic domain encoded by a polynucleotide that hybridizes under medium, medium-high, high, or very high stringency conditions with the full-length complement of nucleotides 133 to 1401 of SEQ ID NO: 46;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 133 to 1401 of SEQ ID NO: 46; and
(d) a fragment of the catalytic domain of (a), (b), or (c) that has alpha-amylase activity;
(viii)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 50;
(b) a catalytic domain encoded by a polynucleotide that hybridizes under medium, medium-high, high, or very high stringency conditions with the full-length complement of nucleotides 133 to 1401 of SEQ ID NO: 49;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 133 to 1401 of SEQ ID NO: 49; and
(d) a fragment of the catalytic domain of (a), (b), or (c) that has alpha-amylase activity;
(ix)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 53;
(b) a catalytic domain encoded by a polynucleotide that hybridizes under medium, medium-high, high, or very high stringency conditions with the full-length complement of nucleotides 133 to 1401 of SEQ ID NO: 52;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 133 to 1401 of SEQ ID NO: 52; and
(d) a fragment of the catalytic domain of (a), (b), or (c) that has alpha-amylase activity;
(x)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 56;
(b) a catalytic domain encoded by a polynucleotide that hybridizes under medium, medium-high, high, or very high stringency conditions with the full-length complement of nucleotides 133 to 1401 of SEQ ID NO: 55;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 133 to 1401 of SEQ ID NO: 55; and
(d) a fragment of the catalytic domain of (a), (b), or (c) that has alpha-amylase activity; and
(xi)
(a) a catalytic domain having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 45 to 467 of SEQ ID NO: 59;
(b) a catalytic domain encoded by a polynucleotide that hybridizes under medium, medium-high, high, or very high stringency conditions with the full-length complement of nucleotides 133 to 1401 of SEQ ID NO: 58;
(c) a catalytic domain encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 133 to 1401 of SEQ ID NO: 58; and
(d) a fragment of the catalytic domain of (a), (b), or (c) that has alpha-amylase activity.

7. The polypeptide of claim 6, further comprising at least a first starch binding module, and optionally further comprising: (i) a linker positioned between and connecting the catalytic domain to the first starch binding module; and (ii) at least a second starch binding module connected to the first starch binding module.

8. An isolated or purified polypeptide comprising a starch binding module and a catalytic domain, wherein the binding module is selected from the group consisting of:
(i)
(a) a starch binding module having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 532 to 632 of SEQ ID NO: 5;
(b) a starch binding module encoded by a polynucleotide that hybridizes under medium, medium-high, high, or very high stringency conditions with the full-length complement of nucleotides 2046 to 2348 of SEQ ID NO: 4, or the cDNA sequence thereof;
(c) a starch binding module encoded by a polynucleotide having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 2046 to 2348 of SEQ ID NO: 4, or the cDNA sequence thereof; and
(d) a fragment of the starch binding module of (a), (b), or (c) that has binding activity;
(ii)
(a) a starch binding module having at least 91%, at least 92%, at least 93%, at least 94%, at least 95%, at least 96%, at least 97%, at least 98%, at least 99%, or 100% sequence identity to amino acids 532 to 632 of SEQ ID NO: 8;
(b) a starch binding module encoded by a polynucleotide that hybridizes under medium, medium-high, high, or very high stringency conditions with the full-length complement of nucleotides 2034 to 2336 of SEQ ID NO: 7, or the cDNA sequence thereof;
(c) a starch binding module encoded by a polynucleotide having sat least 91%, at least 92%, at least 93%, at least 94%, at least 95%, at least 96%, at least 97%, at least 98%, at least 99%, or 100% sequence identity to nucleotides 2034 to 2336 of SEQ ID NO: 7, or the cDNA sequence thereof; and
(d) a fragment of the starch binding module of (a), (b), or (c) that has binding activity;
(iii)
(a) a starch binding module having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 508 to 601 of SEQ ID NO: 11;
(b) a starch binding module encoded by a polynucleotide that hybridizes under medium, medium-high, high, or very high stringency conditions with the full-length complement of nucleotides 1504 to 1827 of SEQ ID NO: 10;
(c) a starch binding module encoded by a polynucleotide having at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1504 to 1827 of SEQ ID NO: 10; and
(d) a fragment of the starch binding module of (a), (b), or (c) that has binding activity;
(iv)
(a) a starch binding module having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 554 to 653 of SEQ ID NO: 41;
(b) a starch binding module encoded by a polynucleotide that hybridizes under medium, medium-high, high, or very high stringency conditions with the full-length complement of nucleotides 1660 to 1959 of SEQ ID NO: 40;
(c) a starch binding module encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1660 to 1959 of SEQ ID NO: 40; and
(d) a fragment of the starch binding module of (a), (b), or (c) that has binding activity
(v)
(a) a starch binding module having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 478 to 569 of SEQ ID NO: 44;
(b) a starch binding module encoded by a polynucleotide that hybridizes under medium, medium-high, high, or very high stringency conditions with the full-length complement of nucleotides 1432 to 1707 of SEQ ID NO: 43;
(c) a starch binding module encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1432 to 1707 of SEQ ID NO: 43; and
(d) a fragment of the starch binding module of (a), (b), or (c) that has binding activity;
(vi)
(a) a starch binding module having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 470 to 565 of SEQ ID NO: 47, amino acids 475 to 571 of SEQ ID NO: 50, or amino acids 655 to 750 of SEQ ID NO: 53;
(b) a starch binding module encoded by a polynucleotide that hybridizes under medium, medium-high, high, or very high stringency conditions with the full-length complement of nucleotides 1408 to 1695 of SEQ ID NO: 46, nucleotides 1423 to 1713 of SEQ ID NO: 49, or nucleotides 1963 to 2250 of SEQ ID NO: 52;
(c) a starch binding module encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1408 to 1695 of SEQ ID NO: 46, nucleotides 1423 to 1713 of SEQ ID NO: 49, or nucleotides 1963 to 2250; and
(d) a fragment of the starch binding module of (a), (b), or (c) that has binding activity;
(vii)
(a) a starch binding module having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 563 to 654 of SEQ ID NO: 53;
(b) a starch binding module encoded by a polynucleotide that hybridizes under medium, medium-high, high, or very high stringency conditions with the full-length complement of nucleotides 1687 to 1962 of SEQ ID NO: 52;
(c) a starch binding module encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1687 to 1962 of SEQ ID NO: 52; and
(d) a fragment of the starch binding module of (a), (b), or (c) that has binding activity;
(viii)
(a) a starch binding module having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 476 to 569 of SEQ ID NO: 56;
(b) a starch binding module encoded by a polynucleotide that hybridizes under medium, medium-high, high, or very high stringency conditions with the full-length complement of nucleotides 1426 to 1707 of SEQ ID NO: 55;
(c) a starch binding module encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1426 to 1707 of SEQ ID NO: 55; and
(d) a fragment of the starch binding module of (a), (b), or (c) that has binding activity; and
(ix)
(a) a starch binding module having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to amino acids 479 to 572 of SEQ ID NO: 59;
(b) a starch binding module encoded by a polynucleotide that hybridizes under medium, medium-high, high, or very high stringency conditions with the full-length complement of nucleotides 1435 to 1716 of SEQ ID NO: 58;
(c) a starch binding module encoded by a polynucleotide having at least 70%, at least 71%, at least 72%, at least 73%, at least 74%, at least 75%, at least 76%, at least 77%, at least 78%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to nucleotides 1435 to 1716 of SEQ ID NO: 58; and
(d) a fragment of the starch binding module of (a), (b), or (c) that has binding activity.

9. The polypeptide of claim 8, wherein the catalytic domain is obtained from a hydrolase, isomerase, ligase, lyase, oxidoreductase, transferase, an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

10. A granule, which comprises:
(a) a core comprising the polypeptide of claim 1, and, optionally
(b) a coating consisting of one or more layer(s) surrounding the core.

11. A granule, which comprises:
(a) a core, and
(b) a coating consisting of one or more layer(s) surrounding the core, wherein the coating comprises the polypeptide of claim 1.

12. A composition comprising the polypeptide of claim 1.

13. A whole broth formulation or cell culture composition comprising the polypeptide of claim 1.

14. An isolated or purified polynucleotide encoding the polypeptide of claim 1.

15. A nucleic acid construct or expression vector comprising the polynucleotide of claim 14, wherein the polynucleotide is operably linked to one or more control sequences that direct the production of the polypeptide in an expression host.

16. A recombinant host cell comprising the polynucleotide of claim 14 operably linked to one or more control sequences that direct the production of the polypeptide.

17. The host cell of claim 16, which is a yeast host cell.

18. A composition comprising the recombinant host cell of claim 17 and one or more naturally occurring and/or non-naturally occurring components, such as components are selected from the group consisting of: surfactants, emulsifiers, gums, swelling agents, and antioxidants.

19. A method of producing a polypeptide having alpha-amylase activity, comprising cultivating the recombinant host cell of claim 16 under conditions conducive for production of the polypeptide, and optionally recovering the polypeptide.

20. A process for producing fermentation products from starch-containing material comprising the steps of:
i) liquefying the starch-containing material at a temperature above the initial gelatinization temperature using an alpha-amylase;
ii) saccharifying using a carbohydrate-source generating enzyme;
iii) fermenting using a fermenting organism;
wherein: (i) the polypeptide of claim 1, (ii) a composition comprising the polypeptide, (iii) a whole broth formulation or cell culture composition comprising the polypeptide, or (iv) a yeast host cell expressing the polypeptide, or a composition comprising the yeast host cell and one or more components selected from the group consisting of surfactants, emulsifiers, gums, swelling agents, and antioxidants, is present or added during fermentation or simultaneous saccharification and fermentation.


SEQUENCE LISTING (65 sequences)

SEQ ID NO: 1 - DNA, 1812 bp, Artificial Sequence
  Hybrid alpha-amylase Penicillium oxalicum catalyic domain and Aspergillus rolfsii starch binding domain
atgaaattccttggactagctgctttgtttcttgcccagaccgtggcgggtctgacggctgcccaatggcgcagccaatcgatctacttcctcatgactgatcggtttggtcgaaccgacaagtccgtgactgccccatgcaacacaaatgaccgagtatactgtggtggaacttggcaaggcattatcaatcaattggattacatccaaggaatgggatttactgcgatttggatcactccagtcacagagcagctccctcaagatactggggacggtgaagcataccacggatattggcaacaggaaatatacaatgtcaacaacaactacggcactgctgccgatctcaaggctctttcgcaagcccttcacagtcgtggaatgtacctcatggttgatgttgtcgcgaaccacatgggctacgcgggggccggaaacaccgtcgactacagtgtgttcaagccattcagctcctcttcgtacttccacccgtattgtttgatcagcgattactcaaatcagacaaatgtcgaggactgttggcttggggacaccaccgtctcactccccgatcttgataccactctttcttccgtgcagacgatctggtataattgggtcagtgacctagtttcaaactattccatcgacggtcttcggatcgatactgtcaagcacgttcaaaagtcattctggcctggctatcagagtgctgctggtgtctactgtgttggagaggtcttcagtggtgatccggcttacacttgcccttatcaaaactaccttgatggggttttgaattatccaatctattaccaattactcggcgcatttaaatcaaccagtggaagcatcagcagcctttacaacatgatcaactctgttgcgtccgactgtgctgacccaaccctgctgggtaactttattgaaaaccatgataacccacgctttgcttcgtacacaagcgactactcgcaagcgaaaaatgtcatctcgttcattttcttgtctgatggcattccgatcgtctatgccggacaggaacagcactacagtggcggtaacgaccctgctaaccgtgaagcgacttggctttccggatactcaaagaacgctcaactgtaccaacacattgcttcgacgaacaaaatccgcagtcttgcgatttcaaaggatgccaactacatcacttccaagaacaatgctttctacactgacagtaacaccatcgccatgaagaaaggatcctccggatcgcaggtcgtgacagttctctcaaaccgtggctcgtcaggtagctcgtacactttgagtctcagcggcagcggctatgcggctggcaccaagctggtggaaatgtacacctgcaccgctgttaccgttgattcgaatggcaacatcgcagtttccatgacctctgggctgcctcgagtgtttatgctggcctcctctgcatgctctctttgcagttccgcggaattcggtgctacaagcccgggtggctcctcgggtagtgtcgaggtcactttcgacgtttacgctaccacagtatatggccagaacatctatatcaccggtgatgtgagtgagctcggcaactggacacccgccaatggtgttgcactctcttctgctaactaccccacctggagtgccacgatcgctctccccgctgacacgacaatccagtacaagtatgtcaacattgacggcagcaccgtcatctgggaggatgctatcagcaatcgcgagatcacgacgcccgccagcggcacatacaccgaaaaagacacttgggatgaatcttag

SEQ ID NO: 2 - protein, 603 aa, Artificial Sequence
  Hybrid alpha-amylase Penicillium oxalicum catalyic domain and Aspergillus rolfsii starch binding domain
MKFLGLAALFLAQTVAGLTAAQWRSQSIYFLMTDRFGRTDKSVTAPCNTNDRVYCGGTWQGIINQLDYIQGMGFTAIWITPVTEQLPQDTGDGEAYHGYWQQEIYNVNNNYGTAADLKALSQALHSRGMYLMVDVVANHMGYAGAGNTVDYSVFKPFSSSSYFHPYCLISDYSNQTNVEDCWLGDTTVSLPDLDTTLSSVQTIWYNWVSDLVSNYSIDGLRIDTVKHVQKSFWPGYQSAAGVYCVGEVFSGDPAYTCPYQNYLDGVLNYPIYYQLLGAFKSTSGSISSLYNMINSVASDCADPTLLGNFIENHDNPRFASYTSDYSQAKNVISFIFLSDGIPIVYAGQEQHYSGGNDPANREATWLSGYSKNAQLYQHIASTNKIRSLAISKDANYITSKNNAFYTDSNTIAMKKGSSGSQVVTVLSNRGSSGSSYTLSLSGSGYAAGTKLVEMYTCTAVTVDSNGNIAVSMTSGLPRVFMLASSACSLCSSAEFGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 3 - protein, 583 aa, Artificial Sequence
  Hybrid alpha-amylase Penicillium oxalicum catalyic domain and Aspergillus rolfsii starch binding domain
AQWRSQSIYFLMTDRFGRTDKSVTAPCNTNDRVYCGGTWQGIINQLDYIQGMGFTAIWITPVTEQLPQDTGDGEAYHGYWQQEIYNVNNNYGTAADLKALSQALHSRGMYLMVDVVANHMGYAGAGNTVDYSVFKPFSSSSYFHPYCLISDYSNQTNVEDCWLGDTTVSLPDLDTTLSSVQTIWYNWVSDLVSNYSIDGLRIDTVKHVQKSFWPGYQSAAGVYCVGEVFSGDPAYTCPYQNYLDGVLNYPIYYQLLGAFKSTSGSISSLYNMINSVASDCADPTLLGNFIENHDNPRFASYTSDYSQAKNVISFIFLSDGIPIVYAGQEQHYSGGNDPANREATWLSGYSKNAQLYQHIASTNKIRSLAISKDANYITSKNNAFYTDSNTIAMKKGSSGSQVVTVLSNRGSSGSSYTLSLSGSGYAAGTKLVEMYTCTAVTVDSNGNIAVSMTSGLPRVFMLASSACSLCSSAEFGATSPGGSSGSVEVTFDVYATTVYGQNIYITGDVSELGNWTPANGVALSSANYPTWSATIALPADTTIQYKYVNIDGSTVIWEDAISNREITTPASGTYTEKDTWDES

SEQ ID NO: 4 - DNA, 2351 bp, Penicillium sclerotiorum
atgaatcgagttctattagcttcgacactattccttgcccaggccttgagccttacacccgcggaatggcgcagtcaatctatctactttctgttgacggaccgattcgctcggacggacggttcaactacagcgacatgcgatgtctctgatagagtaggatctgaacttcgttacttgaccagaaacatcaagatcaaatctgacatggcattgatagatctactgcggaggcagctggcaagggataataaatcaggtattattccctttaaatatatacaaggattgattcgaatgctaagattatgctagctggactatatccaagggatgggctttactgccatctggattacccccgtcactgagcaattgtctcaggacacaggagacggtgaggcatatcacggatattggcagcagaagatgtaggagccagttgttaccattgatatatactgctcctactaacttgctcagctacgatgtcaacacgaactacggaacggcagctgatctcttagccttatccgaggccttacatgcccgagacatgtatctaatggtggacgttgttgcaaaccacatggtaagtcctcatttcctgtaaaaaacatgaatcatctcttcttatgcttttaatagggctatgatggagcgggcagtagtgtggactacagcgtctttgatccgttcgattcttcctcttactttcattcttactgcctcatcagtaactatgataaccagaccaatgtcgaggattgctggcttggagatacaacggtctctctgccggatctcaataccgagctgtcctctgttgagaccatatggtatgactggattgcagatctagtctccaactactccagtaagtaatcggtcactaaagaacataatgctgcctggcgagatctgacgttaattacagttgatggacttcgaatcgataccgtgaaacatgtccaagaatccttttggccgggctacaatgatgccgcaggtgtttactgtgttggggaagtttttgatggcgatccggcatacacctgcccttaccagaattatctggacggagtgctgaactacccaatgtaagtccctttccgtctactcttccactaagctaatatattgcaaagttactaccagttgctgtacgctttcgaagattcaagcggaagcatcagtgacctgtacaacatgatcaactcggtcgcttctgactgcgctgatcccacactgcttggaaacttcattgagaaccacgacaacccaagatttgcctcgtaagtactctgaagctttattcggctcttactgactggtgaaaagctacactgatgattactcccaagcgaagaatgtaatctcctttatattcctctctgacggcattccgatcgtctacgcaggccaggagcagcactacagcggcggtgctgacccctataaccgagaagcaacttggctgtcgggatactctaccactgctgagctgtacgagtacatcgcaacgaccaacaaaatccgcagcctcgccatctcctctgattcctcctacattacttccgaggtaaagtcaacctacaccccattcctcatgaattcacttcgagtcataactaacgttaaggaatattaagaacgatgctttctaccaggatagccacaccttggctatgaagaagggatccagtggctcccaagtcattaccgtgctctccaactacggtgcctctggtagctcatacactttgaccctgagcggtagtgggtattcctcaggtacccagcttatggagttgtacacatgtacctccgtgacagtagactcgagcggcaatatcgccgtacccatggcatctggtctaccccgggtctttatgcttgcctctgctgtatccagtagcagcatttgcggtgggtcgtcatccgctacaacaacatcgtcctcaactaagacatccactacattgacgacgacaacttctaagacgacaactgcgtcttcctgcacccaggccacggctttgcctgttttgtttgaggagatcgtgacgacaacctatggtgaggatatctacatctccggttcgatcagtcagctaggctcgtgggataccgatgatgctattgccttgtctgcggatgagtacacttcatcgaatcatctatggtacgttgttgtcaccattccagtgggaacgtccttccagtacaagtttattgaaaagacggacggttcatcttctgtgagttgggaaagcgatccaaatagatcttactctgtcccgaccggatgcgtgggctctactgcaactgtgacggcaacctggagatga

SEQ ID NO: 5 - protein, 632 aa, Penicillium sclerotiorum
MNRVLLASTLFLAQALSLTPAEWRSQSIYFLLTDRFARTDGSTTATCDVSDRIYCGGSWQGIINQLDYIQGMGFTAIWITPVTEQLSQDTGDGEAYHGYWQQKIYDVNTNYGTAADLLALSEALHARDMYLMVDVVANHMGYDGAGSSVDYSVFDPFDSSSYFHSYCLISNYDNQTNVEDCWLGDTTVSLPDLNTELSSVETIWYDWIADLVSNYSIDGLRIDTVKHVQESFWPGYNDAAGVYCVGEVFDGDPAYTCPYQNYLDGVLNYPIYYQLLYAFEDSSGSISDLYNMINSVASDCADPTLLGNFIENHDNPRFASYTDDYSQAKNVISFIFLSDGIPIVYAGQEQHYSGGADPYNREATWLSGYSTTAELYEYIATTNKIRSLAISSDSSYITSENDAFYQDSHTLAMKKGSSGSQVITVLSNYGASGSSYTLTLSGSGYSSGTQLMELYTCTSVTVDSSGNIAVPMASGLPRVFMLASAVSSSSICGGSSSATTTSSSTKTSTTLTTTTSKTTTASSCTQATALPVLFEEIVTTTYGEDIYISGSISQLGSWDTDDAIALSADEYTSSNHLWYVVVTIPVGTSFQYKFIEKTDGSSSVSWESDPNRSYSVPTGCVGSTATVTATWR

SEQ ID NO: 6 - protein, 615 aa, Penicillium sclerotiorum
LTPAEWRSQSIYFLLTDRFARTDGSTTATCDVSDRIYCGGSWQGIINQLDYIQGMGFTAIWITPVTEQLSQDTGDGEAYHGYWQQKIYDVNTNYGTAADLLALSEALHARDMYLMVDVVANHMGYDGAGSSVDYSVFDPFDSSSYFHSYCLISNYDNQTNVEDCWLGDTTVSLPDLNTELSSVETIWYDWIADLVSNYSIDGLRIDTVKHVQESFWPGYNDAAGVYCVGEVFDGDPAYTCPYQNYLDGVLNYPIYYQLLYAFEDSSGSISDLYNMINSVASDCADPTLLGNFIENHDNPRFASYTDDYSQAKNVISFIFLSDGIPIVYAGQEQHYSGGADPYNREATWLSGYSTTAELYEYIATTNKIRSLAISSDSSYITSENDAFYQDSHTLAMKKGSSGSQVITVLSNYGASGSSYTLTLSGSGYSSGTQLMELYTCTSVTVDSSGNIAVPMASGLPRVFMLASAVSSSSICGGSSSATTTSSSTKTSTTLTTTTSKTTTASSCTQATALPVLFEEIVTTTYGEDIYISGSISQLGSWDTDDAIALSADEYTSSNHLWYVVVTIPVGTSFQYKFIEKTDGSSSVSWESDPNRSYSVPTGCVGSTATVTATWR

SEQ ID NO: 7 - DNA, 2339 bp, Penicillium wotroi
atgaaagcacttgcattgaccgcggtgttgcttacgaaggctgtcgtcggcctgacgcctgcagaatggcgaagtcagtcgatttactttcttctgactgatcgatttgggcgtacagataattctacaacagcaacatgcaatgttagcgaccgtgtaaggactcaaatacccccttccccggtacagctcgtattactaaatgactgcattagatttactgcggtggtagctggcaaggaatcatcaaccacgtacgctgctcttccacctcctaattctcaagcactgctaagtctccgtcaagttggactatattcaagacatgggattcactgctatttggattacccccgtcacagaacagctttctcaagatactggagatggcgaagcatatcatggatactggcaacaggagatgttcgtgggctctgttgaccctgaagctacgattgaactgacgcttttttattagatacaatgtcaacacgaactatggcactgcttctgatcttttggcactctctgaagctctacatagccgtggcatgtatctcatggtagatgtggttgcaaaccatatggtatgcgctctattcataacaaagcgatctggtctctgactaaatatacagggatacgatggagctggtaacactgttgactacagtgttttcaacccatttgactcttcctcttacttccattcgtattgtgagatcagcgattactctaaccagacaaacgtggaagactgttggctaggagacactacggtctcgcttcccgatcttgacacgacactttcttctgttcaaacaatctggtatgactgggtggccgctttggtgtcgaattattcaagtatgtacaaattgcgaggtattttacaaagtcctgagatccgctaacatactacgacaaagttgatggattacgagttgatacggtcaagcacgtccagaagtcattttggcccggctacaatagtgcggcaggcgtctactgtgttggagaggtctttgatggagacccagcatacacatgcccttaccaggactacatggatggcgttttgaactatccgatgtatgttttcacccctgggactgttattttgatggatgttttcctgacctacaccagttattatcaactgctgtatgcattcgagtcaacaagcggcagtatcagcaacctatacaatatgatcagctccgttgcgtctgattgctctgacactaccttgctgggaaacttcattgaaaatcatgacaacccacgatttgcttcgtatgacccccaagtgtggattaacgttcgctactaacacctccactttagctacacaagtgactactctcaagcgaagaatgtgctttctttcatcttcttctctgatggaatccctatcgtctatgctggccaggaacaacactatagcggcggtagcgatccggctaatcgtgaggcaacctggctatccggatacgatacaagcgcggaactctacacgtggattgcctccaccaacaagatccgcagtctggctatctctaaggatactgcctacgtaacatccaaggtacgcattggcaacttatgtcttatacgacttaactaacaacagcatgcagaatgatgcattctacaccgacagcagtaccatcgccatgaagaaaggatcgagtggctcacaagtcatcactgttctgtcaaaccttggctcatcgggtagctcgtataccttgactctcagtggaagcggctattcatccggcacgaagctgatggagatgtacacctgcactgccgttactgtggactctagtggtggcattgcggtacccatggcttctgggttgcctcgagtatatatgctggcttcttcggcttgttcgatctgtagttctgcctgctcgacgactacaacttcatcgacgacctcgactgcgacaacgacgtcaaccactcttaaaactactacatctaccacctctacgagctgtacacaggctactgctttgcctgttctgtttcaagaaattgttaccacttcatacggacaaaacatctatatctcgggctcgatcagtgagctcggtgactgggacacttctaacgccgttgccctctctgccaacaagtacacatcatccgatcatctttggtatgttgttgtgacaattcctgtgggaacgtcattcgagtacaagtttatcgaggagacgagtgggtctagtagtatcacttgggagagtgatcctaaccgctcgtacacggtcccaactggctgtgccggctcaacagcaaccgttacggcaacatggagatag

SEQ ID NO: 8 - protein, 632 aa, Penicillium wotroi
MKALALTAVLLTKAVVGLTPAEWRSQSIYFLLTDRFGRTDNSTTATCNVSDRIYCGGSWQGIINHLDYIQDMGFTAIWITPVTEQLSQDTGDGEAYHGYWQQEIYNVNTNYGTASDLLALSEALHSRGMYLMVDVVANHMGYDGAGNTVDYSVFNPFDSSSYFHSYCEISDYSNQTNVEDCWLGDTTVSLPDLDTTLSSVQTIWYDWVAALVSNYSIDGLRVDTVKHVQKSFWPGYNSAAGVYCVGEVFDGDPAYTCPYQDYMDGVLNYPIYYQLLYAFESTSGSISNLYNMISSVASDCSDTTLLGNFIENHDNPRFASYTSDYSQAKNVLSFIFFSDGIPIVYAGQEQHYSGGSDPANREATWLSGYDTSAELYTWIASTNKIRSLAISKDTAYVTSKNDAFYTDSSTIAMKKGSSGSQVITVLSNLGSSGSSYTLTLSGSGYSSGTKLMEMYTCTAVTVDSSGGIAVPMASGLPRVYMLASSACSICSSACSTTTTSSTTSTATTTSTTLKTTTSTTSTSCTQATALPVLFQEIVTTSYGQNIYISGSISELGDWDTSNAVALSANKYTSSDHLWYVVVTIPVGTSFEYKFIEETSGSSSITWESDPNRSYTVPTGCAGSTATVTATWR

SEQ ID NO: 9 - protein, 611 aa, Penicillium wotroi
EWRSQSIYFLLTDRFGRTDNSTTATCNVSDRIYCGGSWQGIINHLDYIQDMGFTAIWITPVTEQLSQDTGDGEAYHGYWQQEIYNVNTNYGTASDLLALSEALHSRGMYLMVDVVANHMGYDGAGNTVDYSVFNPFDSSSYFHSYCEISDYSNQTNVEDCWLGDTTVSLPDLDTTLSSVQTIWYDWVAALVSNYSIDGLRVDTVKHVQKSFWPGYNSAAGVYCVGEVFDGDPAYTCPYQDYMDGVLNYPIYYQLLYAFESTSGSISNLYNMISSVASDCSDTTLLGNFIENHDNPRFASYTSDYSQAKNVLSFIFFSDGIPIVYAGQEQHYSGGSDPANREATWLSGYDTSAELYTWIASTNKIRSLAISKDTAYVTSKNDAFYTDSSTIAMKKGSSGSQVITVLSNLGSSGSSYTLTLSGSGYSSGTKLMEMYTCTAVTVDSSGGIAVPMASGLPRVYMLASSACSICSSACSTTTTSSTTSTATTTSTTLKTTTSTTSTSCTQATALPVLFQEIVTTSYGQNIYISGSISELGDWDTSNAVALSANKYTSSDHLWYVVVTIPVGTSFEYKFIEETSGSSSITWESDPNRSYTVPTGCAGSTATVTATWR

SEQ ID NO: 10 - DNA, 1830 bp, Talaromyces helicus
atgaaactgtcaaccctttccctcctctggggctatgtcgccaacaccgctctcggactcagcgccagcggctggcgcagccagtccatctacttcctcatgaccgaccggtttggtcttaccaacggctctacgactgctccgtgcaatgctgccgatggcatttactgcggtggcagctggcagggcgtcattaaccatttggactatatccagggaatgggcttcaccgcactatggatcaccccagtcaccgagaacttcttgggcaacacacaggacggtgagagttaccacggatactggcagcaaaacgcctatgcagtcaacgcccagttcgggagcagctccgaccttctcgcgctgtcgaaggcgctgcatgcccgtggcatgtatttgatggttgacatcgtggtcaataacatggcgtacgatggtcctggcacaaacgtcgattactccattttcaacccattcccctccgagtcctactaccatccgtactgcctcatcagcaattacaacaaccagaccaacgtggaggactgttgggagggcgacacaattgtttcgcttcccgatttggacaccacgcagacctacgtgcaggacacctggaatgcctgggtgaagagcttcgtctccaactactcgatcgatggcctgaggattgactccgctaaacacatgcaaaaagactttttccccaatttcgaggattccgcaaatgtgtactgcataggggaagttcttgatggtgacccgacttatgtgtgcccgtatcaggattatctgaacggcgtgttgaactaccctatctactaccaactgctctacgcatttgagtcctcgtccggatccatctccaacctctacgacatgattaacagcgtcaagagcgattgtgccgacagtacccttcttggcaatttcgccgagaaccacgacaatcctcgtttcgcccattacacctcggacttcagccaggctaagtcggtgatcagcttcattttccttacagacggcatcccaatcctctactatggccaagagcagcacttcagtggtggcaatgttccgctcaaccgcgagcctctctggcccagcgactattcgacgaccgctgagctctacacccatactgcgacgaccaatgctatccgaacgcttgctatgaagaaggacgcaggttttctcacatacaagaataccccgttctaccaagattccaataccatcgcgatgcgcaaaggtaccactggactccagctcatcactgtactttcgaacctaggttcctcggggtcgtcgtacacgctgtcgctatcaggctccgggtacacttccggcacgcttctcactgagctctacacgtgtaccaacgttacagtatcctcatccggcacgattgctgtccccatgtcctctggcgaacctcgcgctttcctgccttggtccagcgtgtcaggcagctccctctgccagggaactgctgtgagctcctgtaccactgcgtcttctgtcgcggtcactttcaatgagctcgtcaccacttcttacggccaggaggtataccttgccggttccatctctcagctggggagctggtccacctcgaacgccgtgttgctctccgctagcaagtatacttcctccaaccctttgtggttcgtcaccgtgaaccttcccgctggcactacattccagtacaagttcatcatcaagaacaccgacggttccttcacttgggaaagcgatcccaaccgatcctacaccgtcccaacgggctgccagggcctcactgccacagtgaacgacacctggcgctaa

SEQ ID NO: 11 - protein, 609 aa, Talaromyces helicus
MKLSTLSLLWGYVANTALGLSASGWRSQSIYFLMTDRFGLTNGSTTAPCNAADGIYCGGSWQGVINHLDYIQGMGFTALWITPVTENFLGNTQDGESYHGYWQQNAYAVNAQFGSSSDLLALSKALHARGMYLMVDIVVNNMAYDGPGTNVDYSIFNPFPSESYYHPYCLISNYNNQTNVEDCWEGDTIVSLPDLDTTQTYVQDTWNAWVKSFVSNYSIDGLRIDSAKHMQKDFFPNFEDSANVYCIGEVLDGDPTYVCPYQDYLNGVLNYPIYYQLLYAFESSSGSISNLYDMINSVKSDCADSTLLGNFAENHDNPRFAHYTSDFSQAKSVISFIFLTDGIPILYYGQEQHFSGGNVPLNREPLWPSDYSTTAELYTHTATTNAIRTLAMKKDAGFLTYKNTPFYQDSNTIAMRKGTTGLQLITVLSNLGSSGSSYTLSLSGSGYTSGTLLTELYTCTNVTVSSSGTIAVPMSSGEPRAFLPWSSVSGSSLCQGTAVSSCTTASSVAVTFNELVTTSYGQEVYLAGSISQLGSWSTSNAVLLSASKYTSSNPLWFVTVNLPAGTTFQYKFIIKNTDGSFTWESDPNRSYTVPTGCQGLTATVNDTWR

SEQ ID NO: 12 - protein, 590 aa, Talaromyces helicus
LSASGWRSQSIYFLMTDRFGLTNGSTTAPCNAADGIYCGGSWQGVINHLDYIQGMGFTALWITPVTENFLGNTQDGESYHGYWQQNAYAVNAQFGSSSDLLALSKALHARGMYLMVDIVVNNMAYDGPGTNVDYSIFNPFPSESYYHPYCLISNYNNQTNVEDCWEGDTIVSLPDLDTTQTYVQDTWNAWVKSFVSNYSIDGLRIDSAKHMQKDFFPNFEDSANVYCIGEVLDGDPTYVCPYQDYLNGVLNYPIYYQLLYAFESSSGSISNLYDMINSVKSDCADSTLLGNFAENHDNPRFAHYTSDFSQAKSVISFIFLTDGIPILYYGQEQHFSGGNVPLNREPLWPSDYSTTAELYTHTATTNAIRTLAMKKDAGFLTYKNTPFYQDSNTIAMRKGTTGLQLITVLSNLGSSGSSYTLSLSGSGYTSGTLLTELYTCTNVTVSSSGTIAVPMSSGEPRAFLPWSSVSGSSLCQGTAVSSCTTASSVAVTFNELVTTSYGQEVYLAGSISQLGSWSTSNAVLLSASKYTSSNPLWFVTVNLPAGTTFQYKFIIKNTDGSFTWESDPNRSYTVPTGCQGLTATVNDTWR

SEQ ID NO: 13 - DNA, 1428 bp, Lactobacillus amylovorus
atgaaacaacaaaaacggctttacgcccgattgctgacgctgttatttgcgctcatcttcttgctgcctcattctgcagcagcggcggctagtgatacgacatcaactgatcactcaagcaatgatacagctgattctgttagcgacggtgttattttgcatgcatggtgctggtcgttcaacacgattaaaaacaacttgaaacagattcatgacgccggctacacagcggttcaaacttcacctgttaatgaagttaaagttggaaatagcgggtctaagtcattaaataactggtattggctatatcagccaactaaatatagtattggtaactattatttaggaacggaagctgaatttaagtcaatgtgcgctgctgctaaagaatataatatcaggatcattgtcgatgcaactctgaatgatacaacaagtgattatagtgcaatttcggatgaaattaaaagtattccaaattggacacatggtaacaaacaaatttcgaattggagtgatcgtgaagatgttactcaaaattcgttgttaggtttatatgattggaatactcaaaattctcaagtccagacgtatttgaagaattatttggaacgcttgatttctgacggagcttcaggctttcgttatgatgcagctaagcatattgaacttccaagtcaatatgatggcagctatggcagcaatttctggccaaatattactgataatgggtctgaatttcagtatggtgaagttttgcaggactcgatttcaaaagaatcagattatgctaattacatgagtgttacagcttcaaattacggcaatacgattcgcaatgcgttaaagaatcgtgattttaccgcaagtactttgcagaatttcaacatcagtgttccagcttctaaattagtaacttgggtcgaatcgcatgataattatgctaacgatgatcaagtttcgacttggatgaataatagtgatattaaattaggctgggctgttgttgcttcgcgttctggtagtgttccgctgttctttgaccgtccagttgatggtggtaatggtactcggttccctggcagttcagaaattggtgatgctggcagcagtttgtattatgataaagcagttgtagctgttaataaattccataatgcaatggctggtcaatctgaatatatttctaatccaaatggcaataccaagatttttgaaaatgaacgtggcagcaaaggggttgtttttgcaaacgcttccgacagttcatatagtttgaatgttaaaactagtttagctgatgggacttatgaaaacaaggctggttcagatgaacttaccgttaaaaatggttatttaaccggtacaattcaaggacgtgaagttgttgttctttacggggatccacatcatcaccatcaccactaa

SEQ ID NO: 14 - protein, 475 aa, Lactobacillus amylovorus
MKQQKRLYARLLTLLFALIFLLPHSAAAAASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPHHHHHH

SEQ ID NO: 15 - protein, 446 aa, Lactobacillus amylovorus
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPHHHHHH

SEQ ID NO: 16 - protein, 586 aa, Valsaria rubricosa
MRSFLALSALLLLYPLQLLAASNSDWRSRNIYFALTDRVANPSTTTACSDLSNYCGGTWSGLSSKLDYIQGMGFDSIWITPVVENCDGGYHGYWAKALYNVNTNYGSADDLKNFVAAAHAKGMYVMVDVVANHMGSCGIANLSPPPLNEQSSYHTQCDIDYSSQSSIETCWISGLPDLDTTDSTIRSLFQTWVHGLVSNYSFDGLRVDTVKHVEKDYWPGFVSAAGTYAIGEVFSGDTSYVAGYQSVMPGLLNYPIYYPLIRVFAQGASFTDLVNNHDTVGSTFSDPTLLGNFIDNHDNPRFLSYTSDHALLKNALAYVILARGIPIVYYGTEQGYSGSSDPANREDLWRSGYSTTGDIYTTIAALSAARTAAGGLAGNDHVHLYTTDNAYAWSRASGKLIVVTSNRGSSDSSTICFSTQQASGTTWTSTITGNSYTADSNGQICVQLSSGGPEALVVSTATGTATATTLSTTTKTSTSTASCAATVAVTFNELVTTNYGDTIRLTGSISQLSSWSATSGLALSASAYTSSNPLWSVTVSLPAGTSFEYKFVRITSDGTVTWESDPNRSYTVPTCASTATISNTWR

SEQ ID NO: 17 - protein, 566 aa, Valsaria rubricosa
ASNSDWRSRNIYFALTDRVANPSTTTACSDLSNYCGGTWSGLSSKLDYIQGMGFDSIWITPVVENCDGGYHGYWAKALYNVNTNYGSADDLKNFVAAAHAKGMYVMVDVVANHMGSCGIANLSPPPLNEQSSYHTQCDIDYSSQSSIETCWISGLPDLDTTDSTIRSLFQTWVHGLVSNYSFDGLRVDTVKHVEKDYWPGFVSAAGTYAIGEVFSGDTSYVAGYQSVMPGLLNYPIYYPLIRVFAQGASFTDLVNNHDTVGSTFSDPTLLGNFIDNHDNPRFLSYTSDHALLKNALAYVILARGIPIVYYGTEQGYSGSSDPANREDLWRSGYSTTGDIYTTIAALSAARTAAGGLAGNDHVHLYTTDNAYAWSRASGKLIVVTSNRGSSDSSTICFSTQQASGTTWTSTITGNSYTADSNGQICVQLSSGGPEALVVSTATGTATATTLSTTTKTSTSTASCAATVAVTFNELVTTNYGDTIRLTGSISQLSSWSATSGLALSASAYTSSNPLWSVTVSLPAGTSFEYKFVRITSDGTVTWESDPNRSYTVPTCASTATISNTWR

SEQ ID NO: 18 - protein, 253 aa, Penicillium emersonii
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 19 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 20 - protein, 532 aa, Aspergillus fumigatus
MLASTFSYRMYKTALILAALLGSGQAQQVGTSQAEVHPSMTWQSCTAGGSCTTNNGKVVIDANWRWVHKVGDYTNCYTGNTWDTTICPDDATCASNCALEGANYESTYGVTASGNSLRLNFVTTSQQKNIGSRLYMMKDDSTYEMFKLLNQEFTFDVDVSNLPCGLNGALYFVAMDADGGMSKYPTNKAGAKYGTGYCDSQCPRDLKFINGQANVEGWQPSSNDANAGTGNHGSCCAEMDIWEANSISTAFTPHPCDTPGQVMCTGDACGGTYSSDRYGGTCDPDGCDFNSFRQGNKTFYGPGMTVDTKSKFTVVTQFITDDGTSSGTLKEIKRFYVQNGKVIPNSESTWTGVSGNSITTEYCTAQKSLFQDQNVFEKHGGLEGMGAALAQGMVLVMSLWDDHSANMLWLDSNYPTTASSTTPGVARGTCDISSGVPADVEANHPDAYVVYSNIKVGPIGSTFNSGGSNPGGGTTTTTTTQPTTTTTTAGNPGGTGVAQHYGQCGGIGWTGPTTCASPYTCQKLNDYYSQCL

SEQ ID NO: 21 - protein, 454 aa, Aspergillus fumigatus
MKHLASSIALTLLLPAVQAQQTVWGQCGGQGWSGPTSCVAGAACSTLNPYYAQCIPGATATSTTLTTTTAATTTSQTTTKPTTTGPTTSAPTVTASGNPFSGYQLYANPYYSSEVHTLAMPSLPSSLQPKASAVAEVPSFVWLDVAAKVPTMGTYLADIQAKNKAGANPPIAGIFVVYDLPDRDCAALASNGEYSIANNGVANYKAYIDAIRAQLVKYSDVHTILVIEPDSLANLVTNLNVAKCANAQSAYLECVDYALKQLNLPNVAMYLDAGHAGWLGWPANLGPAATLFAKVYTDAGSPAAVRGLATNVANYNAWSLSTCPSYTQGDPNCDEKKYINAMAPLLKEAGFDAHFIMDTSRNGVQPTKQNAWGDWCNVIGTGFGVRPSTNTGDPLQDAFVWIKPGGESDGTSNSTSPRYDAHCGYSDALQPAPEAGTWFQAYFEQLLTNANPSF

SEQ ID NO: 22 - protein, 515 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 23 - protein, 177 aa, Thermoascus aurantiacus
TRISSCSGSRQSALTTALRNAASLANAAADAAQSGSASKFSEYFKTTSSSTRQTVAARLRAVAREASSSSSGATTYYCDDPYGYCSSNVLAYTLPSYNIIANCDIFYTYLPALTSTCHAQDQATTALHEFTHAPGVYSPGTDDLAYGYQAAMGLSSSQAVMNADTYALYANAIYLGC

SEQ ID NO: 24 - protein, 412 aa, Pyrococcus furiosus
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYDFEKVGYYNPTDGTWTIKVVSYSGSANYQVDVVSDGSLSQPGSS

SEQ ID NO: 25 - protein, 325 aa, Palaeococcus ferrophilus
GKPKPSQPPQEVPWGIERVKAPSVWSTTDGSSNGVIQVAILDTGIDYDHPDLAANLAWGVSTLRGRVSTKPKDYRDQNGHGTHVAGTIAALNNDIGVVGVAPGVQIYAIRVLDASGRGSYSDIAIGIEQAILGPDGVADKDGDGIIAGDPDDDAAEVISMSLGGSADDSYLHDMIIQAYNAGIVIVAASGNEGASSPSYPAAYPEVIAVGASDINDNIASFSNRQPEVSAPGVDVLSTYPDDTYKTLSGTSMATPHVSGVVALIQAAHFNKYGTILPVGTFDDMSKNTVRGILHITADDLGSPGWDVDYGYGIVRADLAVQAALG

SEQ ID NO: 26 - protein, 595 aa, Penicillium oxalicum
RPDPKGGNLTPFIHKEGERSLQGILDNLGGRGKKTPGTAAGLFIASPNTENPNYYYTWTRDSALTAKCLIDLFEDSRAKFPIDRKYLETGIRDYKSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYQGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 27 - protein, 556 aa, Trametes cingulata
QSSAADAYVASESPIAKAGVLANIGPSGSKSNGAKAGIVIASPSTSNPNYLYTWTRDSSLVFKALIDQFTTGEDTSLRTLIDEFTSAEAILQQVPNPSGTVSTGGLGEPKFNIDETAFTDAWGRPQRDGPALRATAIITYANWLLDNKNTTYVTNTLWPIIKLDLDYVASNWNQSTFDLWEEINSSSFFTTAVQHRALREGATFANRIGQTSVVSGYTTQANNLLCFLQSYWNPTGGYITANTGGGRSGKDANTVLTSIHTFDPAAGCDAVTFQPCSDKALSNLKVYVDAFRSIYSINSGIASNAAVATGRYPEDSYMGGNPWYLTTSAVAEQLYDALIVWNKLGALNVTSTSLPFFQQFSSGVTVGTYASSSSTFKTLTSAIKTFADGFLAVNAKYTPSNGGLAEQYSRSNGSPVSAVDLTWSYAAALTSFAARSGKTYASWGAAGLTVPTTCSGSGGAGTVAVTFNVQATTVFGENIYITGSVPALQNWSPDNALILSAANYPTWSITVNLPASTTIEYKYIRKFNGAVTWESDPNNSITTPASGTFTQNDTWR

SEQ ID NO: 28 - protein, 591 aa, Talaromyces emersonii
ATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 29 - protein, 555 aa, Pycnoporus sanguineus
QSSAVDAYVASESPIAKQGVLNNIGPNGSKAHGAKAGIVVASPSTENPDYLYTWTRDSSLVFKLLIDQFTSGDDTSLRGLIDDFTSAEAILQQVSNPSGTVSTGGLGEPKFNIDETAFTGAWGRPQRDGPALRATSIIRYANWLLDNGNTTYVSNTLWPVIQLDLDYVADNWNQSTFDLWEEVDSSSFFTTAVQHRALREGATFASRIGQSSVVSGYTTQADNLLCFLQSYWNPSGGYVTANTGGGRSGKDSNTVLTSIHTFDPAAGCDAATFQPCSDKALSNLKVYVDAFRSIYTINNGIASNAAVATGRYPEDSYMGGNPWYLTTSAVAEQLYDALYVWDQLGGLNVTSTSLAFFQQFASGLSTGTYSASSSTYATLTSAIRSFADGFLAINAKYTPADGGLAEQYSRNDGTPLSAVDLTWSYAAALTAFAAREGKTYGSWGAAGLTVPASCSGGGGATVAVTFNVQATTVFGENIYITGSVAALQNWSPDNALILSAANYPTWSITVNLPANTVVQYKYIRKFNGQVTWESDPNNQITTPSGGSFTQNDVWR

SEQ ID NO: 30 - protein, 556 aa, Gloeophyllum sepiarium
QSVDSYVSSEGPIAKAGVLANIGPNGSKASGASAGVVVASPSTSDPDYWYTWTRDSSLVFKSLIDQYTTGIDSTSSLRTLIDDFVTAEANLQQVSNPSGTLTTGGLGEPKFNVDETAFTGAWGRPQRDGPALRSTALITYGNWLLSNGNTSYVTSNLWPIIQNDLGYVVSYWNQSTYDLWEEVDSSSFFTTAVQHRALREGAAFATAIGQTSQVSSYTTQADNLLCFLQSYWNPSGGYITANTGGGRSGKDANTLLASIHTYDPSAGCDAATFQPCSDKALSNLKVYVDSFRSVYSINSGVASNAAVATGRYPEDSYQGGNPWYLTTFAVAEQLYDALNVWESQGSLEVTSTSLAFFQQFSSGVTAGTYSSSSSTYSTLTSAIKNFADGFVAINAKYTPSNGGLAEQYSKSDGSPLSAVDLTWSYASALTAFEARNNTQFAGWGAAGLTVPSSCSGNSGGPTVAVTFNVNAETVWGENIYLTGSVDALENWSADNALLLSSANYPTWSITVNLPASTAIEYKYIRKNNGAVTWESDPNNSITTPASGSTTENDTWR

SEQ ID NO: 31 - protein, 559 aa, Gloeophyllum trabeum
QSVDSYVGSEGPIAKAGVLANIGPNGSKASGAAAGVVVASPSKSDPDYWYTWTRDSSLVFKSLIDQYTTGIDSTSSLRSLIDSFVIAEANIQQVSNPSGTLTTGGLGEPKFNVDETAFTGAWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGAPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 32 - protein, 583 aa, Rhizomucor pusillus
ATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFGDASLYHPKCTIDYNDQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 33 - protein, 1020 aa, Talaromyces funiculosus
LPFNERVDQVLRSYEVTSKLDSRSTKPSKHGHTYQTQFLGVTWDQRNWRLQSTVLDQGHYESRGSIANGYIGLNVAGAGPLFELDSPVDGDVINGWPLFSRRQTFAGLAGFYDLQPRTNGTNFPWLSQYGDDSAISGVPHWGGMVLDLGDGEYLDATVDNSTISDYTTTYDYKAGVLSWDYKWTPKNANGSFGISYKIFANKLDVNQAVVQLSITPSTNGSASVVNVIDGYAAVRTDFVSSGNESDVVYTAVKPNGVTNVTAWIYTALDGDDAFDISSAALVNDKPYVHQNDSSIAQSVNVTFTAGTTITINKFVGAASTDAFPDPQSTAREAALSARRRGFDDLFRSHISEWAQVMPDDSVDDFTLANGTLPNDTFIIESAVMAVVNPYYLLQNTVGPNALRRVNNAPVNDWSIPVGGLTSDSYAGQIFWDADVWMQPGLVAAFPESAKRITNYRAAKYSQALENAKTAYTSSQNQTWFSPDAAIYSWTSGRVGNCTATGPCWDYEYHLNGDIGISLVNEWVVSGDNETFKNKHFPIYNSIATLYGDLLKKNGSYYTLTNMTDPDEYANNVDAGGYTMTLISQTLSNANAFRKQFGMNENTTWTEMADNILLIRENDVTLEYTTMNNSVAVKQADVILSTFPLDYTKNYTTSAALNDLDYYALKQSPDGPGMTYAIFSIVANDVSPSGCSAYTYAQYSYDPYIRGPFFQFSEQLLDDYTINGGTHPAFPFLTGHGGANQVVLYGYLGLRLLPDDMLHIDPNLPPQIPSIKYRTFYWRGWPIQAASNYTHTTIQRATTVAPLSTADPTYANKSIHVSVGHNTVNSTTYSLSANGSALVVPNRQIGSINTVAGNVVQCKSVLSTDAYQKGQYPISAVDGAASTKWQPEFAANISSLTVDLTGSNVSSVSGFYFDWAQAPPTNITVLLHNSSSAALASSGDKPGSSAVTLNITISNPYNASTYNANIIALPSSNSTNYTFPAPVPKPRYATLFVQGNQALDETDTKSGNGTGATVAEWAILS

SEQ ID NO: 34 - protein, 694 aa, Myceliophthora sepedonium
MALRHIAAAAIAGLASRTAALYINGSVTAPCDSPIYCQGELLKAVELARPFVDSKTFVDMPTIKPVDEVLAAFSKLSLPLSNNSELNAFLYENFAQAGHELEEVPDSELETDAKFLDKLEDRTIKEFVGKVIDIWPDLTRRYAGPSNCTECANSFIPVNRTFVVAGGRFREPYYWDSYWIVEGLLRTGGAFTHISKNIIENFLDFVDTIGFIPNGARIYYLNRSQPPLLTLMVKSYVDYTNDTSILDRALPLLIKEHEFFMNNRTVSITGSNGKEYTLNRYHVENNQPRPESFREDYITANNGSYYASSGIIYPVKTPLNETEKAALYSNLATGAESGWDYTSRWLGVPSDAARDVYFPLRSLNVRDIVPVDLNSILYQNEVIIAEYLEKAGNSSAAKRFATAAEQRSEAMYSLMWNATHWSYFDYNLTDNTQHIFVPADEDTAPQDRIEAPPGQQVFFHIAQLYPFWTGAAPASLKANPLAVQQAYARVARMLDIKKGAIPATNYRTGQQWDQPNVWPPLQHILMKGLLNTPATFGKSDPAYQSVQNLALRLAQRYLDSTFCTWYATGGSTSDFPQLEGVTPGATGVMFEKYADNATNVAGGGGEYEVVEGFGWTNGVLIWAADVFGNKLKRPDCGNITAAHTHSSAKRGLEENKLPRRAVELDPWDAAWTKMFGRSKLRRREAEDVRKRWMS

SEQ ID NO: 35 - protein, 186 aa, Thermobifida cellulosilytica
ADVIGGNPYYFGGYRCSIGFSVRKGSDTGFATAGHCGETGTLTRSPEGVVAGSYFPGRDMGWVRLTGADTVTPLVNRYDGGTVTVTGSQEAVTGSSVCRSGSTTGWRCGIIQSKNQTVRYAEGTVTGLTRTTACAEAGDSGGPWLTGSQAQGVTSGGSGNCRTGGITYFQPINPLLSYFGLELVTG

SEQ ID NO: 36 - protein, 459 aa, Trichoderma reesei
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 37 - protein, 186 aa, Thermobifida fusca
AAIIGGNPYYFGNYRCSIGFSVRQGSQTGFATAGHCGSTGTRVSSPSGTVAGSYFPGRDMGWVRITSADTVTPLVNRYNGGTVTVTGSQEAATGSSVCRSGATTGWRCGTIQSKNQTVRYAEGTVTGLTRTTACAEGGDSGGPWLTGSQAQGVTSGGTGDCRSGGITFFQPINPLLSYFGLQLVTG

SEQ ID NO: 38 - protein, 186 aa, Thermobifida halotolerans
TDIIGGNPYYFDGYRCSIGFSVRRGSESGFATAGHCGEEGTETSDPEGTVAGAYFPGRDMGWVRITDADTVTPLVNRYNGENVTVAGSREAATGSSVCRSGSTTGWRCGTIRSKNQTVRYIEGTVTGLTRTTACAEGGDSGGPWLTGSQGQGVTSGGSGNCTLGGVTYFQPLNPLLSHFDLDLVTG

SEQ ID NO: 39 - protein, 325 aa, Thermococcus nautili
LVPEPTQPAQEIPWGIERVKAPDTWSITTGASNGVIEVAILDTGIDWDHPDLKDNIAWGVSTIGGEVSTDPADWYDGNGHGTHVAGTIAALNNDIGVVGVAPNVEIYAIKVLDDRGSGAYTDIAIGIEQALLGPDGILDKDGDGIIVGDPDDDAAEVISMSLGGPSDDEYLHDMIIKAYNYGVVIVAASGNEAADQPSYPAIYPEVIAVGATDSSDAVAYFSNLQPEVSAPGVDVLSTYPDDTYESLSGTSMATPHVSGVVALIQAAYYNKYGKVLPVGTFDDMNTNTVRGILHVTADDLGDPGWDIYYGYGIVRADLAVQAALG

SEQ ID NO: 40 - DNA, 1980 bp, Bacillus amyloliquefaciens
atgaaaaaaccgctggggaaaattgtcgcaagcaccgcactactcatttctgttgcttttagttcatcgatcgcatcggctgaaactgcaaacaaatcgaataaggtgaccgcgtcatcggtcaaaaacgggaccatcctccatgcatggaattggtcgttcaatacgttaacacaaaatatgaaagatattcgtgatgcgggctatgcagccattcagacgtctccgattaaccaagtaaaggaagggaaccaaggagataaaagcatgaggaactggtactggctgtatcagccgacatcgtaccaaatcggcaaccgttacttaggcactgaacaagaatttaaggacatgtgtgcagccgcggaaaaatatggcgtaaaagtcattgtcgatgcggttatcaatcatactaccagcgattatggcgcgatttctgacgagattaagcgtattccaaactggacccatggaaacacacaaattaaaaattggtcggatcgatgggacgtcactcaaaattcattgcttgggctgtatgattggaatacacagaatactgaggtgcaggcatatctgaaacgtttcttggaaagagcattgaatgacggagcagacgggttccgctatgatgccgccaagcatatagagcttccggatgatgggaattacggcagccaattttggccgaatatcacaaatacatcggcggagttccaatacggagaaatcctgcaagacagcgcgtccagagatacagcttatgcgaattatatgaatgtgacggcttctaactacgggcattccatcagatccgctttaaagaaccgtaatctgagtgtgtcgaatatctcccattatgcatctgacgtgtctgcggacaagttagtcacatgggtggaatcacatgatacgtatgccaatgatgatgaagagtccacatggatgagtgatgacgatattcgtttaggctgggcagtgattggttcccgctcaggaagcacgcctcttttcttttccagacctgagggcggaggaaatggtgtaagatttcccggaaaaagtcaaataggagatcgcgggagcgccttatttaaagatcaggcgatcactgcggtcaatacatttcacaatgtaatggccgggcagcctgaggaactctcgaatccgaatgggaacaaccaaatctttatgaatcagcgcggctcaaaaggcgttgtgctggcaaatgcgggatcgtcttctgtcaccatcaatacttcagcgaaattacctgacggcaggtatgataatagggccggcgccggttcatttcaagtagcgaacggcaaactgacaggtacgatcaatgccagatcggcggctgttctttatcctgatgatattggaaatgcgcctcatgtctttcttgagaattaccaaacgggggcagtccattctttcaatgatcagctgacggtcaccctgcgtgcaaatgcgaaaacaacaaaagccgtttaccaaatcaataatgggcagcagacagcatttaaggatggagaccgactaacgatcgggaaaggagatccaatcggcacgacatacaacatcaaattaaccggaacgaacggcgagggtgcagcgaggacccaagaatacaagtttgttaaaaaagacccgtcccaaaccaacatcattggctatcaaaatccggaccattggggccaggtaaatgcttatatttacaaacatgatggaggcagggccatagaattaaccggatcgtggccggggaaagccatgactaagaatgcagatggaatgtacacgctgacgctgcctgagaatgcggatacggccaacgccaaagtgatttttaacaatggcagcgcccaagtgcccggacagaaccagcccggctttgattatgtgcagaatggtttgtataacaactccggtttaaacggttatcttccgcatcatcatcaccatcaccactaa

SEQ ID NO: 41 - protein, 659 aa, Bacillus amyloliquefaciens
MKKPLGKIVASTALLISVAFSSSIASAETANKSNKVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQAYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQIFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYKFVKKDPSQTNIIGYQNPDHWGQVNAYIYKHDGGRAIELTGSWPGKAMTKNADGMYTLTLPENADTANAKVIFNNGSAQVPGQNQPGFDYVQNGLYNNSGLNGYLPHHHHHHH

SEQ ID NO: 42 - protein, 633 aa, Bacillus amyloliquefaciens
AETANKSNKVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQAYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQIFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYKFVKKDPSQTNIIGYQNPDHWGQVNAYIYKHDGGRAIELTGSWPGKAMTKNADGMYTLTLPENADTANAKVIFNNGSAQVPGQNQPGFDYVQNGLYNNSGLNGYLPHHHHHHH

SEQ ID NO: 43 - DNA, 1725 bp, Lactobacillus amylovorus
atgaagaaaccgttggggaaaattgtcgcaagcaccgcactactcatttctgttgcttttagttcatcgatagcatcagcagctagtgatacgacatcaactgatcactcaagcaatgatacagctgattctgttagcgacggtgttattttgcatgcatggtgctggtcgttcaacacgattaaaaacaacttgaaacagattcatgacgccggctacacagcggttcaaacttcacctgttaatgaagttaaagttggaaatagcgggtctaagtcattaaataactggtattggctatatcagccaactaaatatagtattggtaactattatttaggaacggaagctgaatttaagtcaatgtgcgctgctgctaaagaatataatatcaggatcattgtcgatgcaactctgaatgatacaacaagtgattatagtgcaatttcggatgaaattaaaagtattccaaattggacacatggtaacaaacaaatttcgaattggagtgatcgtgaagatgttactcaaaattcgttgttaggtttatatgattggaatactcaaaattctcaagtccagacgtatttgaagaattatttggaacgcttgatttctgacggagcttcaggctttcgttatgatgcagctaagcatattgaacttccaagtcaatatgatggcagctatggcagcaatttctggccaaatattactgataatgggtctgaatttcagtatggtgaagttttgcaggactcgatttcaaaagaatcagattatgctaattacatgagtgttacagcttcaaattacggcaatacgattcgcaatgcgttaaagaatcgtgattttaccgcaagtactttgcagaatttcaacatcagtgttccagcttctaaattagtaacttgggtcgaatcgcatgataattatgctaacgatgatcaagtttcgacttggatgaataatagtgatattaaattaggctgggctgttgttgcttcgcgttctggtagtgttccgctgttctttgaccgtccagttgatggtggtaatggtactcggttccctggcagttcagaaattggtgatgctggcagcagtttgtattatgataaagcagttgtagctgttaataaattccataatgcaatggctggtcaatctgaatatatttctaatccaaatggcaataccaagatttttgaaaatgaacgtggcagcaaaggggttgtttttgcaaacgcttccgacagttcatatagtttgaatgttaaaactagtttagctgatgggacttatgaaaacaaggctggttcagatgaacttaccgttaaaaatggttatttaaccggtacaattcaaggacgtgaagttgttgttctttacggggatccaacgagcagtagcagcacaacaacagaaacgaaaaaggtgtactttgaaaaaccaagcagttggggaagcagagtttacgcgtatgtctacaataagaacacaaataaagcaattacctcggcttggccgggcaaaaaaatgacggcgcttggaaatgataaatacgaattagatctggacaccgatgaagatgactccgatttagcggtcattttcaccgatgggacaaaacaaacgccggccgctaatgaagcaggcttcacattcacagcggatgcgacgtacgatcaaaatggcgttgtgaaacaccatcaccatcatcac

SEQ ID NO: 44 - protein, 575 aa, Lactobacillus amylovorus
MKKPLGKIVASTALLISVAFSSSIASAASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPTSSSSTTTETKKVYFEKPSSWGSRVYAYVYNKNTNKAITSAWPGKKMTALGNDKYELDLDTDEDDSDLAVIFTDGTKQTPAANEAGFTFTADATYDQNGVVKHHHHHH

SEQ ID NO: 45 - protein, 548 aa, Lactobacillus amylovorus
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPTSSSSTTTETKKVYFEKPSSWGSRVYAYVYNKNTNKAITSAWPGKKMTALGNDKYELDLDTDEDDSDLAVIFTDGTKQTPAANEAGFTFTADATYDQNGVVKHHHHHH

SEQ ID NO: 46 - DNA, 1716 bp, Artificial Sequence
  Hybrid polypeptide comprising Lactobacillus amylovorus catalytic domain and starch binding module from Anoxybacillus contaminans
atgaagaaaccgttggggaaaattgtcgcaagcaccgcactactcatttctgttgcttttagttcatcgatagcatcagcagctagtgatacgacatcaactgatcactcaagcaatgatacagctgattctgttagcgacggtgttattttgcatgcatggtgctggtcgttcaacacgattaaaaacaacttgaaacagattcatgacgccggctacacagcggttcaaacttcacctgttaatgaagttaaagttggaaatagcgggtctaagtcattaaataactggtattggctatatcagccaactaaatatagtattggtaactattatttaggaacggaagctgaatttaagtcaatgtgcgctgctgctaaagaatataatatcaggatcattgtcgatgcaactctgaatgatacaacaagtgattatagtgcaatttcggatgaaattaaaagtattccaaattggacacatggtaacaaacaaatttcgaattggagtgatcgtgaagatgttactcaaaattcgttgttaggtttatatgattggaatactcaaaattctcaagtccagacgtatttgaagaattatttggaacgcttgatttctgacggagcttcaggctttcgttatgatgcagctaagcatattgaacttccaagtcaatatgatggcagctatggcagcaatttctggccaaatattactgataatgggtctgaatttcagtatggtgaagttttgcaggactcgatttcaaaagaatcagattatgctaattacatgagtgttacagcttcaaattacggcaatacgattcgcaatgcgttaaagaatcgtgattttaccgcaagtactttgcagaatttcaacatcagtgttccagcttctaaattagtaacttgggtcgaatcgcatgataattatgctaacgatgatcaagtttcgacttggatgaataatagtgatattaaattaggctgggctgttgttgcttcgcgttctggtagtgttccgctgttctttgaccgtccagttgatggtggtaatggtactcggttccctggcagttcagaaattggtgatgctggcagcagtttgtattatgataaagcagttgtagctgttaataaattccataatgcaatggctggtcaatctgaatatatttctaatccaaatggcaataccaagatttttgaaaatgaacgtggcagcaaaggggttgtttttgcaaacgcttccgacagttcatatagtttgaatgttaaaactagtttagctgatgggacttatgaaaacaaggctggttcagatgaacttaccgttaaaaatggttatttaaccggtacaattcaaggacgtgaagttgttgttctttacggggatccaacttcccaaataacatttactgtaaataacgccacaaccgtttggggacaaaatgtatacgttgtcgggaatatttcgcagctggggaactgggatccagtccacgcagttcaaatgacgccgtcttcttatccaacatggactgtaacaatccctcttcttcaagggcaaaacatacaatttaaatttatcaaaaaagattcagctggaaatgtcatttgggaagatatatcgaatcgaacatacaccgtcccaactgctgcatccggagcatatacagccagctggaacgtgccccaccatcaccatcatcac

SEQ ID NO: 47 - protein, 572 aa, Artificial Sequence
  Synthetic Construct
MKKPLGKIVASTALLISVAFSSSIASAASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPTSQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVPHHHHHH

SEQ ID NO: 48 - protein, 545 aa, Artificial Sequence
  Hybrid polypeptide comprising  Lactobacillus amylovorus catalytic domain and  Anoxybacillus contaminans starch binding module
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPTSQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVPHHHHHH

SEQ ID NO: 49 - DNA, 1731 bp, Artificial Sequence
  Hybrid polypeptide comprising  Lactobacillus amylovorus catalytic domain and Anoxybacillus contaminans starch binding module fused to the C-terminal of the catalytic domain via a synthetic linker
atgaagaaaccgttggggaaaattgtcgcaagcaccgcactactcatttctgttgcttttagttcatcgatagcatcagcagctagtgatacgacatcaactgatcactcaagcaatgatacagctgattctgttagcgacggtgttattttgcatgcatggtgctggtcgttcaacacgattaaaaacaacttgaaacagattcatgacgccggctacacagcggttcaaacttcacctgttaatgaagttaaagttggaaatagcgggtctaagtcattaaataactggtattggctatatcagccaactaaatatagtattggtaactattatttaggaacggaagctgaatttaagtcaatgtgcgctgctgctaaagaatataatatcaggatcattgtcgatgcaactctgaatgatacaacaagtgattatagtgcaatttcggatgaaattaaaagtattccaaattggacacatggtaacaaacaaatttcgaattggagtgatcgtgaagatgttactcaaaattcgttgttaggtttatatgattggaatactcaaaattctcaagtccagacgtatttgaagaattatttggaacgcttgatttctgacggagcttcaggctttcgttatgatgcagctaagcatattgaacttccaagtcaatatgatggcagctatggcagcaatttctggccaaatattactgataatgggtctgaatttcagtatggtgaagttttgcaggactcgatttcaaaagaatcagattatgctaattacatgagtgttacagcttcaaattacggcaatacgattcgcaatgcgttaaagaatcgtgattttaccgcaagtactttgcagaatttcaacatcagtgttccagcttctaaattagtaacttgggtcgaatcgcatgataattatgctaacgatgatcaagtttcgacttggatgaataatagtgatattaaattaggctgggctgttgttgcttcgcgttctggtagtgttccgctgttctttgaccgtccagttgatggtggtaatggtactcggttccctggcagttcagaaattggtgatgctggcagcagtttgtattatgataaagcagttgtagctgttaataaattccataatgcaatggctggtcaatctgaatatatttctaatccaaatggcaataccaagatttttgaaaatgaacgtggcagcaaaggggttgtttttgcaaacgcttccgacagttcatatagtttgaatgttaaaactagtttagctgatgggacttatgaaaacaaggctggttcagatgaacttaccgttaaaaatggttatttaaccggtacaattcaaggacgtgaagttgttgttctttacggggatccaccggaaccgacgccggagccgcaaataacatttactgtaaataacgccacaaccgtttggggacaaaatgtatacgttgtcgggaatatttcgcagctggggaactgggatccagtccacgcagttcaaatgacgccgtcttcttatccaacatggactgtaacaatccctcttcttcaagggcaaaacatacaatttaaatttatcaaaaaagattcagctggaaatgtcatttgggaagatatatcgaatcgaacatacaccgtcccaactgctgcatccggagcatatacagccagctggaacgtgccccaccatcaccatcatcac

SEQ ID NO: 50 - protein, 577 aa, Artificial Sequence
  Synthetic Construct
MKKPLGKIVASTALLISVAFSSSIASAASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPPEPTPEPQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVPHHHHHH

SEQ ID NO: 51 - protein, 550 aa, Artificial Sequence
  Hybrid polypeptide comprising Lactobacillus amylovorus catalytic domain and Anoxybacillus contaminans starch binding domain fused via a synthetic linker
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPPEPTPEPQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVPHHHHHH

SEQ ID NO: 52 - DNA, 2271 bp, Artificial Sequence
  Hybrid polypeptide comprising Lactobacillus amylovorus catalytic domain, synthetic linker, Bacillus amyloliquefaciens starch binding module, and Anoxybacillus contaminans starch binding module
atgaagaaaccgttggggaaaattgtcgcaagcaccgcactactcatttctgttgcttttagttcatcgatagcatcagcagctagtgatacgacatcaactgatcactcaagcaatgatacagctgattctgttagcgacggtgttattttgcatgcatggtgctggtcgttcaacacgattaaaaacaacttgaaacagattcatgacgccggctacacagcggttcaaacttcacctgttaatgaagttaaagttggaaatagcgggtctaagtcattaaataactggtattggctatatcagccaactaaatatagtattggtaactattatttaggaacggaagctgaatttaagtcaatgtgcgctgctgctaaagaatataatatcaggatcattgtcgatgcaactctgaatgatacaacaagtgattatagtgcaatttcggatgaaattaaaagtattccaaattggacacatggtaacaaacaaatttcgaattggagtgatcgtgaagatgttactcaaaattcgttgttaggtttatatgattggaatactcaaaattctcaagtccagacgtatttgaagaattatttggaacgcttgatttctgacggagcttcaggctttcgttatgatgcagctaagcatattgaacttccaagtcaatatgatggcagctatggcagcaatttctggccaaatattactgataatgggtctgaatttcagtatggtgaagttttgcaggactcgatttcaaaagaatcagattatgctaattacatgagtgttacagcttcaaattacggcaatacgattcgcaatgcgttaaagaatcgtgattttaccgcaagtactttgcagaatttcaacatcagtgttccagcttctaaattagtaacttgggtcgaatcgcatgataattatgctaacgatgatcaagtttcgacttggatgaataatagtgatattaaattaggctgggctgttgttgcttcgcgttctggtagtgttccgctgttctttgaccgtccagttgatggtggtaatggtactcggttccctggcagttcagaaattggtgatgctggcagcagtttgtattatgataaagcagttgtagctgttaataaattccataatgcaatggctggtcaatctgaatatatttctaatccaaatggcaataccaagatttttgaaaatgaacgtggcagcaaaggggttgtttttgcaaacgcttccgacagttcatatagtttgaatgttaaaactagtttagctgatgggacttatgaaaacaaggctggttcagatgaacttaccgttaaaaatggttatttaaccggtacaattcaaggacgtgaagttgttgttctttacggggatccaccggaaccgacgccggagccgcctcatgtctttcttgagaattaccaaacgggggcagtccattctttcaatgatcagctgacggtcaccctgcgtgcaaatgcgaaaacaacaaaagccgtttaccaaatcaataatgggcagcagacagcatttaaggatggagaccgactaacgatcgggaaaggagatccaatcggcacgacatacaacatcaaattaaccggaacgaacggcgagggtgcagcgaggacccaagaatacaagtttgttaaaaaagacccgaaaaaggtgtactttgaaaaaccaagcagttggggaagcagagtttacgcgtatgtctacaataagaacacaaataaagcaattacctcggcttggccgggcaaaaaaatgacggcgcttggaaatgataaatacgaattagatctggacaccgatgaagatgactccgatttagcggtcattttcaccgatgggacaaaacaaacgccggccgctaatgaagcaggcttcacattcacagcggatgcgacgtacgatcaaaatggcgttgtgaaacaaataacatttactgtaaataacgccacaaccgtttggggacaaaatgtatacgttgtcgggaatatttcgcagctggggaactgggatccagtccacgcagttcaaatgacgccgtcttcttatccaacatggactgtaacaatccctcttcttcaagggcaaaacatacaatttaaatttatcaaaaaagattcagctggaaatgtcatttgggaagatatatcgaatcgaacatacaccgtcccaactgctgcatccggagcatatacagccagctggaacgtgccccaccatcaccatcatcac

SEQ ID NO: 53 - protein, 757 aa, Artificial Sequence
  Synthetic Construct
MKKPLGKIVASTALLISVAFSSSIASAASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPPEPTPEPPHVFLENYQTGAVHSFNDQLTVTLRANAKTTKAVYQINNGQQTAFKDGDRLTIGKGDPIGTTYNIKLTGTNGEGAARTQEYKFVKKDPKKVYFEKPSSWGSRVYAYVYNKNTNKAITSAWPGKKMTALGNDKYELDLDTDEDDSDLAVIFTDGTKQTPAANEAGFTFTADATYDQNGVVKQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVPHHHHHH

SEQ ID NO: 54 - protein, 730 aa, Artificial
  Hybrid polypeptide comprising Lactobacillus amylovorus carbohydrate binding module, synthetic linker, Bacillus amyloliquefaciens domain of unknown function, Bacillus amyloliquefaciens starch binding module, and Anoxybacillus
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPPEPTPEPPHVFLENYQTGAVHSFNDQLTVTLRANAKTTKAVYQINNGQQTAFKDGDRLTIGKGDPIGTTYNIKLTGTNGEGAARTQEYKFVKKDPKKVYFEKPSSWGSRVYAYVYNKNTNKAITSAWPGKKMTALGNDKYELDLDTDEDDSDLAVIFTDGTKQTPAANEAGFTFTADATYDQNGVVKQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVPHHHHHH

SEQ ID NO: 55 - DNA, 1728 bp, Artificial Sequence
  encoding hybrid alpha-amylase comprising Lactobacillus amylovorus core and heterologous CBM20
atgaaaaaaccgctggggaaaattgtcgcaagcaccgcactactcatttctgttgcttttagttcatcgatcgcatcggctgctagtgatacgacatcaactgatcactcaagcaatgatacagctgattctgttagcgacggtgttattttgcatgcatggtgctggtcgttcaacacgattaaaaacaacttgaaacagattcatgacgccggctacacagcggttcaaacttcacctgttaatgaagttaaagttggaaatagcgggtctaagtcattaaataactggtattggctatatcagccaactaaatatagtattggtaactattatttaggaacggaagctgaatttaagtcaatgtgcgctgctgctaaagaatataatatcaggatcattgtcgatgcaactctgaatgatacaacaagtgattatagtgcaatttcggatgaaattaaaagtattccaaattggacacatggtaacaaacaaatttcgaattggagtgatcgtgaagatgttactcaaaattcgttgttaggtttatatgattggaatactcaaaattctcaagtccagacgtatttgaagaattatttggaacgcttgatttctgacggagcttcaggctttcgttatgatgcagctaagcatattgaacttccaagtcaatatgatggcagctatggcagcaatttctggccaaatattactgataatgggtctgaatttcagtatggtgaagttttgcaggactcgatttcaaaagaatcagattatgctaattacatgagtgttacagcttcaaattacggcaatacgattcgcaatgcgttaaagaatcgtgattttaccgcaagtactttgcagaatttcaacatcagtgttccagcttctaaattagtaacttgggtcgaatcgcatgataattatgctaacgatgatcaagtttcgacttggatgaataatagtgatattaaattaggctgggctgttgttgcttcgcgttctggtagtgttccgctgttctttgaccgtccagttgatggtggtaatggtactcggttccctggcagttcagaaattggtgatgctggcagcagtttgtattatgataaagcagttgtagctgttaataaattccataatgcaatggctggtcaatctgaatatatttctaatccaaatggcaataccaagatttttgaaaatgaacgtggcagcaaaggggttgtttttgcaaacgcttccgacagttcatatagtttgaatgttaaaactagtttagctgatgggacttatgaaaacaaggctggttcagatgaacttaccgttaaaaatggttatttaaccggtacaattcaaggacgtgaagttgttgttctttacggggatccatccggcaactctggtggcccaactgtagcagtaacattcaacgttaacgcagagactgtatggggtgagaacatctaccttacgggcagcgttgacgcacttgagaactggtcagcagacaacgctcttcttctttcttcagctaactacccaacgtggtctatcacggttaaccttcctgctagcacggctatcgagtacaaatacatccgcaaaaacaatggcgcagttacatgggagtctgacccaaacaactctatcactacaccagcttcaggctctacaacagagaacgacacatggcgccatcatcaccatcaccactaa

SEQ ID NO: 56 - protein, 575 aa, Artificial Sequence
  Synthetic Construct
MKKPLGKIVASTALLISVAFSSSIASAASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPSGNSGGPTVAVTFNVNAETVWGENIYLTGSVDALENWSADNALLLSSANYPTWSITVNLPASTAIEYKYIRKNNGAVTWESDPNNSITTPASGSTTENDTWRHHHHHH

SEQ ID NO: 57 - protein, 548 aa, Artificial Sequence
  hybrid polypeptide comprising Lactobacillus amylovorus core and heterologous CBM20 starch binding domain
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPSGNSGGPTVAVTFNVNAETVWGENIYLTGSVDALENWSADNALLLSSANYPTWSITVNLPASTAIEYKYIRKNNGAVTWESDPNNSITTPASGSTTENDTWRHHHHHH

SEQ ID NO: 58 - DNA, 1737 bp, Artificial Sequence
  encoding hybrid polypeptide comprising Lactobacillus amylovorus catalytic domain and heterologous CMB20
atgaaaaaaccgctggggaaaattgtcgcaagcaccgcactactcatttctgttgcttttagttcatcgatcgcatcggctgctagtgatacgacatcaactgatcactcaagcaatgatacagctgattctgttagcgacggtgttattttgcatgcatggtgctggtcgttcaacacgattaaaaacaacttgaaacagattcatgacgccggctacacagcggttcaaacttcacctgttaatgaagttaaagttggaaatagcgggtctaagtcattaaataactggtattggctatatcagccaactaaatatagtattggtaactattatttaggaacggaagctgaatttaagtcaatgtgcgctgctgctaaagaatataatatcaggatcattgtcgatgcaactctgaatgatacaacaagtgattatagtgcaatttcggatgaaattaaaagtattccaaattggacacatggtaacaaacaaatttcgaattggagtgatcgtgaagatgttactcaaaattcgttgttaggtttatatgattggaatactcaaaattctcaagtccagacgtatttgaagaattatttggaacgcttgatttctgacggagcttcaggctttcgttatgatgcagctaagcatattgaacttccaagtcaatatgatggcagctatggcagcaatttctggccaaatattactgataatgggtctgaatttcagtatggtgaagttttgcaggactcgatttcaaaagaatcagattatgctaattacatgagtgttacagcttcaaattacggcaatacgattcgcaatgcgttaaagaatcgtgattttaccgcaagtactttgcagaatttcaacatcagtgttccagcttctaaattagtaacttgggtcgaatcgcatgataattatgctaacgatgatcaagtttcgacttggatgaataatagtgatattaaattaggctgggctgttgttgcttcgcgttctggtagtgttccgctgttctttgaccgtccagttgatggtggtaatggtactcggttccctggcagttcagaaattggtgatgctggcagcagtttgtattatgataaagcagttgtagctgttaataaattccataatgcaatggctggtcaatctgaatatatttctaatccaaatggcaataccaagatttttgaaaatgaacgtggcagcaaaggggttgtttttgcaaacgcttccgacagttcatatagtttgaatgttaaaactagtttagctgatgggacttatgaaaacaaggctggttcagatgaacttaccgttaaaaatggttatttaaccggtacaattcaaggacgtgaagttgttgttctttacggggatccatccagcaacagttgcggaggcggaggatcgactgtcgccgtgacgttcaacgtgaacgcccaaacggtttggggcgaaaacatctacatcactggctcggttgacgctctgagtaactggtctcccgacaacgccctcttgctctcgtctgccaactacccgacctggagcattaccgtgaatttacccgcgagcactgccattcagtataagtatatccgcaagatgaacggagctgtcacctgggaatccgatcccaacaacagcataactactccagccagcggctcctggaccgagaatgacacttggcgtcatcatcaccatcaccactaa

SEQ ID NO: 59 - protein, 578 aa, Artificial Sequence
  Synthetic Construct
MKKPLGKIVASTALLISVAFSSSIASAASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPSSNSCGGGGSTVAVTFNVNAQTVWGENIYITGSVDALSNWSPDNALLLSSANYPTWSITVNLPASTAIQYKYIRKMNGAVTWESDPNNSITTPASGSWTENDTWRHHHHHH

SEQ ID NO: 60 - protein, 551 aa, Artificial Sequence
  hybrid polypeptide comprising Lactobacillus amylovorus catalytic domain and heterologous CBM20
ASDTTSTDHSSNDTADSVSDGVILHAWCWSFNTIKNNLKQIHDAGYTAVQTSPVNEVKVGNSGSKSLNNWYWLYQPTKYSIGNYYLGTEAEFKSMCAAAKEYNIRIIVDATLNDTTSDYSAISDEIKSIPNWTHGNKQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNNSDIKLGWAVVASRSGSVPLFFDRPVDGGNGTRFPGSSEIGDAGSSLYYDKAVVAVNKFHNAMAGQSEYISNPNGNTKIFENERGSKGVVFANASDSSYSLNVKTSLADGTYENKAGSDELTVKNGYLTGTIQGREVVVLYGDPSSNSCGGGGSTVAVTFNVNAQTVWGENIYITGSVDALSNWSPDNALLLSSANYPTWSITVNLPASTAIQYKYIRKMNGAVTWESDPNNSITTPASGSWTENDTWRHHHHHH

SEQ ID NO: 61 - protein, 27 aa, Bacillus clausii
MKKPLGKIVASTALLISVAFSSSIASA

SEQ ID NO: 62 - protein, 6 aa, Artificial Sequence
  His tag
HHHHHH

SEQ ID NO: 63 - protein, 5 aa, Artificial Sequence
  synthetic linker
PPPGP

SEQ ID NO: 64 - protein, 8 aa, Artificial Sequence
  synthetic linker
SGNSGGPT

SEQ ID NO: 65 - protein, 11 aa, Artificial Sequence
  synthetic linker
SSNSCGGGGST